(19) United States
(12) Patent Application Publication
(10) Pub. No.: US 2026/0116989 A1
(43) Pub. Date: Apr. 30, 2026

(54) SELECTIVE TARGETING OF HOST CD70+ ALLOREACTIVE CELLS TO PROLONG ALLOGENEIC CAR T CELL PERSISTENCE

(71) Applicant: Allogene Therapeutics, Inc., South San Francisco, CA (US)

(72) Inventors: Elvin J. LAURON, Milpitas, CA (US); Siler PANOWSKI, Berkeley, CA (US); Barbra Johnson SASU, San Francisco, CA (US); Cesar Adolfo SOMMER, San Mateo, CA (US); Surabhi SRIVATSA SRINIVASAN, San Francisco, CA (US); Thomas John VAN BLARCOM, Oakland, CA (US); Shanshan LANG, San Mateo, CA (US)

(73) Assignee: Allogene Therapeutics, Inc., South San Francisco, CA (US)

(21) Appl. No.: 19/353,269

(22) Filed: Oct. 8, 2025

Related U.S. Application Data
(62) Division of application No. 17/841,041, filed on Jun. 15, 2022.
(60) Provisional application No. 63/210,979, filed on Jun. 15, 2021.
(60) Provisional application No. 63/351,223, filed on Jun. 10, 2022.

(51) Int. Cl. C07K 16/28; A61K 40/11; A61K 40/22; A61K 40/31; A61K 40/41; A61K 40/42; A61K 40/50; A61P 35/00

(52) U.S. Cl. CPC C07K 16/2875; A61K 40/11; A61K 40/22; A61K 40/31; A61K 40/418; A61K 40/4211; A61K 40/4232; A61P 35/00; A61K 40/50; A61K 2239/28; C07K 2317/31

(57) ABSTRACT

Provided herein are CD70-binding proteins comprising a CD70-binding domain and a transmembrane domain, engineered immune cells comprising the CD70-binding proteins, and methods of making and using the same. Also provided herein are engineered immune cells e.g. CAR (chimeric antigen receptor) T cells for administration to patients to treat cancer (e.g., solid tumors and hematologic tumors) and other unwanted conditions. The cells are engineered to functionally express a first antigen binding molecule e.g. a CD70 CAR and a second antigen binding molecule e.g. a second CAR that binds a target molecule characteristic of the cancer or other disease or unwanted condition. The cells may be further engineered to reduce the functional expression level of one or more of TRAC, CD52 and CD70. Also provided are methods of making and using the engineered cells, compositions and kits comprising them, and methods of treating by administering them.

### CROSS REFERENCE TO RELATED APPLICATIONS

The present application is a divisional of the co-pending application Ser. No. 17/841,041 filed on Jun. 15, 2022, which claims the benefit of priority to U.S. Provisional Application No. 63/210,979, filed on Jun. 15, 2021; and U.S. Provisional Application No. 63/351,223 filed on Jun. 10, 2022, the contents of both of which are hereby incorporated by reference in their entireties.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Oct. 9, 2025, is entitled “AT-049/04US_ST26.xml”, and is 809,631 bytes in size.

### FIELD

The present disclosure relates generally to the use of engineered immune cells (e.g., T cells) in therapeutic applications.

### BACKGROUND

Adoptive transfer of immune cells genetically modified to recognize malignancy-associated antigens is showing promise as a new approach to treating cancer (see, e.g., Brenner et al., Current Opinion in Immunology, 22(2): 251-257 (2010); Rosenberg et al., Nature Reviews Cancer, 8(4): 299-308 (2008)). Immune cells can be genetically modified to express chimeric antigen receptors (CARs), fusion proteins comprised of an antigen recognition moiety and T cell activation domains (see, e.g., Eshhar et al., Proc. Natl. Acad. Sci. USA, 90(2): 720-724 (1993)). Immune cells that contain CARs, e.g., CAR-T cells (CAR-Ts), are engineered to endow them with antigen specificity while retaining or enhancing their ability to recognize and kill a target cell.

However, the generation of CAR-modified autologous cell therapies is expensive, requires weeks of process and quality testing, and yields product of variable potency depending on the initial quality and quantity of patient-specific T cells employed. Allogeneic CAR-modified cell therapies—in which cells from a healthy donor are modified with CAR and then administered to multiple patients-promises a cheaper and more robust product than autologous therapies that can be delivered immediately upon need (see, e.g., Graham et al., Cells 2018, 7, 155; doi:10.3390/cells7100155). Additionally, allogeneic therapies enable selection on desirable product characteristics (e.g. gene editing efficiency, site of integration, lack of deleterious off-target gene edits, haplotype, etc.), and facilitate more sophisticated cell engineering (e.g. multiple gene edits improving potency, persistence, homing, etc.). A major hurdle to implementing allogeneic CAR-modified cell therapies is the potential for rejection of the product (donor) by the immune system of the patient (host).

While allogeneic cell therapies present a number of advantages over autologous cell therapies, allogeneic cells also face rejection by host or recipient immune system cells reactive with T and NK epitope determinants on the surface of the allogeneic cell product that are distinct from host. The present disclosure provides the advantages of improved allogeneic therapies that provide increased persistence of the administered cells despite the recipients' natural defenses.

### SUMMARY

Provided herein are immune cells that have been engineered e.g. genetically engineered to improve their persistence in a host or recipient into which or whom the cells have been introduced, compositions and populations comprising the engineered cells and methods of treating a condition e.g. a cancer in a patient using the same. The immune cells of the instant disclosure are engineered to functionally express a first antigen binding protein and a second, different antigen binding protein, e.g. a first CAR and a second CAR. The first antigen binding protein e.g. CAR is selected for its suitability for therapeutic treatment of a condition e.g. a cancer in a patient. For example, the first antigen binding protein may be a CAR that recognizes an antigen that is expressed by e.g. that is characteristic of cells that cause the condition, e.g. cancer cells and/or tumor cells. The second antigen binding protein e.g. a CD70-binding protein such as a CAR has binding specificity for CD70, which is expressed on recipient immune cells in response to, for example, allogeneic CAR T cells. The engineered cell thus can defend itself against the recipient's immune response, thereby prolonging the survival of the allogeneic cells after administration. Alternatively, one antigen binding protein e.g. one CAR may comprise both the “therapeutic” binding activity and the CD70 binding activity. The present disclosure thus provides a method of increasing persistence of e.g. allogeneic CAR T cells or any other allogeneic CAR immune cell (e.g. CAR NK cell), the method comprising engineering the CAR cell e.g. CAR T cell, CAR NK cell, CAR monocyte or CAR macrophages to functionally express an anti-CD70 antigen binding protein e.g. a CD70-binding protein such as an anti-CD70 CAR in addition to the first antigen binding protein e.g. CAR that the cell functionally expresses.

This approach potentially provides a safer alternative to deep lymphodepletion regimens since CD70-negative immune cells are not expected to be targeted by the CD70 CAR, thus avoiding long-term immunosuppression. Additionally, CD70 is also expressed by malignant cells in a variety of tumors, including renal cell carcinoma, lymphoma and acute myeloid leukemia. Engineering T cells to express CARs against CD70 and a second tumor-specific target, such as CD19 or CD20, either as separate CARs or as a single CAR with dual specificity, may therefore enhance the antitumor efficacy and persistence of CAR T cells and delay rejection by the host immune system.

Thus, in one aspect, the present disclosure provides a method of inhibiting proliferation and/or activity of CD70 positive cells in vitro or in a patient, comprising the step of contacting the CD70 positive cells with engineered immune cells that comprise or functionally express a CD70-binding protein comprising an extracellular ligand-binding domain that binds to CD70 (or a CD70 binding domain) and a transmembrane domain. In a related aspect, the present disclosure provides a method of lymphodepletion in a patient in need thereof. comprising the step of administering engineered immune cells to the patient, wherein the engineered immune cells comprise or functionally express a CD70-binding protein comprising an extracellular ligand-binding domain that binds to CD70 (or a CD70 binding domain) and a transmembrane domain, and wherein the engineered immune cells inhibit proliferation and/or activity of CD70 positive cells in the patient. In some embodiments, the CD70 positive cells are T cells, B cells or NK cells. In some embodiments, the CD70 positive cells are normal or non-cancerous lymphocytes. In some embodiments, the CD70 positive cells are activated lymphocytes. In some embodiments, the CD70 positive cells are activated T cells.

In another aspect, the present disclosure provides an engineered immune cell that functionally expresses a protein comprising a first antigen binding domain and a protein comprising a second antigen binding domain, wherein the first antigen binding domain specifically binds a target of interest and the second antigen binding domain specifically binds CD70 (e.g., a CD70-binding protein). In various embodiments, the protein comprising the first antigen binding domain is a first protein and the protein comprising the second antigen binding domain is a second protein separate and distinct from the first protein. In various embodiments, the protein comprising the first antigen binding domain is a first CAR and the protein comprising the second antigen binding domain is a second CAR, i.e. a CD70 CAR. In various embodiments, one antigen binding protein e.g. one CAR comprises both the first antigen binding domain and the second antigen binding domain, e.g. the CAR is a bispecific CAR that recognizes both a target of interest and CD70. In other embodiments, the protein comprising the first antigen binding domain recognizing a target of interest and the protein comprising the second antigen binding domain, i.e., the CD70-binding protein, are different proteins.

In some embodiments, the target of interest of the first antigen binding domain, e.g. the target of interest of the first CAR, is any molecule of interest, including, for example, without limitation, CD70, BCMA, EGFRvIII, Flt-3, WT-1, CD20, CD22, CD23, CD30, CD38, CD33, CD133, WT1, TSPAN10, MHC-PRAME, HER2, MSLN, PSMA, PSCA, GPC3, Liv1, ADAM10, CHRNA2, LeY, NKG2D, CS1, CD44v6, ROR1, CD19, Claudin-18.2 (also referred to as Claudin-18A2 or Claudin18 isoform 2), DLL3 (also referred to as Delta-like protein 3, Drosophila Delta homolog 3, Delta3), Muc17, Muc3, Muc16, FAP alpha (Fibroblast Activation Protein alpha), Ly6G6D (also referred to as Lymphocyte antigen 6 complex locus protein G6d, c6orf23, G6D, MEGT1, NG25), or RNF43 (also referred to as E3 ubiquitin-protein ligase RNF43, RING finger protein 43), specifically including the human form of any of the listed exemplary targets. The target of interest can be, for example, any molecule, e.g. any protein, that is expressed on the surface of a cell and is characteristic in some respect of a condition or disease, such as any form of cancer, the inhibition, reduction or elimination of which would be therapeutically beneficial or otherwise desirable.

In some embodiments, the protein comprising the second antigen binding domain that specifically binds CD70 (e.g., the CD70-binding protein) is or comprises a CD70 CAR. In some embodiments, the protein comprising the second antigen binding domain comprises an anti-CD70 scFv, an anti-CD70 VH or a receptor for CD70, or comprises any two or more of these. In some embodiments, the receptor for CD70 is CD27 or a fragment of CD27 that retains binding specificity for CD70, e.g. a CD70-binding fragment of CD27. In some embodiments, CD27 has or comprises the amino acid sequence of human CD27 as disclosed in UniProtKB entry P26842. In some embodiments, the protein comprising the second antigen binding domain is a CAR that comprises a CD3 intracellular signaling domain. In some embodiments, the protein comprising the second antigen binding domain is a CAR that comprises one or more than one costimulatory domain. In some embodiments, the protein comprising the second antigen binding domain is a CAR that comprises a CD3 intracellular signaling domain without a costimulatory domain (a first-generation CAR). In some embodiments, the protein comprising the second antigen binding domain is a CAR that comprises a CD3 intracellular signaling domain and a costimulatory domain (a second-generation CAR). In some embodiments, the protein comprising the second antigen binding domain is a CAR that does not comprise a costimulatory domain. In some embodiments, the protein comprising the second antigen binding domain, e.g., the CD70-binding protein, does not comprise an intracellular signaling domain. In some embodiments, the protein comprising the second antigen binding domain is a CAR that optionally comprises one or more than one costimulatory domain. In various embodiments of the protein comprising the second antigen binding domain, the protein further comprises an intracellular signaling domain such as a CD3ζ intracellular signaling domain and/or one or more costimulatory domains. In some embodiments, the protein comprising the second antigen binding domain comprises full-length CD27 or a CD70-binding fragment of CD27 and further comprises a CD3ζ intracellular signaling domain.

In various embodiments, the engineered immune cell functionally expresses one or more of CD70, TRAC and CD52 at a reduced level. In some embodiments, the reduced level of expression, stated relative to the expression level in a corresponding but non-engineered immune cell, is 0%, for example when both chromosomal copies of a gene are knocked out, or 50% (i.e. 50% of the level in a non-engineered control immune cell), for example when one of the two chromosomal copies of a gene is knocked out and there is no compensatory increase in expression of the other chromosomal copy of that gene. In some embodiments, the cell expresses one or more of CD70, TRAC and CD52 at a level not greater than 90%, not greater than 75%, not greater than 50%, not greater than 25%, or not greater than 10% of the expression level in a non-engineered immune cell. In some embodiments, the level of expression of one or more of CD70, TRAC and CD52 in the engineered immune cell is any value between 0% and 90% of the level in a control cell not correspondingly engineered with respect to CD70, TRAC and/or CD52. In some embodiments, the expression level in the engineered cell is, for example, between 10% and 90%, between 25% and 90%, between 25% and 75%, between 10% and 50%, between 25% and 50%, between 50% and 90%, or between 50% and 75% of the level in a control cell. In some embodiments, a reduced level of expression other than 0% or 50% is obtained when, for example, only one chromosomal copy of a gene is knocked out and a compensatory mechanism causes an increase in the level of expression of the remaining chromosomal copy, or reduction in expression is achieved by a method other than gene knockout, such as known knockdown methods e.g. those that employ any of various RNA-based techniques (e.g. antisense RNA, miRNA, shRNA, siRNA; see, e.g., Lam et al., Mol. Ther.-Nucleic Acids 4:e252 (2015), doi:10.1038/mtna.2015.23; Sridharan and Gogtay, Brit. J. Clin. Pharmacol. 82: 659-72 (2016)). In some embodiments, the engineered immune cell disclosed herein exhibits a reduced level of expression of an MHC class I protein or complex at the cell surface relative to a suitable control. In various embodiments, the cell is a T cell e.g. a human T cell. In some embodiments, the cell comprises a mutation in one or more of the CD70, CD52 and TRAC locus or gene and/or a disruption in one or more of the CD70, CD52 and TRAC locus or gene that causes a reduction in functional expression of the disrupted locus or gene. In an embodiment, the mutation or disruption is introduced into one or more of the CD70, CD52 and TRAC genes or loci by any gene mutation or gene editing technique, including but not limited to known homologous recombination techniques and techniques that employ any one or more of meganucleases, TALEN, zinc fingers, shRNA, Cas-CLOVER, and a CRISPR/Cas system. In some embodiments, the cell is a non-human cell, e.g. a primate cell or a non-primate mammalian cell. In some embodiments, the cell is a human cell. In some embodiments, the mutation or disruption is produced by knocking in a nucleic acid e.g. a nucleic acid that encodes one or more proteins or gene products to be expressed in the cell. In some embodiments, the nucleic acid encodes either or both of the first CAR and the second CAR.

Provided herein is an engineered immune cell that functionally expresses a protein comprising a first antigen binding domain and a protein comprising a second antigen binding domain, wherein the first antigen binding domain specifically binds a target of interest and the second antigen binding domain specifically binds CD70 (or a CD70-binding protein), and the uses thereof.

In various embodiments, the protein comprising the first antigen binding domain is a first CAR and the protein comprising the second antigen binding domain is a second CAR. In some embodiments, the second antigen binding domain is a CAR e.g. second CAR that comprises one or more than one costimulatory domains. In some embodiments, the second antigen binding domain is a CAR e.g. second CAR that does not comprise a costimulatory domain. In some embodiments, the second antigen binding domain is a CAR e.g. second CAR that optionally comprises one or more than one costimulatory domains.

In various embodiments, one protein comprises both the first antigen binding domain and the second antigen binding domain. In certain embodiments, the one protein is a bispecific CAR.

In certain embodiments, the target of interest is a protein selected from the group consisting of CD70, BCMA, EGFRvIII, Flt-3, WT-1, CD20, CD22, CD23, CD30, CD38, CD33, CD133, WT1, TSPAN10, MHC-PRAME, HER2, MSLN, PSMA, PSCA, GPC3, Liv1, ADAM10, CHRNA2, LeY, NKG2D, CS1, CD44v6, ROR1, CD19, Claudin-18.2 (Claudin-18A2, or Claudin18 isoform 2), DLL3 (Delta-like protein 3, Drosophila Delta homolog 3, Delta3), Muc17, Muc3, Muc16, FAP alpha (Fibroblast Activation Protein alpha), Ly6G6D (Lymphocyte antigen 6 complex locus protein G6d, c6orf23, G6D, MEGT1, NG25), or RNF43 (E3 ubiquitin-protein ligase RNF43, RING finger protein 43). In certain embodiments, the target of interest is the human form of a protein selected from the group consisting of CD70, BCMA, EGFRvIII, Flt-3, WT-1, CD20, CD22, CD23, CD30, CD38, CD33, CD133, WT1, TSPAN10, MHC-PRAME, HER2, MSLN, PSMA, PSCA, GPC3, Liv1, ADAM10, CHRNA2, LeY, NKG2D, CS1, CD44v6, ROR1, CD19, Claudin-18.2 (Claudin-18A2, or Claudin18 isoform 2), DLL3 (Delta-like protein 3, Drosophila Delta homolog 3, Delta3), Muc17, Muc3, Muc16, FAP alpha (Fibroblast Activation Protein alpha), Ly6G6D (Lymphocyte antigen 6 complex locus protein G6d, c6orf23, G6D, MEGT1, NG25), or RNF43 (E3 ubiquitin-protein ligase RNF43, RING finger protein 43).

In various embodiments, the engineered immune cell disclosed herein further comprises one or more genomic modifications of one or more of an endogenous TRAC gene, an endogenous CD52 gene, and an endogenous CD70 gene. In certain embodiments, the cell comprises a knockout at one or both alleles of TRAC, a knockout at one or both alleles of CD52, or a knockout at one or both alleles of CD70, or a knockout at one or both alleles of one or more of TRAC, CD52 and CD70. In various embodiments, the engineered immune cell expresses one or more of TRAC, CD52 and CD70 at a level not greater than 90%, not greater than 75%, not greater than 50%, not greater than 25%, or not greater than 10% of the expression level in a non-engineered immune cell.

In various embodiments, the engineered immune cell disclosed herein is an engineered T cell. In various embodiments, the engineered immune cell disclosed herein is an engineered NK cell.

In various embodiments, the engineered immune cell disclosed herein comprises a first nucleic acid encoding the protein comprising a first antigen binding domain and a second nucleic acid encoding the protein comprising a second antigen binding domain. In certain embodiments, a first vector comprises the first nucleic acid and a second vector comprises the second nucleic acid. In some embodiments, one or both vectors are lentiviral vectors. In some embodiments, one or both vectors are adeno-associated virus (AAV) vectors. In certain embodiments, the first nucleic acid and/or the second nucleic acid is located within a disrupted TRAC, CD52 or CD70 locus. In certain embodiments, one vector comprises both the first nucleic acid and the second nucleic acid. In some embodiments, the vector is a lentiviral vector or an AAV vector.

In certain embodiments, the engineered immune cell comprises one nucleic acid that encodes both the protein comprising a first antigen binding domain and the protein comprising a second antigen binding domain. In some embodiments, a vector comprises the one nucleic acid. In some embodiments, the vector is a lentiviral vector or an AAV vector. In certain embodiments, the one nucleic acid is located within a disrupted TRAC, CD52 or CD70 locus.

In various embodiments, the engineered immune cell comprises or further comprises one or more genomic modifications, e.g. a modification of an endogenous genetic locus, for example, of one or both alleles of one or more of the following: an endogenous CD70 gene, an endogenous TCRa gene and an endogenous CD52 gene. In various embodiments, the one or more genomic modifications cause a reduction or absence of functional expression of the gene that contains the modification. In various embodiments, the modification comprises a disruption of the locus, e.g. a knockout, knockdown or knock-in. In various embodiments, the disruption comprises the insertion of a first nucleic acid encoding the protein comprising a first antigen binding domain and/or a second nucleic acid encoding the protein comprising a second antigen binding domain. In various embodiments, the modification causes a reduction or absence of functional expression of the gene by knockdown, e.g., antisense RNA, siRNA, miRNA, shRNA-mediated knockdown.

The engineered immune cell provided herein can be derived from or prepared from cells from any of various sources. The engineered immune cell can be prepared or derived from one or more cells e.g. stem cells or immune cells from a person other than the person to whom the engineered immune cells will be administered, e.g. a donor (e.g. a healthy volunteer) other than the recipient, or can be prepared or derived from cells e.g. stem cells or immune cells from the person to whom the engineered immune cells will be administered (the recipient), or can be derived from one or more induced pluripotent stem cells (iPSCs). In various embodiments, the engineered immune cell disclosed herein is or is derived from an immune cell obtained from a healthy volunteer, is obtained from a patient, or is derived from an iPSC.

Also provided herein is a population of engineered immune cells as disclosed herein. In various embodiments, the population comprises between 10³ and 10¹⁰ engineered immune cells provided herein, e.g. 10³, 10⁴, 10⁵, 10⁶, 10⁷, 10⁸, 10⁹, or 10¹⁰ engineered immune cells, or any range between any two of those values. In certain embodiments, no more than 75%, no more than 50%, or no more than 25% of the engineered immune cells of the population functionally express any one of TRAC, CD52 and CD70 or any two or more of TRAC, CD52 and CD70. In certain embodiments, no more than 75%, no more than 50%, or no more than 25% of the engineered immune cells of the population functionally express TRAC. In certain embodiments, no more than 75%, no more than 50%, or no more than 25% of the engineered immune cells of the population functionally express CD52. In certain embodiments, no more than 75%, no more than 50%, or no more than 25% of the engineered immune cells of the population functionally express TRAC, and no more than 75%, no more than 50%, or no more than 25% of the engineered immune cells of the population functionally express CD52.

Also provided herein is a population of cells, wherein the population of cells comprises at least 10% engineered immune cells disclosed herein, at least 20% engineered immune cells disclosed herein, at least 30% engineered immune cells disclosed herein, at least 40% engineered immune cells disclosed herein, at least 50% engineered immune cells disclosed herein, at least 75% engineered immune cells disclosed herein, or at least 90% engineered immune cells disclosed herein. In certain embodiments, at least 10%, at least 20%, at least 30%, at least 40%, at least 50%, at least 75%, or at least 90% of the engineered cells comprise one or more genomic modifications of one or more of an endogenous TCRa (or TRAC) gene, an endogenous CD52 gene and an endogenous CD70 gene. In various embodiments, the population of cells comprises between 10³ and 10¹⁰ cells.

In various embodiments, the populations of cells and of engineered immune cells disclosed herein are derived from one or more immune cells obtained from a healthy volunteer, from one or more immune cells obtained from a patient, or from one or more induced pluripotent stem cells (iPSCs).

Also provided herein is a pharmaceutical composition comprising one or more of the engineered immune cells disclosed herein or comprising a population of cells or of engineered immune cells disclosed herein, and further comprising at least one pharmaceutically acceptable carrier or excipient.

Further provided herein is a method of making the engineered immune cell disclosed herein that comprises one or more genomic modifications of one or more of an endogenous TCRa gene, an endogenous CD52 gene and an endogenous CD70 gene, the method comprising the use of one or more gene editing technologies selected from the group consisting of TALENs, zinc fingers, Cas-CLOVER, and a CRISPR/Cas system and/or the use of any known gene knockdown methods e.g. those that employ any of various RNA-based techniques (e.g. shRNA, antisense RNA, miRNA, siRNA; see, e.g., Lam et al., Mol. Ther.-Nucleic Acids 4:e252 (2015), doi:10.1038/mtna.2015.23; Sridharan and Gogtay, Brit. J. Clin. Pharmacol. 82: 659-72 (2016)) to reduce functional expression of one or more than one of TRAC, CD52 and CD70 in an engineered immune cell disclosed herein, and/or functional expression from any other locus.

Further provided herein is a method of making the engineered immune cell disclosed herein comprising introducing into an immune cell a first nucleic acid encoding the protein comprising a first antigen binding domain and a second nucleic acid encoding the protein comprising a second antigen binding domain. In certain embodiments, one vector comprises both the first nucleic acid and the second nucleic acid. In various embodiments, the vector is a lentiviral vector. In various embodiments, a first vector comprises the first nucleic acid and a second vector comprises the second nucleic acid. In certain embodiments, either or both of the first vector and the second vector are lentiviral vectors. In various embodiments, the protein comprising a first antigen binding domain is a CAR and/or the protein comprising a second antigen binding domain is a CD70 CAR. In various embodiments, the protein comprising a first antigen binding domain and the protein comprising a second antigen binding domain (i.e. CD70 binding domain) are physically separate and distinct proteins. In certain embodiments, the separate and distinct proteins are expressed from a bicistronic expression cassette, separated by, for example, a P2A or T2A peptide. In some embodiments, the protein comprising the first antigen binding domain is N-terminal to the protein comprising the CD70 binding domain. In some embodiments, the protein comprising the CD70 binding domain is N-terminal to the protein comprising the first antigen binding domain. In various embodiments, the protein comprising a first antigen binding domain and the protein comprising a second antigen binding domain (i.e. CD70 binding domain) are the same protein, e.g. a bispecific CAR.

Also provided herein is a method of treating a condition or disease in a patient comprising administering to the patient one or more of any one or more of the engineered immune cells disclosed herein, or a population of cells or of engineered immune cells as disclosed herein, or a composition as disclosed herein. In various embodiments, the condition or disease is a solid tumor or a hematological tumor. In various embodiments, the condition or disease is a viral disease, a bacterial disease, a cancer, an inflammatory disease, an immune disease, or an aging-associated disease. In various embodiments, the condition or disease is selected from the group consisting of gastric cancer, sarcoma, lymphoma (including Non-Hodgkin's lymphoma), leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In various embodiments, the patient is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In various embodiments, the method comprises administering about 10³ or 10⁴ to about 10⁹ engineered immune cells disclosed herein per kg body weight, or about 10⁵ to about 10⁶ cells of engineered immune cells disclosed herein per kg body weight, or between 0.1×10⁶ and 5×10⁶ engineered immune cells disclosed herein per kg body weight.

In various embodiments, the cells are administered as a single dose. In various embodiments, the cells are administered as more than one dose over a period of time.

### BRIEF DESCRIPTION OF THE DRAWINGS

FIG. 1 illustrates the functions of an allogeneic CAR T cell that comprises both a first CAR that can recognize and bind to a tumor antigen and a second CAR, termed “CD70 Dagger” in the drawing, that can recognize and bind to CD70. The first, anti-tumor antigen CAR, causes the recognized tumor cell to lyse. The second, anti-CD70 CAR, a.k.a. CD70 Dagger, causes the lysis of immune cells (e.g. NK and/or T cells) of the patient (recipient of the allogeneic CAR T cell) that respond to and/or attack the allogeneic CAR T cell. The allogeneic CAR T cell thereby persists longer by preventing or limiting the attack by the patient's immune cells.

FIGS. 2A-2D. Graft donor CD70 CAR T cells are protected from killing by recipient alloreactive T cells. PBMCs from eight recipient donors were co-cultured with irradiated graft donor T cells for 7 days to allow for priming and expansion of alloreactive recipient T cells (RTCs). FIG. 2A. Primed alloreactive RTCs were then isolated and co-cultured either with graft donor T cell receptor alpha constant (TRAC) knockout (KO) CD70 CAR T cells or with graft donor T cell receptor alpha constant (TRAC) knockout (KO) non-transduced T cells (NTD) at a ratio of 1:1 for 48 hrs. Killing of graft donor cells was assessed by flow cytometry. FIG. 2B. Percent killing of non-transduced and CD70 CAR T cells after co-culturing with low alloreactivity RTCs and with high alloreactivity RTCs. FIG. 2C. Representative flow cytometry plots showing killing of CD70+ RTCs. FIG. 2D. Absolute numbers (Abs. No.) and survival of CD4 and CD8 RTCs. Symbols represent individual RTC donors. Data are representative of two independent experiments.

FIGS. 3A-3B. Graft donor CD70 CAR T cells from multiple donors show killing of RTCs. PBMCs from a recipient donor were co-cultured with graft donor TRAC KO CD70 CAR T cells generated from three different donors at a ratio of 1:1 for 6 days. Killing of recipient T cells was assessed by flow cytometry. FIG. 3A, Absolute numbers and FIG. 3B, percent survival of CD4 and CD8 RTCs. Symbols represent individual CAR T cell graft donors.

FIGS. 4A-4B. Graft donor CD70 CAR T cells show killing of CD70-positive recipient B cells, T cells, and NK cells. PBMCs from a recipient donor were co-cultured with graft donor TRAC KO CD19 or CD70 CAR T cells at a ratio of 2:1 for 6 days. Killing of CD70-positive recipient cells was assessed by flow cytometry. FIG. 4A. Flow cytometry plots showing upregulation of CD70 on activated B, T, and NK cells and killing of CD70+ recipient cells by CD70 CAR T cells. FIG. 4B. Absolute numbers of CD70+ recipient B cells, T cells, and NK cells.

FIGS. 5A-5C. Activated human T cells were transduced with LVV encoding a range of CD70 dagger proteins (CD70dg) and cells were analyzed by flow cytometry. Percentage of CD70 dagger+ cells on day 14 post-activation is shown in FIG. 5A. CD4:CD8 ratio on day 14 post-activation is shown in FIG. 5B. Activation status of the transduced cells was assessed by measuring the expression of CD25 and 4-1BB on day 9 post-activation (FIG. 5C). Circles represent either NTD control or CD70 daggers that are first-generation CARs, and triangles indicate CD70 daggers that are second-generation CARs. Error bars represent means±SEM. MFI: Mean Fluorescence Intensity.

FIGS. 6A-F. T cells expressing a CD70 dagger (or a CD70 binding protein) deplete alloreactive T cells and resist T cell-mediated rejection. Alloreactive T cell MLRs (FIG. 6A) and PBMC MLRs (FIG. 6B) were performed using TRAC^{KO} graft donor T cells expressing a CD70 dagger. Cells were analyzed by flow cytometry at day 9 and the absolute number of remaining host T and NK cells was measured. The data in FIGS. 6C-D show that activated host T and NK cells were eliminated by T cells expressing a CD70 dagger in PBMC MLRs. Data are representative of two independent experiments. Symbols represent unique graft-host donor pairs. FIGS. 6E-F depict results of PBMC MLR assays of additional CD70 dagger constructs that are second-generation CARs that carry variant CD3z signaling domains (bbz1XX and bbzXX3). Error bars represent means±SEM.

FIGS. 7A-G. T cells expressing a CD70 dagger introduced by site-specific integration. Activated human T cells were modified to express different CD70 dagger proteins encoded from an AAV vector introduced by site-specific integration, and cells were analyzed by flow cytometry. FIG. 7A shows the percentage of CD70 dagger positive cells, and FIG. 7B shows CD70 dagger expression level, both on day 14 post-activation. The expression of activation markers CD25 and 4-1BB on day 9 post-activation is shown in FIG. 7C, and the CD4:CD8 ratio as determined by flow cytometry on day 14 post-activation is shown in FIG. 7D. Dotted lines indicate average values for cells expressing the CD70 dagger that is a second-generation CAR with a CD3ζ and 4-1BB signaling domains, as indicated as “CD70 CAR”. Circles and triangles represent CD70 daggers that are first- and second-generation CARs, respectively (FIGS. 7E-G). PBMC MLRs were performed using TRAC-KO T cells expressing different CD70 dagger proteins and cell counts were measured over time. The data in FIG. 7E show that CD70 dagger T cells resisted host rejection and expanded in PBMC MLR assays. The data in FIGS. 7F-G show that host T and NK cell expansions were inhibited by the CD70 dagger T cells. Data are the combined results from 4 unique graft-host donor pairs for panels FIGS. 7E-G. Error bars represent means±SEM.

FIGS. 8A-C depict amino acid residues of CD70 important for different antibodies binding to the CD70 trimer complex. FIGS. 8D-E show results of resistance of host rejection and inhibition of host T cells by the graft cells expressing CD70 dagger proteins containing a CD70 binding domain derived from anti-CD70 antibody clone 4F11, 8C8, or 8F8.

FIG. 9 shows cytotoxicity results of T cells expressing an anti-tumor antigen CAR, e.g., a CD19 CAR, alone or in conjunction with a CD70 dagger.

### DETAILED DESCRIPTION

The instant disclosure provides a strategy for providing a therapeutic allogeneic cell (e.g. CAR T cell) product that can overcome or mitigate the effects of rejection by the recipient's immune system. This permits the cell product to persist longer in the recipient and thus promotes and/or improves the therapeutic effect. The present strategy provides allogeneic cells with a defense against host immune cells that become activated against the allogeneic cells. This defense comprises expression by the allogeneic cells of a CD70-binding protein, e.g., a CD70 CAR. This strategy thus selectively targets the CD70 (cluster of differentiation 70) protein on a host's or recipient's e.g. patient's T and NK cells.

### General Techniques

The practice of the instant disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Gene editing techniques using TALENs, CRISPR/Cas9, and megaTAL nucleases, for example, are within the skill of the art and explained fully in the literature, such as T. Gaj et al., Genome-Editing Technologies: Principles and Applications, Cold Spring Harb Perspect Biol 2016; 8:a023754 and citations therein.

### Definitions

As used herein “autologous” means that cells, a cell line, or population of cells used for treating subjects that are obtained from said subject.

As used herein “allogeneic” means that cells or population of cells used for treating subjects that are not obtained from said subject, but instead from a donor.

As used herein, the term “endogenous” refers to any material from or produced inside an organism, cell, tissue or system.

As used herein, the term “exogenous” refers to any material introduced from or produced outside an organism, cell, tissue or system.

As used herein, “immune cell” refers to a cell of hematopoietic origin functionally involved in the initiation and/or execution of innate and/or adaptative immune response. Examples of immune cells include T cells, e.g., alpha/beta T cells and gamma/delta T cells, Regulatory T (Treg) cells, B cells, natural killer (NK) cells, natural killer T (NKT) cells, mast cells, and myeloid-derived phagocytes.

As used herein, the term “expression” refers to the transcription and/or translation of a particular nucleotide sequence driven by a promoter.

As used herein, “expression vector” refers to a vector comprising a recombinant polynucleotide comprising expression control sequences operatively linked to a nucleotide sequence to be expressed. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In various embodiments, engineered immune cells of the present disclosure functionally express a first antigen binding protein and a second, CD70-binding protein e.g. first CAR and second, CD70 CAR and optionally further comprise additional features. For example, they can comprise genomic modifications e.g. mutations at endogenous genes such as one or more of CD70, TCRa and CD52 that decreases or eliminates functional expression of the gene, and/or they can express one or more additional proteins. They can also comprise one or more other genomic modifications that decrease or eliminate functional expression of the gene by, for example, gene knockdown. The antigen binding proteins and one or more additional proteins may be expressed from an exogenous nucleic acid encoding the protein (with or without signal sequence) that is introduced into the cell by techniques described herein. As described herein, engineered immune cells of the present disclosure can derive, e.g., be prepared, from cells, e.g., immune cells obtained from various sources.

As used herein, to “functionally express” a gene means that a gene is expressed and that expression yields a functioning gene end product. For example, if a gene encodes a protein, then a cell functionally expresses the gene if expression of the gene ultimately produces a properly functioning protein. Thus, if a gene is not transcribed, or expression of the gene ultimately produces an RNA that is not translated or translation yields only a non-functioning protein e.g. the protein does not fold correctly or is not transported to its site of action (e.g. membrane, for membrane-bound proteins), for example, then the gene is not functionally expressed. Functional expression can be measured directly (e.g. by assaying for the gene product itself) or indirectly (e.g. by assaying for the effects of the gene product).

As used herein, “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter).

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

“Promoter” and “promoter sequence” are used interchangeably and refer to a DNA sequence capable of controlling the expression of a coding sequence or functional RNA. In general, a coding sequence is located 3′ to a promoter sequence. It is understood by those skilled in the art that different promoters can direct the expression of a gene in different tissues or cell types, or at different stages of development, or in response to different environmental or physiological conditions.

In any of the vectors of the present disclosure, the vector optionally comprises a promoter disclosed herein.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the instant disclosure.

The term “extracellular ligand-binding domain” as used herein refers to an oligo- or polypeptide that is capable of binding a ligand. Preferably, the domain will be capable of interacting with a cell surface molecule. For example, the extracellular ligand-binding domain can be chosen to recognize a ligand that acts as a cell surface marker on target cells associated with a particular disease state. The term “stalk domain” is used herein to refer to any oligo- or polypeptide that functions to link the transmembrane domain to the extracellular ligand-binding domain. In particular, stalk domains are used to provide more flexibility and accessibility for the extracellular ligand-binding domain.

The term “intracellular signaling domain” refers to the portion of a protein which transduces the effector signal function signal and directs the cell to perform a specialized function.

A “co-stimulatory molecule” as used herein refers to the cognate binding partner on a T cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, CD8, 4-1BB (CD137), OX40, CD30, CD40, ICOS, lymphocyte function-associated antigen-1 (LFA-1), CD2, CD7, LIGHT, NKG2C, B7-H3 and a ligand that specifically binds with CD83 and the like.

A “co-stimulatory ligand” refers to a molecule on an antigen presenting cell that specifically binds a cognate co-stimulatory signal molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), 4-1 BBL, OX40L, inducible costimulatory ligand (ICOS-L), intercellular adhesion molecule (ICAM, CD30L, CD40, CD70, CD83, HLA-G, MICA, M1 CB, HVEM, lymphotoxin R receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1 BB, OX40, CD30, CD40, ICOS, lymphocyte function-associated antigen-1 (LFA-1), CD2, CD7, LTGHT, NKG2C, B7-H3, a ligand that specifically binds with CD83.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen-binding fragments thereof (such as Fab, Fab′, F(ab′)2, and Fv), and any other modified configuration of the immunoglobulin molecule that comprises an antigen recognition site including, for example without limitation, single chain (scFv) and domain antibodies (including, for example, shark and camelid antibodies), and fusion proteins comprising an antibody. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen-binding fragment” or “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (see, e.g., Ward et al., Nature 341:544-546, 1989), and an isolated complementarity determining region (CDR).

An antibody, an antibody conjugate, or a polypeptide that “specifically binds” to a target is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. It is also understood that under this definition, for example, an antibody (or moiety or epitope) that specifically binds to a first target may or may not specifically bind to a second target. “Specific binding” therefore does not necessarily require (although it can include) exclusive binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are several techniques for determining CDRs, e.g., an approach based on cross-species sequence variability (i.e., Kabat et al. Sequences of Proteins of Immunological Interest, (5th ed., 1991, National Institutes of Health, Bethesda MD)); an approach based on crystallographic studies of antigen-antibody complexes (Al-lazikani et al., 1997, J. Molec. Biol. 273:927-948), the Chothia system (i.e., Chothia and Lesk, J. Mol. Biol. (1987) 196(4):901-917. As used herein, a CDR can refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs can be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs can also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs can be identified as the residues that make enthalpic contributions to antigen binding. See, e.g., Makabe et al., Journal of Biological Chemistry, 283:1 156-1 166, 2008. Still other CDR boundary definitions may not strictly follow one of the above approaches, but will nonetheless overlap with at least a portion of the Kabat CDRs, although they can be shortened or lengthened in light of prediction or experimental findings that particular residues or groups of residues or even entire CDRs do not significantly impact antigen binding. As used herein, a CDR can refer to CDRs defined by any approach known in the art, including combinations of approaches. The methods used herein can utilize CDRs defined according to any of these approaches. For any given embodiment containing more than one CDR, the CDRs can be defined in accordance with any of Kabat, Chothia, extended, AbM, contact, AHo and/or conformational definitions.

Antibodies (and CARs) of the instant disclosure can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4):924-40, 2007).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide can comprise modified nucleotides, such as methylated nucleotides and their analogs. If present, modification to the nucleotide structure can be imparted before or after assembly of the chain. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

As used herein, “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure. The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the instant disclosure. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining a beneficial or desired clinical result. For purposes of the instant disclosure, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a disease (e.g., cancer), decreasing symptoms resulting from a disease (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolong survival of subjects having a disease (e.g., cancer).

“Ameliorating” means a lessening or improvement of one or more symptoms as compared with not administering a treatment. “Ameliorating” also includes shortening or reduction in duration of a symptom. As used herein, an “effective dosage” or “effective amount” of drug, compound, or pharmaceutical composition is an amount sufficient to affect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of the instant disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” can be considered in the context of administering one or more therapeutic agents, and a single agent can be considered to be given in an effective amount if, in conjunction with one or more other agents, a desirable result can be or is achieved.

As used herein, a “subject” is any mammal, e.g a human, or a monkey. Mammals include, but are not limited to, farm animals, sport animals, pets, primates, horses, dogs, cats, mice and rats. In an exemplary embodiment, the subject is a human. In an exemplary embodiment, the subject is a monkey, e.g. a cynomolgus monkey.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “pharmaceutically acceptable carrier” or “pharmaceutically acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions of the instant disclosure comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 21st Ed. Mack Publishing, 2005).

As used herein, “alloreactivity” refers to the ability of T cells to recognize MHC complexes that were not encountered during thymic development. Alloreactivity manifests itself clinically as host-versus-graft rejection or reaction and graft-versus-host disease.

Reference to “about” a value or parameter herein includes (and describes) embodiments that are directed to plus or minus 1, 2, 3, 4, 5, 6, 7, 8, 9 or 10% that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the instant disclosure are described in terms of a Markush group or other grouping of alternatives, the instant disclosure encompasses not only the entire group listed as a whole, but also each member of the group individually and all possible subgroups of the main group, and also the main group absent one or more of the group members. The instant disclosure also envisages the explicit exclusion of one or more of any of the group members in the disclosed and/or claimed embodiments.

Unless otherwise defined, all technical and scientific terms used herein have the same meaning as commonly understood by one of ordinary skill in the art to which the present disclosure belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the instant disclosure. The materials, methods, and examples are illustrative only and not intended to be limiting.

An “antigen binding protein” comprises one or more antigen binding domains. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease or to a viral or bacterial antigen.

Antigen binding domains include, but are not limited to, antibody binding regions that are immunologically functional fragments. The term “immunologically functional fragment” (or “fragment”) of an antigen binding domain is a species of antigen binding domain comprising a portion (regardless of how that portion is obtained or synthesized) of an antibody that lacks at least some of the amino acids present in a full-length chain, but which is still capable of specifically binding to a target antigen. Such fragments are biologically active in that they bind to the target antigen and can compete with other antigen binding domains, including intact antibodies, for binding to a given epitope.

Immunologically functional immunoglobulin fragments include, but are not limited to, scFv fragments, Fab fragments (Fab′, F(ab′)2, and the like), one or more complementarity determining regions (“CDRs”), a diabody (heavy chain variable domain on the same polypeptide as a light chain variable domain, connected via a short peptide linker that is too short to permit pairing between the two domains on the same chain), domain antibodies, bivalent antigen binding domains (comprises two antigen binding sites), multispecific antigen binding domains, and single-chain antibodies. These fragments can be derived from any mammalian source, including but not limited to human, mouse, rat, camelid or rabbit. As will be appreciated by one of skill in the art, an antigen binding domain can include non-protein components.

The variable regions typically exhibit the same general structure of relatively conserved framework regions (FR) joined by the 3 hypervariable regions (CDRs). The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3.

In some embodiments, antigen binding domains comprise one or more complementarity binding regions (CDRs) present in the full-length light or heavy chain of an antibody, and in some embodiments comprise a single heavy chain and/or light chain or portion thereof. These fragments can be produced by recombinant DNA techniques or can be produced by enzymatic or chemical cleavage of antigen binding domains, including intact antibodies.

In some embodiments, the antigen binding domain is an antibody or fragment thereof, including one or more of the complementarity determining regions (CDRs) thereof. In some embodiments, the antigen binding domain is a single chain variable fragment (scFv), comprising light chain CDRs: CDR1, CDR2 and CDR3, and heavy chain CDRs: CDR1, CDR2 and CDR3.

The assignment of amino acids to each of the framework, CDR, and variable domains is typically in accordance with numbering schemes of Kabat numbering (see, e.g., Kabat et al. in Sequences of Proteins of Immunological Interest, 5th Ed., NIH Publication 91-3242, Bethesda Md. 1991), Chothia numbering (see, e.g., Chothia & Lesk, (1987), J Mol Biol 196: 901-917; Al-Lazikani et al., (1997) J Mol Biol 273: 927-948; Chothia et al., (1992) J Mol Biol 227: 799-817; Tramontano et al., (1990) J Mol Biol 215(1): 175-82; and U.S. Pat. No. 7,709,226), contact numbering, the AbM scheme (Antibody Modeling program, Oxford Molecular) or the AHo system (Honneger and Pluckthun, J Mol Biol (2001) 309(3):657-70).

In some embodiments, the antigen binding domain is a recombinant antigen receptor. The term “recombinant antigen receptor” as used herein refers broadly to a non-naturally occurring surface receptor that comprises an extracellular antigen-binding domain or an extracellular ligand-binding domain, a transmembrane domain and an intracellular domain. In some embodiments, the recombinant antigen receptor is a chimeric antigen receptor (CAR). Chimeric antigen receptors (CARs) are well-known in the art. A CAR is a fusion protein that comprises an antigen recognition moiety, a transmembrane domain and T cell activation domains (see, e.g., Eshhar et al., Proc. Natl. Acad. Sci. USA, 90(2): 720-724 (1993)).

In some embodiments, the intracellular domain of a recombinant antigen receptor comprises a co-stimulatory domain and an ITAM-containing domain. In some embodiments, the intracellular domain of a recombinant antigen receptor comprises an intracellular protein or a functional variant thereof (e.g., truncation(s), insertion(s), deletion(s) or substitution(s)).

The term “extracellular ligand-binding domain” or “extracellular antigen-binding domain” as used herein refers to a polypeptide that is capable of binding a ligand or an antigen or capable of interacting with a cell surface molecule, such as a ligand or a surface antigen. For example, the extracellular ligand-binding or antigen-binding domain can be chosen to recognize a ligand that acts as a cell surface marker on target cells associated with a particular disease state, e.g., a tumor-specific antigen. In some embodiments, the antigen-binding domain comprises an antibody, or an antigen binding fragment or an antigen binding portion of an antibody. In some embodiments, the antigen binding domain comprises an Fv or scFv, an Fab or scFab, an F(ab′)2 or a scF(ab′)2, an Fd, a monobody, a affibody, a camelid antibody, a VHH antibody, a single domain antibody, or a darpin. In some embodiments, the ligand-binding domain comprises a partner of a binding pair, such as a ligand that binds to a surface receptor, or an ectodomain of a surface receptor that binds to a ligand.

The term “stalk domain” or “hinge domain” are used interchangeably herein to refer to any polypeptide that functions to link the transmembrane domain to the extracellular ligand-binding domain. In particular, stalk domains are often used to provide more flexibility and accessibility for the extracellular ligand-binding domain.

The term “intracellular signaling domain” refers to the portion of a protein which transduces the effector signal function signal and directs the cell to perform a specialized function.

### Vectors

Expression vectors and methods for the administration of polynucleotide compositions are known in the art and further described herein.

In another aspect, the instant disclosure provides a method of making any of the polynucleotides described herein.

Polynucleotides complementary to any such sequences are also encompassed by the instant disclosure. Polynucleotides can be single-stranded (coding or antisense) or double-stranded, and can be DNA (genomic, cDNA or synthetic) or RNA molecules. RNA molecules include hnRNA molecules, which contain introns and correspond to a DNA molecule in a one-to-one manner, and mRNA molecules, which do not contain introns. Additional coding or non-coding sequences can, but need not, be present within a polynucleotide of the instant disclosure, and a polynucleotide can, but need not, be linked to other molecules and/or support materials.

Polynucleotides can comprise a native sequence (i.e., an endogenous sequence that encodes an antibody or a portion thereof) or can comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide can generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof. Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window,” as used herein, refers to a segment of at least about 20 contiguous positions, usually 30 to about 75, or 40 to about 50, in which a sequence can be compared to a reference sequence of the same number of contiguous positions after the two sequences are optimally aligned.

Optimal alignment of sequences for comparison can be conducted using the Megalign program in the Lasergene suite of bioinformatics software (DNASTAR, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:1 1-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window can comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants can also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the instant disclosure. Further, alleles of the genes comprising the polynucleotide sequences provided herein are within the scope of the instant disclosure. Alleles are endogenous genes that are altered as a result of one or more mutations, such as deletions, additions and/or substitutions of nucleotides. The resulting mRNA and protein can, but need not, have an altered structure or function. Alleles can be identified using standard techniques (such as hybridization, amplification and/or database sequence comparison).

The polynucleotides of the instant disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further described herein. Polynucleotides can be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in, e.g., U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors can be constructed according to standard techniques, or can be selected from a large number of cloning vectors available in the art. While the cloning vector selected can vary according to the host cell intended to be used, useful cloning vectors will generally have the ability to self-replicate, can possess a single target for a particular restriction endonuclease, and/or can carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the instant disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components can generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

A polynucleotide encoding an antigen binding protein, e.g., a CAR, can exist in an expression cassette or expression vector (e.g., a plasmid for introduction into a bacterial host cell, or a viral vector such as a baculovirus vector for transfection of an insect host cell, or a plasmid or viral vector such as a lentivirus for transfection of a mammalian host cell). In some embodiments, a polynucleotide or vector can include a nucleic acid sequence encoding ribosomal skip sequences such as, for example without limitation, a sequence encoding a 2A peptide. 2A peptides, which were identified in the Aphthovirus subgroup of picomaviruses, cause a ribosomal “skip” from one codon to the next without the formation of a peptide bond between the two amino acids encoded by the codons (see, e.g., Donnelly and Elliott 2001; Atkins, Wills et al. 2007; Doronina, Wu et al. 2008). By “codon” is meant three nucleotides on an mRNA (or on the sense strand of a DNA molecule) that are translated by a ribosome into one amino acid residue. Thus, two polypeptides can be synthesized from a single, contiguous open reading frame within an imRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptides into the secretory pathway of a host cell, in some embodiments, a secretory signal sequence (also known as a leader sequence, prepro-sequence or pre-sequence) is provided in a polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences can be positioned elsewhere in the nucleic acid sequence of interest (see, e.g., Welch et al., U.S. Pat. No. 5,037,743; Holland et al., U.S. Pat. No. 5,143,830). Those skilled in the art will recognize that, in view of the degeneracy of the genetic code, considerable sequence variation is possible among these polynucleotide molecules. In some embodiments, nucleic acid sequences of the instant disclosure are codon-optimized for expression in mammalian cells, preferably for expression in human cells. Codon-optimization refers to the exchange in a sequence of interest of codons that are generally rare in highly expressed genes of a given species for codons that are generally frequent in highly expressed genes of such species, such codons encoding the same amino acids as the codons that are being exchanged.

Methods of preparing immune cells for use in immunotherapy are provided herein. In some embodiments, the methods comprise introducing an antigen binding protein e.g. a CAR into one or more immune cells, or introducing a polynucleotide encoding the antigen binding protein e.g. CAR, and expanding the cells. In some embodiments, the instant disclosure relates to a method of engineering an immune cell comprising: providing an immune cell and expressing at the surface of the cell at least one antigen binding protein e.g. a CAR. In some embodiments, the method comprises: transfecting the cell with at least one polynucleotide encoding an antigen binding protein e.g. a CAR, and expressing the at least one polynucleotide in the cell.

In some embodiments, the polynucleotides encoding the antigen binding protein e.g. a CAR are present in one or more expression vectors for stable expression in the cells. In some embodiments, the polynucleotides are present in viral vectors for stable expression in the cells. In some embodiments, the viral vectors can be for example, lentiviral vectors or adenoviral vectors.

In some embodiments, polynucleotides encoding polypeptides according to the present disclosure can be mRNA which is introduced directly into the cells, for example by electroporation. In some embodiments, CytoPulse technology can be used to transiently permeabilize living cells for delivery of material into the cells. Parameters can be modified in order to determine conditions for high transfection efficiency with minimal mortality.

Also provided herein are methods of transfecting an immune cell e.g. a T cell. In general, any conventional method known to the person of ordinary skill in the art can be used, such as introducing any of RNA, DNA or protein into a cell by means of electroporation. See, e.g., Luft and Ketteler, J. Biomolec Screening 20(8): 932 (2015) (DOI: 10.1177/1087057115579638). In some embodiments, the method comprises: contacting a T cell with RNA and applying to the T cell an agile pulse sequence consisting of: (a) an electrical pulse with a voltage range from about 2250 to 3000 V per centimeter; (b) a pulse width of 0.1 ms; (c) a pulse interval of about 0.2 to 10 ms between the electrical pulses of step (a) and (b); (d) an electrical pulse with a voltage range from about 2250 to 3000 V per centimeter with a pulse width of about 100 ms and a pulse interval of about 100 ms between the electrical pulse of step (b) and the first electrical pulse of step (c); and (e) four electrical pulses with a voltage of about 325 V with a pulse width of about 0.2 ms and a pulse interval of 2 ms between each of 4 electrical pulses. In some embodiments, a method of transfecting a T cell comprises contacting said T cell with RNA and applying to the T cell an agile pulse sequence comprising: (a) an electrical pulse with a voltage of about 1600, 2250, 2300, 2350, 2400, 2450, 2500, 2550, 2400, 2450, 2500, 2600, 2700, 2800, 2900 or 3000V per centimeter; (b) a pulse width of 0.1 ms; (c) and a pulse interval of about 0.2, 0.5, 1, 2, 3, 4, 5, 6, 7, 8, 9 or 10 ms between the electrical pulses of step (a) and (b); (d) one electrical pulse with a voltage range from about 2250 to 3000 V per centimeter, e.g. of 2250, 2300, 2350, 2400, 2450, 2500, 2550, 2400, 2450, 2500, 2600, 2700, 2800, 2900 or 3000V per centimeter with a pulse width of 100 ms and a pulse interval of 100 ms between the electrical pulse of step (b) and the first electrical pulse of step (c); and (e) 4 electrical pulses with a voltage of about 325 V with a pulse width of about 0.2 ms and a pulse interval of about 2 ms between each of 4 electrical pulses. Any values included in the value range described above are disclosed in the present application. Electroporation medium can be any suitable medium known in the art. In some embodiments, the electroporation medium has conductivity in a range spanning about 0.01 to about 1.0 milliSiemens.

In some embodiments, the method can further comprise a step of genetically engineering a cell by inactivating (e.g. knocking out) or reducing the expression level of at least one gene expressing, for example without limitation, a component of the TCR (e.g. TRAC) and/or a target for an immunosuppressive agent. By inactivating a gene it is intended that the gene of interest is not expressed in a functional protein form. In some embodiments, the gene to be inactivated is selected from the group consisting of, for example without limitation, TCRα, TCRβ, CD52 and CD70. In some embodiments the method comprises inactivating or reducing the expression level of one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease or TALEN®), a megaTAL nuclease or a Cas9 endonuclease.

In another aspect, a step of genetically modifying or engineering immune cells e.g. T cells can comprise: modifying immune cells e.g. T cells by inactivating at least one gene expressing a target for an immunosuppressive agent, and expanding the cells, optionally in the presence of the immunosuppressive agent. An immunosuppressive agent is an agent that suppresses immune function by one of several mechanisms of action. An immunosuppressive agent can diminish the extent and/or voracity of an immune response. Non-limiting examples of immunosuppressive agents include calcineurin inhibitors, targets of rapamycin, interleukin-2 α-chain blockers, inhibitors of inosine monophosphate dehydrogenase, inhibitors of dihydrofolic acid reductase, corticosteroids, and immunosuppressive antimetabolites. Some cytotoxic immunosuppressants act by inhibiting DNA synthesis. Others can act through activation of T cells or by inhibiting the activation of helper cells. The methods according to the instant disclosure allow conferring immunosuppressive resistance to e.g. T cells for immunotherapy by inactivating the target of the immunosuppressive agent in the T cells. As non-limiting examples, targets for an immunosuppressive agent can be a receptor for an immunosuppressive agent such as for example without limitation CD52, glucocorticoid receptor (GR), FKBP family gene members, and cyclophilin family gene members.

Compositions and methods for functionally expressing in a cell (e.g. engineered immune cell) an antigen binding protein e.g. a CAR in conjunction with functionally expressing, in the same cell, a CD70-binding protein, e.g., a CD70 CAR (CAR that specifically recognizes CD70) are provided herein. Also provided are uses of such compositions and methods for improving the functional activities of immune cells e.g. T cells, such as CAR-T cells. The methods and compositions provided herein are useful for improving in vivo persistence and therapeutic efficacy of immune cells e.g. allogeneic immune cells (e.g. allogeneic T cells, allogeneic CAR-T cells).

In various embodiments, engineered immune cells e.g. engineered T cells provided herein functionally express a first antigen binding protein e.g. a first chimeric antigen receptor (CAR) and a second antigen binding protein e.g. a second CAR, wherein the second CAR is a CD70 CAR. Advantageously, the engineered immune cells provided herein exhibit improved in vivo persistence and/or increased resistance to rejection by the recipient's immune system, relative to non-engineered cells. For example, a population of cells comprising a first CAR and a second, CD70 CAR persist longer than a population of cells comprising the same first CAR and either no second CAR or a second CAR that does not specifically bind CD70.

One or more antigen binding proteins e.g. one or more CARs can be synthesized in situ in the cell after introduction of a polynucleotide construct encoding the proteins into the cell. Alternatively, an antigen binding protein e.g. CAR can be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as, for example, recombinant viral vectors (e.g. retroviruses, including lentiviruses, adenoviruses), liposomes, and the like. Transient transformation methods include, for example without limitation, microinjection, electroporation or particle bombardment. Polynucleotides can be included in vectors, such as for example plasmid vectors or viral vectors.

In some embodiments of an engineered immune cell e.g. T cell provided herein, each CAR that the cell expresses can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR.

The extracellular ligand-binding domain specifically binds to a target of interest. In some embodiments, the target of interest e.g. the target of interest of the first CAR can be any molecule of interest, including, for example, without limitation, CD70, BCMA, EGFRvIII, Flt-3, WT-1, CD20, CD22, CD23, CD30, CD38, CD33, CD133, WT1, TSPAN10, MHC-PRAME, HER2, MSLN, PSMA, PSCA, GPC3, Liv1, ADAM10, CHRNA2, LeY, NKG2D, CS1, CD44v6, ROR1, CD19, Claudin-18.2 (Claudin-18A2, or Claudin18 isoform 2), DLL3 (Delta-like protein 3, Drosophila Delta homolog 3, Delta3), Muc17, Muc3, Muc16, FAP alpha (Fibroblast Activation Protein alpha), Ly6G6D (Lymphocyte antigen 6 complex locus protein G6d, c6orf23, G6D, MEGT1, NG25), or RNF43 (E3 ubiquitin-protein ligase RNF43, RING finger protein 43), specifically including the human form of any of the listed exemplary targets.

In some embodiments, an extracellular ligand-binding domain comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 16), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid or other vector containing a polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The intracellular signaling domain of a CAR according to the instant disclosure is responsible for intracellular signaling following the binding of the extracellular ligand-binding domain to the target resulting in the activation of the immune cell and immune response. The intracellular signaling domain has the ability to activate at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Examples of ITAM used in the instant disclosure can include as non-limiting examples those derived from TCRζ, FcRγ, FcRβ, FcRε, CD3γ, CD3δ, CD3ε, CD5, CD22, CD79a, CD79b and CD66d, or a variant thereof. In some embodiments, the intracellular signaling domain of the CAR can comprise the CD3ζ signaling domain or a variant of the CD3ζ domain. In some embodiments the intracellular signaling domain of a CAR of the instant disclosure comprises a domain of a co-stimulatory molecule.

In some embodiments, the intracellular signaling domain of a CAR of the instant disclosure comprises a part of a co-stimulatory molecule selected from the group consisting of fragment of 4-1BB (GenBank: AAA53133) and CD28 (NP_006130 and isoforms thereof).

CARs are expressed on the surface membrane of the cell. Thus, each CAR can comprise a transmembrane domain. Suitable transmembrane domains for a CAR disclosed herein have the ability to (a) be expressed at the surface of a cell, for example an immune cell such as, for example without limitation, lymphocyte cells (e.g. T cells) or Natural killer (NK) cells, and (b) interact with the ligand-binding domain and intracellular signaling domain for directing a cellular response of an immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a domain of the T cell receptor such as α, β, γ or δ, polypeptide constituting CD3 complex, IL-2 receptor e.g. p55 (α chain), p75 (β chain or γ chain), subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1) or is the human CD8α chain transmembrane domain. In some embodiments said transmembrane domain comprises the human CD28 transmembrane domain or is derived from the human CD28 protein transmembrane domain. The transmembrane domain can further comprise a stalk domain between the extracellular ligand-binding domain and said transmembrane domain. A stalk domain can comprise up to 300 amino acids, for example, from 10 to 100 amino acids or 25 to 50 amino acids. The stalk region can be derived from all or part of naturally occurring molecules, such as from all or part of the extracellular region of CD8, CD4, or CD28, or from all or part of an antibody constant region. Alternatively, the stalk domain can be a synthetic sequence that corresponds to a naturally occurring stalk sequence or can be an entirely synthetic stalk sequence. In some embodiments said stalk domain is a part of human CD8α chain (e.g., NP_001139345 and isoforms thereof). In another particular embodiment, the transmembrane domain comprises a part of the human CD8α chain. In some embodiments, CARs disclosed herein and functionally expressed in the engineered immune cells disclosed herein can comprise an extracellular ligand-binding domain that specifically binds CD70 or any of the targets of interest disclosed herein, CD8α human stalk and transmembrane domains, the CD3ζ signaling domain, and 4-1BB signaling domain. In some embodiments, CARs disclosed herein and functionally expressed in the engineered immune cells disclosed herein can comprise an extracellular ligand-binding domain that specifically binds CD70 (e.g., a CD70 antigen-binding domain or a CD70-binding domain) and a transmembrane domain, with or without one or more intracellular signaling domains. In some embodiments, a nucleic acid encoding a CAR can be introduced into an immune cell as a transgene via a vector e.g. a plasmid vector or lentiviral vector. In some embodiments, the vector e.g. plasmid vector can also contain, for example, a selection marker which provides for identification and/or selection of cells which received the vector.

CAR polypeptides can be synthesized in situ in the cell after introduction of polynucleotides encoding the CAR polypeptides into the cell. Alternatively, CAR polypeptides can be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as, for example, recombinant viral vectors (e.g. retroviruses (e.g. lentiviruses), adenoviruses), liposomes, and the like. Transient transformation methods include, for example without limitation, microinjection, electroporation or particle bombardment. Polynucleotides can be included in vectors, such as for example plasmid vectors or viral vectors.

Also provided herein are immune cells e.g. T cells such as isolated T cells or peripheral blood mononuclear cells (PBMCs) obtained according to any one of the methods described herein. Any immune cell capable of expressing heterologous DNAs can be used for the purpose of expressing the antigen binding protein e.g. CAR of interest and further for engineering to express a reduced level of NLRC5 and/or TAP2. In some embodiments, the immune cell is a T cell. In some embodiments, an immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The isolated cell can also be a dendritic cell, killer dendritic cell, a mast cell, a NK− cell, a B-cell or a T cell selected from the group consisting of inflammatory T-lymphocytes, cytotoxic T-lymphocytes, regulatory T-lymphocytes or helper T-lymphocytes. In some embodiments, the cell can be derived from the group consisting of CD4+ T-lymphocytes and CD8+ T-lymphocytes. In some embodiments, the immune cells e.g. T cells such as isolated T cells are further modified e.g. genetically engineered by methods described herein (e.g. known gene editing techniques that employ, for example, TALENs, CRISPR/Cas9, or megaTAL nucleases to partially or wholly delete or disrupt one or more loci e.g. CD70, TRAC and CD52) so that they express a reduced level of the corresponding functional protein relative to comparable cells not so engineered.

The engineered immune cells provided herein can comprise one or more mimotope sequences that enable sorting of cells to enrich a population for cells engineered as described herein, e.g. cells that express the antigen binding protein, and/or that provide a safety switch mechanism to inactivate the immune cell after the cells have been administered to the patient or recipient, e.g. to limit adverse effects. Such mimotope sequences and their application in cell sorting and as safety switches are known in the art and described, for example, in US2018/0002435, which is incorporated herein by reference in its entirety.

Prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art, can be used. In some embodiments, cells can be derived from a healthy donor, from a subject diagnosed with cancer or from a subject diagnosed with an infection. In some embodiments, cells can be part of a mixed population of cells which present different phenotypic characteristics.

Also provided herein are cell lines obtained from a modified e.g. transformed or engineered immune cell e.g. engineered T cell according to any of the methods described herein. In some embodiments, an engineered immune cell e.g. engineered T cell according to the instant disclosure comprises a first polynucleotide encoding a first antigen binding protein e.g. a CAR and a second polynucleotide encoding a second, CD70-binding protein e.g. a CD70 CAR and optionally further modified or engineered e.g. genetically modified to express one or more of CD70, TRAC and CD52 at a reduced level (e.g. modified to comprise a knockout of either or both loci). In some embodiments, one polynucleotide encodes both a first antigen binding protein e.g. a CAR and a second, CD70-binding protein e.g. a CD70 CAR.

The immune cells, e.g. T cells of the instant disclosure, can be activated and expanded, either prior to or after modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. Immune cells e.g. T cells can be expanded in vitro or in vivo. Generally, the immune cells of the instant disclosure can be expanded, for example, by contact with an agent that stimulates a CD3 TCR complex and a co-stimulatory molecule on the surface of the immune cells to create an activation signal for the cell. For example, chemicals such as calcium ionophore A23187, phorbol 12-myristate 13-acetate (PMA), or mitogenic lectins like phytohemagglutinin (PHA) can be used to create an activation signal for the immune cell, e.g., a T cell.

In some embodiments, T cell populations can be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate medium (e.g., Minimal Essential Media, RPMI Media 1640 or, X-VIVO™ 5, (Lonza)) that can contain factors necessary for proliferation and viability, including serum (e.g., fetal bovine or human serum), interleukin-2 (IL-2), insulin, IFN-γ, IL-4, IL-7, GM-CSF, IL-10, IL-2, IL-15, a TGFβ, and TNF, or any other additives for the growth of cells known to the skilled artisan. Other additives for the growth of cells include, but are not limited to, surfactant, Plasmanate®, and reducing agents such as N-acetyl-cysteine and 2-mercaptoethanol. Media can include RPMI 1640 (as noted herein), AIM V, DMEM, MEM, α-MEM, F-12, X-VIVO™ 10, X-VIVO™ 15 and X-VIVO™ 20, OpTmizer™, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). Immune cells e.g. T cells that have been exposed to varied stimulation times can exhibit different characteristics.

In some embodiments, the cells of the instant disclosure can be expanded by co-culturing with tissue or cells. The cells can also be expanded in vivo, for example in the subject's blood after administrating the cell into the subject.

In another aspect, the instant disclosure provides compositions (such as pharmaceutical compositions) comprising any of the cells of the instant disclosure or a population comprising such cells. In some embodiments, the composition comprises an engineered immune cell e.g. engineered T cell according to the instant disclosure comprises a first polynucleotide encoding a first antigen binding protein e.g. a CAR and a second polynucleotide encoding a second, CD70-binding protein e.g. a CD70 CAR and optionally further modified or engineered e.g. genetically modified to express one or more of CD70, TRAC and CD52 at a reduced level relative to cells not so further modified (e.g. modified to comprise a knockout of either or both loci). In some embodiments, one polynucleotide encodes both a first antigen binding protein e.g. a CAR and a second, CD70-binding protein e.g. a CD70 CAR. In some embodiments, the composition comprises a population of such engineered immune cells e.g. engineered T cells, for example 10³, 10⁴, 10⁵, 10⁶, 10⁷, 10⁸, 10⁹, or 10¹⁰ of such engineered immune cells e.g. engineered T cells, or some number of cells in between any two of these values. In various embodiments, the compositions disclosed herein further comprise one or more pharmaceutically acceptable carriers or excipients.

In some embodiments, primary cells isolated from a donor are engineered as described herein to provide a population of cells of which a subpopulation (e.g., a proportion less than 100%, such as 10%, 20%, 30%, 40%, 50%, 60%, 70% 80% or 90%) of the resulting cells comprise all of the desired modifications. Such a resulting population, comprising a mixture of cells that comprise all of the modifications and cells that do not, can be used in the methods of treatment of the instant disclosure and to prepare the compositions of the instant disclosure. Alternatively, this population of cells (the “starting population”) can be manipulated by known methods e.g. cell sorting and/or expansion of cells that have the desired modifications, to provide a population of cells that is enriched for those cells comprising one or more of the desired modifications (e.g. enriched for cells that express both of the desired antigen binding proteins and optionally further enriched for cells that express one or more of CD70, TRAC and CD52 at a reduced level relative to comparable cells not engineered with respect to CD70, TRAC and/or CD52), that is, that comprises a higher percentage of such modified or engineered cells than did the starting population. The population enriched for the modified cells can then be used in the methods of treatment of the instant disclosure and to prepare the compositions of the instant disclosure, for example. In some embodiments, the enriched population of cells contains, or contains at least, for example, 20%, 30%, 40%, 50%, 60%, 70%, 80%, 90%, 95%, or 99% cells that have one or more of the modifications. In other embodiments, the proportion of cells of the enriched population of cells that comprise one or more of the modifications is at least 30% higher than the proportion of cells of the starting population of cells that comprise the desired modifications.

### CD70-Binding Proteins or CD70-Specific CARs and Methods of Making Thereof

In a related aspect, the current disclosure provides CD70-binding proteins as described herein, wherein the CD70-binding proteins comprise an extracellular ligand or antigen binding domain that binds to CD70 (or a CD70 binding domain) and a transmembrane domain. The CD70-binding proteins can comprise no to one or more intracellular signaling domains as described herein.

The instant disclosure provides CD70-binding proteins, including without limitation CARs that bind to CD70 (e.g., human CD70 (e.g., SEQ ID NO: 601), such as those deposited under the provisions of the Budapest Treaty and assigned accession number: P32970-1. CD70-specific CARs provided herein include single chain CARs and multichain CARs. In some embodiments, the CARs have the ability to redirect T cell specificity and reactivity toward CD70 in a non-MHC-restricted manner, exploiting the antigen-binding properties of monoclonal antibodies. The non-MHC-restricted antigen recognition gives T cells expressing CARs the ability to recognize an antigen independent of antigen processing, thus bypassing a major mechanism of tumor escape.

In some embodiments, the CD70-binding proteins provided herein comprise an extracellular domain (e.g., a single chain variable fragment (scFv)) and a transmembrane domain. In some embodiments, the CD70-binding protein or CD70 CARs provided herein comprise an extracellular ligand-binding domain (e.g., scFv), a transmembrane domain, and an intracellular signaling domain. In some embodiments, the CD70-binding protein comprises one or more intracellular signaling domains selected from the group consisting of a CD3ζ signaling domain, a CD3δ signaling domain, a CD3γ signaling domain, a CD3ε signaling domain, a CD28 signaling domain, a CD2 signaling domain, an OX40 signaling domain, and a 4-1BB signaling domain, or a variant thereof. In some embodiments, the intracellular signaling domain comprises the amino acid sequence of one or more of SEQ ID NOs: 265, 271-278, 281-295, 311-337, 580-591, or 616-617. In some embodiments, the intracellular signaling domain comprises the amino acid sequence of one or more of SEQ ID NO: 271, 616, 272, 617, 276, 275, 582, 583, 585, or 586. In some embodiments, the CD70 binding protein comprises a CD3ζ or a CD3γ signaling domain, or a variant thereof, and does not comprise a costimulatory domain. In some embodiments, the CD70 binding protein comprises a 4-1BB signaling domain, or a variant thereof, and does not comprise a CD3 signaling domain. In some embodiments, the CD70 binding protein comprises a 4-1BB signaling domain and a CD3ζ signaling domain. In some embodiments, the CD70 binding protein does not comprise an intracellular signaling domain. Different intracellular signaling domain or combination thereof can confer different signaling strength that can contribute to T cell proliferation, potency, survival, persistence, and/or resistant to host immune cell rejection. Described herein are CD70-binding proteins comprising no to one or more intracellular signaling domains.

In some embodiments, engineered immune cells comprising the CD70-binding proteins described herein can exhibit different levels of persistence and/or resistance to rejection by host immune cells and can be suitable for use in lymphodepletion in vivo when administered to a patient. In some embodiments, engineered immune cells comprising the CD70-binding proteins described herein can inhibit proliferation and/or activities of host immune cells to different degrees that can allow for fine-tuning of the depth of lymphodepletion in vivo when administered to a patient. For example, engineered immune cells comprising a CD70-binding protein that demonstrated extended expansion and/or inhibition of host immune cells proliferation or activities, in e.g., an MLR assay, can be used for an extended lymphodepletion. In contrast, engineered immune cells comprising a CD70-binding protein that demonstrated less extended expansion and/or inhibition of host immune cells in the same or similar assay can be used when a less complete or a less thorough lymphodepletion is desired.

In some embodiments, the CARs provided herein further comprises a “hinge” or “stalk” domain, which can be situated between the extracellular ligand-binding domain and the transmembrane domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR. In some embodiments, the CARs are inducible, such as by small molecule (e.g., AP1903) or protein (e.g., Epo, Tpo, or PD-1). In some embodiments, a CD70-specific multichain CAR is based on the high affinity receptor for IgE (FcεRI). The FcεRI expressed on mast cells and basophiles triggers allergic reactions. FcεRI is a tetrameric complex composed of a single α subunit, a single β subunit, and two disulfide-linked γ subunits. The α subunit contains the IgE-binding domain. The β and γ subunits contain ITAMs that mediate signal transduction. In some embodiments, the extracellular domain of the FcRα chain is deleted and replaced by a CD70-specific extracellular ligand-binding domain. In some embodiments, the multichain CD70-specific CAR comprises an scFv that binds specifically to CD70, the CD8α hinge, and the ITAM of the FcRβ chain. In some embodiments, the CAR may or may not comprise the FcRγ chain.

In some embodiments, the extracellular ligand-binding domain comprises an scFv comprising the light chain variable (VL) region and the heavy chain variable (VH) region of a target antigen (i.e., CD70) specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₃ (SEQ ID NO: 296), which bridges approximately 3.5 nm between the carboxy terminus of one variable region and the amino terminus of the other variable region. Linkers of other sequences have been designed and used (Bird et al., 1988, supra). Other exemplary linkers can generally include other GS linkers can generally include (GGGGS)x, where x is 1, 2, 3, 4, 5 (SEQ ID NO: 604). In some embodiments, x is 6, 7, 8, 9, 10, 11, 12, 13, 14, 15, or any integer less than about 20. In some embodiments, the linker is (GGGGS)₄ (SEQ ID NO: 602). In some embodiments the linker is GSTSGSGKPGSGEGSTKG (SEQ ID NO: 603), as described in Whitlow et al, Protein Eng. (1993) 6(8): 989-895. In general, linkers can be short, flexible polypeptides, which in some embodiments are comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In another aspect, provided is a CAR, which specifically binds to CD70, wherein the CAR comprises an extracellular ligand-binding domain comprising: a VH region comprising a VH CDR1, VH CDR2, and VH CDR3 of the VH sequence shown in SEQ ID NO: 2, 4, 6, 8, 10, 12, 14, 16, 18, 20, 22, 24, 26, 28, 30, 32, 34, 36, 38, 40, 42, 44, 46 or 48; and/or a VL region comprising VL CDR1, VL CDR2, and VL CDR3 of the VL sequence shown in SEQ ID NO: 1, 3, 5, 7, 9, 11, 13, 15, 17, 19, 21, 23, 25, 27, 29, 31, 33, 35, 37, 39, 41, 43, 45 or 47. In some embodiments, the VH and VL are linked together by a flexible linker. In some embodiments a flexible linker comprises the amino acid sequence shown in SEQ ID NO: 296.

In some embodiments, a CAR of the disclosure comprises an extracellular ligand-binding domain having any one of partial light chain sequence as listed in Table 1 and/or any one of partial heavy chain sequence as listed in Table 1. In Table 1, the underlined sequences are CDR sequences according to Kabat and in bold according to Chothia.

TABLE 1

| mAb | Light Chain | Heavy Chain |
|---|---|---|
| 31H1 | DIVMTQNPLSSPVTLGQPASISCRSSQS | QVQLVQSGAEVKKPGSSVKVSCKASGG |
| | LVHSDGNTYLSWLQQRPGQSPRLLIYK | TFSSYGFSWVRQAPGQGLEWMGGIIPIF |
| | ISNRFSGVPDRFSGSGAGTDFTLKISRV | GSANYAQKFQGRVTITADKSTSTVYME |
| | EAEDVGVYYCMQATQFPLTIGGGSKV | LISLRSEDTAVYYCARGGSSSPFAYWG |
| | EIK | QGTLVTVSS |
| | (SEQ ID NO: 1) | (SEQ ID NO: 2) |
| 63B2 | DIVMTQTPLSSPVTLGQPASISCRSSQSL | QVQLVQSGAEVKKPGSSVKVSCKASGG |
| | VHSDGNTYLSWLQQRPGQSPRLLIYKI | TFSSYGFSWVRQAPGQGLEWMGGIIPIF |
| | SNRFSGVPDRFSGSGAGTDFTLKISRVE | GTANYAQKFQGRVTITADKSTSTVFME |
| | AEDVGVYYCMQATQFPLTIGGGSKVE | LISLRSEYTAVYYCARGGSSSPFAYWG |
| | IK | QGTLVTVSS |
| | (SEQ ID NO: 3) | (SEQ ID NO: 4) |
| 40E3 | DIQMTQSPSSLSASVGDRVTITCRASQG | QVQLQESGPGLVKPSETLSLTCTVSGGS |
| | ISNYLAWFQQKPGKAPKSLIYAASSLQ | ISSYYWNWIRQPPGKGLEWIGYIYYSGS |
| | SGVPSKFSGSGSGTDFTLTISSLQPEDFA | TNYNPSLKSRVTISVDTSKNQFSLKLRS |
| | TYYCQQYNSYPLTFGGGTKVEIK | VTAADTAVYYCARDIRTWGQGTLVTV |
| | (SEQ ID NO: 5) | SS |
| | | (SEQ ID NO: 6) |
| 42C3 | DVVMTQSPLSLPVTLGQPASISCRSSQS | EVQLVESGGGLVQPGGSLRLSCAASGF |
| | LVYSDENTYLNWFQQRPGQSLRRLIY | TFRNSWMSWVRQAPGKGLEWVANIKR |
| | QVSNRDSGVPDRFSGSGSGTDFTLKISR | DGSEKYYVDSVKGRFTISRDNAKNSLY |
| | VEAEDVGVYFCMQGTYWPPTFGGGT | LQMNSLRAEDTAVYYCARDQTGSFDY |
| | KVEIK | WGQGTLVTVSS |
| | (SEQ ID NO: 7) | (SEQ ID NO: 8) |
| 45F11 | EIVMTQSPATLSMSLGERATLSCRASQS | QVQLRGSGPGLVKPSETLSLTCTVSDDS |
| | VSSSLAWYQQKPGQAPRLLIYGASTRA | ISVYYWSWIRQPAGKGLEWIGRVYSSG |
| | TGIPARFGGSGSGTEFTLTISSLQSEDFA | NINYNPSLESRVTMSVDTSKSRFSLNLS |
| | VYYCQQYINWPHFGGGTKVEIK | SVTAADTAVYYCARGLDAFDIWGQGTM |
| | (SEQ ID NO: 9) | VTVSS |
| | | (SEQ ID NO: 10) |
| 64F9 | DIQMTQSPSSLSASVGDRVTITCQASQD | EVQLLESGGGLVQPGESLRLSCEVSGFT |
| | ISNYLNWYQQKPGKAPKILIYGASNLE | FTSYAMSWVRQVPGKGLEWVSIISGVA |
| | TGVPSRFSGSGSGTDFTFAISSLQPEDV | FTTYYADSVKGRFTISRDHSKNTLYLQ |
| | ATYYCQQYDNFPITFGQGTRLEIK | MNGLRAEDTAVYYCVKVDGEVYWGQ |
| | (SEQ ID NO: 11) | GTLVTVSS |
| | | (SEQ ID NO: 12) |
| 72C2 | EIVMTQSPDTLSVSPGERAILSCRASQS | QVQLVQSGAEVKKPGSSVKVSCEASGG |
| | VSSNLAWYQQKPGQAPRLLIYSASTRA | TFITYAISWVRQAPGQGLEWMGGIIPFF |
| | SGIPARFSGSGSGTEFTLSISSLQSEDF | GTANYAQKFQGRVTITADKSTSTASME |
| | AVYYCQQYDNWPPLTFGGGTKVEIK | LRSLRSEDTAMYYCAQWELFFFDFWG |
| | (SEQ ID NO: 13) | QGTPVTVSS |
| | | (SEQ ID NO: 14) |
| 2F10 | EIVLTQSPGTLSLSPGERATLSCRASQS | AVQLVESGGGLVQPGGSLRLSCAASGF |
| | VSSSYLAWYQQQPGQAPRLLIYGASSR | TFTYYSMNWVRQAPGKGLEWVSHISIR |
| | ATGIPDRFSGSGSGTDFTLTISRLEPE | SSTIYFADSAKGRFTISRDNAKNSLYLQ |
| | DFAIYYCQQYGSSPLTFGGGTKVEIK | MNSLRDEDTAVYYCARGSGWYGDYFD |
| | (SEQ ID NO: 15) | YWGQGTLVTVSS |
| | | (SEQ ID NO: 16) |
| 4F11 | DIQMTQSPSAMSASVGDRVTITCRASQ | QVTLKESGPVLVKPTETLTLTCTVSGFS |
| | DISNYLAWFQQKPGKVPKRLIYAASSL | LSNARMGVTWIRQPPGKALEWLAHIFS |
| | QSGVPSRFSGSGSGTEFTLTISSLLPEDF | NDEKSYSTSLKSRLTISKDTSKTQVVLT |
| | ATYYCLQLNSFPFTFGGGTKVEIN | MTNMDPVDTATYYCARIRDYYDISSYY |
| | (SEQ ID NO: 17) | DYWGQGTLVSVSS |
| | | (SEQ ID NO: 18) |
| 10H10 | DIQMTQSPSSVSASVGDRVTITCRASQ | EVQLVESGGGLVQPGGSLRLSCAVSGF |
| | GISSWLAWYQQKPGKAPKVLIYAASS | TFSNHNIHWVRQAPGKGLEWISYISRSS |
| | LQSGVPSRFSGSGSGTDFTLTISSLQPED | STIYYADSVKGRFTISRDNAKNSLYLQM |
| | FATYYCQQAFSFPFTFGPGTKVDIK | NSLRDEDTAVYYCARDHAQWYGMDV |
| | (SEQ ID NO: 19) | WGQGTTVTVSS |
| | | (SEQ ID NO: 20) |
| 17G6 | DIVMTQSPDSLAVSLGERATINCKSSQS | EVQLVESGGGLVQPGGSLRLSCVASGF |
| | VLYSYNNKNYVAWYQQKPGQPPNLLI | TFSSYWMSWVRQAPGKGLEWVASIKQ |
| | FWASTRESGVPDRFSGSGSGTDFTLTIS | DGSEKYYVDSVKGRFTISRDNAKNSVY |
| | SLQAEDVAVYYCQQYYSTLTFGGGTK | LQMNSLRAEDTGVYYCAREGVNWGW |
| | VEIK | RLYWHFDLWGRGTLVTVSS |
| | (SEQ ID NO: 21) | (SEQ ID NO: 22) |
| 65E11 | EIVLTQSPGTLSLSPGERVTLSCRASQS | EVQVVESGGGLVQPGGSLRLSCAASGF |
| | VSSSYLAWYQQKPGQAPRLLIYDASSR | TFSSYSMNWVRQAPGKGLEWVSHSSIS |
| | ATGIPDRFSGSGSGTDFTLTISRLEPE | RGNIYFADSVKGRFTISRDNAKNSLYLQ |
| | DFAVYYCQQYGSSPLTFGGGTKVEIK | MNSLRDEDTAVYYCARGSGWYGDYFD |
| | (SEQ ID NO: 23) | YWGQGTLVTVSS |
| | | (SEQ ID NO: 24) |
| P02B10 | ELQSVLTQPPSASGTPGQRVTISCSGSSS | EVQLLESGGGLVQPGGSLRLSCAASGFA |
| | NIGSNYVYWYQQLPGTAPKLLIYRNN | FSNYAMSWVRQAPGKGLEWVSAIRGG |
| | QRPSGVPDRFSGSKSGTSASLAISGLRS | GGSTYYADSVKGRFTISRDNSKNTLYL |
| | EDEADYYCAAWDDSLSGVVFGGGTKL | QMNSLRAEDTAVYYCARDFISGTWYP |
| | TVL | DYWGQGTLVTVSS |
| | (SEQ ID NO: 25) | (SEQ ID NO: 26) |
| P07D03 | ELQSVLTQPPSASGTPGQRVTISCSGSR | EVQLVQSGAEVKKPGESLKISCKGSGYR |
| | SNIGSNYVYWYQQLPGTAPKLLIYRNN | FTSYWIGWVRQMPGKGLEWMGSIYPD |
| | QRPSGVPDRFSGSKSGTSASLAISGLRS | DSDTRYSPSFQGQVTISADKSISTAYLQ |
| | EDEADYYCASWDGSLSAVVFGTGTKL | WSSLKASDTAMYYCASSTVDYPGYSYF |
| | TVL | DYWGQGTLVTVSS |
| | (SEQ ID NO: 27) | (SEQ ID NO: 28) |
| P08A02 | ELQSVLTQPPSASGTPGQRVTISCSGSSS | EVQLVQSGAEVKKPGESLKISCKGSGYT |
| | NIGSNYVYWYQQLPGTAPKLLIYRNN | FTNYWIAWVRQMPGKGLEWMGIIYPD |
| | QRPSGVPDRFSGSKSGTSASLAISGLR | GSDTRYSPSFQGQVTISADKSISTAYLQ |
| | SEDEADYYCATWDDSLGSPVFGTGTKL | WSSLKASDTAMYYCARDITSWYYGEP |
| | TVL | AFDIWGQGTLVTVSS |
| | (SEQ ID NO: 29) | (SEQ ID NO: 30) |
| P08E02 | ELDIQMTQSPSSLSASVGDRVTITCRAS | EVQLVQSGAEVKKPGESLKISCKGSGYS |
| | QSISRYLNWYQQKPGKAPKLLIYAASI | FTSSWIGWVRQMPGKGLEWMGIIYPD |
| | LQTGVPSRFSGSGSGTDFTLTISSLQPE | SDTRYSPSFQGQVTISADKSISTAYLQWS |
| | DFATYYCQQSYSTTMWTFGQGTKVEI | SLKASDTAMYYCAKGLSQAMTGFGFD |
| | K | YWGQGTLVTVSS |
| | (SEQ ID NO: 31) | (SEQ ID NO: 32) |
| P08F08 | ELQSVLTQPPSASGTPGQRVTISCSGSSS | EVQLVQSGAEVKKPGESLKISCKGSGY |
| | NIGSNYVNWYQQLPGTAPKLLIYGDY | GFTSYWIGWVRQMPGKGLEWMGIIYPD |
| | QRPSGVPDRFSGSKSGTSASLAISGLR | DSDTKYSPSFQGQVTISADKSISTAYLQ |
| | SEDEADYYCATRDDSLSGSVVFGTGTK | WSSLKASDTAMYYCASSYLRGLWGGY |
| | LTVL | FDYWGQGTLVTVSS |
| | (SEQ ID NO: 33) | (SEQ ID NO: 34) |
| P08G02 | ELDIQMTQSPSSLSASVGDRVTITCRAS | EVQLVQSGAEVKKPGESLKISCKGSGYT |
| | QSIYDYLHWYQQKPGKAPKLLIYDAS | FPSSWIGWVRQMPGKGLEWMGIIYPDT |
| | NLQSGVPSRFSGSGSGTDFTLTISSLQPE | SHTRYSPSFQGQVTISADKSISTAYLQW |
| | DFATYYCQQSYTTPLFTFGQGTKVEIK | SSLKASDTAMYYCARASYFDRGTGYSS |
| | (SEQ ID NO: 35) | WWMDVWGQGTLVTVSS |
| | | (SEQ ID NO: 36) |
| P12B09 | ELDIQMTQSPSSLSASVGDRVTITCRAS | EVQLLESGGGLVQPGGSLRLSCAASGFT |
| | QYIGRYLNWYQQKRGKAPKLLIHGAT | FSQYSMSWVRQAPGKGLEWVSAISGG |
| | SLASGVPSRFSGSGSGTDFTLTISSLQPE | GVSTYYADSVKGRFTISRDNSKNTLYLQ |
| | DFATYYCQQSYSTTSPTFGQGTKVEIK | MNSLRAEDTAVYYCASDISDSGGSHW |
| | (SEQ ID NO: 37) | YFDYWGQGTLVTVSS |
| | | (SEQ ID NO: 38) |
| P12F02 | ELQSVLTQPPSASGTPGQRVTISCSGST | EVQLLESGGGLVQPGGSLRLSCAASGFT |
| | SNIGRNYVYWYQQLPGTAPKLLIYRTN | FSSYAMSWVRQAPGKGLEWVSTISGTG |
| | QRPSGVPDRFSGSKSGTSASLAISGLRS | GTTYYADSVKGRFTISRDNSKNTLYLQ |
| | EDEADYYCAAWDDSLSGRVFGTGTKL | MNSLRAEDTAVYYCAKVRAGIDPTASD |
| | TVL | VWGQGTLVTVSS |
| | (SEQ ID NO: 39) | (SEQ ID NO: 40) |
| P12G07 | ELQSVLTQPPSASGTPGQRVTISCSGSSS | EVQLLESGGGLVQPGGSLRLSCAASGFT |
| | NIGSNYVYWYQQLPGTAPKPLIYMNN | FNNFAMSWVRQAPGKGLEWVSGISGS |
| | QRPSGVPDRFSGSKSGTSASLAISGLRS | GDNTYYADSVKGRFTISRDNSKNTLYL |
| | EDEADYYCAAWDDSLSAVVFGTGTKL | QMNSLRAEDTAVYYCAKDRDIGLGWY |
| | TVL | SYYLDVWGQGTLVTVSS |
| | ((SEQ ID NO: 41) | (SEQ ID NO: 42) |
| P13F04 | ELQSVLTQPPSASGTPGQRVTISCSGSN | QVQLVQSGAEVKKPGSSVKVSCKASGG |
| | SNIGTNYVSWYQQLPGTAPKLLIYRSS | TFSSYAISWVRQAPGQGLEWMGEIIPIF |
| | RRPSGVPDRFSGSKSGTSASLAISGLRS | GTASYAQKFQGRVTITADESTSTAYME |
| | EDEADYYCAAWDGSLSGHWVFGTGT | LSSLRSEDTAVYYCARAGWDDSWFDY |
| | KLTVL | WGQGTLVTVSS |
| | (SEQ ID NO: 43) | (SEQ ID NO: 44) |
| P15D02 | ELDIQMTQSPSSLSASVGDRVTITCRAS | EVQLVQSGAEVKKPGESLKISCKGSGYS |
| | QSIDTYLNWYQQKPGKAPKLLIYSASS | FASYWIGWVRQMPGKGLEWMGVIYPG |
| | LHSGVPSRFSGSGSGTDFTLTISSLQPED | TSETRYSPSFQGQVTISADKSISTAYLQ |
| | FATYYCQQSYSTTAWTFGQGTKVEIK | WSSLKASDTAMYYCAKGLSASASGYSF |
| | (SEQ ID NO: 45) | QYWGQGTLVTVSS |
| | | (SEQ ID NO: 46) |
| P16C05 | ELDIQMTQSPSSLSASVGDRVTITCRAS | EVQLVQSGAEVKKPGESLKISCKGSGYS |
| | QSIGQSLNWYQQKPGKAPKLLIYGASS | FTDYWIGWVRQMPGKGLEWMGMISPG |
| | LQSGVPSRFSGSGSGTDFTLTISSLQPED | GSTTIYRPSFQGQVTISADKSISTAYLQW |
| | FATYYCQQSYSTPITFGQGTKVEIK | SSLKASDTAMYYCAREMYTGGYGGS |
| | (SEQ ID NO: 47) | WYFDYWGQGTLVTVSS (SEQ ID NO: 48) |
| 10A1 | DIQMTQSPSTLSASVGDRVTITCRASQS | QVQLQESGPGLVKPSETLSLTCTVSGGS |
| | ISTWLAWYQQKPGKAPKVLIYKASSL | ISYYYWTWIRQPPGKGLEWIGHIYYSGS |
| | ESGVPSRFSGSGSGTEFILTINSLQPDDF | TNYNPSLKSRVTISIDTSKNLFSLKLSSV |
| | ASYYCQQYKSYSHTFGQGTKLEIK | TAADTAVYYCARAEGSIDAFDFWGQGT |
| | (SEQ ID NO: 338) | MVTVSS |
| | | (SEQ ID NO: 339) |
| 10E2 | DIQMTQSPSTLSASVGDRVTITCRASQS | EVQLVESGGGLIQPGGSLRLSCAASGFT |
| | ISSWLAWYQQKPGKAPKVLIYKASSLE | VSSNYMTWVRQAPGKGLEWVSVIYSG |
| | SGVPSRFSGSGSGTEFTLTINSLQPDDFA | GSTYYADSVKGRFTISRDNSKNTLYLQ |
| | TYYCQQYKSFSLTFGQGTKLEIK | MNSLRAEDTAVYYCARNWGDYWGQG |
| | (SEQ ID NO: 340) | TLVTVSS |
| | | (SEQ ID NO: 341) |
| HAI | DIQMTQSPSTLSASVGDRVTITCRASQS | QVQLQESGPGLVKPSGTLSLTCTVSGGS |
| | ISSWLAWYQQKPGKAPKVLIYKASTL | IDYYFWNWFRQSPVKGLEWIGHVYDIG |
| | ESGVPSRFSGSGSGTEFTLTISSLQPDDF | NTKYNPSLKSRVTISIDTSENQFSLKLNS |
| | ATYYCQQYNSYSYTFGHGTKLEIK | VTAADTAVYYCARGEGAIDAFDIWGQ |
| | (SEQ ID NO: 342) | GTMVTVSS |
| | | (SEQ ID NO: 343) |
| 11C1 | DIQMTQSPSILSASVGDRVTITCRASQS | QVQLQESGPGLVKPSETLSLNCTVSGGS |
| | VSSWLAWYQQKPGKAPKVLIYKASSL | ISYYYWTWIRQPPGKGLEWIGHVIYSGT |
| | ESGVPSRFSGTGSGTEFTLTISSLQSDDF | TNYNPSLKSRVTISVDTSKNQFSLKLNS |
| | ATYYCQQYNTYSHTFGQGTKLEIK | VTAADTAVYYCVRAEGSIDAFDLWGQ |
| | (SEQ ID NO: 344) | GTMVTVSS |
| | | (SEQ ID NO: 345) |
| 11D1 | AIQMTQSPSSLSASVGDRVTITCRASQG | QVQLVESGGGVVQPGRSLRLSCVASGF |
| | IRNDLGWYQQKPGKAPKLLIYAASSLQ | TFSDYGIHWVRQAPGMGQEWVAVIWY |
| | SGVPSRFSGSGSGTDFTLTISSLQPEDFA | DGSiKKYSDSVKGRFIISRDNSENTVYLQ |
| | TYYCLQDYNYPFTFGPGTKVDIK | MNSLRGEDTAIYYCARDEVGtfGAFDF |
| | (SEQ ID NO: 346) | WGQGTKVTVSS |
| | | (SEQ ID NO: 347) |
| 11E1 | DIQMTQSPSSLSASVGDSITITCRASQDI | QVQLQESGPGLVKPLQTLSLTCTVSGGS |
| | DNYLAWYQQKTGKVPKVLIYAASALQ | ISSdgYYWSWIRQNPGKGLEWIGYMYYS |
| | SGVPSRFSGSGSGTDFTLTISSLQPEDVA | GSTYYNPSLKSRVTISVDTSKNQFSLKL |
| | TYYCQNYNSGPRTFGQGTKVEIK | RSVTAADTAVYYCTRDFGWYFDLWGR |
| | (SEQ ID NO: 348) | GTLVTVSS |
| | | (SEQ ID NO: 349) |
| 12A2 | DIQMTQSPSSLSASVGDRVTITCRASQD | QVQLQESGPGLVKPSQSLSLTCSVSGGS |
| | ISNYLTWYQQKPGRVPEVLIYAASALQ | VSSdgYYWSWIRQHPGKGLEWIGYIYYR |
| | SGVPSRFSGSGSGTDFTLTISSLQPEDVA | RITDYNPSLKSRVNISLDTSKNQFSLKLS |
| | TYYCQNYNSAPRTFGQGTKVEIK | SVTAADTAVYYCARDFGWYFDLWGR |
| | (SEQ ID NO: 350) | GTLVAVSS |
| | | (SEQ ID NO: 351) |
| 12C4 | DIVMTQSPLSLPVTPGEPASISCRSSQSL | QVQLVQSGAEVKKPGASVKVSCKASG |
| | LHSNGYNYLDWYLQKPGQSPQVLILL | YTFTGYYLHWVRQAPGQGLEWMGWI |
| | GSNRASGVPDRVSASGSGTDFTLKISR | NpNSGGTNYAQKFQGRVTMTRDTSITT |
| | MQAEDVGIYYCMQTLQTPFTFGQGTK | AYMELSRLRIDDTAVYYCARDRGVtmiv |
| | LEIK | DGMDDWGQGTTVTVSS |
| | (SEQ ID NO: 352) | (SEQ ID NO: 353) |
| 12C5 | DIQLTQSPSFLSASVGDRVIITCRASQGI | EVELVESGGGMVQPGRSLRLSCAASGF |
| | NSHLAWYQQKPGKAPKLLIYYASTLPS | TFSDYGMHWVRQAPGMGLEWVTVIW |
| | GVPSRFSGSGSGTEFTLTVTSLQPEDFA | YDGSnKYYADSVKGRFTISRDNSKNTVF |
| | TYYCQQLNHYPITFGQGTRLDIN | LQMNSLRAEDTAVYYCARDEVGfvGAF |
| | (SEQ ID NO: 354) | DIWGQGTMVTVSS |
| | | (SEQ ID NO: 355) |
| 12C6 | DIQLTQSPSFLSASVGDRVIITCRASQGI | EVELVESGGGMVQPGRSLRLSCAASGF |
| | NSHLAWYQQKPGKAPKLLIYYASTLPS | TFSDYGMHWVRQAPGMGLEWVTVIW |
| | GVPSRFSGSGSGTEFTLTVTSLQPEDFA | YDGSnKYYADSVKGRFTISRDNSKNTVF |
| | TYYCQQLNHYPITFGQGTRLEIK | LQMNSLRAEDTAVYYCARDEVGfvGAF |
| | (SEQ ID NO: 605) | DIWGQGTMVTVSS |
| | | (SEQ ID NO: 606) |
| 12D3 | DIQMTQSPSSLSASVGDRVTITCRASQG | QVQLQESGPGLVKPSQTLSLTCTVSGGS |
| | ISNYLAWYQQKPGKVPKLLIYAASTLH | ISSdgYYWSWIRQHPGKGLEWIGYMYYS |
| | SGVPSRFSGSGSGTDFTLTISSLQPEDVA | GITYHNPSLKSRVTISVDTSKNQFSLRLS |
| | TYYCQKYNSAPRTFGQGTKVEIK | SVTAADTAVYYCARDFGWYFDLWGR |
| | (SEQ ID NO: 356) | GTLVTVSS |
| | | (SEQ ID NO: 357) |
| 12D6 | DIQMTQSPSSLSASVGDRVTITCRASQD | QVQLQESGPGLVKPSQTLSLTCTVSGGS |
| | ISNYLAWYQQKPGKVPKLLIYAASTLH | ISSdaYYWSWIRQHPGKGLEWIGYMYYS |
| | SGVPSRFSGSGSGTDFTLTISSLQPDDFA | GITYYNPSLKSRVTISVDTSKNQFSLKLS |
| | AYYCQKYNSAPRTFGQGTKVEIK | SVTAADTAVYYCARDFGWYFDLWGR |
| | (SEQ ID NO: 358) | GTLVTVSS |
| | | (SEQ ID NO: 359) |
| 12D7 | DIQLTQSPSFLSASVGDRVSITCRASQDI | QVQLVESGGGVVQPGRSLRLSCVASGF |
| | SSFLAWYQQKPGKAPVLLIYVASTLQS | TFSDYGIHWVRQAPGMGQEWVAVIWY |
| | GVPSRFSGSGSGTEFTLTVSSLQPEDFA | DGSiKKYSDSVKGRFIISRDNSENTVYLQ |
| | TYYCQQLHVYPITFGQGTRLEIR | MNSLRGEDTAIYYCARDEVGtfGAFDF |
| | (SEQ ID NO: 360) | WGQGTKVTVSS |
| | | (SEQ ID NO: 361) |
| 12F5 | DIVMTQTPLSLPVTPGEPASISCRSSQSL | EVQLVESGGGLVKPGGSLRLSCAASGF |
| | LDSDDGNtYLDWYLQKPGQSPQLLIYT | TFSNAWMSWVRQAPGKGLEWVGRIKs |
| | LSYRASGVPDRFSGSGSGTDFTLKISRV | ktGGGTTDYAAPVKGRFTISRDDSKNTL |
| | EAEDVGVYYCMQRIEFPFTFGPGTKV | YLQMNSLKTEDTAVYYCTSLIVGaiSLF |
| | DIK | DYWGQGTLVTVSS |
| | (SEQ ID NO: 362) | (SEQ ID NO: 363) |
| 12H4 | DIQMTQSPSALSASVGDRVAITCRASQ | QVQLRESGPGLVKPSETLSLTCTISGGSI |
| | TISTWLAWYQQKPGKAPKVLIYKASN | SYYFWTWIRQPPGRGLEWIGQIYYSGNT |
| | LESGVPSRFSGSGSGTEFTLTINSLQPDD | NSNPSLKSRVTISIDTSKNQFSLKLTSVT |
| | FATYYCQQYQTFSHTFGQGTKLEIK | VADTAVYYCVRAEGSIDAFDIWGQGT |
| | (SEQ ID NO: 364) | MVAVSS |
| | | (SEQ ID NO: 365) |
| 8C8 | DMQMTQSPSSLSASVGDRVTLTCRASQ | EVQLVESGGGLVKPGGSLRLSCVASGF |
| | GISNYLAWFQLKPGKVPKLLIYAASTL | TFSSYSMNWVRQFPGKGLEWVSSIStSS |
| | QSGVPSRFSGSGSGTDFALTISSLQPED | NYIHYADSLQGRFTISRDNAKNSLYLQM |
| | VATYYCQKYNSAPLTFGGGTKVEIK | SSLRVEDTAVYYCVRDKGTtltnWYFDL |
| | (SEQ ID NO: 366) | WGRGTLVTVSS |
| | | (SEQ ID NO: 367) |
| 8F7 | DIVMTQSPLSLPVTPGEPASISCRSSQTL | QVQLVESGGGVVQPGRSLRLSCGASGF |
| | VHSNGYNYLNWYLQKPGQSPQLLIYL | TFSSYGMHWVRQAPGKGLEWVAVIWY |
| | GSNRASGVPDRFSGSGSGSDFTLKISRM | DGSnKYYADSLKGRFTISRDNSKNTLYL |
| | EAEDVGVYYCMQAIQTPYTFGQGTNV | QMNSLRAEDTAVYYCARDGYSgssDAF |
| | EIK | DIWGQGTMVTVSS |
| | (SEQ ID NO: 368) | (SEQ ID NO: 369) |
| 8F8 | DIQMTQSPSTLSASVGDRVTITCRASQS | QVQLQESGPGLVQPSETLSLTCTVSGGS |
| | ISSWLAWYQQKPGKAPKVLIYKASNL | ISYYYWSWIRQPPGKGLEWIGNINYMG |
| | ESGVPSRFSGSGSGTEFTLTISSLQPDDF | NTIYNPSLKSRVTISVDTSKDQFSLKLTS |
| | ATYYCQQYNSYSCTFGQGTKLEIK | VSAADTAVYYCVRAEGSIDAFDFWGQ |
| | (SEQ ID NO: 370) | GTLVAVSL |
| | | (SEQ ID NO: 371) |
| 9D8 | DIQMTQSPSSLSASVGDRIIFTCQASQDI | QVQLVQSGAEVTKPGASVKVSCKASGY |
| | NNYLHWYQQKPGKAPKLLIYDASDWE | IFTGYYIYWVRQAPGQGLEWMGWINpS |
| | TGVPSRFSGSGSGTDFTFTISSLQPEDIA | SGGTNYAQKFQGRVTMARDTSISTAYM |
| | TYYCQQYDHLPITFGQGTRVEIK | ELSSLRSDDTAVYYCARDRKReyyynFG |
| | (SEQ ID NO: 372) | MDVWGQGTTVTVST |
| | | (SEQ ID NO: 373) |
| 9E10 | DIQMTQSPSSLSASVGDRVILTCQASQD | QVQLVQSGAEVTKPGASVKVSCKASGY |
| | ISNYLHWYQQKPGKAPKLLIYDASDLE | TFTSHYIYWVRQAPGQGLEWMGWINp |
| | TGVPSRFSGSGSGADFTFTISNLQPEDF | NSGGTNYAQKFQDRVTMARDTSISTAY |
| | ATYYCQQYDHLPITFGQGTRLEIK | MELSRLRSDDTAVYYCAKDRKReyyynF |
| | (SEQ ID NO: 374) | GMDVWGQGTTVTVSA |
| | | (SEQ ID NO: 375) |
| 9E5 | DIQMTQSPSSLSASVGDRVILTCQASQD | QVQLVQFGVEVRKPGASVKVSCKVSGF |
| | ISNYLHWYQQKPGKAPKLLIYDASDLE | TFTSHYIYWVRQAPGQGLEWMGWINp |
| | TGVPSRFSGSGSGADFTFTISNLQPEDF | NSGGTKYAQKFQDRVTMARDTSISTAY |
| | ATYYCQQYDHLPITFGQGTRLEIK | MELSRLRSDDTSVYYCVKDRKReyyynF |
| | (SEQ ID NO: 376) | GMDVWGQGTTVTVSS |
| | | (SEQ ID NO: 377) |
| 9F4 | DIQMTQSPSSLSASVGDRVTITCQASQD | EVQMLESGGGLIQPGGSLRLSCKTSGFT |
| | ISNYLNWYQQKPGKAPKLLIYDASNLE | LSIYAIHWVRQAPGRGLEWVSSFGgRG |
| | TGVPSRFSGSGSGTDFTFTISSLQPEDIA | SSTYFADSVKGRFTISRDASENSLYLHM |
| | TYYCQQYDNLPYTFGQGTKLEIK | NSLRAEDTAVYYCAKEKDWgRGFDYW |
| | (SEQ ID NO: 378) | GQGTLVTVSS |
| | | (SEQ ID NO: 379) |
| 9F8 | DIVMTQSPLSLPVTPGEPASISCRSSQSL | EVQLVESGGGLVKPGGSLRLSCAASGF |
| | LYSNGYNYLDWYLQKPGQSPQLLIFLN | TFSNYSMNWVRQAPGKGLEWVSSISsS |
| | SNRASGVPDRFSGSGSGTDFTLKISRVE | TIYIYYADSVKGRFTISRDNAKKSLYLQ |
| | AEDVGVYFCMQALQTPLTFGGGTKVE | MNSLRAEDTAVYYCARDIGWevftLGFD |
| | IK | YWGQGTQVTVSS |
| | (SEQ ID NO: 380) | (SEQ ID NO: 381) |

Also provided herein are CDR portions of extracellular ligand-binding domains of CARs to CD7 (including Chothia, Kabat CDRs, and CDR contact regions). Determination of CDR regions is well within the skill of the art. It is understood that in some embodiments, CDRs can be a combination of the Kabat and Chothia CDR (also termed “combined CRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof. Tables 2A-2B3 provide examples of CDR sequences provided herein.

TABLE 2A
Heavy Chain

| mAb | CDRH1 | CDRH2 | CDRH3 |
|---|---|---|---|
| 31H1 | SYGFS (SEQ ID NO: 49) | GIIPIFGSANYAQKF | GGSSSPFAY (SEQ |
| | (Kabat); | QG (SEQ ID NO: 52) | ID NO: 54) |
| | GGTFSSY (SEQ ID NO: 50) | (Kabat); | |
| | (Chothia); | IPIFGS (SEQ ID NO: | |
| | GGTFSSYGFS (SEQ ID NO: | 53) (Chothia) | |
| | 51) (Extended) | | |
| 63B2 | SYGFS (SEQ ID NO: 55) | GIIPIFGTANYAQKF | GGSSSPFAY (SEQ |
| | (Kabat); | QG (SEQ ID NO: 58) | ID NO: 60) |
| | GGTFSSY (SEQ ID NO: 56) | (Kabat); | |
| | (Chothia) | IPIFGT (SEQ ID NO: | |
| | GGTFSSYGFS (Extended) | 59) (Chothia) | |
| | (SEQ ID NO: 57) | | |
| 40E3 | SYYWN (SEQ ID NO: 61) | YIYYSGSTNYNPSL | DIRTW (SEQ ID NO: |
| | (Kabat); | KS (SEQ ID NO: 64) | 66) |
| | GGSISSY (SEQ ID NO: 62) | (Kabat); | |
| | (Chothia); | YYSGS (SEQ ID NO: | |
| | GGSISSYYWN (SEQ ID NO: | 65) (Chothia) | |
| | 63) (Extended) | | |
| 42C3 | NSWMS (SEQ ID NO: 67) | NIKRDGSEKYYVDS | DQTGSFDY (SEQ ID |
| | (Kabat); | VKG (SEQ ID NO: 70) | NO: 72) |
| | GFTFRNS (SEQ ID NO: 68) | (Kabat); | |
| | (Chothia); | KRDGSE (SEQ ID | |
| | GFTFRNSWMS (SEQ ID NO: | NO: 71) (Chothia) | |
| | 69) (Extended) | | |
| 45F11 | VYYWS (SEQ ID NO: 73) | VYSSGNINYNPSLE | GLDAFDI (SEQ ID |
| | (Kabat); | S (SEQ ID NO: 76) | NO: 78) |
| | DDSISVY (SEQ ID NO: 74) | (Kabat); | |
| | (Chothia); | YSSGN (SEQ ID NO: | |
| | DDSISVYYWS (SEQ ID NO: | 77) (Chothia) | |
| | 75) (Extended) | | |
| 64F9 | SYAMS (SEQ ID NO: 79) | RVYSSGNINYNPSL | GLDAFDI (SEQ ID |
| | (Kabat); | ES (SEQ ID NO: 82) | NO: 84) |
| | GFTFTSY (SEQ ID NO: 80) | (Kabat); | |
| | (Chothia); | YSSGN (SEQ ID NO: | |
| | GFTFTSYAMS (SEQ ID NO: | 83) (Chothia) | |
| | 81) (Extended) | | |
| 72C2 | TYAIS (SEQ ID NO: 85) | GIIPFFGTANYAQKF | WELFFFDF (SEQ ID |
| | (Kabat); | QG (SEQ ID NO: 88) | NO: 90) |
| | GGTFITY (SEQ ID NO: 86) | (Kabat); | |
| | (Chothia); | IPFFGT (SEQ ID NO: | |
| | GGTFITYAIS (SEQ ID NO: | 89) (Chothia) | |
| | 87) (Extended) | | |
| 2F10 | YYSMN (SEQ ID NO: 91) | HISIRSSTIYFADSA | GSGWYGDYFDY |
| | (Kabat); | KG (SEQ ID NO: 94) | (SEQ ID NO: 96) |
| | GFTFTYY (SEQ ID NO: 92) | (Kabat); | |
| | (Chothia); | SIRSST (SEQ ID NO: | |
| | GFTFTYYSMN (SEQ ID NO: | 95) (Chothia) | |
| | 93) (Extended) | | |
| 4F11 | NARMGVT (SEQ ID NO: 97) | HIFSNDEKSYSTSLK | IRDYYDISSYYDY |
| | (Kabat); | S (SEQ ID NO: 100) | (SEQ ID NO: 102) |
| | GFSLSNARM (SEQ ID NO: | (Kabat); | |
| | 98) (Chothia); | FSNDE (SEQ ID NO: | |
| | GFSLSNARMGVT (SEQ ID | 101) (Chothia) | |
| | NO: 99) (Extended) | | |
| 10H10 | NHNIH (SEQ ID NO: 103) | YISRSSSTIYYADSV | DHAQWYGMDV |
| | (Kabat); | KG (SEQ ID NO: 106) | (SEQ ID NO: 108) |
| | GFTFSNH (SEQ ID NO: 104) | (Kabat); | |
| | (Chothia); | SRSSST (SEQ ID NO: | |
| | GFTFSNHNIH (SEQ ID NO: | 107) (Chothia) | |
| | 105) (Extended) | | |
| 17G6 | SYWMS (SEQ ID NO: 109) | SIKQDGSEKYYVDS | EGVNWGWRLYWH |
| | (Kabat); | VKG (SEQ ID NO: | FDL (SEQ ID NO: |
| | GFTFSSY (SEQ ID NO: 110) | 112) (Kabat); | 114) |
| | (Chothia); | KQDGSE (SEQ ID | |
| | GFTFSSYWMS (SEQ ID NO: | NO: 113) (Chothia) | |
| | 111) (Extended) | | |
| 65E11 | SYSMN (SEQ ID NO: 115) | HSSISRGNIYFADSV | GSGWYGDYFDY |
| | (Kabat); | KG (SEQ ID NO: 118) | (SEQ ID NO: 120) |
| | GFTFSSY (SEQ ID NO: 116) | (Kabat); | |
| | (Chothia); | SISRGN (SEQ ID NO: | |
| | GFTFSSYSMN (SEQ ID NO: | 119) (Chothia) | |
| | 117) (Extended) | | |
| P02B10 | NYAMS (SEQ ID NO: 121) | AIRGGGGSTYYADS | DFISGTWYPDY |
| | (Kabat); | VKG (SEQ ID NO: | (SEQ ID NO: 126) |
| | GFAFSNY (SEQ ID NO: 122) | 124) (Kabat); | |
| | (Chothia); | RGGGGS (SEQ ID | |
| | GFAFSNYAMS (SEQ ID NO: | NO: 125) (Chothia) | |
| | 123) (Extended) | | |
| P07D03 | SYWIG (SEQ ID NO: 127) | SIYPDDSDTRYSPSF | STVDYPGYSYFDY |
| | (Kabat); | QG (SEQ ID NO: 130) | (SEQ ID NO: 132) |
| | GYRFTSY (SEQ ID NO: 128) | (Kabat); | |
| | (Chothia); | YPDDSD (SEQ ID | |
| | GYRFTSYWIG (SEQ ID NO: | NO: 131) (Chothia) | |
| | 129) (Extended) | | |
| P08A02 | NYWIA (SEQ ID NO: 133) | IIYPDGSDTRYSPSF | DITSWYYGEPAFDI |
| | (Kabat); | QG (SEQ ID NO: 136) | (SEQ ID NO: 138) |
| | GYTFTNY (SEQ ID NO: 134) | (Kabat); | |
| | (Chothia); | YPDGSD (SEQ ID | |
| | GYTFTNYWIA (SEQ ID NO: | NO: 137) (Chothia) | |
| | 135) (Extended) | | |
| P08E02 | SSWIG (SEQ ID NO: 139) | IIYPGDSDTRYSPSF | GLSQAMTGFGFDY |
| | (Kabat); | QG (SEQ ID NO: 142) | (SEQ ID NO: 144) |
| | GYSFTSS (SEQ ID NO: 140) | (Kabat); | |
| | (Chothia); | YPGDSD (SEQ ID | |
| | GYSFTSSWIG (SEQ ID NO: | NO: 143) (Chothia) | |
| | 141) (Extended) | | |
| P08F08 | SYWIG (SEQ ID NO: 145) | IIHPDDSDTKYSPSF | SYLRGLWGGYFDY |
| | (Kabat); | QG (SEQ ID NO: 148) | (SEQ ID NO: 150) |
| | GYGFTSY (SEQ ID NO: 146) | (Kabat); | |
| | (Chothia); | HPDDSD (SEQ ID | |
| | GYGFTSYWIG (SEQ ID NO: | NO: 149) (Chothia) | |
| | 147) (Extended) | | |
| P08G02 | SSWIG (SEQ ID NO: 151) | IIYPDTSHTRYSPSF | ASYFDRGTGYSSW |
| | (Kabat); | Q (SEQ ID NO: 154) | WMDV (SEQ ID NO: |
| | GYTFPSS (SEQ ID NO: 152) | (Kabat); | 156) |
| | (Chothia); | YPDTSH (SEQ ID | |
| | GYTFPSSWIG (SEQ ID NO: | NO: 155) (Chothia) | |
| | 153) (Extended) | | |
| P12B09 | QYSMS (SEQ ID NO: 157) | AISGGGVSTYYADS | DISDSGGSHWYFD |
| | (Kabat); | VKG (SEQ ID NO: | Y (SEQ ID NO: 162) |
| | GFTFSQY (SEQ ID NO: 158) | 160) (Kabat); | |
| | (Chothia); | SGGGVS (SEQ ID | |
| | GFTFSQYSMS (SEQ ID NO: | NO: 161) (Chothia) | |
| | 159) (Extended) | | |
| P12F02 | SYAMS (SEQ ID NO: 163) | TISGTGGTTYYADS | VRAGIDPTASDV |
| | (Kabat); | VKG (SEQ ID NO: | (SEQ ID NO: 168) |
| | GFTFSSY (SEQ ID NO: 164) | 166) (Kabat); | |
| | (Chothia); | SGTGGT (SEQ ID | |
| | GFTFSSYAMS (SEQ ID NO: | NO: 167) (Chothia) | |
| | 165) (Extended) | | |
| P12G07 | NFAMS (SEQ ID NO: 169) | GISGSGDNTYYADS | DRDIGLGWYSYYL |
| | (Kabat); | VKG (SEQ ID NO: | DV (SEQ ID NO: 174) |
| | GFTFNNF (SEQ ID NO: 170) | 172) (Kabat); | |
| | (Chothia); | SGSGDN (SEQ ID | |
| | GFTFNNFAMS (SEQ ID NO: | NO: 173) (Chothia) | |
| | 171) (Extended) | | |
| P13F04 | SYAIS (SEQ ID NO: 175) | EIIPIFGTASYAQKF | AGWDDSWFDY |
| | (Kabat); | QG (SEQ ID NO: 178) | (SEQ ID NO: 180) |
| | GGTFSSY (SEQ ID NO: 176) | (Kabat); | |
| | (Chothia); | IPIFGT (SEQ ID NO: | |
| | GGTFSSYAIS (SEQ ID NO: | 179) (Chothia) | |
| | 177) (Extended) | | |
| P15D02 | SYWIG (SEQ ID NO: 181) | VIYPGTSETRYSPSF | GLSASASGYSFQY |
| | (Kabat); | QG (SEQ ID NO: 184) | (SEQ ID NO: 186) |
| | GYSFASY (SEQ ID NO: 182) | (Kabat); | |
| | (Chothia); | YPGTSE (SEQ ID | |
| | GYSFASYWIG (SEQ ID NO: | NO: 185) (Chothia) | |
| | 183) (Extended) | | |
| P16C05 | DYWIG (SEQ ID NO: 187) | MISPGGSTTIYRPSF | MYTGGYGGSWYF |
| | (Kabat); | QG (SEQ ID NO: 190) | DY (SEQ ID NO: 192) |
| | GYSFTDY (SEQ ID NO: 188) | (Kabat); | |
| | (Chothia); | SPGGST (SEQ ID NO: | |
| | GYSFTDYWIG (SEQ ID NO: | 191) (Chothia) | |
| | 189) (Extended) | | |
| 10A1 | YYYWT (SEQ ID NO: 382) | HIYYSGSTNYNPSL | AEGSIDAFDF (SEQ |
| | (Kabat); | KS (SEQ ID NO: 385) | ID NO: 387) |
| | GGSISYY (SEQ ID NO: 383) | (Kabat); | |
| | (Chothia); | YYSGS (SEQ ID NO: | |
| | GGSISYYYWT (SEQ ID NO: | 386) (Chothia) | |
| | 384) (Extended) | | |
| 10E2 | SNYMT (SEQ ID NO: 388) | VIYSGGSTYYADSV | NWGDYW (SEQ ID |
| | (Kabat); | KG (SEQ ID NO: 391) | NO: 393) |
| | GFTVSSN (SEQ ID NO: 389) | (Kabat); | |
| | (Chothia); | YSGGS (SEQ ID NO: | |
| | GFTVSSNYMT (SEQ ID NO: | 392) (Chothia) | |
| | 390) (Extended) | | |
| 11A1 | YYFWN (SEQ ID NO: 394) | HVYDIGNTKYNPSL | GEGAIDAFDI (SEQ |
| | (Kabat); | KS (SEQ ID NO: 397) | ID NO: 399) |
| | GGSIDYY (SEQ ID NO: 395) | (Kabat); | |
| | (Chothia); | YDIGN (SEQ ID NO: | |
| | GGSIDYYFWN (SEQ ID NO: | 398) (Chothia) | |
| | 396) (Extended) | | |
| 11C1 | YYYWT (SEQ ID NO: 400) | HVIYSGTTNYNPSL | AEGSIDAFDL (SEQ |
| | (Kabat); | KS (SEQ ID NO: 403) | ID NO: 405) |
| | GGSISYY (SEQ ID NO: 401) | (Kabat); | |
| | (Chothia); | IYSGT (SEQ ID NO: | |
| | GGSISYYYWT (SEQ ID NO: | 404) (Chothia) | |
| | 402) (Extended) | | |
| 11D1 | DYGH (SEQ ID NO: 406) | VIWYDGSiKKYSDS | DEVGtfGAFDF (SEQ |
| | (Kabat); | VKG (SEQ ID NO: | ID NO: 411) |
| | GFTFSDY (SEQ ID NO: 407) | 409) (Kabat); | |
| | (Chothia); | WYDGSi (SEQ ID | |
| | GFTFSDYGIH (SEQ ID NO: | NO: 410) (Chothia) | |
| | 408) (Extended) | | |
| 11E1 | SdgYYWS (SEQ ID NO: 412) | YMYYSGSTYYNPS | DFGWYFDL (SEQ |
| | (Kabat); | LKS (SEQ ID NO: | ID NO: 417) |
| | GGSISSdgY (SEQ ID NO: | 415) (Kabat); | |
| | 413) (Chothia); | YYSGS (SEQ ID NO: | |
| | GGSISSdgYYWS (SEQ ID | 416) (Chothia) | |
| | NO: 414) (Extended) | | |
| 12A2 | SdgYYWS (SEQ ID NO: 418) | YIYYRRITDYNPSL | DFGWYFDL (SEQ |
| | (Kabat); | KS (SEQ ID NO: 421) | ID NO: 423) |
| | GGSVSSdgY (SEQ ID NO: | (Kabat); | |
| | 419) (Chothia); | YYRRI (SEQ ID NO: | |
| | GGSVSSdgYYWS (SEQ ID | 422) (Chothia) | |
| | NO: 420) (Extended) | | |
| 12C4 | GYYLH (SEQ ID NO: 424) | WINpNSGGTNYAQ | DRGVtmivDGMDD |
| | (Kabat); | KFQG (SEQ ID NO: | (SEQ ID NO: 429) |
| | GYTFTGY (SEQ ID NO: 425) | 427) (Kabat); | |
| | (Chothia); | NpNSGG (SEQ ID | |
| | GYTFTGYYLH (SEQ ID NO: | NO: 428) (Chothia) | |
| | 426) (Extended) | | |
| 12C5 | DYGMH (SEQ ID NO: 430) | VIWYDGSnKYYAD | DEVGfVGAFDI (SEQ |
| | (Kabat); | SVKG (SEQ ID NO: | ID NO: 435) |
| | GFTFSDY (SEQ ID NO: 431) | 433) (Kabat); | |
| | (Chothia); | WYDGSn (SEQ ID | |
| | GFTFSDYGMH (SEQ ID NO: | NO: 434) (Chothia) | |
| | 432) (Extended) | | |
| 12C6 | DYGMH (SEQ ID NO: 607) | VIWYDGSnKYYAD | DEVGfVGAFDI (SEQ |
| | (Kabat); | SVKG (SEQ ID NO: | ID NO: 612) |
| | GFTFSDY (SEQ ID NO: 608) | 610) (Kabat); | |
| | (Chothia); | WYDGSn (SEQ ID | |
| | GFTFSDYGMH (SEQ ID NO: | NO: 611) (Chothia) | |
| | 609) (Extended) | | |
| 12D3 | SdgYYWS (SEQ ID NO: 436) | YMYYSGITYHNPSL | DFGWYFDL (SEQ |
| | (Kabat); | KS (SEQ ID NO: 439) | ID NO: 441) |
| | GGSISSdgY (SEQ ID NO: | (Kabat); | |
| | 437) (Chothia); | YYSGI (SEQ ID NO: | |
| | GGSISSdgYYWS (SEQ ID | 440) (Chothia) | |
| | NO: 438) (Extended) | | |
| 12D6 | SdaYYWS (SEQ ID NO: 442) | YMYYSGITYYNPSL | DFGWYFDL (SEQ |
| | (Kabat); | KS (SEQ ID NO: 445) | ID NO: 447) |
| | GGSISSdaY (SEQ ID NO: | (Kabat); | |
| | 443) (Chothia); | YYSGI (SEQ ID NO: | |
| | GGSISSdaYYWS (SEQ ID | 446) (Chothia) | |
| | NO: 444) (Extended) | | |
| 12D7 | DYGH (SEQ ID NO: 448) | VIWYDGSiKKYSDS | DEVGtfGAFDF (SEQ |
| | (Kabat); | VKG (SEQ ID NO: | ID NO: 453) |
| | GFTFSDY (SEQ ID NO: 449) | 451) (Kabat); | |
| | (Chothia); | WYDGSi (SEQ ID | |
| | GFTFSDYGIH (SEQ ID NO: | NO: 452) (Chothia) | |
| | 450) (Extended) | | |
| 12F5 | NAWMS (SEQ ID NO: 454) | RIKsktGGGTTDYAA | LIVGaiSLFDY (SEQ |
| | (Kabat); | PVKG (SEQ ID NO: | ID NO: 459) |
| | GFTFSNA (SEQ ID NO: 455) | 457) (Kabat); | |
| | (Chothia); | KsktGGGT (SEQ ID | |
| | GFTFSNAWMS (SEQ ID NO: | NO: 458) (Chothia) | |
| | 456) (Extended) | | |
| 12H4 | YYFWT (SEQ ID NO: 460) | QIYYSGNTNSNPSL | AEGSIDAFDI (SEQ |
| | (Kabat); | KS (SEQ ID NO: 463) | ID NO: 465) |
| | GGSISYY (SEQ ID NO: 461) | (Kabat); | |
| | (Chothia); | YYSGN (SEQ ID NO: | |
| | GGSISYYFWT (SEQ ID NO: | 464) (Chothia) | |
| | 462) (Extended) | | |
| 8C8 | SYSMN (SEQ ID NO: 466) | SIStSSNYIHYADSL | DKGTtltnWYFDL |
| | (Kabat); | QG (SEQ ID NO: 469) | (SEQ ID NO: 471) |
| | GFTFSSY (SEQ ID NO: 467) | (Kabat); | |
| | (Chothia); | StSSNY (SEQ ID NO: | |
| | GFTFSSYSMN (SEQ ID NO: | 470) (Chothia) | |
| | 468) (Extended) | | |
| 8F7 | SYGMH (SEQ ID NO: 472) | VIWYDGSnKYYAD | DGYSgssDAFDI |
| | (Kabat); | SLKG (SEQ ID NO: | (SEQ ID NO: 477) |
| | GFTFSSY (SEQ ID NO: 473) | 475) (Kabat); | |
| | (Chothia); | WYDGSn (SEQ ID | |
| | GFTFSSYGMH (SEQ ID NO: | NO: 476) (Chothia) | |
| | 474) (Extended) | | |
| 8F8 | YYYWS (SEQ ID NO: 478) | NINYMGNTIYNPSL | AEGSIDAFDF (SEQ |
| | (Kabat); | KS (SEQ ID NO: 481) | ID NO: 483) |
| | GGSISYY (SEQ ID NO: 479) | (Kabat); | |
| | (Chothia); | NYMGN (SEQ ID | |
| | GGSISYYYWS (SEQ ID NO: | NO: 482) (Chothia) | |
| | 480) (Extended) | | |
| 9D8 | GYYIY (SEQ ID NO: 484) | WINpSSGGTNYAQK | DRKReyyynFGMDV |
| | (Kabat); | FQG (SEQ ID NO: | (SEQ ID NO: 489) |
| | GYIFTGY (SEQ ID NO: 485) | 487) (Kabat); | |
| | (Chothia); | NpSSGG (SEQ ID | |
| | GYIFTGYYIY (SEQ ID NO: | NO: 488) (Chothia) | |
| | 486) (Extended) | | |
| 9E10 | SHYIY (SEQ ID NO: 490) | WINpNSGGTNYAQ | DRKReyyynFGMDV |
| | (Kabat); | KFQD (SEQ ID NO: | (SEQ ID NO: 495) |
| | GYTFTSH (SEQ ID NO: 491) | 493) (Kabat); | |
| | (Chothia); | NpNSGG (SEQ ID | |
| | GYTFTSHYIY (SEQ ID NO: | NO: 494) (Chothia) | |
| | 492) (Extended) | | |
| 9E5 | SHYIY (SEQ ID NO: 496) | WINpNSGGTKYAQ | DRKReyyynFGMDV |
| | (Kabat); | KFQD (SEQ ID NO: | (SEQ ID NO: 501) |
| | GFTFTSH (SEQ ID NO: 497) | 499) (Kabat); | |
| | (Chothia); | NpNSGG (SEQ ID | |
| | GFTFTSHYIY (SEQ ID NO: | NO: 500) (Chothia) | |
| | 498) (Extended) | | |
| 9F4 | IYAIH (SEQ ID NO: 502) | SFGgRGSSTYFADS | EKDWgRGFDY |
| | (Kabat); | VKG (SEQ ID NO: | (SEQ ID NO: 507) |
| | GFTLSIY (SEQ ID NO: 503) | 505) (Kabat); | |
| | (Chothia); | GgRGSS (SEQ ID NO: | |
| | GFTLSIYAIH (SEQ ID NO: | 506) (Chothia) | |
| | 504) (Extended) | | |
| 9F8 | NYSMN (SEQ ID NO: 508) | SISsSTIYIYYADSVK | DIGWevftLGFDY |
| | (Kabat); | G (SEQ ID NO: 511) | (SEQ ID NO: 513) |
| | GFTFSNY (SEQ ID NO: 509) | (Kabat); | |
| | (Chothia); | SsSTIY (SEQ ID NO: | |
| | GFTFSNYSMN (SEQ ID NO: | 512) (Chothia) | |
| | 510) (Extended) | | |

TABLE 2B
Light Chain

| mAb | CDRL1 | CDRL2 | CDRL3 |
|---|---|---|---|
| 31H1 | RSSQSLVHSDGNTYLS | KISNRFS (SEQ ID | MQATQFPLT (SEQ |
| | (SEQ ID NO: 193); | NO: 194) | ID NO: 195) |
| 63B2 | RSSQSLVHSDGNTYLS | KISNRFS (SEQ ID | MQATQFPLT (SEQ |
| | (SEQ ID NO: 196); | NO: 197) | ID NO: 198) |
| 40E3 | RASQGISNYLA (SEQ ID | AASSLQS (SEQ ID | QQYNSYPLT (SEQ |
| | NO: 199); | NO: 200) | ID NO: 201) |
| 42C3 | RSSQSLVYSDENTYLN | QVSNRDS (SEQ ID | MQGTYWPPT (SEQ |
| | (SEQ ID NO: 202); | NO: 203) | ID NO: 204) |
| 45F11 | RASQSVSSSLA (SEQ ID | GASTRAT (SEQ ID | QQYINWPH (SEQ |
| | NO: 205); | NO: 206) | ID NO: 207) |
| 64F9 | QASQDISNYLN (SEQ ID | GASNLET (SEQ ID | QQYDNFPIT (SEQ |
| | NO: 208); | NO: 209) | ID NO: 210) |
| 72C2 | RASQSVSSNLA (SEQ ID | SASTRAS (SEQ ID | QQYDNWPPLT |
| | NO: 211); | NO: 212) | (SEQ ID NO: 213) |
| 2F10 | RASQSVSSSYLA (SEQ ID | GASSRAT (SEQ ID | QQYGSSPLT (SEQ |
| | NO: 214); | NO: 215) | ID NO: 216) |
| 4F11 | RASQDISNYLA (SEQ ID | AASSLQS (SEQ ID | LQLNSFPFT (SEQ ID |
| | NO: 217); | NO: 218) | NO: 219) |
| 10H10 | RASQGISSWLA (SEQ ID | AASSLQS (SEQ ID | QQAFSFPFT (SEQ |
| | NO: 220); | NO: 221) | ID NO: 222) |
| 17G6 | KSSQSVLYSYNNKNYVA | WASTRES (SEQ ID | QQYYSTLT (SEQ |
| | (SEQ ID NO: 223); | NO: 224) | ID NO: 225) |
| 65E11 | RASQSVSSSYLA (SEQ ID | DASSRAT (SEQ ID | QQYGSSPLT (SEQ |
| | NO: 226); | NO: 227) | ID NO: 228) |
| P02B10 | SGSSSNIGSNYVY (SEQ ID | RNNQRPS (SEQ ID | AAWDDSLSGVV |
| | NO: 229); | NO: 230) | (SEQ ID NO: 231) |
| P07D03 | SGSRSNIGSNYVY (SEQ ID | RNNQRPS (SEQ ID | ASWDGSLSAVV |
| | NO: 232); | NO: 233) | (SEQ ID NO: 234) |
| P08A02 | SGSSSNIGSNYVY (SEQ ID | RNNQRPS (SEQ ID | ATWDDSLGSPV |
| | NO: 235); | NO: 236) | (SEQ ID NO: 237) |
| P08E02 | RASQSISRYLN (SEQ ID NO: | AASILQT (SEQ ID | QQSYSTTMWT |
| | 238); | NO: 239) | (SEQ ID NO: 240) |
| P08F08 | SGSSSNIGSNYVN (SEQ ID | GDYQRPS (SEQ ID | ATRDDSLSGSVV |
| | NO: 241); | NO: 242) | (SEQ ID NO: 243) |
| P08G02 | RASQSIYDYLH (SEQ ID | DASNLQS (SEQ ID | QQSYTTPLFT (SEQ |
| | NO: 244); | NO: 245) | ID NO: 246) |
| P12B09 | RASQYIGRYLN (SEQ ID | GATSLAS (SEQ ID | QQSYSTTSPT (SEQ |
| | NO: 247); | NO: 248) | ID NO: 249) |
| P12F02 | SGSTSNIGRNYVY (SEQ ID | RTNQRPS (SEQ ID | AAWDDSLSGRV |
| | NO: 250); | NO: 251) | (SEQ ID NO: 252) |
| P12G07 | SGSSSNIGSNYVY (SEQ ID | MNNQRPS (SEQ ID | AAWDDSLSAVV |
| | NO: 253); | NO: 254) | (SEQ ID NO: 255) |
| P13F04 | SGSNSNIGTNYVS (SEQ ID | RSSRRPS (SEQ ID | AAWDGSLSGHWV |
| | NO: 256); | NO: 257) | (SEQ ID NO: 258) |
| P15D02 | RASQSIDTYLN (SEQ ID NO: | SASSLHS (SEQ ID | QQSYSTTAWT |
| | 259); | NO: 260) | (SEQ ID NO: 261) |
| P16C05 | RASQSIGQSLN (SEQ ID NO: | GASSLQS (SEQ ID | QQSYSTPIT (SEQ ID |
| | 262); | NO: 263) | NO: 264) |
| 10A1 | RASQSISTWLA (SEQ ID | KASSLES (SEQ ID | QQYKSYSHT (SEQ |
| | NO: 514); | NO: 515) | ID NO: 516) |
| 10E2 | RASQSISSWLA (SEQ ID NO: | KASSLES (SEQ ID | QQYKSFSLT (SEQ |
| | 517); | NO: 518) | ID NO: 519) |
| 11A1 | RASQSISSWLA (SEQ ID NO: | KASTLES (SEQ ID | QQYNSYSYT (SEQ |
| | 520); | NO: 521) | ID NO: 522) |
| 11C1 | RASQSVSSWLA (SEQ ID | KASSLES (SEQ ID | QQYNTYSHT (SEQ |
| | NO: 523); | NO: 524) | ID NO: 525) |
| 11D1 | RASQGIRNDLG (SEQ ID | AASSLQS (SEQ ID | LQDYNYPFT (SEQ |
| | NO: 526); | NO: 527) | ID NO: 528) |
| 11E1 | RASQDIDNYLA (SEQ ID | AASALQS (SEQ ID | QNYNSGPRT (SEQ |
| | NO: 529); | NO: 530) | ID NO: 531) |
| 12A2 | RASQDISNYLT (SEQ ID NO: | AASALQS (SEQ ID | QNYNSAPRT (SEQ |
| | 532); | NO: 533) | ID NO: 534) |
| 12C4 | RSSQSLLHSNGYNYLD | LGSNRAS (SEQ ID | MQTLQTPFT (SEQ |
| | (SEQ ID NO: 535); | NO: 536) | ID NO: 537) |
| 12C5 | RASQGINSHLA (SEQ ID | YASTLPS (SEQ ID | QQLNHYPIT (SEQ |
| | NO: 538); | NO: 539) | ID NO: 540) |
| 12C6 | RASQGINSHLA (SEQ ID | YASTLPS (SEQ ID | QQLNHYPIT (SEQ |
| | NO: 613); | NO: 614) | ID NO: 615) |
| 12D3 | RASQGISNYLA (SEQ ID | AASTLHS (SEQ ID | QKYNSAPRT (SEQ |
| | NO: 541); | NO: 542) | ID NO: 543) |
| 12D6 | RASQDISNYLA (SEQ ID | AASTLHS (SEQ ID | QKYNSAPRT (SEQ |
| | NO: 544); | NO: 545) | ID NO: 546) |
| 12D7 | RASQDISSFLA (SEQ ID NO: | VASTLQS (SEQ ID | QQLHVYPIT (SEQ |
| | 547); | NO: 548) | ID NO: 549) |
| 12F5 | RSSQSLLDSDDGNTYLD | TLSYRAS (SEQ ID | MQRIEFPFT (SEQ ID |
| | (SEQ ID NO: 550); | NO: 551) | NO: 552) |
| 12H4 | RASQTISTWLA (SEQ ID | KASNLES (SEQ ID | QQYQTFSHT (SEQ |
| | NO: 553); | NO: 554) | ID NO: 555) |
| 8C8 | RASQGISNYLA (SEQ ID | AASTLQS (SEQ ID | QKYNSAPLT (SEQ |
| | NO: 556); | NO: 557) | ID NO: 558) |
| 8F7 | RSSQTLVHSNGYNYLN | LGSNRAS (SEQ ID | MQAIQTPYT (SEQ |
| | (SEQ ID NO: 559); | NO: 560) | ID NO: 561) |
| 8F8 | RASQSISSWLA (SEQ ID NO: | KASNLES (SEQ ID | QQYNSYSCT (SEQ |
| | 562); | NO: 563) | ID NO: 564) |
| 9D8 | QASQDINNYLH (SEQ ID | DASDWET (SEQ ID | QQYDHLPIT (SEQ |
| | NO: 565); | NO: 566) | ID NO: 567) |
| 9E10 | QASQDISNYLH (SEQ ID | DASDLET (SEQ ID | QQYDHLPIT (SEQ |
| | NO: 568); | NO: 569) | ID NO: 570) |
| 9E5 | QASQDISNYLH (SEQ ID | DASDLET (SEQ ID | QQYDHLPIT (SEQ |
| | NO: 571); | NO: 572) | ID NO: 573) |
| 9F4 | QASQDISNYLN (SEQ ID | DASNLET (SEQ ID | QQYDNLPYT (SEQ |
| | NO: 574); | NO: 575) | ID NO: 576) |
| 9F8 | RSSQSLLYSNGYNYLD | LNSNRAS (SEQ ID | MQALQTPLT (SEQ |
| | (SEQ ID NO: 577); | NO: 578) | ID NO: 579) |

The disclosure encompasses modifications to the CARs and polypeptides comprising the sequences shown in Tables 1 or 2A-2B, including functionally equivalent CARs having modifications which do not significantly affect their properties and variants which have enhanced or decreased activity and/or affinity. For example, the amino acid sequence may be mutated to obtain an antibody with the desired binding affinity to CD70. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 3 under the heading of “conservative substitutions.” If such substitutions result in a change in biological activity, then more substantial changes, denominated “exemplary substitutions” in Table 3, or as further described below in reference to amino acid classes, may be introduced and the products screened.

TABLE 3
Amino Acid Substitutions

| Original Residue | | |
|---|---|---|
| (naturally occurring | Conservative | Exemplary |
| amino acid) | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp: Phe: Thr; Ser |
| Val (V) | Leu | Ile: Leu: Met; Phe: Ala; |
| | | Norleucine |

In some embodiments, the disclosure provides a CD70-binding protein, e.g., a CD70 CAR comprising an extracellular ligand-binding domain that binds to CD70 and competes for binding to CD70 with a CAR described herein, including CAR comprising an extracellular domain comprising an ScFv comprising the sequences of 31H1, 63B2, 40E3, 42C3, 45F11, 64F9, 72C2, 2F10, 4F11, 10H10, 17G6, 65E11, P02B10, P07D03, P08A02, P08E02, P08F08, P08G02, P12B09, P12F02, P12G07, P13F04, P15D02, P16C05, 10A1, 10E2, 11A1, 11C1, 11D1, IFE1, 12A2, 12C4, 12C5, 12D3, 12D6, 12D7, 12F5, 12H4, 8C8, 8F7, 8F8, 9D8, 9E10, 9E5, 9F4 or 9F8.

In some embodiments, the extracellular ligand-binding domain that binds to CD70 (or the CD70 binding domain) comprises an scFv comprising the amino acid sequence of SEQ ID NO: 599 or an antibody, optionally as an scFv, that competes for binding to CD70 with the scFv comprising the amino acid sequence SEQ ID NO: 599. In some embodiments, the extracellular ligand-binding domain that binds to CD70 (or the CD70 binding domain) comprises an scFv comprising the amino acid sequence of SEQ ID NO: 600 or an scFv that competes for binding to CD70 with the scFv comprising the amino acid sequence of SEQ ID NO: 600. In some embodiments, the extracellular ligand-binding domain that binds to CD70 (or the CD70 binding domain) comprises an scFv comprising the amino acid sequences of SEQ ID NOs: 370 and 371 or an scFv that competes for binding to CD70 with the scFv comprising the amino acid sequence of SEQ ID NOs: 370 and 371. Methods for determining binding competition are known in the art, including, for example, ELISA or Biacore SPR assay.

In some embodiments, the CD70 binding domain comprises an anti-CD70 antibody that binds to the membrane distal site of CD70. In some embodiments, the CD70 binding domain comprises an anti-CD70 antibody that binds to the membrane proximal site of CD70. The CD70 structure analysis can be found, e.g., Liu et al., 2021, J. Biol. Chem. Structural delineation and phase-dependent activation of the costimulatory CD27:CD70 complex. 297(4):101102.

In some embodiments, the disclosure provides a CAR, which specifically binds to CD70, wherein the CAR comprises a VH region comprising a sequence shown in SEQ ID NO: 20; and/or a VL region comprising a sequence shown in SEQ ID NO: 19. In some embodiments, the disclosure provides a CAR, which specifically binds to CD70, wherein the CAR comprises a VH region comprising a sequence shown in SEQ ID NO: 22; and/or a VL region comprising a sequence shown in SEQ ID NO: 21. In some embodiments, the disclosure provides a CAR, which specifically binds to CD70, wherein the CAR comprises a VH region comprising a sequence shown in SEQ ID NO: 28; and/or a VL region comprising a sequence shown in SEQ ID NO: 27. In some embodiments, the disclosure provides a CAR, which specifically binds to CD70, wherein the CAR comprises a VH region comprising a sequence shown in SEQ ID NO: 36; and/or a VL region comprising a sequence shown in SEQ ID NO: 35. In some embodiments, the disclosure provides a CAR, which specifically binds to CD70, wherein the CAR comprises a VH region comprising a sequence shown in SEQ ID NO: 46; and/or a VL region comprising a sequence shown in SEQ ID NO: 45. In some embodiments, the disclosure provides a CAR, which specifically binds to CD70, wherein the CAR comprises a VH region comprising a sequence shown in SEQ ID NO: 18; and/or a VL region comprising a sequence shown in SEQ ID NO: 17. In some embodiments, the disclosure provides a CAR, which specifically binds to CD70, wherein the CAR comprises a VH region comprising a sequence shown in SEQ ID NO: 34; and/or a VL region comprising a sequence shown in SEQ ID NO: 33. In some embodiments, the disclosure also provides CARs comprising CDR portions of antibodies to CD70 antibodies based on CDR contact regions. CDR contact regions are regions of an antibody that imbue specificity to the antibody for an antigen. In general, CDR contact regions include the residue positions in the CDRs and Vernier zones which are constrained in order to maintain proper loop structure for the antibody to bind a specific antigen. See, e.g., Makabe et al., J. Biol. Chem., 283:1156-1166, 2007. Determination of CDR contact regions is well within the skill of the art.

The binding affinity (KD) of the ligand binding domain of the CD70 specific CAR as described herein to CD70 (such as human CD70) can be for example about 0.1 to about 1000 nM, for example between about 0.5 nM to about 500 nM, or for example between about lnM to about 250 nM. In some embodiments, the binding affinity is about any of 1000 nm, 750 nm, 500 nm, 400 nm, 300 nm, 250 nm, 200 nM, 100 nM, 90 nM, 80 nM, 70 nM, 60 nM, 50 nM, 45 nM, 40 nM, 35 nM, 30 nM, 25 nM, 20 nM, 19 nm, 18 nm, 17 nm, 16 nm, 15 nM, 10 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM or 0.1 nM.

In some embodiments, the binding affinity (KD) of the scFv of the ligand binding domain of the CD70-specific CAR as described herein to CD70 is about 10 nM to about 100 nM, about 10 nM to about 90 nM, about 10 nM to about 80 nM, about 20 nM to about 70 nM, about 25 nM to about 75 nM, or about 40 nM to about 110 nM. In some embodiments, the binding affinities of the scFv described in this paragraph are for human CD70.

In some embodiments, the binding affinity is less than about any of 1000 nm, 900 nm, 800 nm, 250 nM, 200 nM, 100 nM, 50 nM, 30 nM, 20 nM, 10 nM, 7.5 nM, 7 nM, 6.5 nM, 6 nM, 5 nM.

The intracellular signaling domain of a CAR according to the disclosure is responsible for intracellular signaling following the binding of extracellular ligand-binding domain to the target resulting in the activation of the immune cell and immune response. The intracellular signaling domain has the ability to activate of at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the disclosure can include as non limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3ζ signaling domain which has amino acid sequence with at least about 70%, at least 80%, at least 90%, 95%, 97%, or 99% sequence identity with an amino acid sequence shown in SEQ ID NO: 272 or 617. In some embodiments the intracellular signaling domain of a CAR of the disclosure comprises a domain of a co-stimulatory molecule.

In some embodiments, the intracellular signaling domain of a CAR of the disclosure comprises a part of co-stimulatory molecule selected from the group consisting of fragment of 41BB (GenBank: AAA53133.) and CD28 (NP_006130.1). In some embodiments, the intracellular signaling domain of the CAR of the disclosure comprises amino acid sequence which comprises at least 70%, at least 80%, at least 90%, 95%, 97%, or 99% sequence identity with an amino acid sequence shown in SEQ ID NO: 271 or 616. In some embodiments, the intracellular signaling domain of the CAR of the disclosure comprises amino acid sequence which comprises at least 70%, at least 80%, at least 90%, 95%, 97%, or 99% sequence identity with an amino acid sequence shown in SEQ ID NO: 271 or 616 and/or at least 70%, at least 80%, at least 90%, 95%, 97%, or 99% sequence identity with an amino acid sequence shown in SEQ ID NO: 276.

CARs are expressed on the surface membrane of the cell. Thus, the CAR can comprise a transmembrane domain. Suitable transmembrane domains for a CAR disclosed herein have the ability to (a) be expressed at the surface of a cell, which is in some embodiments an immune cell such as, for example without limitation, a lymphocyte cell, such as a T helper (Tₕ) cell, cytotoxic T (Tc) cell, T regulatory (T_{reg}) cell, or Natural killer (NK) cells, and/or (b) interact with the ligand-binding domain and intracellular signaling domain for directing cellular response of an immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subsequence or subunit of the T cell receptor such as α, β, γ or δ, polypeptide constituting CD3 complex, IL-2 receptor p55 (a chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1). The transmembrane domain can further comprise a stalk domain between the extracellular ligand-binding domain and said transmembrane domain. A stalk domain may comprise up to 300 amino acids—in some embodiments 10 to 100 amino acids or in some embodiments 25 to 50 amino acids. Stalk region may be derived from all or part of naturally occurring molecules, such as from all or part of the extracellular region of CD8, CD4, CD28, 4-1BB, or IgG (in particular, the hinge region of an IgG), or from all or part of an antibody heavy-chain constant region. Alternatively the stalk domain may be a synthetic sequence that corresponds to a naturally occurring stalk sequence, or may be an entirely synthetic stalk sequence. In some embodiments said stalk domain is a part of human CD8α chain (e.g., NP_001139345.1). In another particular embodiment, said hinge and transmembrane domains comprise a part of human CD8α chain, which in some embodiments comprises at least 70%, at least 80%, at least 90%, 95% 97%, or 99% sequence identity with amino acid sequence selected from the group consisting of SEQ ID NO: 268 and 270. In some embodiments, the stalk domain of CARs described herein comprises a subsequence of CD8a, an IgG1, or an FcγRIIIα, in particular the hinge region of any of an CD8α, an IgG1, or an FcγRIIIα. In some embodiments, the stalk domain comprises a human CD8α hinge, a human IgG1 hinge, or a human FcγRIIIα hinge In some embodiments, CARs disclosed herein can comprise an extracellular ligand-binding domain that specifically binds CD70. In some embodiments the CARs disclosed herein comprise an scFv, CD8α human hinge and transmembrane domains, the CD3ζ signaling domain, and 4-1BB signaling domain.

Table 4 provides exemplary sequences of domains which can be used in the CARs disclosed herein.

TABLE 4
Other exemplary sequences

| | | SEQ ID |
|---|---|---|
| Domain | Amino Acid Sequence | NO: |
| CD8α signal peptide | MALPVTALLLPLALLLHAARP | 266 |
| FcγRIIIα hinge | GLAVSTISSFFPPGYQ | 267 |
| CD8α hinge | TTTPAPRPPTPAPTIASQPLSLRPEACRPAAGGAVHTRGLD | 268 |
| | FACD | |
| IgG1 hinge | EPKSPDKTHTCPPCPAPPVAGPSVFLFPPKPKDTLMIARTP | 269 |
| | EVTCVVVDVSHEDPEVKFNWYVDGVEVHNAKTKPREEQYNS | |
| | TYRVVSVLTVLHQDWLNGKEYKCKVSNKALPAPIEKTISKA | |
| | KGQPREPQVYTLPPSRDELTKNQVSLTCLVKGFYPSDIAVE | |
| | WESNGQPENNYKTTPPVLDSDGSFFLYSKLTVDKSRWQQGN | |
| | VFSCSVMHEALHNHYTQKSLSLSPGK | |
| CD8α | IYIWAPLAGTCGVLLLSLVITLYC | 270 |
| transmembrane | | |
| (TM) domain | | |
| 41BB intracellular | KRGRKKLLYIFKQPFMRPVQTTQEEDGCSCRFPEEEEGGC | 271 |
| signaling domain | EL | |
| (ISD) | | |
| 41BB intracellular | GRKKLLYIFKQPFMRPVQTTQEEDGCSCRFPEEEEGGCE | 616 |
| signaling domain | L | |
| (ISD) | | |
| CD3ζ intracellular | RVKFSRSADAPAYQQGQNQLYNELNLGRREEYDVLDKRR | 272 |
| signaling domain | GRDPEMGGKPRRKNPQEGLYNELQKDKMAEAYSEIGMK | |
| (ISD) | GERRRGKGHDGLYQGLSTATKDTYDALHMQALPPR | |
| CD3ζ intracellular | RVKFSRSADAPAYKQGQNQLYNELNLGRREEYDVLDKRR | 617 |
| signaling domain | GRDPEMGGKPRRKNPQEGLYNELQKDKMAEAYSEIGMK | |
| (ISD) | GERRRGKGHDGLYQGLSTATKDTYDALHMQALPPR | |
| FcϵRI α-TM-IC | FFIPLLVVILFAVDTGLFISTQQQVTFLLKIKRTRKGFRL | 273 |
| (FcϵRI α chain | LNPHPKPNPKNN | |
| transmembrane and | | |
| intracellular | | |
| domain) | | |
| FcϵRIβ-ΔITAM | MDTESNRRANLALPQEPSSVPAFEVLEISPQEVSSGRLLKS | 274 |
| (FcϵRI β chain | ASSPPLHTWLTVLKKEQEFLGVTQILTAMICLCFGTVVCS | |
| without ITAM) | VLDISHIEGDIFSSFKAGYPFWGAIFFSISGMLSIISERR | |
| | NATYLVRGSLGANTASSIAGGTGITILIINLKKSLAYIHI | |
| | HSCQKFFETKCFMASFSTEIVVMMLFLTILGLGSAVSLTI | |
| | CGAGEELKGNKVPE | |
| CD28-IC (CD28 co- | RSKRSRGGHSDYMNMTPRRPGPTRKHYQPYAPPRDFAAY | 276 |
| stimulatory domain) | RS | |
| FcϵRIγ-SP (signal | MIPAVVLLLLLLVEQAAA | 277 |
| peptide) | | |
| FcϵRI γ-ΔITAM | LGEPQLCYILDAILFLYGIVLTLLYCRLKIQVRKAAITSYEK | 278 |
| (FcϵRI γ chain | S | |
| without ITAM) | | |
| GSG-P2A (GSG- | GSGATNFSLLKQAGDVEENPGP | 279 |
| P2A ribosomal skip | | |
| polypeptide) | | |
| GSG-T2A (GSG- | GSGEGRGSLLTCGDVEENPGP | 280 |
| T2A ribosomal skip | | |
| polypeptide) | | |
| CD3zeta zeta | RVKFSRSADAPAYQQGQNQLYNELNLGRREEYDVLDKRR | 265 |
| concatenated | GRDPEMGGKPRRKNPQEGLYNELQKDKMAEAYSEIGMK | |
| cytoplasmic domain | GERRRGKGHDGLYQGLSTATKDTYDALHMQALPPRGRV | |
| | KFSRSADAPAYQQGQNQLYNELNLGRREEYDVLDKRRGR | |
| | DPEMGGKPRRKNPQEGLYNELQKDKMAEAYSEIGMKGE | |
| | RRRGKGHDGLYQGLSTATKDTYDALHMQALPPR | |
| CD3 delta | GHETGRLSGAADTQALLRNDQVYQPLRDRDDAQYSHLG | 275 |
| cytoplasmic domain | GNWARNK | |
| CD3 epsilon | KNRKAKAKPVTRGAGAGGRQRGQNKERPPPVPNPDYEPI | 281 |
| cytoplasmic domain | RKGQRDLYSGLNQRRI | |
| CD3 gamma | GQDGVRQSRASDKQTLLPNDQLYQPLKDREDDQYSHLQG | 282 |
| cytoplasmic domain | NQLRRN | |
| CD3 zeta ITAM | APAYQQGQNQLYNELNLGRREEYDVLDKR | 283 |
| zeta1 | | |
| CD3 zeta ITAM | PRRKNPQEGLYNELQKDKMAEAYSEIGM | 284 |
| zeta2 | | |
| CD3 zeta ITAM | ERRRGKGHDGLYQGLSTATKDTYDALHMQ | 285 |
| zeta3 | | |
| CD3 delta ITAM | DTQALLRNDQVYQPLRDRDDAQYSHLGGN | 286 |
| delta | | |
| CD3 epsilon ITAM | ERPPPVPNPDYEPIRKGQRDLYSGLNQR | 287 |
| epsilon | | |
| CD3 gamma ITAM | DKQTLLPNDQLYQPLKDREDDQYSHLQGN | 288 |
| gamma | | |
| CD3 zeta variant | RVKFSRSADDTQALLRNDQVYQPLRDRDDAQYSHLGGN | 289 |
| (dzz) | RGRDPEMGGKPRRKNPQEGLYNELQKDKMAEAYSEIGM | |
| | KGERRRGKGHDGLYQGLSTATKDTYDALHMQALPPR | |
| CD3 zeta variant | RVKFSRSADERPPPVPNPDYEPIRKGQRDLYSGLNQRRG | 290 |
| (ezz) | RDPEMGGKPRRKNPQEGLYNELQKDKMAEAYSEIGMKGE | |
| | RRRGKGHDGLYQGLSTATKDTYDALHMQALPPR | |
| CD3 zeta variant | RVKFSRSADDKQTLLPNDQLYQPLKDREDDQYSHLQGNR | 291 |
| (gzz) | GRDPEMGGKPRRKNPQEGLYNELQKDKMAEAYSEIGMK | |
| | GERRRGKGHDGLYQGLSTATKDTYDALHMQALPPR | |
| CD3 zeta variant | RVKFSRSADDTQALLRNDQVYQPLRDRDDAQYSHLGGN | 292 |
| (deg) | RGRDPEMGGKERPPPVPNPDYEPIRKGQRDLYSGLNQR | |
| | KGDKQTLLPNDQLYQPLKDREDDQYSHLQGNALPPR | |
| CD3 zeta zeta | RVKFSRSADAPAYQQGQNQLYNELNLGRREEYDVLDKRR | 293 |
| (zdzezg) | GRDPEMGGKDTQALLRNDQVYQPLRDRDDAQYSHLGGN | |
| | KGPRRKNPQEGLYNELQKDKMAEAYSEIGMALPPRGRVK | |
| | FSRSADERPPPVPNPDYEPIRKGQRDLYSGLNQRRGRDP | |
| | EMGGKERRRGKGHDGLYQGLSTATKDTYDALHMQKGDK | |
| | QTLLPNDQLYQPLKDREDDQYSHLQGNALPPR | |
| CD3 zeta zeta | RVKFSRSADDTQALLRNDQVYQPLRDRDDAQYSHLGGN | 294 |
| (dzezgz) | RGRDPEMGGKAPAYQQGQNQLYNELNLGRREEYDVLDK | |
| | RKGERPPPVPNPDYEPIRKGQRDLYSGLNQRALPPRGR | |
| | VKFSRSADPRRKNPQEGLYNELQKDKMAEAYSEIGMRG | |
| | RDPEMGGKDKQTLLPNDQLYQPLKDREDDQYSHLQGNK | |
| | GERRRGKGHDGLYQGLSTATKDTYDALHMQALPPR | |
| CD3 zeta zeta | RVKFSRSADAPAYQQGQNQLYNELNLGRREEYDVLDKRR | 295 |
| (zzzdeg) | GRDPEMGGKPRRKNPQEGLYNELQKDKMAEAYSEIGMK | |
| | GERRRGKGHDGLYQGLSTATKDTYDALHMQALPPRGRV | |
| | KFSRSADDTQALLRNDQVYQPLRDRDDAQYSHLGGNRG | |
| | RDPEMGGKERPPPVPNPDYEPIRKGQRDLYSGLNQRKGD | |
| | KQTLLPNDQLYQPLKDREDDQYSHLQGNALPPR | |
| CD3 zeta zeta | RVKFSRSADDTQALLRNDQVYQPLRDRDDAQYSHLGGN | 311 |
| (degzzz) | RGRDPEMGGKERPPPVPNPDYEPIRKGQRDLYSGLNQRK | |
| | GDKQTLLPNDQLYQPLKDREDDQYSHLQGNALPPRGRVK | |
| | FSRSADAPAYQQGQNQLYNELNLGRREEYDVLDKRRGRD | |
| | PEMGGKPRRKNPQEGLYNELQKDKMAEAYSEIGMKGER | |
| | RRGKGHDGLYQGLSTATKDTYDALHMQALPPR | |
| CD3 zeta YA | RVKFSRSADAPAYQQGQNQLYAELNLGRREEYDVLDKRR | 312 |
| | GRDPEMGGKPRRKNPQEGLYNELQKDKMAEAYSEIGMK | |
| | GERRRGKGHDGLYQGLSTATKDTYDALHMQALPPR | |
| CD3 zeta | RVKFSRSADAPAYQQGQNQLYAELNLGRREEYDVLDKRR | 313 |
| YAYAYA | GRDPEMGGKPRRKNPQEGLYAELQKDKMAEAYSEIGMK | |
| | GERRRGKGHDGLYAGLSTATKDTYDALHMQALPPR | |
| CD3 zeta YA trunc | RVKFSRSADAPAYQQGQNQLYAELNLGRREEYDVLDKR | 314 |
| | ALPPR | |
| CD3 zeta zeta | RVKFSRSADAPAYQQGQNQLYAELNLGRREEYDVLDKRR | 315 |
| (zdzezg-6xYA) | GRDPEMGGKDTQALLRNDQVYAPLRDRDDAQYSHLGGN | |
| | KGPRRKNPQEGLYAELQKDKMAEAYSEIGMALPPRGRVK | |
| | FSRSADERPPPVPNPDYAPIRKGQRDLYSGLNQRRGRDP | |
| | EMGGKERRRGKGHDGLYAGLSTATKDTYDALHMQKGDK | |
| | QTLLPNDQLYAPLKDREDDQYSHLQGNALPPR | |
| CD3 zeta zeta | RVKFSRSADDTQALLRNDQVYAPLRDRDDAQYSHLGGN | 316 |
| (dzezgz-6xYA) | RGRDPEMGGKAPAYQQGQNQLYAELNLGRREEYDVLDK | |
| | RKGERPPPVPNPDYAPIRKGQRDLYSGLNQRALPPRGR | |
| | VKFSRSADPRRKNPQEGLYAELQKDKMAEAYSEIGMRG | |
| | RDPEMGGKDKQTLLPNDQLYAPLKDREDDQYSHLQGNK | |
| | GERRRGKGHDGLYAGLSTATKDTYDALHMQALPPR | |
| CD3 zeta ITAM | APAYQQGQNQLYAELNLGRREEYDVLDKR | 317 |
| zeta1 YA | | |
| CD3 zeta ITAM | PRRKNPQEGLYAELQKDKMAEAYSEIGM | 318 |
| zeta2 YA | | |
| CD3 zeta ITAM | ERRRGKGHDGLYAGLSTATKDTYDALHMQ | 319 |
| zeta3 YA | | |
| CD3 delta ITAM | DTQALLRNDQVYAPLRDRDDAQYSHLGGN | 320 |
| delta YA | | |
| CD3 epsilon ITAM | ERPPPVPNPDYAPIRKGQRDLYSGLNQR | 321 |
| epsilon YA | | |
| CD3 gamma ITAM | DKQTLLPNDQLYAPLKDREDDQYSHLQGN | 322 |
| gamma YA | | |
| CD3 zeta ITAM | APAYQQGQNQLYAELNLGRREEYDVLDKR | 323 |
| zeta1 YA | | |
| CD3 zeta ITAM | PRRKNPQEGLYAELQKDKMAEAYSEIGM | 324 |
| zeta2 YA | | |
| CD3 zeta ITAM | ERRRGKGHDGLYAGLSTATKDTYDALHMQ | 325 |
| zeta3 YA | | |
| CD3 delta ITAM | DTQALLRNDQVYAPLRDRDDAQYSHLGGN | 326 |
| delta YA | | |
| CD3 epsilon ITAM | ERPPPVPNPDYAPIRKGQRDLYSGLNQR | 327 |
| epsilon YA | | |
| CD3 gamma ITAM | DKQTLLPNDQLYAPLKDREDDQYSHLQGN | 328 |
| gamma YA | | |
| CD3 zeta ITAM | APAYQQGQNQLYAELNLGRREEYDVLDKR | 329 |
| zeta1 YA | | |
| CD3 zeta ITAM | PRRKNPQEGLYAELQKDKMAEAYSEIGM | 330 |
| zeta2 YA | | |
| CD3 zeta ITAM | ERRRGKGHDGLYAGLSTATKDTYDALHMQ | 331 |
| zeta3 YA | | |
| CD3 delta ITAM | DTQALLRNDQVYAPLRDRDDAQYSHLGGN | 332 |
| delta YA | | |
| CD3 epsilon ITAM | ERPPPVPNPDYAPIRKGQRDLYSGLNQR | 333 |
| epsilon YA | | |
| CD3 gamma ITAM | DKQTLLPNDQLYAPLKDREDDQYSHLQGN | 334 |
| gamma YA | | |
| CD3 zeta ITAM | APAYQQGQNQLYAELNLGRREEYDVLDKR | 335 |
| zeta1 YA | | |
| CD28 intracellular | RSKRSRLLHSDYMNMTPRRPGPTRKHYQPYAPPRDFAAY | 336 |
| | RS | |
| CD28.YMFM | RSKRSRLLHSDYMFMTPRRPGPTRKHYQPYAPPRDFAAY | 337 |
| intracellular | RS | |
| CD28.AYAA | RSKRSRLLHSDYMNMTPRRPGPTRKHYQAYAAPRDFAAY | 580 |
| intracellular | RS | |
| CD28 variant | RSKRSRLLHSDFMNMTARRAGPTRKHYQPYAPPRDFAAY | 581 |
| | RS | |
| CD2 intracellular | KRKKQRSRRNDEELETRAHRVATEERGRKPHQIPASTPQN | 582 |
| (foil); | PATSQHPPPPPGHRSQAPSHRPPPPGHRVQHQPQKRPPAPS | |
| | GTQVHQQKGPPLPRPRVQPKPPHGAAENSLSPSSN | |
| CD2 intracellular | KRKKQTPQNPATSQHPPPPPGHRSQAPSHRPPPPGHRVQH | 583 |
| (truncated) | QPQKRPPAPSGTQVHQQKGPPLPRPRVQPKPPHGAAENSL | |
| | SPSSN | |
| OX40 intracellular | ALYLLRRDQRLPPDAHKPPGGGSFRTPIQEEQADAHSTLA | 584 |
| | KI | |
| CD3 zeta variant | RVKFSRSADAPAYQQGQNQLYNELNLGRREEYDVLDKRR | 585 |
| (IXX) | GRDPEMGGKPRRKNPQEGLFNELQKDKMAEAFSEIGMKG | |
| | ERRRGKGHDGLFQGLSTATKDTFDALHMQALPPR | |
| CD3 zeta variant | RVKFSRSADAPAYQQGQNQLFNELNLGRREEFDVLDKRR | 586 |
| (XX3) | GRDPEMGGKPRRKNPQEGLFNELQKDKMAEAFSEIGMKG | |
| | ERRRGKGHDGLYQGLSTATKDTYDALHMQALPPR | |
| CD27 intracellular | QRRKYRSNKGESPVEPAEPCHYSCPREEEGSTIPIQEDY | 587 |
| | RKPEPACSP | |
| CD3 zeta variant | RVKFSRSADAPAYQQGQNQLFNELNLGRREEFDVLDKRR | 588 |
| (X2X) | GRDPEMGGKPRRKNPQEGLYNELQKDKMAEAYSEIGMK | |
| | GERRRGKGHDGLFQGLSTATKDTFDALHMQALPPR | |
| CD3 zeta variant | RVKFSRSADERRRGKGHDGLYQGLSTATKDTYDALHMQ | 589 |
| (delta12) | RGRDPEMGGK | |
| CD3 zeta variant | RVKFSRSADAPAYQQGQNQLYNELNLGRREEYDVLDKRR | 590 |
| (delta23) | GRDPEMGGK | |
| CD3 zeta variant | RVKFSRSADGVYNALQKDKMAEAYSEIRGRDPEMGGK | 591 |
| (delta13) | | |
| R, rituximab | CPYSNPSLC | 592 |
| mimotope | | |
| (GGGGS)₃ | GGGGSGGGGSGGGGS | 296 |
| GGGGS linker | GGGGS | 604 |
| (GGGGS)4 | GGGGSGGGGSGGGGSGGGGS | 602 |
| Witlow linker | GSTSGSGKPGSGEGSTKG | 603 |
| Q, QBEND-10 | ELPTQGTFSNVSTNVS | 593 |
| epitope | | |
| Q, QBEND-10 | ELPTQGTFSNVSTNVSPAKPTTTA | 594 |
| epitope | | |
| QR3 | GSGGGGSCPYSNPSLCSGGGGSELPTQGTFSNVSTNVSPA | 595 |
| | KPTTTACPYSNPSLC | |
| QQ | GSGGGGSELPTQGTFSNVSTNVSPAKPTTTAGSGGGGSEL | 596 |
| | PTQGTFSNVSTNVSPAKPTTTA | |
| CD28 hinge | IEVMYPPPYLDNEKSNGTIIHVKGKHLCPSPLFPGPSKP | 597 |
| CD28 TM | FWVLVVVGGVLACYSLLVTVAFIIFWV | 598 |
| 4F11scFv | QVTLKESGPVLVKPTETLTLTCTVSGFSLSNARMGVTWIR | 599 |
| | QPPGKALEWLAHIFSNDEKSYSTSLKSRLTISKDTSKTQV | |
| | VLTMTNMDPVDTATYYCARIRDYYDISSYYDYWGQGTLVS | |
| | VSSGGGGSGGGGSGGGGSGGGGSDIQMTQSPSAMSASVG | |
| | DRVTITCRASQDISNYLAWFQQKPGKVPKRLIYAASSLQS | |
| | GVPSRFSGSGSGTEFTLTISSLLPEDFATYYCLQLNSFPF | |
| | TFGGGTKVEIN | |
| 8F8 scFv | QVQLQESGPGLVQPSETLSLTCTVSGGSISYYYWSWIRQP | 600 |
| | PGKGLEWIGNINYMGNTIYNPSLKSRVTISVDTSKDQFSL | |
| | KLTSVSAADTAVYYCVRAEGS1DAFDFWGQGTLVAVSLGG | |
| | GGSGGGGSGGGGSGGGGSDIQMTQSPSTLSASVGDRVTIT | |
| | CRASQSISSWLAWYQQKPGKAPKVLIYKASNLESGVPSRF | |
| | SGSGSGTEFTLTISSLQPDDFATYYCQQYNSYSCTFGQGT | |
| | KLEIK | |
| CD70 | MPEEGSGCSVRRRPYGCVLRAALVPLVAGLVICLVVCIQR | 601 |
| | FAQAQQQLPLESLGWDVAELQLNHTGPQQDPRLYWQGGPA | |
| | LGRSFLHGPELDKGQLRIHRDGIYMVHIQVTLAICSSTTA | |
| | SRHHPTTLAVGICSPASRSISLLRLSFHQGCTIASQRLTP | |
| | LARGDTLCTNLTGTLLPSRNTDETFFGVQWVRP | |

Downregulation or mutation of target antigens is commonly observed in cancer cells, creating antigen-loss escape variants. Thus, to offset tumor escape and render immune cell more specific to target, the CD70-specific CAR can comprise one or more additional extracellular ligand-binding domains, to simultaneously bind different elements in target thereby augmenting immune cell activation and function. In some embodiments, the extracellular ligand-binding domains can be placed in tandem on the same transmembrane polypeptide, and optionally can be separated by a linker. In some embodiments, said different extracellular ligand-binding domains can be placed on different transmembrane polypeptides composing the CAR. In some embodiments, the disclosure relates to a population of CARs, each CAR comprising a different extracellular ligand-binding domain. In a particular, the disclosure relates to a method of engineering immune cells comprising providing an immune cell and expressing at the surface of the cell a population of CARs, each CAR comprising different extracellular ligand-binding domains. In another particular embodiment, the disclosure relates to a method of engineering an immune cell comprising providing an immune cell and introducing into the cell polynucleotides encoding polypeptides composing a population of CAR each one comprising different extracellular ligand-binding domains. By population of CARs, it is meant at least two, three, four, five, six or more CARs each one comprising different extracellular ligand-binding domains. The different extracellular ligand-binding domains according to the disclosure can, in some embodiments, simultaneously bind different elements in target thereby augmenting immune cell activation and function. The disclosure also relates to an isolated immune cell which comprises a population of CARs each one comprising different extracellular ligand-binding domains.

In another aspect, the disclosure provides polynucleotides encoding any of the CARs and polypeptides described herein. Polynucleotides can be made and expressed by procedures known in the art.

In another aspect, the disclosure provides compositions (such as a pharmaceutical compositions) comprising any of the cells of the disclosure. In some embodiments, the composition comprises a cell comprising a polynucleotide encoding any of the CARs described herein. In still other embodiments, the composition comprises either or both of the polynucleotides shown in SEQ ID NO: 297 and SEQ ID NO:298, SEQ ID NO: 299 and SEQ ID NO:300, SEQ ID NO: 301 and SEQ ID NO:302, SEQ ID NO: 303 and SEQ ID NO:304, SEQ ID NO: 305 and SEQ ID NO:306, SEQ ID NO: 307 and SEQ ID NO:308 or SEQ ID NO: 309 and SEQ ID NO:310, below:

4F11 heavy chain variable region
(SEQ ID NO: 297)
CAGGTCACCTTGAAGGAGTCTGGTCCTGTGCTGGT
GAAACCCACAGAGACCCTCACGCTGACCTGCACCG
TCTCTGGGTTCTCACTCAGTAATGCTAGAATGGGT
GTGACCTGGATCCGTCAGCCCCCAGGGAAGGCCCT
GGAGTGGCTTGCACACATTTTTTCGAATGACGAAA
AATCCTACAGTACATCTCTGAAGAGCAGGCTCACC
ATCTCCAAGGACACTTCCAAAACCCAGGTGGTCCT
TACCATGACCAACATGGACCCTGTGGACACAGCCA
CATATTACTGTGCACGGATACGAGATTACTATGAC
ATTAGTAGTTATTATGACTACTGGGGCCAGGGAAC
CCTGGTCAGCGTCTCCTCA
4F11 light chain variable region
(SEQ ID NO: 298)
GACATCCAGATGACCCAGTCTCCATCTGCCATGTC
TGCATCTGTAGGAGACAGAGTCACCATCACTTGTC
GGGCGAGTCAGGACATTAGCAATTATTTAGCCTGG
TTTCAGCAGAAACCAGGGAAAGTCCCTAAGCGCCT
GATCTATGCTGCATCCAGTTTGCAAAGTGGGGTCC
CATCAAGGTTCAGCGGCAGTGGATCGGGGACAGAA
TTCACTCTCACAATCAGCAGCCTGCTGCCTGAAGA
TTTTGCAACTTATTACTGTCTACAGCTTAATAGTT
TCCCGTTCACTTTTGGCGGAGGGACCAAGGTGGAG
ATCAAC

In still other embodiments, the composition comprises either or both of the polynucleotides shown in SEQ ID NO: 299 and SEQ ID NO:300 below:

17G6 heavy chain variable region
(SEQ ID NO: 299)
GAGGTGCAGCTGGTGGAGTCTGGGGGAGGCTTGGT
CCAGCCTGGGGGGTCCCTGAGACTCTCCTGTGTAG
CCTCTGGATTCACCTTTAGTAGTTATTGGATGAGC
TGGGTCCGCCAGGCTCCAGGGAAGGGGCTGGAGTG
GGTGGCCAGCATAAAGCAAGATGGAAGTGAGAAAT
ACTATGTGGACTCTGTGAAGGGCCGATTCACCATC
TCCAGAGACAACGCCAAGAACTCAGTGTATCTGCA
AATGAACAGCCTGAGAGCCGAGGACACGGGTGTGT
ATTACTGTGCGAGAGAAGGAGTCAACTGGGGATGG
AGACTCTACTGGCACTTCGATCTCTGGGGCCGTGG
AACCCTGGTCACTGTCTCCTCA
17G6 light chain variable region
(SEQ ID NO: 300)
GACATCGTGATGACCCAGTCTCCAGACTCCCTGGC
TGTGTCTCTGGGCGAGAGGGCCACCATCAACTGCA
AGTCCAGCCAGAGTGTTTTATACAGCTACAACAAT
AAGAACTACGTAGCTTGGTACCAGCAGAAACCAGG
ACAACCTCCTAACCTACTCATTTTCTGGGCATCTA
CCCGGGAATCCGGGGTCCCTGACCGATTCAGTGGC
AGCGGGTCTGGGACAGATTTCACTCTCACCATCAG
CAGCCTGCAGGCTGAAGATGTGGCAGTTTACTACT
GTCAGCAATATTATAGTACGCTCACTTTCGGCGGA
GGGACCAAGGTGGAGATCAAA.

In still other embodiments, the composition comprises either or both of the polynucleotides shown in SEQ ID NO: 301 and SEQ ID NO:302 below:

10H10 heavy chain variable region
(SEQ ID NO: 301)
GAGGTGCAGCTGGTGGAGTCTGGGGGAGGCTTGGT
ACAGCCTGGGGGGTCCCTGAGACTCTCCTGTGCAG
TCTCTGGATTCACCTTCAGTAACCATAACATACAC
TGGGTCCGCCAGGCTCCAGGGAAGGGGCTGGAGTG
GATTTCATACATTAGTCGAAGTAGTAGTACCATAT
ATTACGCAGACTCTGTGAAGGGCCGATTCACAATC
TCCAGAGACAATGCCAAGAACTCACTGTATCTGCA
AATGAACAGCCTGAGAGACGAAGACACGGCTGTGT
ATTACTGTGCGAGAGATCACGCTCAGTGGTACGGT
ATGGACGTTTGGGGCCAAGGGACCACGGTCACCGT
CTCCTCA.
10H10 light chain variable region
(SEQ ID NO: 302)
GACATCCAGATGACCCAGTCTCCATCTTCCGTGTC
TGCATCGGTAGGAGACAGAGTCACCATCACTTGTC
GGGCGAGTCAGGGTATTAGCAGCTGGTTAGCCTGG
TATCAGCAGAAACCAGGGAAAGCCCCTAAGGTCCT
GATCTATGCTGCATCCAGTTTGCAAAGTGGGGTCC
CATCAAGGTTCAGCGGCAGTGGATCTGGGACAGAT
TTCACTCTCACCATCAGCAGCCTGCAGCCTGAAGA
TTTTGCAACTTACTATTGTCAACAGGCTTTCAGTT
TCCCATTCACTTTCGGCCCTGGGACCAAAGTGGAT
ATCAAA.

In still other embodiments, the composition comprises either or both of the polynucleotides shown in SEQ ID NO: 303 and SEQ ID NO:304 below:

P07D03 heavy chain variable region
(SEQ ID NO: 303)
GAAGTGCAGCTTGTCCAGAGCGGAGCCGAAGTGAA
GAAGCCTGGCGAGAGCCTGAAGATCAGCTGCAAGG
GCTCCGGATATCGCTTCACAAGTTACTGGATAGGG
TGGGTGCGCCAGATGCCTGGTAAGGGACTGGAATG
GATGGGCTCTATATATCCTGATGATTCCGACACAC
GTTATAGCCCAAGCTTTCAGGGCCAGGTCACAATC
AGCGCTGACAAGAGCATCAGCACCGCCTACCTTCA
GTGGTCGTCTCTGAAGGCCAGCGACACCGCAATGT
ACTACTGCGCCTCTAGCACAGTTGACTACCCGGGA
TACAGTTACTTCGACTACTGGGGCCAAGGTACACT
GGTCACCGTCAGCAGC
P07D03 light chain variable region
(SEQ ID NO: 304)
GAGCTCCAGAGCGTGCTGACCCAGCCTCCTAGCGC
AAGCGGCACCCCTGGACAGCGTGTGACAATTAGCT
GTAGCGGAAGTCGTAGCAATATCGGATCAAACTAT
GTGTATTGGTATCAGCAATTGCCCGGTACAGCACC
CAAATTGCTCATATATAGAAATAATCAGAGACCTA
GCGGAGTGCCTGATCGTTTTAGCGGTAGCAAAAGC
GGCACCAGCGCATCACTGGCAATTTCAGGCCTGCG
TAGCGAAGATGAGGCGGATTATTACTGTGCGAGTT
GGGATGGTTCGCTGAGTGCTGTTGTGTTCGGCACC
GGTACAAAACTGACCGTTCTG

In still other embodiments, the composition comprises either or both of the polynucleotides shown in SEQ ID NO: 305 and SEQ ID NO:306 below:

P08G02 heavy chain variable region
(SEQ ID NO: 305)
GAAGTGCAGCTTGTCCAGAGCGGAGCCGAAGTGAA
GAAGCCTGGCGAGAGCCTGAAGATCAGCTGCAAGG
GCTCCGGATACACCTTTCCTTCATCATGGATAGGT
TGGGTGCGCCAGATGCCTGGTAAGGGACTGGAATG
GATGGGCATCATATACCCTGATACTAGCCATACCC
GTTACAGCCCAAGCTTTCAGGGCCAGGTCACAATC
AGCGCTGACAAGAGCATCAGCACCGCCTACCTTCA
GTGGTCGTCTCTGAAGGCCAGCGACACCGCAATGT
ACTACTGTGCCCGTGCGAGCTATTTCGATCGTGGA
ACAGGGTATAGTTCTTGGTGGATGGATGTGTGGGG
CCAAGGTACACTGGTCACCGTCAGCAGC
P08G02 light chain variable region
(SEQ ID NO: 306)
GAGCTCGATATTCAGATGACCCAGAGCCCTAGCAG
CCTGAGCGCAAGCGTGGGCGATAGAGTGACCATTA
CCTGTAGGGCCTCACAATCCATATACGACTATTTG
CACTGGTATCAGCAGAAACCCGGGAAAGCACCCAA
ACTGCTGATTTACGATGCTTCCAACCTACAGAGTG
GCGTTCCTTCACGTTTTAGCGGTAGCGGTTCAGGC
ACCGATTTCACCCTGACCATTAGCAGCCTTCAGCC
CGAAGATTTCGCTACGTATTATTGCCAGCAATCAT
ACACCACGCCGTTGTTTACATTCGGCCAGGGTACC
AAAGTGGAAATCAAA

In still other embodiments, the composition comprises either or both of the polynucleotides shown in SEQ ID NO: 307 and SEQ ID NO: 308 below:

P08F08 heavy chain variable region
(SEQ ID NO: 307)
GAAGTGCAGCTTGTCCAGAGCGGAGCCGAAGTGAA
GAAGCCTGGCGAGAGCCTGAAGATCAGCTGCAAGG
GCTCCGGATACGGATTCACAAGTTATTGGATAGGT
TGGGTGCGCCAGATGCCTGGTAAGGGACTGGAATG
GATGGGTATCATTCATCCCGATGATAGCGACACCA
AATACAGCCCAAGCTTTCAGGGCCAGGTCACAATC
AGCGCTGACAAGAGCATCAGCACCGCCTACCTTCA
GTGGTCGTCTCTGAAGGCCAGCGACACCGCAATGT
ACTACTGTGCCTCTAGCTATTTGCGTGGCTTGTGG
GGAGGCTATTTTGACTATTGGGGCCAAGGTACACT
GGTCACCGTCAGCAGC
P08F08 light chain variable region
(SEQ ID NO: 308)
GAGCTCCAGAGCGTGCTGACCCAGCCTCCTAGCGC
AAGCGGCACCCCTGGACAGCGTGTGACAATTAGCT
GTAGCGGATCAAGCTCAAACATTGGCTCAAATTAT
GTGAATTGGTATCAGCAATTGCCCGGTACAGCACC
CAAACTGCTCATTTATGGAGATTATCAACGACCTA
GCGGAGTGCCTGATCGTTTTAGCGGTAGCAAAAGC
GGCACCAGCGCATCACTGGCAATTTCAGGCCTGCG
TAGCGAAGATGAGGCGGATTATTACTGTGCTACCC
GCGACGATTCGTTATCTGGGTCTGTCGTTTTTGGC
ACCGGTACAAAACTGACCGTGCTG

In still other embodiments, the composition comprises either or both of the polynucleotides shown in SEQ ID NO: 309 and SEQ ID NO:310 below:

P15D02 heavy chain variable region
(SEQ ID NO: 309)
GAAGTGCAGCTTGTCCAGAGCGGAGCCGAAGTGAA
GAAGCCTGGCGAGAGCCTGAAGATCAGCTGCAAGG
GCTCCGGATACAGTTTTGCCTCATACTGGATCGGT
TGGGTGCGCCAGATGCCTGGTAAGGGACTGGAATG
GATGGGCGTAATTTACCCCGGAACTAGCGAGACAC
GTTACAGCCCAAGCTTTCAGGGCCAGGTCACAATC
AGCGCTGACAAGAGCATCAGCACCGCCTACCTTCA
GTGGTCGTCTCTGAAGGCCAGCGACACCGCAATGT
ACTACTGCGCTAAAGGGTTGAGTGCGAGTGCAAGT
GGATATTCTTTCCAATATTGGGGCCAAGGTACACT
GGTCACCGTCAGCAGC
P15D032 light chain variable region
(SEQ ID NO: 310)
GAGCTCGATATTCAGATGACCCAGAGCCCTAGCAG
CCTGAGCGCAAGCGTGGGCGATAGAGTGACCATTA
CCTGTAGGGCCTCACAAAGCATCGACACATATTTA
AACTGGTATCAGCAGAAACCCGGGAAAGCACCCAA
ACTGCTGATTTATTCAGCTAGTAGCCTACACAGTG
GCGTTCCTTCACGTTTTAGCGGTAGCGGTTCAGGC
ACCGATTTCACCCTGACCATTAGCAGCCTTCAGCC
CGAAGATTTCGCTACGTATTATTGCCAACAATCAT
ACAGCACAACTGCTTGGACATTCGGCCAGGGTACC
AAAGTGGAAATCAAA

Expression vectors, and administration of polynucleotide compositions are further described herein.

In another aspect, the disclosure provides a method of making any of the polynucleotides described herein.

Polynucleotides complementary to any such sequences are also encompassed by the disclosure. Polynucleotides may be single-stranded (coding or antisense) or double-stranded, and may be DNA (genomic, cDNA or synthetic) or RNA molecules. RNA molecules include hnRNA molecules, which contain introns and correspond to a DNA molecule in a one-to-one manner, and mRNA molecules, which do not contain introns. Additional coding or non-coding sequences may, but need not, be present within a polynucleotide of the disclosure, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants embodiments exhibit at least about 70% identity, at least about 80% identity, at least about 90% identity, or at least about 95% identity to a polynucleotide sequence that encodes a native antibody or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

The “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the disclosure. Further, alleles of the genes comprising the polynucleotide sequences provided herein are within the scope of the disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO87/04462, or the lentiviral pLVX vector available from Clonetech. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

A polynucleotide encoding a CD70-specific CAR disclosed herein may exist in an expression cassette or expression vector (e.g., a plasmid for introduction into a bacterial host cell, or a viral vector such as a baculovirus vector for transfection of an insect host cell, or a plasmid or viral vector such as a lentivirus for transfection of a mammalian host cell). In some embodiments, a polynucleotide or vector can include a nucleic acid sequence encoding ribosomal skip sequences such as, for example without limitation, a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptides into the secretory pathway of a host cell, in some embodiments, a secretory signal sequence (also known as a leader sequence, prepro sequence or pre sequence) is provided in a polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). In some embodiments the signal peptide comprises the amino acid sequence shown in SEQ ID NO: 266 or 277. Those skilled in the art will recognize that, in view of the degeneracy of the genetic code, considerable sequence variation is possible among these polynucleotide molecules. In some embodiments, nucleic acid sequences of the disclosure are codon-optimized for expression in mammalian cells, or in some embodiments for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

### Immune Cells Comprising or Functionally Expressing CD70-Binding Proteins and Methods of Use Thereof

Provided herein are engineered immune cells comprising or functionally expressing a CD70-binding protein, e.g., a CD70 CAR, as described herein, and methods of using thereof.

In one aspect, the disclosure provides methods of lymphodepletion in a patient in need thereof comprising administering to the patient engineered immune cells comprising or functionally expressing the CD70-binding protein, wherein the engineered immune cells inhibit proliferation and/or activity of CD70 positive cells in the patient. In some embodiments, the engineered immune cells are derived or developed or prepared from peripheral blood mononuclear cells (PBMC), T cells, NK cells, monocytes or macrophages, or a mixture thereof, or derived or developed or prepared from iPSCs. In some embodiments, the engineered immune cells are autologous or allogeneic to the patient. In some embodiments, the patient has or is expected to have a host v. graft rejection or a host v. graft reaction. In some embodiments, the patient is in need for a transplant, including without limitation, a bone marrow transplant, stem cell transplant, or tissue transplant, wherein the transplant exhibits longer persistence or more resistance to host rejection in the patient as compared to a control without being administered the engineered immune cells. In some embodiments, the patient is receiving adoptive cell therapy, optionally wherein the adoptive cell therapy is chimeric antigen receptor (CAR) T cell therapy. In some embodiments, the patient is receiving allogeneic CAR T therapy.

In some embodiments, administering an engineered immune cell as disclosed herein, or administering a population of cells comprising such engineered immune cells as disclosed herein, reduces host rejection of, e.g., a transplant or adoptive cell therapy, in a patient relative to a control receiving a comparable but non-engineered cell or comparable population that does not comprise such engineered cells. In some embodiments, the engineered immune cells comprising or functionally expressing the CD70-binding protein as described herein are administered in a patient in conjunction with (e.g., before, simultaneously or following) one or more lymphodepletion agents, and optionally some of the lymphodepletion agents can be omitted or administered at a lower level than in patient who is not administered the engineered immune cells as described herein. In some embodiments, the one or more lymphodepletion agents are a chemotherapy agent or an antibody. In some embodiments, the one or more lymphodepletion agents are fludarabine, cyclophosphamide or an anti-CD52 antibody, for example alemtuzumab.

In certain embodiments, the engineered immune cells further comprise or functionally express an additional antigen binding domain specific for a target of interest, optionally the antigen binding domain comprises an antibody that binds to the target of interest. In some embodiments, one protein comprises the additional antigen binding domain and the CD70 binding protein, and wherein the additional antigen binding domain comprises an antibody that binds to the target of interest, and optionally the additional antigen binding domain comprises an scFv. In some embodiments, the additional antigen binding protein is expressed as a separate protein from the CD70 binding protein. In some embodiments, the engineered immune cells comprising or functionally expressing the CD70-binding protein as described herein are administered in a patient in conjunction with (e.g., before, simultaneously or following) one or more lymphodepletion agents, and advantageously some of the lymphodepletion agents can be omitted or administered at a lower level than in a control who is not administered the engineered immune cells as described here.

### CD70-Specific Antibodies and Methods of Making Thereof

Provided herein are CD70 antibodies.

In some embodiments, a CD70 antibody of the disclosure comprises any one of the partial light chain sequence as listed in Table 1 and/or any one of the partial heavy chain sequence as listed in Table 1. In Table 1, the underlined sequences are CDR sequences according to Kabat and in bold according to Chothia.

Tables 2A-2B provide examples of CDR sequences of the CD70 antibodies provided herein.

In some embodiments, the disclosure provides an antibody (e.g. including antibody fragments, such as single chain variable fragments (scFvs) which specifically binds to Cluster of Differentiation 70 (CD70), wherein the antibody comprises (a) a heavy chain variable (VH) region comprising (i) a VH complementarity determining region one (CDR1) comprising the sequence shown in SEQ ID NO: 49, 50, 51, 55, 56, 57, 61, 62, 63, 67, 68, 69, 73, 74, 75, 79, 80, 81, 85, 86, 87, 91, 92, 93, 97, 98, 99, 103, 104, 105, 109, 110, 111, 115, 116, 117, 121, 122, 123, 127, 128, 129, 133, 134, 135, 139, 140, 141, 145, 146, 147, 151, 152, 153, 157, 158, 159, 163, 164, 165, 169, 170, 171, 175, 176, 177, 181, 182, 183, 187, 188, 189, 382, 383, 384, 388, 389, 390, 394, 395, 396, 400, 401, 402, 406, 407, 408, 412, 413, 414, 418, 419, 420, 424, 425, 426, 430, 431, 432, 607, 608, 609, 436, 437, 438, 442, 443, 444, 448, 449, 450, 454, 455, 456, 460, 461, 462, 466, 467, 468, 472, 473, 474, 478, 479, 480, 484, 485, 486, 490, 491, 492, 496, 497, 498, 502, 503, 504, 508, 509, or 510; (ii) a VH CDR2 comprising the sequence shown in SEQ ID NO: 52, 53, 58, 59, 64, 65, 70, 71, 76, 77, 82, 83, 88, 89, 94, 95, 100, 101, 106, 107, 112, 113, 118, 119, 124, 125, 130, 131, 136, 137, 142, 143, 148, 149, 154, 155, 160, 161, 166, 167, 172, 173, 178, 179, 184, 185, 190, 191, 385, 386, 391, 392, 397, 398, 403, 404, 409, 410, 415, 416, 421, 422, 427, 428, 433, 434, 610, 661, 439, 440, 445, 446, 451, 452, 457, 458, 463, 464, 469, 470, 475, 476, 481, 482, 487, 488, 493, 494, 499, 500, 505, 506, 511, or 512; and iii) a VH CDR3 comprising the sequence shown in SEQ ID NO: 54, 60, 66, 72, 78, 84, 90, 96, 102, 108, 114, 120, 126, 132, 138, 144, 150, 156, 162, 168, 174, 180, 186, 192, 387, 393, 399, 405, 411, 417, 423, 429, 435, 612, 441, 447, 453, 459, 465, 471, 477, 483, 489, 495, 501, 507, or 513; and/or a light chain variable (VL) region comprising (i) a VL CDR1 comprising the sequence shown in SEQ ID NO: 193, 196, 199, 202, 205, 208, 211, 214, 217, 220, 223, 226, 229, 232, 235, 238, 241, 244, 247, 250, 253, 256, 259, 262, 514, 517, 520, 523, 526, 529, 532, 535, 538, 613, 541, 544, 547, 550, 553, 556, 559, 562, 565, 568, 571, 574, or 577; (ii) a VL CDR2 comprising the sequence shown in SEQ ID NO: 194, 197, 200, 203, 206, 209, 212, 215, 218, 221, 224, 227, 230, 233, 236, 239, 242, 245, 248, 251, 254, 257, 260, 263, 515, 518, 521, 524, 527, 530, 533, 536, 539, 614, 542, 545, 548, 551, 554, 557, 560, 563, 566, 569, 572, 575, or 578; and (iii) a VL CDR3 comprising the sequence shown in SEQ ID NO: 195, 198, 201, 204, 207, 210, 213, 216, 219, 222, 225, 228, 231, 234, 237, 240, 243, 246, 249, 252, 255, 258, 261, 264, 516, 519, 522, 525, 528, 531, 534, 537, 540, 615, 543, 546, 549, 552, 555, 558, 561, 564, 567, 570, 573, 576, or 579.

In some embodiments, the disclosure provides an antibody (e.g. a scFv), which specifically binds to Cluster of Differentiation 70 (CD70), wherein the antibody comprises a heavy chain variable (VH) region comprising a VH CDR1, VH CDR2, and VH CDR3 of the VH sequence shown in SEQ ID NO: 2, 4, 6, 8, 10, 12, 14, 16, 18, 20, 22, 24, 26, 28, 30, 32, 34, 36, 38, 40, 42, 44, 46, 48, 339, 341, 343, 345, 347, 349, 351, 353, 355, 606, 357, 359, 361, 363, 365, 367, 369, 371, 373, 375, 377, 379, or 381; and/or a light chain variable (VL) region comprising VL CDR1, VL CDR2, and VL CDR3 of the VL sequence shown in SEQ ID NO: 1, 3, 5, 7, 9, 11, 13, 15, 17, 19, 21, 23, 25, 27, 29, 31, 33, 35, 37, 39, 41, 43, 45, 47, 338, 340, 342, 344, 346, 348, 350, 352, 354, 605, 356, 358, 360, 362, 364, 366, 368, 370, 372, 374, 376, 378, or 380.

In some embodiments, the disclosure provides an isolated antibody which specifically binds to CD70 and competes with any of the foregoing antibodies.

In some embodiments, the present invention provides an antibody that binds to CD70 and competes with an antibody as described herein, including 31H1, 63B2, 40E3, 42C3, 45F11, 64F9, 72C2, 2F10, 4F11, 10H10, 17G6, 65E11, P02B10, P07D03, P08A02, P08E02, P08F08, P08G02, P12B09, P12F02, P12G07, P13F04, P15D02, P16C05, 10A1, 10E2, 11A1, 11C1, 11D1, 11E1, 12A2, 12C4, 12C5, 12D3, 12D6, 12D7, 12F5, 12H4, 8C8, 8F7, 8F8, 9D8, 9E10, 9E5, 9F4 or 9F8.

In some embodiments, the invention also provides CDR portions of antibodies to CD70 antibodies based on CDR contact regions. CDR contact regions are regions of an antibody that imbue specificity to the antibody for an antigen. In general, CDR contact regions include the residue positions in the CDRs and Vernier zones which are constrained in order to maintain proper loop structure for the antibody to bind a specific antigen. See, e.g., Makabe et al., J. Biol. Chem., 283:1156-1166, 2007. Determination of CDR contact regions is well within the skill of the art.

The binding affinity (K_{D}) of the CD70 antibody as described herein to CD70 (such as human CD70 (e.g., (SEQ ID NO: 601)) can be about 0.001 to about 5000 nM. In some embodiments, the binding affinity is about any of 5000 nM, 4500 nM, 4000 nM, 3500 nM, 3000 nM, 2500 nM, 2000 nM, 1789 nM, 1583 nM, 1540 nM, 1500 nM, 1490 nM, 1064 nM, 1000 nM, 933 nM, 894 nM, 750 nM, 705 nM, 678 nM, 532 nM, 500 nM, 494 nM, 400 nM, 349 nM, 340 nM, 353 nM, 300 nM, 250 nM, 244 nM, 231 nM, 225 nM, 207 nM, 200 nM, 186 nM, 172 nM, 136 nM, 113 nM, 104 nM, 101 nM, 100 nM, 90 nM, 83 nM, 79 nM, 74 nM, 54 nM, 50 nM, 45 nM, 42 nM, 40 nM, 35 nM, 32 nM, 30 nM, 25 nM, 24 nM, 22 nM, 20 nM, 19 nM, 18 nM, 17 nM, 16 nM, 15 nM, 12 nM, 10 nM, 9 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM, 0.1 nM, 0.01 nM, or 0.001 nM. In some embodiments, the binding affinity is less than about any of 5000 nM, 4000 nM, 3000 nM, 2000 nM, 1000 nM, 900 nM, 800 nM, 250 nM, 200 nM, 100 nM, 50 nM, 30 nM, 20 nM, 10 nM, 7.5 nM, 7 nM, 6.5 nM, 6 nM, 5 nM, 4.5 nM, 4 nM, 3.5 nM, 3 nM, 2.5 nM, 2 nM, 1.5 nM, 1 nM, or 0.5 nM.

In some embodiments, the disclosure provides a nucleic acid encoding any of the foregoing isolated antibodies. In some embodiments, the disclosure provides a vector comprising such a nucleic acid. In some embodiments, the disclosure provides a host cell comprising such a nucleic acid.

The disclosure further provides any of the antibodies of the foregoing antibodies for use as a medicament. In some embodiments, the medicament is for us in treatment of a CD70-related cancer selected from the group consisting of Renal Cell Carcinoma, Glioblastoma, glioma such as low grade glioma, Non-Hodgkin's Lymphoma (NHL), Hodgkin's Disease (HD), Waldenstrom's macroglobulinemia, Acute Myeloid Leukemia, Multiple Myeloma, diffuse large-cell lymphoma, follicular lymphoma or Non-Small Cell Lung Cancer.

In some embodiments, the disclosure provides a method of treating a subject in need thereof, comprising providing any of the foregoing antibodies, and administering said antibody to said subject.

In some embodiments, the disclosure provides a pharmaceutical composition comprising any of the foregoing antibodies.

In some embodiments, the disclosure provides a method of treating a condition associated with malignant cells expressing CD70 in a subject comprising administering to a subject in need thereof an effective amount of any one of the foregoing antibodies or a pharmaceutical composition comprising any one of the foregoing antibodies. In some embodiments, the condition is cancer. In some embodiments, the cancer is a CD70 related cancer selected from the group consisting of Renal Cell Carcinoma, Glioblastoma, glioma such as low grade glioma, Non-Hodgkin's Lymphoma (NHL), Hodgkin's Disease (HD), Waldenstrom's macroglobulinemia, Acute Myeloid Leukemia, Multiple Myeloma, diffuse large-cell lymphoma, follicular lymphoma or Non-Small Cell Lung Cancer.

In some embodiments, the disclosure provides, a method of inhibiting tumor growth or progression in a subject who has malignant cells expressing CD70, comprising administering to the subject in need thereof an effective amount of a pharmaceutical composition of the disclosure to the subject.

In some embodiments, the disclosure provides, a method of inhibiting metastasis of malignant cells expressing CD70 in a subject, comprising administering to the subject in need thereof an effective amount of a pharmaceutical composition of the disclosure to the subject.

In some embodiments, the disclosure provides, a method of inducing tumor regression in a subject who has malignant cells expressing CD70, comprising administering to the subject in need thereof an effective amount of a pharmaceutical composition of the disclosure to the subject.

In some embodiments, the antibody, comprising culturing the host cell of the disclosure under conditions that result in production of the antibody, and isolating the antibody from the host cell or culture.

The antibodies useful in the present invention can encompass monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the CD70 monospecific antibody as described herein is a monoclonal antibody. For example, the CD70 monospecific antibody is a human monoclonal antibody.

The disclosure further provides the following illustrative embodiments:

1. An isolated antibody, which specifically binds to Cluster of Differentiation 70 (CD70), wherein the antibody comprises
(a) a heavy chain variable (VH) region comprising (i) a VH complementarity determining region one (CDR1) comprising the sequence shown in SEQ ID NO: 49, 50, 51, 55, 56, 57, 61, 62, 63, 67, 68, 69, 73, 74, 75, 79, 80, 81, 85, 86, 87, 91, 92, 93, 97, 98, 99, 103, 104, 105, 109, 110, 111, 115, 116, 117, 121, 122, 123, 127, 128, 129, 133, 134, 135, 139, 140, 141, 145, 146, 147, 151, 152, 153, 157, 158, 159, 163, 164, 165, 169, 170, 171, 175, 176, 177, 181, 182, 183, 187, 188, 189, 382, 383, 384, 388, 389, 390, 394, 395, 396, 400, 401, 402, 406, 407, 408, 412, 413, 414, 418, 419, 420, 424, 425, 426, 430, 431, 432, 607, 608, 609, 436, 437, 438, 442, 443, 444, 448, 449, 450, 454, 455, 456, 460, 461, 462, 466, 467, 468, 472, 473, 474, 478, 479, 480, 484, 485, 486, 490, 491, 492, 496, 497, 498, 502, 503, 504, 508, 509, or 510; (ii) a VH CDR2 comprising the sequence shown in SEQ ID NO: 52, 53, 58, 59, 64, 65, 70, 71, 76, 77, 82, 83, 88, 89, 94, 95, 100, 101, 106, 107, 112, 113, 118, 119, 124, 125, 130, 131, 136, 137, 142, 143, 148, 149, 154, 155, 160, 161, 166, 167, 172, 173, 178, 179, 184, 185, 190, 191, 385, 386, 391, 392, 397, 398, 403, 404, 409, 410, 415, 416, 421, 422, 427, 428, 433, 434, 610, 611, 439, 440, 445, 446, 451, 452, 457, 458, 463, 464, 469, 470, 475, 476, 481, 482, 487, 488, 493, 494, 499, 500, 505, 506, 511, or 512; and iii) a VH CDR3 comprising the sequence shown in SEQ ID NO: 54, 60, 66, 72, 78, 84, 90, 96, 102, 108, 114, 120, 126, 132, 138, 144, 150, 156, 162, 168, 174, 180, 186, 192, 387, 393, 399, 405, 411, 417, 423, 429, 435, 612, 441, 447, 453, 459, 465, 471, 477, 483, 489, 495, 501, 507, or 513; and/or
(b) a light chain variable (VL) region comprising (i) a VL CDR1 comprising the sequence shown in SEQ ID NO: 193, 196, 199, 202, 205, 208, 211, 214, 217, 220, 223, 226, 229, 232, 235, 238, 241, 244, 247, 250, 253, 256, 259, 262, 514, 517, 520, 523, 526, 529, 532, 535, 538, 613, 541, 544, 547, 550, 553, 556, 559, 562, 565, 568, 571, 574, or 577; (ii) a VL CDR2 comprising the sequence shown in SEQ ID NO: 194, 197, 200, 203, 206, 209, 212, 215, 218, 221, 224, 227, 230, 233, 236, 239, 242, 245, 248, 251, 254, 257, 260, 263, 515, 518, 521, 524, 527, 530, 533, 536, 539, 614, 542, 545, 548, 551, 554, 557, 560, 563, 566, 569, 572, 575, or 578; and (iii) a VL CDR3 comprising the sequence shown in SEQ ID NO: 195, 198, 201, 204, 207, 210, 213,216, 219, 222, 225, 228, 231, 234, 237, 240, 243,246, 249, 252, 255, 258, 261, 264, 516, 519, 522, 525, 528, 531, 534, 537, 540, 615, 543, 546, 549, 552, 555, 558, 561, 564, 567, 570, 573, 576, or 579.

2. An isolated antibody which specifically binds to Cluster of Differentiation 70 (CD70), wherein the antibody comprises:
(a) a VH region comprising a VH CDR1, VH CDR2, and VH CDR3 of the VH sequence shown in SEQ ID NO: 2, 4, 6, 8, 10, 12, 14, 16, 18, 20, 22, 24, 26, 28, 30, 32, 34, 36, 38, 40, 42, 44, 46, 48, 339, 341, 343, 345, 347, 349, 351, 353, 355, 606, 357, 359, 361, 363, 365, 367, 369, 371, 373, 375, 377, 379, or 381; and/or
(b) a VL region comprising VL CDR1, VL CDR2, and VL CDR3 of the VL sequence shown in SEQ ID NO: 1, 3, 5, 7, 9, 11, 13, 15, 17, 19, 21, 23, 25, 27, 29, 31, 33, 35, 37, 39, 41, 43, 45, 47, 338, 340, 342, 344, 346, 348, 350, 352, 354, 605, 356, 358, 360, 362, 364, 366, 368, 370, 372, 374, 376, 378, or 380.

3. An isolated antibody which specifically binds to CD70 and competes with the antibody of embodiment 1.

4. A nucleic acid encoding the antibody of any one of embodiments 1-3.

5. A vector comprising the nucleic acid of embodiment 4.

6. A host cell comprising the nucleic acid of embodiment 4.

7. The antibody of any one of embodiments 1-3 for use as a medicament.

8. The antibody of embodiment 7, wherein the medicament is for use in treatment of an CD70 related cancer selecting from the group consisting of Renal Cell Carcinoma, Glioblastoma, glioma such as low grade glioma, Non-Hodgkin's Lymphoma (NHL), Hodgkin's Disease (HD), Waldenstrom's macroglobulinemia, Acute Myeloid Leukemia, Multiple Myeloma, diffuse large-cell lymphoma, follicular lymphoma or Non-Small Cell Lung Cancer.

9. A method of treating a subject in need thereof comprising:
a. providing the antibody according to any one of embodiments 1-3; and
b. administering said antibody to said subject.

10. A pharmaceutical composition comprising the antibody of any one of embodiments 1-3.

11. A method of treating a condition associated with malignant cells expressing CD70 in a subject comprising administering to a subject in need thereof an effective amount of the antibody of any one of embodiments 1-3 or the pharmaceutical composition of embodiment 10.

12. The method of embodiment 11, wherein the condition is a cancer.

13. The method of embodiment 12, wherein the cancer is an CD70 related cancer selected from the group consisting of Renal Cell Carcinoma, Glioblastoma, glioma such as low grade glioma, Non-Hodgkin's Lymphoma (NHL), Hodgkin's Disease (HD), Waldenstrom's macroglobulinemia, Acute Myeloid Leukemia, Multiple Myeloma, diffuse large-cell lymphoma, follicular lymphoma or Non-Small Cell Lung Cancer.

14. A method of inhibiting tumor growth or progression in a subject who has malignant cells expressing CD70, comprising administering to the subject in need thereof an effective amount of the pharmaceutical composition of embodiment 10 to the subject.

15. A method of inhibiting metastasis of malignant cells expressing CD70 in a subject, comprising administering to the subject in need thereof an effective amount of the pharmaceutical composition of embodiment 10 to the subject.

16. A method of inducing tumor regression in a subject who has malignant cells expressing CD70, comprising administering to the subject in need thereof an effective amount of the pharmaceutical composition of embodiment 10 to the subject.

17. A method of producing an antibody, comprising culturing the host cell of embodiment 6 under conditions that result in production of the antibody, and isolating the antibody from the host cell or culture.

### Methods of Treating

Engineered immune cells, e.g. engineered T cells described herein, optionally obtained by the methods described herein, cell lines described herein derived from such engineered immune cells or engineered T cells, and compositions described herein comprising such cells can be used as a medicament. In some embodiments, such a medicament can be used for treating a disorder such as for example a viral disease, a bacterial disease, a cancer, an inflammatory disease, an immune disease, or an aging-associated disease. In some embodiments, the cancer can be selected from the group consisting of gastric cancer, sarcoma, lymphoma (including Non-Hodgkin's lymphoma), leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In some embodiments, engineered immune cells e.g., engineered T cells according to the instant disclosure, or a cell line derived from the engineered immune cells e.g., engineered T cells, can be used in the manufacture of a medicament for treatment of a disorder in a subject in need thereof. In some embodiments, the disorder can be, for example, a cancer, an autoimmune disorder, a host v. graft rejection or reaction, or an infection.

Also provided herein are methods for treating subjects. In some embodiments the method comprises administering or providing an engineered immune cell e.g., an engineered T cell of the instant disclosure or a composition comprising such cells to a subject in need thereof. In some embodiments, the method comprises a step of administering the engineered immune cells e.g., engineered T cells of the instant disclosure, or a composition comprising such cells, to a subject in need thereof.

In some embodiments, engineered immune cells e.g., engineered T cells of the instant disclosure can undergo robust in vivo cell expansion and can persist for an extended amount of time. Methods of treatment of the instant disclosure can be ameliorating, curative or prophylactic. The method of the instant disclosure can be either part of an autologous immunotherapy or part of an allogeneic immunotherapy treatment. The instant disclosure is particularly suitable for allogeneic immunotherapy. Engineered immune cells e.g., engineered T cells provided by a donor, can be transformed into non-alloreactive cells using standard protocols and reproduced as needed, thereby producing e.g. CAR-T cells which can be administered to one or several subjects. Such CAR-T cell therapy can be made available as an allogeneic ALLO CAR T™ therapeutic product.

In another aspect, the instant disclosure provides a method of inhibiting tumor growth or progression in a subject who has a tumor, comprising administering to the subject an effective amount of engineered immune cells e.g. engineered T cells as described herein. In another aspect, the present disclosure provides a method of inhibiting or preventing metastasis of cancer cells in a subject, comprising administering to the subject in need thereof an effective amount of engineered immune cells e.g. engineered T cells as described herein. In another aspect, the instant disclosure provides a method of inducing tumor regression in a subject who has a tumor, comprising administering to the subject an effective amount of engineered immune cells, e.g., engineered T cells as described herein.

In some embodiments, the immune cells, e.g., T cells provided herein can be administered parenterally to a subject. In some embodiments, the subject is a human.

In some embodiments, the method can further comprise administering an effective amount of a second therapeutic agent. In some embodiments, the second therapeutic agent is, for example, crizotinib, palbociclib, an anti-CTLA4 antibody, an anti-4-1 BB antibody, a PD-1 antibody, or a PD-L1 antibody.

Also provided is the use of any of the immune cells e.g. T cells provided herein in the manufacture of a medicament for the treatment of cancer or for inhibiting tumor growth or progression in a subject in need thereof.

In certain embodiments, the functional expression level of any gene that is knocked down or knocked out according to the present disclosure, in an engineered immune cell of the present disclosure is decreased by or by at least about 25%, 30%, 35%, 40%, 45%, 50%, 55%, 60%, 65%, 70%, 75%, 80%, 85%, 90%, 95%, 99%, or 100% relative to the corresponding expression level in an appropriate control cell. Expression levels can be determined by any known method, such as FACS or MACs. In some embodiments, the engineered immune cell disclosed herein functionally expresses any gene that is knocked down or knocked out according to the present disclosure, at a level not greater than 75%, not greater than 50%, not greater than 25%, not greater than 10% or at a level of 0% of the expression level in an appropriate control cell, e.g. in non-engineered immune cells that otherwise are the same as the engineered immune cells, e.g. comprise the same components as the engineered immune cells. In some embodiments, both alleles of one gene are knocked out, so that the gene's expression level in the engineered immune cell disclosed herein is 0% of that of a control cell. In some embodiments, one of the two alleles of a gene is knocked out, so that the gene's expression level in the engineered immune cell disclosed herein is 50% or about 50% (e.g. if a compensatory mechanism causes greater than normal expression of the remaining allele) of that of a corresponding non-engineered cell. Intermediate levels of expression can be observed if, for example, expression is reduced by some means other than knock-out, as described herein.

In some embodiments, the expression level of any gene which is manipulated according to the present disclosure, in the engineered cells of the present disclosure can be measured by assaying the cells for gene products and their properties using standard techniques known to those of skill in the art (e.g. RT-qPCR, nucleic acid sequencing, antibody staining, flow cytometry, or some combination of techniques). These measurements can be compared to corresponding measurements made on comparable cells that have not been engineered to reduce the functional expression level of the corresponding gene. In a population of cells that comprises an engineered cell e.g. engineered immune cell of the invention, a pooled sample of the material being measured, e.g. RNA or protein or cells, will reflect the fact that some of the cells do not express the gene of interest, having had both alleles knocked out, for example, some of the cells express the gene of interest at 50% or about 50% of non-engineered level, having had only one allele knocked out, and, if the population comprises non-engineered cells, that some of the cells express a normal level of the gene of interest.

In some embodiments, administering an engineered immune cell e.g. engineered T cell as disclosed herein, or administering a population of cells comprising such engineered immune cells e.g. engineered T cells, reduces host rejection of the administered cell or population of cells relative to a comparable but non-engineered cell or comparable population that does not comprise such engineered cells. In some embodiments, administering an engineered immune cell, e.g., engineered T cell of the instant disclosure, comprising an antigen binding protein e.g. a CAR and a CD70-binding protein or a CD70 CAR, or administering a population of cells comprising such engineered immune cells, e.g., engineered T cells, reduces host rejection of the administered cell or population of cells relative to a comparable but non-engineered cell or population that does not comprise such engineered cells. For example, such administration reduces host rejection by between 1% and 99%, e.g. between 5% and 95%, between 10% and 90%, between 50% and 90%, e.g. by 10%, 20%, 30%, 40%, 50%, 60%, 70%, 80%, or 90% as compared to host rejection of cells that are the same but which are not engineered to express a CD70 CAR. In some embodiments, host rejection is reduced by over 90%.

In some embodiments, administering an immune cell e.g. T cell of the instant disclosure comprising an antigen binding protein e.g. a CAR and a CD70-binding protein, e.g., a CD70 CAR, or administering a population of cells comprising such immune cells e.g. T cells, enhances or improves the persistence and/or increases the persistence of the cells as compared to the persistence of cells that are the same but which are not engineered to express a CD70 CAR. In some embodiments, persistence is increased by, for example, between 1 and 7 days, by between 1 and 12 weeks (e.g. between 1 and 4 weeks, 4 and 8 weeks, or 8 and 12 weeks), or by between 1 and 12 months, or by a specific length of time that falls within these ranges. In some embodiments, the difference in persistence is measured by comparing the half-life of the administered cells in the population or composition, wherein, for example, the half-life is increased by, for example, between 1 and 7 days, by between 1 and 12 weeks (e.g. between 1 and 4 weeks, 4 and 8 weeks, or 8 and 12 weeks), or by between 1 and 12 months, or by a specific length of time that falls within these ranges. In some embodiments, the difference in persistence is measured by comparing the length of time that the administered cells can be detected after administration. In some embodiments, the improvement in persistence is measured in vitro by comparing the survival of CD70 CAR and non-CD70 CAR cells in the presence of, for example, immune cells such as T cells or NK cells, e.g. at about 72 hours, 5 days, 7 days or 13 days after mixing. In some embodiments, in such an in vitro assay, between about 1.5 and 10 times as many engineered cells survive as do cells that are not engineered at the time of measurement. The degree of improved persistence or survival of the CD70 CAR-expressing cells depends in part on the level of expression of CD70 in the co-incubated (e.g. “attacking” or host) immune cells.

In some embodiments, reduction in host rejection and/or increases in persistence of administered cells as disclosed herein are determined by any of a variety of techniques known to the person of ordinary skill in the art. In some embodiments, any one or a combination of the following is use: flow cytometry, PCR, e.g., quantitative PCR, and ex vivo coincubation with patient or recipient immune cells.

In some embodiments, the treatment can be in combination with one or more therapies against cancer selected from the group of antibodies therapy, chemotherapy, cytokines therapy, dendritic cell therapy, gene therapy, hormone therapy, laser light therapy and radiation therapy.

In some embodiments, treatment can be administered to subjects undergoing an immunosuppressive treatment. Indeed, the instant disclosure can rely on cells or a population of cells which have been made resistant to at least one immunosuppressive agent due to the inactivation of a gene encoding a receptor for such immunosuppressive agent (e.g. in some embodiments, engineered immune cells administered to treat can comprise a CD52 knockout to avoid the effects of anti-CD52 lymphodepleting antibodies such as alemtuzumab). In this aspect, the immunosuppressive treatment can help the selection and expansion of the T cells according to the instant disclosure within the subject.

The administration of the cells or population of cells according to the instant disclosure can be carried out in any convenient manner, including by aerosol inhalation, injection, ingestion, transfusion, implantation or transplantation. The compositions described herein can be administered to a subject subcutaneously, intradermally, intratumorally, intranodally, intramedullary, intramuscularly, by intravenous or intralymphatic injection, or intraperitoneally. In one embodiment, the cell compositions of the instant disclosure are administered by intravenous injection.

In some embodiments, the administration of the cells or population of cells according to the instant disclosure can comprise administration of, for example, from about 10³ or 10⁴ to about 10⁹ engineered immune cells disclosed herein per kg body weight including all integer values of cell numbers within those ranges. In some embodiments the administration of the cells or population of cells can comprise administration of about 10⁵ to about 10⁶ engineered immune cells disclosed herein per kg body weight including all integer values of cell numbers within those range, or administration of between 0.1×10⁶ and 5×10⁶ engineered immune cells disclosed herein per kg body weight, or a total of between 0.1×10⁸ and 5×10⁸ engineered immune cells disclosed herein. The cells or population of cells can be administered in one or more doses. In some embodiments, an effective amount of cells can be administered as a single dose. In some embodiments, an effective amount of cells can be administered as more than one dose over a period time. Timing of administration is within the judgment of the managing physician and depends on the clinical condition of the subject. The cells or population of cells can be obtained from any source, such as a blood bank or a donor. While individual needs vary, determination of optimal ranges of effective amounts of a given cell type for a particular disease or conditions is within the skill of the art. An effective amount means an amount which provides a therapeutic or prophylactic benefit. The dosage administered will be dependent upon the age, health and weight of the recipient, the kind of concurrent treatment, if any, the frequency of treatment and the nature of the effect desired. In some embodiments, an effective amount of cells or composition comprising those cells are administered parenterally. In some embodiments, administration can be an intravenous administration. In some embodiments, administration can be directly done by injection within a tumor.

In some embodiments of the instant disclosure, cells are administered to a subject in conjunction with (e.g., before, simultaneously or following) any number of relevant treatment modalities, including but not limited to treatment with agents such as monoclonal antibody therapy, CCR2 antagonist (e.g., INC-8761), antiviral therapy, cidofovir and interleukin-2, Cytarabine (also known as ARA-C) or nataliziimab treatment for MS subjects or efaliztimab treatment for psoriasis subjects or other treatments for PML subjects. In some embodiments, BCMA specific CAR-T cells are administered to a subject in conjunction with one or more of the following: an anti-PD-1 antibody (e.g., nivolumab, pembrolizumab), an anti-PD-L1 antibody (e.g., avelumab, atezolizumab, or durvalumab), an anti-OX40 antibody, an anti-4-1 BB antibody (e.g., Utolimumab), an anti-MCSF antibody, an anti-GITR antibody, and/or an anti-TIGIT antibody. In further embodiments, the immune cells, e.g. T cells, of the instant disclosure can be used in combination with chemotherapy, radiation, immunosuppressive agents, such as cyclosporin, azathioprine, methotrexate, mycophenolate, and FK506, antibodies, or other immunoablative agents such as CAMPATH (alemtuzumab), anti-CD3 antibodies or other antibody therapies, cytoxan, fludarabine, cyclophosphamide, cyclosporin, FK506, rapamycin, mycoplienolic acid, steroids, FR901228, cytokines, and/or irradiation. These drugs inhibit either the calcium dependent phosphatase calcineurin (cyclosporine and FK506) or inhibit the p70S6 kinase that is important for growth factor induced signaling (rapamycin) (Henderson, Naya et al. Immunology. 1991 July; 73(3): 316-321; Liu, Albers et al. Biochemistry 1992 Apr. 28; 31(16):3896-901; Bierer, Hollander et al. Curr Opin Immunol. 1993 October; 5(5):763-73).

In a further embodiment, the cell compositions of the instant disclosure are administered to a subject in conjunction with (e.g., before, simultaneously or following) bone marrow transplantation, T cell ablative therapy using either chemotherapy agents such as fludarabine, external-beam radiation therapy (XRT), cyclophosphamide, or antibodies such as CAMPATH (alemtuzumab). In some embodiments, the cell compositions of the instant disclosure are administered following B-cell ablative therapy such as agents that react with CD20, e.g., Rituxan. For example, in one embodiment, subjects can undergo standard treatment with high dose chemotherapy followed by peripheral blood stem cell transplantation. In certain embodiments, following the transplant, subjects receive an infusion of expanded immune cells of the instant disclosure. In some embodiments, expanded cells are administered before or following surgery.

### Kits

The instant disclosure also provides kits for use in the instant methods. Kits of the instant disclosure include one or more containers comprising a composition of the instant disclosure or an immune cell, e.g., a T cell of the instant disclosure or a population of cells comprising an immune cell, e.g., an engineered T cell of the instant disclosure. In various embodiments, the immune cell, e.g., T cell comprises one or more polynucleotide(s) encoding a first and a second antigen binding protein, e.g., a first CAR and a second, CD70 CAR as described herein, and further optionally is engineered to express a reduced level of TRAC and/or CD52. The kit further comprises instructions for use in accordance with any of the methods of the instant disclosure described herein. Generally, these instructions comprise a description of administration of the composition, immune cell, e.g., a T cell or population of cells, described herein, for the above described therapeutic treatments.

The instructions relating to the use of the kit components generally include information as to dosage, dosing schedule, and route of administration for the intended treatment. The containers can be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The kits of the present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). At least one active agent in the composition is an immune cell e.g. T cell according to the instant disclosure. The container can further comprise a second pharmaceutically active agent.

Kits can optionally provide additional components such as buffers and interpretive information. Normally, the kit comprises a container and a label or package insert(s) on or associated with the container.

### Methods of Sorting and Depletion

In some embodiments, provided are methods for in vitro sorting of a population of immune cells, wherein a subset of the population of immune cells comprises immune cells engineered as described herein to express one or more antigen binding proteins. In various embodiments the method comprises contacting the population of immune cells with a monoclonal antibody specific for an epitope (e.g., a mimotope such as those provided in US2018/0002435) unique to the engineered cell, e.g. an epitope of the antigen binding protein or a mimotope incorporated into the antigen binding protein, and selecting the immune cells that bind to the monoclonal antibody to obtain a population of cells enriched in engineered immune cells that express the antigen binding protein.

In some embodiments, the monoclonal antibody specific for the epitope is optionally conjugated to a fluorophore. In this embodiment, the step of selecting the cells that bind to the monoclonal antibody can be done by Fluorescence Activated Cell Sorting (FACS).

In some embodiments, said monoclonal antibody specific for said epitope is optionally conjugated to a magnetic particle. In this embodiment, the step of selecting the cells that bind to the monoclonal antibody can be done by Magnetic Activated Cell Sorting (MACS).

In some embodiments, the mAb used in the method for sorting immune cells expressing the antigen binding protein e.g. CAR is chosen from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab. In some embodiments, said mAb is rituximab. In another embodiment, said mAb is QBEND-10.

In some embodiments, the population of CAR-expressing immune cells obtained when using the method for in vitro sorting CAR-expressing immune cells described above, comprises at least 70%, 75%, 80%, 85%, 90%, 95% of CAR-expressing immune cells. In some embodiments, the population of CAR-expressing immune cells obtained when using the method for in vitro sorting CAR-expressing immune cells, comprises at least 85% CAR-expressing immune cells.

In some embodiments, the population of CAR-expressing immune cells obtained when using the method for in vitro sorting CAR-expressing immune cells described above shows increased cytotoxic activity in vitro compared with the initial (non-sorted) cell population. In some embodiments, said cytotoxic activity in vitro is increased by 10%, 20%, 30% or 50%. In some embodiments, the immune cells are T-cells.

The CAR-expressing immune cells to be administered to the recipient can be enriched in vitro from the source population. Methods of expanding source populations can include selecting cells that express an antigen such as CD34 antigen, using combinations of density centrifugation, immuno-magnetic bead purification, affinity chromatography, and fluorescent activated cell sorting.

Flow cytometry can be used to quantify specific cell types within a population of cells. In general, flow cytometry is a method for quantitating components or structural features of cells primarily by optical means. Since different cell types can be distinguished by quantitating structural features, flow cytometry and cell sorting can be used to count and sort cells of different phenotypes in a mixture.

A flow cytometry analysis involves two primary steps: 1) labeling selected cell types with one or more labeled markers, and 2) determining the number of labeled cells relative to the total number of cells in the population. In some embodiments, the method of labeling cell types includes binding labeled antibodies to markers expressed by the specific cell type. The antibodies can be either directly labeled with a fluorescent compound or indirectly labeled using, for example, a fluorescent-labeled second antibody which recognizes the first antibody.

In some embodiments, the method used for sorting T cells expressing a CAR is the Magnetic-Activated Cell Sorting (MACS). Magnetic-activated cell sorting (MACS) is a method for separation of various cell populations depending on their surface antigens (CD molecules) by using superparamagnetic nanoparticles and columns. MACS can be used to obtain a pure cell population. Cells in a single-cell suspension can be magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a washing step, the column is removed from the separator, and the magnetically labeled cells are eluted from the column.

A detailed protocol for the purification of a specific cell population such as T-cells can be found in Basu S et al. (2010). (Basu S, Campbell H M, Dittel B N, Ray A. Purification of specific cell population by fluorescence activated cell sorting (FACS). J Vis Exp. (41): 1546).

### EXAMPLES

### Example 1. A CD70 Dagger May Prevent Allorejection and Improve Anti-Tumor Efficacy of CAR T Cells

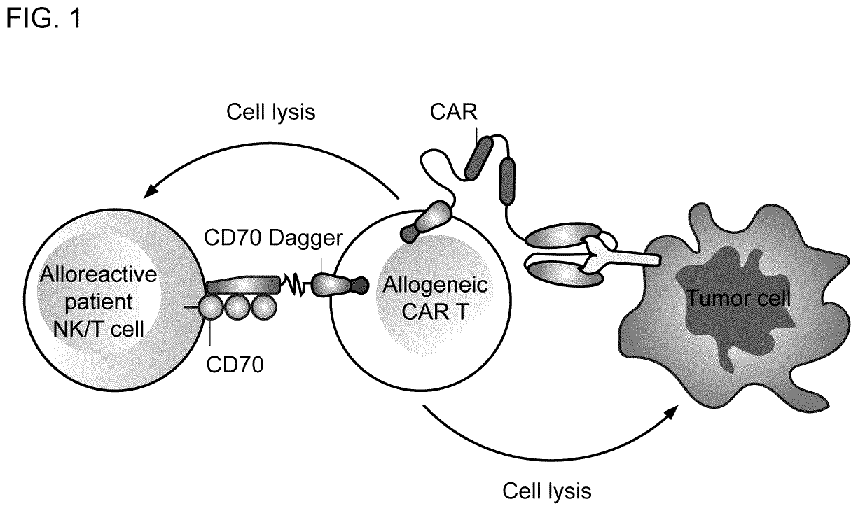

FIG. 1 illustrates how a CD70 dagger (or a CD70 dagger protein or a CD70 binding protein) protects allogeneic CAR T cells from allorejection. Cells armed with a CD70 dagger can recognize CD70 on the cell surface of activated alloreactive cells. Therefore, any activated alloreactive cells that approaches a CD70 dagger CAR T cell will be recognized and killed (FIG. 1). Eliminating alloreactive cells will in turn allow CD70 dagger CAR T cells to persist longer and carry out tumor cell killing.

### Example 2. Demonstrating CD70 CAR Anti-Rejection Functions Against Primed Alloreactive T Cells

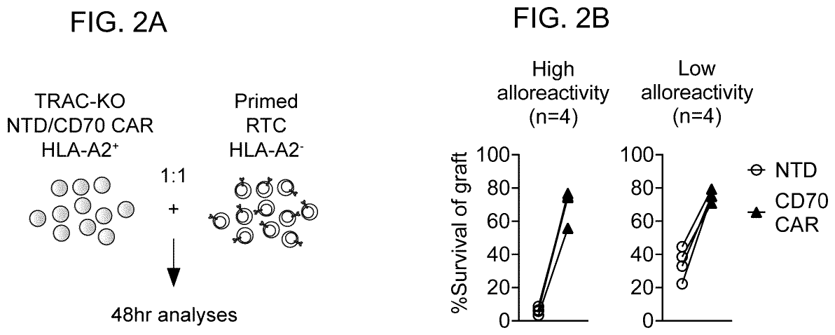

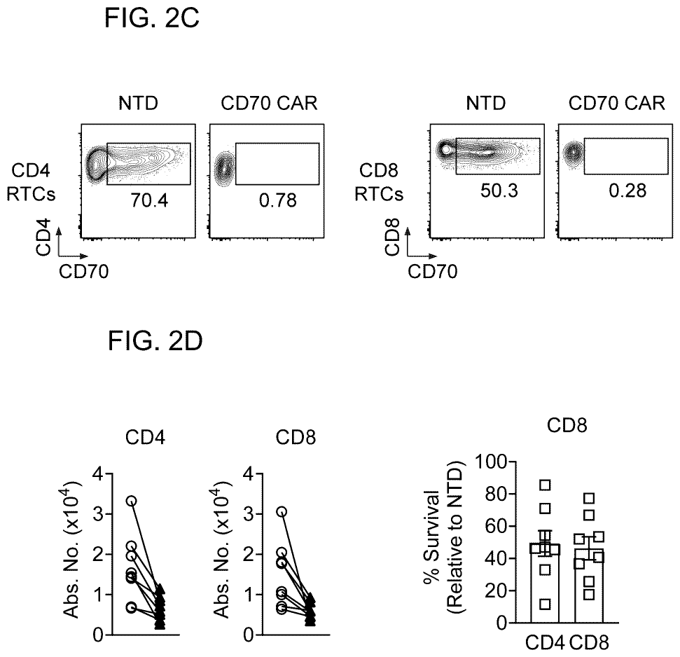

Alloreactive T cells are thought to be the main mediators of allorejection. Therefore, primed alloreactive T cell mixed lymphocyte reactions (MLRs) were performed using CD70 CAR T cells as target cells to evaluate the CD70 CAR as a potential anti-rejection dagger (CD70 dagger). The CD70 CAR tested in this experiment contains a CD70 binding domain derived from the anti-CD70 clone 4F11 in the form of scFv, a CD3ζ signaling domain and a 4-1BB co-stimulatory domain. In brief, PBMCs from eight recipient donors were co-cultured with irradiated graft donor T cells for 7 days to allow for priming and expansion of alloreactive recipient T cells (“RTCs”). Primed alloreactive RTCs (effector cells) were then isolated and co-cultured either with graft donor T cell receptor alpha constant (TRAC) knockout (KO) CD70 CAR T cells or with graft donor T cell receptor alpha constant (TRAC) knockout (KO) non-transduced T cells at a ratio of 1:1 for 48 hrs (FIG. 2A). Killing of graft donor cells was assessed by flow cytometry. Non-transduced control T cells (NTD) were efficiently killed by alloreactive RTCs, while the majority of CD70 CAR T cells survived in the presence of alloreactive RTCs (FIG. 2B). The survival of CD70 CAR T cells correlated with the reduction of CD70+ RTCs (FIGS. 2C and 2D), demonstrating that CD70 CAR expression, functioning as a CD70 dagger, can result in killing of alloreactive cells and improve persistence of graft cells.

### Example 3. Testing CD70 Dagger Activity Against Allogeneic PBMCs

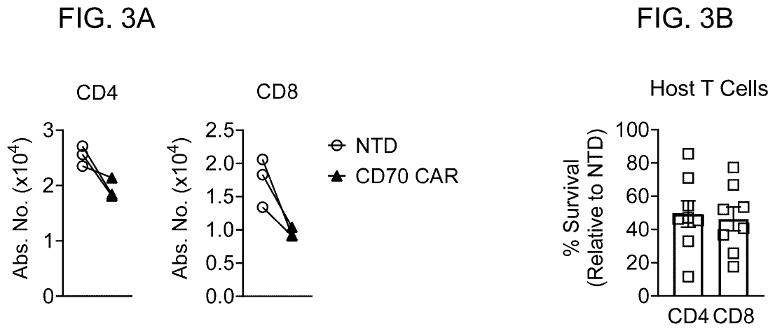

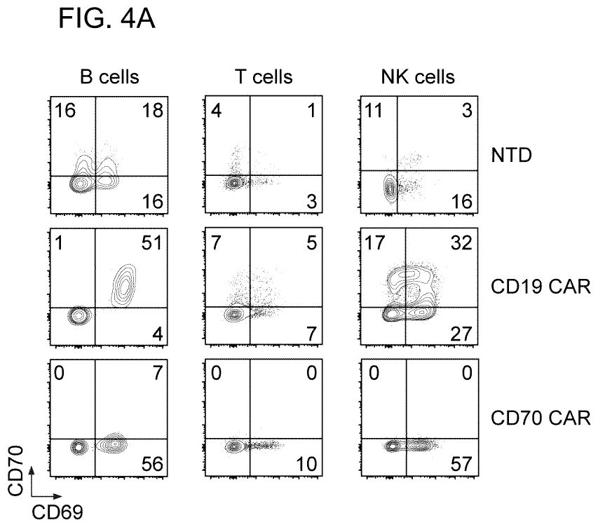

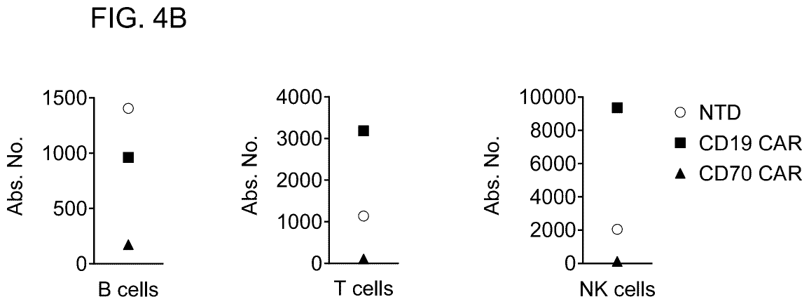

To determine whether the findings from example 2 extend to a more physiologically relevant scenario, a PBMC MLR was performed using CD70 CAR T cells as target cells. Here, CD70 dagger activity was evaluated using multiple graft donors instead of recipient donors. PBMCs from a recipient donor were co-cultured with graft donor TRAC KO CD70 CAR T cells generated from three different donors at a ratio of 1:1 for 6 days. Killing of recipient T cells was assessed by flow cytometry. The results were consistent with the findings of Example 2 in that CD70 CAR T cells, functioning as a CD70 dagger, reduced RTC counts and frequencies (FIGS. 3A and 3B). In addition to eliminating CD70+ RTCs, CD70 CAR T cells also eliminated CD70+ allogeneic B and NK cells (FIGS. 4A and 4B). In summary, these data demonstrate that a CD70 dagger can be used to reduce allorejection of allogeneic CAR T cells by host immune cells.

### Example 4. Examination of Engineered T Cells Expressing Different CD70 Daggers (CD70 Binding Proteins) Generated by LVV Transduction

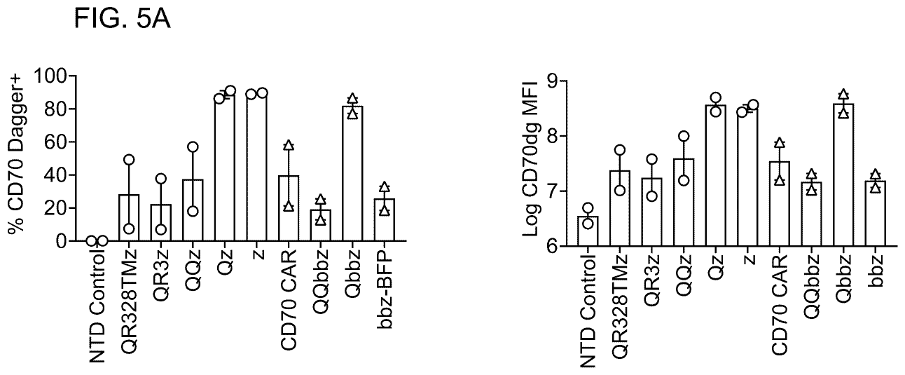

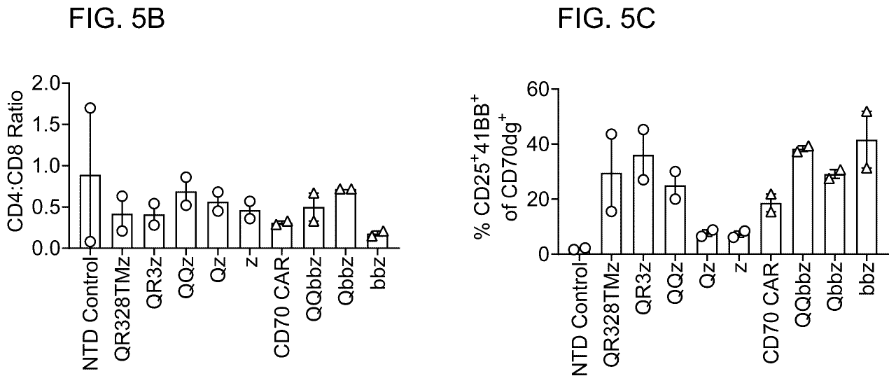

We generated a series of clone 4F11-based CD70 dagger constructs that are first-generation CARs (i.e., without a co-stimulatory domain) or a second-generation CARs (i.e., with a co-stimulatory domain). Additionally, we tested constructs with or without a mimotope-based safety switch. The results are shown in FIGS. 5A-C. All CD70 daggers tested contain a wildtype CD3z signaling domain (“z”), and a CD8 transmembrane domain, unless indicated otherwise. Of the CD70 daggers that are first-generation CARs: QR3z: further containing the QR3 safety switch (a two-part safety switch comprising the rituximab mimotope R (SEQ ID NO: 592) followed by SEQ ID NO: 595); QQz further containing the QQ safety switch (SEQ ID NO: 596); Qz: further containing the Q safety switch (SEQ ID NO: 594); and QR328TMz: in addition to the QR3 safety switch, further containing the CD28 transmembrane domain instead of the CD8 transmembrane domain. All constructs that are second-generation CARs contain a costimulatory 4-1BB signaling domain in addition to a CD8 transmembrane domain and a CD3z signaling domain: among which “CD70 CAR” further containing a QR3 safety switch, and the designations QQbbz, Qbbz and bbz further containing the QQ safety switch, the Q safety switch and no safety switch, respectively. All cells were further modified to knockout the TRAC locus.

Primary T cells were isolated and transduced with lentiviral vectors (LVV) expressing different CD70 dagger constructs. Cells transduced with the constructs tested were all successfully produced, Constructs designated Qz, z and Qbbz showed the highest levels of expression (FIG. 5A), and constructs Qz and z had the lowest levels of activation markers expression (FIG. 5C). In general, the CD70 dagger expressing cells exhibited similar CD4:CD8 T cell ratio (FIG. 5B) and differentiation state (data not shown) as compared to NTD cells.

To evaluate the activities of the CD70 dagger constructs, we performed alloreactive T cell MLRs with selected CD70 dagger expressing cells. For alloreactive T cell MLRs and MLTCs, primed alloreactive T cells were co-cultured with graft T cells or the combination of graft T cells and tumor targets at the indicated ratios in 200 uL RPMI medium supplemented with 10% FBS and 20 U/mL of recombinant human IL-2 in round-bottomed 96-well plates. If MLR co-cultures exceeded 4 days, half the medium was replaced on day 4. Medium was then replaced every 2-3 days. Cells were analyzed by flow cytometry at the indicated time points. For PBMC MLRs, host PBMCs were co-cultured with graft T cells at a ratio of 10:1 in 200 uL RPMI medium supplemented with 10% FBS and 20 U/mL of recombinant human IL-2 in round-bottomed 96-well plates. To prime alloreactive T cells, unedited graft donor T cells were irradiated at 30 Gy and co-cultured with host PBMCs at a ratio of 1:1 in RPMI supplemented with 10% FBS and 20 U/mL of recombinant human IL-2, IL-7, and IL-15. At day 4 of the co-culture, half the media was replaced with fresh RPMI supplemented with 10% FBS. At day 7, T cells were isolated using EasySep™ human T cell isolation kit (STEMCELL Technologies) as instructed by the manufacturer protocol.

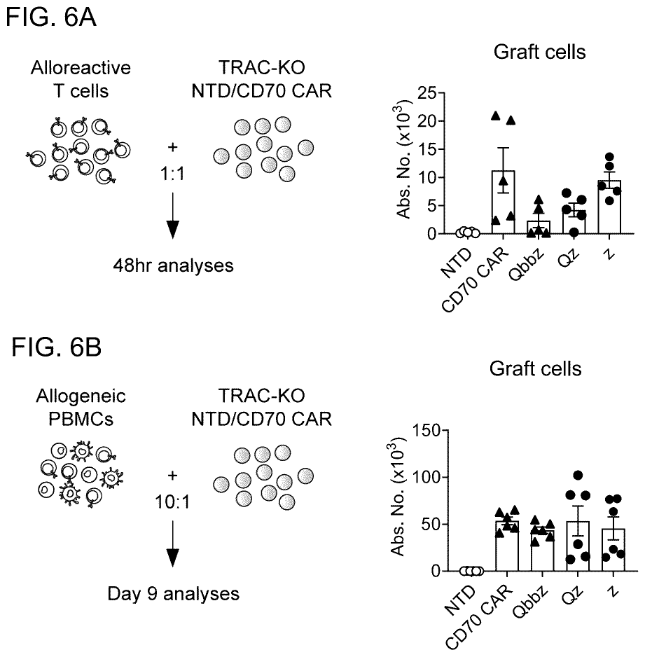

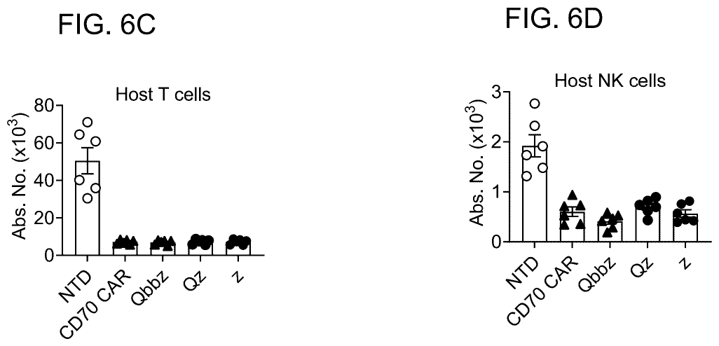

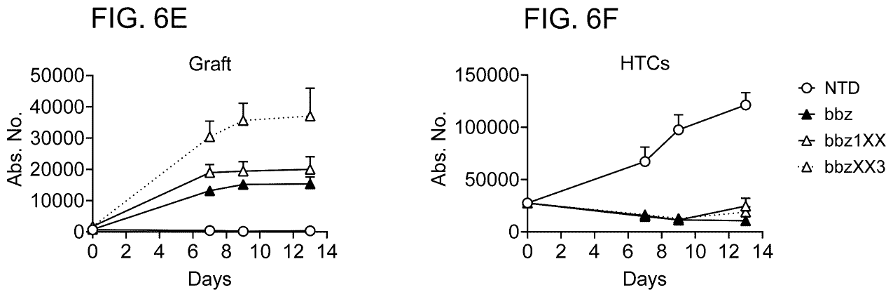

As shown in FIG. 6A, T cells expressing the “CD70 CAR” dagger resisted alloreactive T cell-mediated rejection while NTD T cells were completed rejected. CD70dg-Qbbz, CD70dg-Qz, and CD70dg-z expressing T cells showed improved survival compared to NTD T cells to varying degrees (FIG. 6A). We next tested the activities of the CD70 daggers in PBMC MLRs, in which NK cell alloreactivity can contribute to rejection and the priming kinetics of host T cells occur naturally in response to allogeneic T cells. In the PBMC MLR assay, all CD70 daggers tested significantly increased the survival of graft T cells as compared to the NTD control cells, which were eliminated by day 9 (FIG. 6B). In the same PBMC MLR assays, CD70 dagger T cells significantly reduced the absolute numbers of HTCs and host NK cells, an evidence of effective dagger activity. See FIGS. 6C-D. Conversely, HTCs and host NK cells expanded when co-cultured with NTD T cells.

Additional constructs containing modifications in the CD3z signaling domain were also tested, including the CD3z domain containing ITAM variants designated 1XX and XX3 (SEQ ID NOs: 585 and 586, respectively). See Feucht et al. Nat Med. 2019, 25(1): 82-88. The cells were further modified to knockout the TRAC locus. The CD70 dagger activities were tested in the MLR assay incubated with allogeneic PBMCs. The graft cells expressing the CD3z variants expanded comparably or better as compared to graft cells expressing wild type CD3z (FIG. 6E), and all graft cells demonstrated comparable levels of killing of host T cells (FIG. 6F). Thus, the CD70 dagger activities were not significantly affected by the modifications in the CD3z intracellular domains.

### Example 5. Testing the CD70 Dagger Activities of Engineered Immune Cells Expressing Different CD70 Binding Proteins (CD70 Daggers) Generated by Site-Specific Integration

Next, we generated cells expressing various CD70 daggers in which the constructs were introduced into the T cells by site-specific integration (SSI). In this experiment, the CD70 dagger constructs were introduced into the CD52 locus by homologous recombination. Additional constructs were tested in this experiment: CD28HTMz: containing the CD28 hinge and transmembrane domain and the CD3z signaling domain; CD8H-CD28TM-z: containing the CD8 hinge and CH28 transmembrane domains and the CD3z domain; bbz referring to the 4-1BB signaling domain in addition to the CD3 signaling domain.

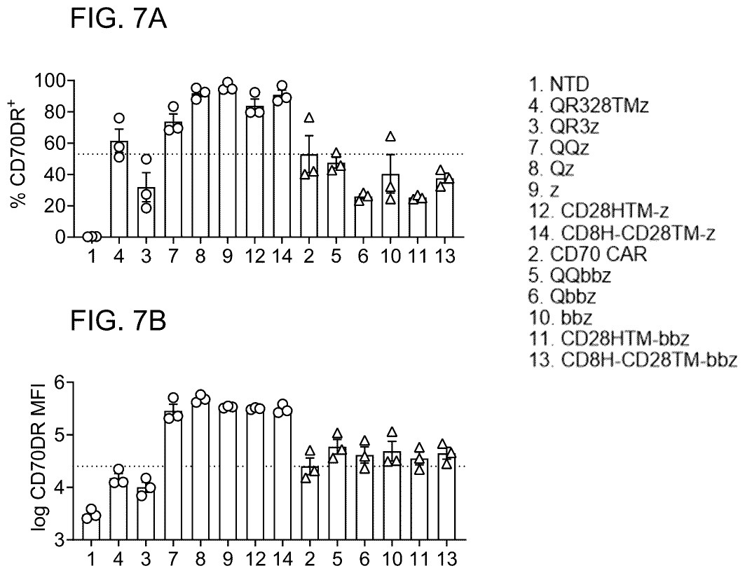

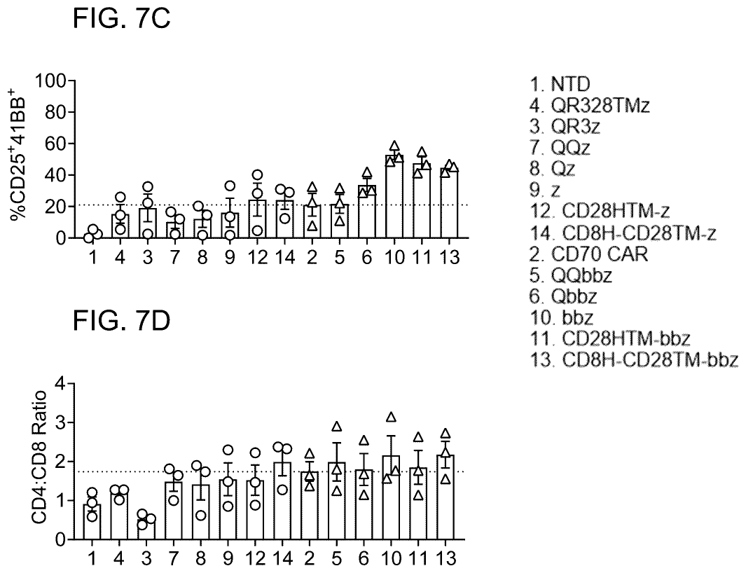

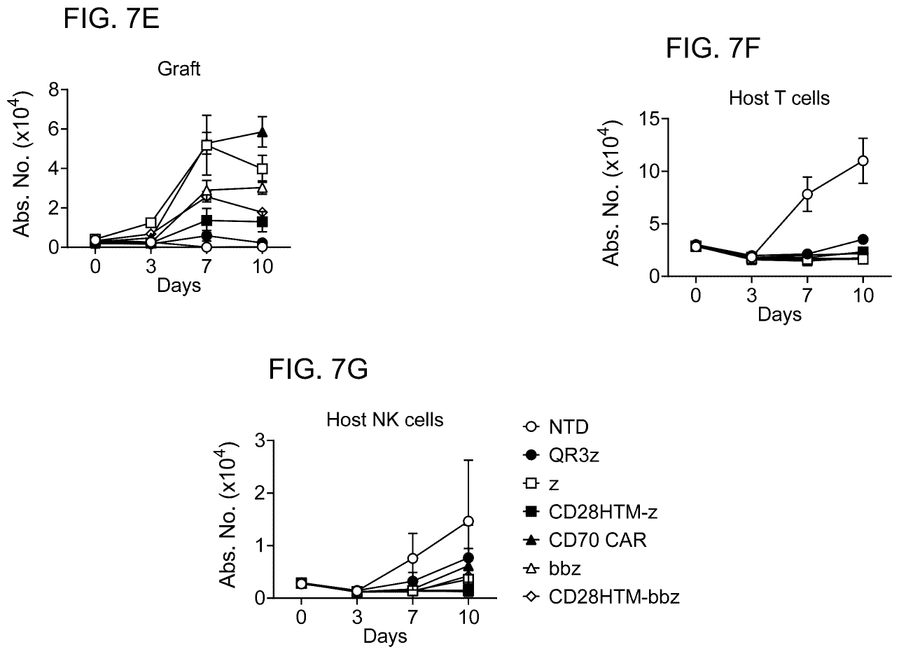

SSI-derived T cells expressing CD70 dagger constructs that are first-generation CARs had higher levels of surface expression and lower levels of activation markers as compared to the second-generation variants (FIGS. 7A-C). A modest increase in CD4:CD8 ratios was observed in SSI-derived CD70 dagger T cells compared to T cells expressing CD70 CAR derived by LVV or the NTD T cell controls (FIG. 7D). Selected CD70 dagger cells were then tested in the PBMC MLR assay. All CD70 dagger T cells tested expanded or persisted on day 13, except for CD70dg-QR3z. See FIG. 7E. CD70dg-QR3z T cells persisted longer than NTD T cell controls, but they were eventually rejected by day 13. All CD70 dagger cells showed activities against host T cells and host NK cells (FIGS. 7F-G). CD70dg-z expanded to the highest degree among all the CD70 dagger constructs tested and showed dagger activity comparable to cells expressing the CD70 CAR.

### Example 6. CD70 Daggers Containing Different CD70 Binding Domains

We next tested CD70 daggers containing CD70 binding domains derived from anti-CD70 antibodies in addition to clone 4F11. The binding affinity of clones 4F11, 8C8 and 8F8 in the form of scFv are within 10 folds of each other. See WO2019/152742.

The binding properties of the anti-CD70 antibodies were further analyzed when expressed as scFv in a second-generation CAR. Luciferase-expressing ACHN cells were transduced with CAR LVVs and stained with His-tagged Fabs at 2.5 μg/mL followed by anti-His antibody at 1:50 dilution. The binding sites (i.e., epitopes) of different anti-CD70 antibodies were analyzed in a masking assay. Briefly, the scFv expressed in the extracellular domain of each CD70 CAR binds to the antigen CD70 protein on the surface of ACHN cells. The binding masks the epitope of the CD70 protein on the cell surface and blocks binding of other anti-CD70 antibodies (Fabs) from binding to the same epitope but permits binding of other anti-CD70 Fabs that bind to different epitopes. The results showed that CAR expressing the 8F8 scFv blocked the binding of 4F11 Fab to the cells, and vice versa (data not shown). Thus, 8F8 and 4F11 competed for binding to CD70, and thus likely binding to the same or overlapping epitopes. On the other hand, CAR expressing the 8C8 scFv did not block the binding of 8F8 or 4F11 Fab to the cells (data not shown). Thus, clone 8C8 did not compete for binding to CD70 with either 4F11 or 8F8, likely binding to a different epitope from 4F11 and 8F8.

The binding sites of each clone were confirmed by protein structural and mutagenesis analysis. Twenty-eight residues on the exposed surface of the CD70 trimer complex were individually mutated to create a panel of CD70 trimer mutants. The binding regions of anti-CD70 chimeric antigen receptors (CARs) were reformatted as antibody fragments and evaluated for binding to the various trimer mutants by biosensor analysis. SPR analysis of CD70 antigen single point mutation supernatant samples were determined on a Biacore T200 SPR instrument (Cytiva, Marlborough, MA). All CD70 antigen single point mutation samples were site-specifically biotinylated via co-expression with BirA, followed by dialysis and filter prior to SPR analysis. All these biotinylated CD70 samples were captured as neat supernatants on the Biacore's CAP surface. All flow cells were then blocked with 20 μM Amine-PEG2-Biotin (APB). Buffer and 100 nM of all anti-CD70 Fabs tested were injected as analytes for 2 minutes and dissociation was monitored for 15 minutes at 30 μL/min. The surface was regenerated according to the manufacturer protocol. All interactions were measured in triplicate using three independent analyte dilution series and captured samples. All sensorgrams were double-referenced with the data from the buffer analyte injections (Myszka, 1999). Data was fitted to a 1:1 Langmuir binding model with mass transport using Biacore T200 Evaluation Software (version 2.0).

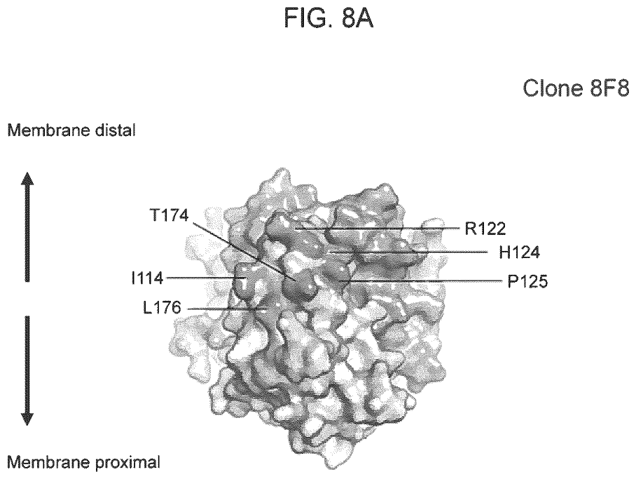

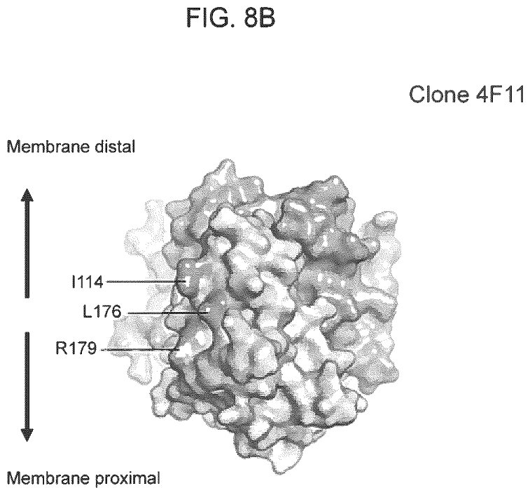

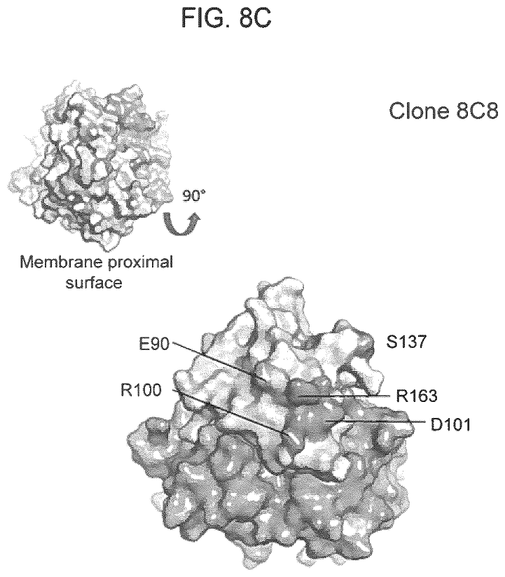

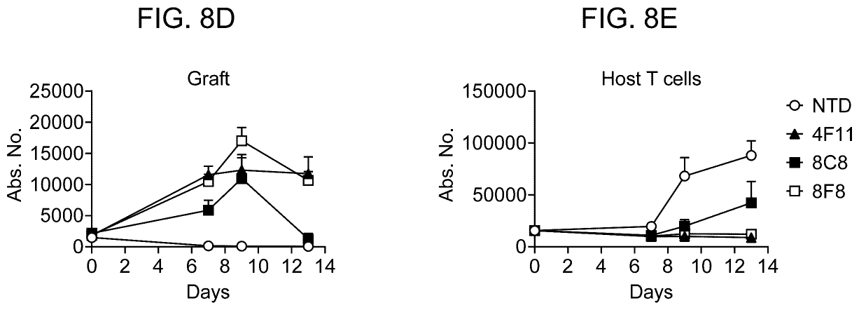

Residues important for the binding of each CAR were identified and mapped onto a structure of the CD70 trimer complex for CARs binding either distal from the membrane or for CARs binding proximal to the membrane. The structural illustrations in FIGS. 8A-C indicate the residues important for binding to the CD70 trimer complex, i.e., the binding sites or epitopes, of clone 8F8, 4F11 and 8C8, respectively. Mutagenesis studies demonstrated that clones 4F11 and 8F8 bind to the membrane distal site of the CD70 protein, and clone 8C8 binds to the membrane proximal site of the CD70 rimer complex.

Next, CD70 dagger constructs containing anti-CD70 antibody clones 8C8, 8F8, and 4F11, in the form of scFv were generated. T cells expressing different CD70 daggers by LVV transduction were tested in a T cell MLR assay. All three CD70 daggers contain a CD3ζ intracellular signaling domain and a 4-1BB co-stimulatory domain. As shown in FIGS. 8D-E, CD70 dagger expressing T cells all expanded in the MLR assay though exhibiting varying degrees of persistence. All three CD70 dagger constructs inhibited host T cells proliferation with the 4F11 and 8F8-derived CD70 daggers that bind to membrane distal site of CD70 being more effective than 8C8-derived CD70 dagger.

The results show that all CD70 dagger constructs tested, with different CD70 binding domains that bind to different epitopes, and/or different intracellular signaling domains, generated either by LVV transduction or site-specific integration, exhibited dagger activities by reducing host T cells and/or NK cells. The varying degrees of expansion and dagger activities exhibited by different constructs suggest that the CD70 dagger activity may be fine-tuned to achieve a desired level of lymphodepletion for use in, for example, adoptive cell therapy or CAR T cell therapy.

### Example 7. CD70 Dagger Expressed in Conjunction with a CAR Specific for a Non-CD70 Target

Next, we examine the CD70 dagger activities when expressed in conjunction with a CAR specific for a non-CD70 tumor target. A tandem CAR was constructed comprising the anti-CD70 clone 4F11 scFv and the anti-CD19 clone 4G7 scFv (U.S. Pat. No. 10,874,693). CAR T cells expressing the tandem CAR are tested in the T cell MLR or PBMC MLR assay or MLTC assay. The expansion of CD70 dagger expressing CAR T cells and inhibition of host T cells and/or NK cells are measured in the MLR assays.

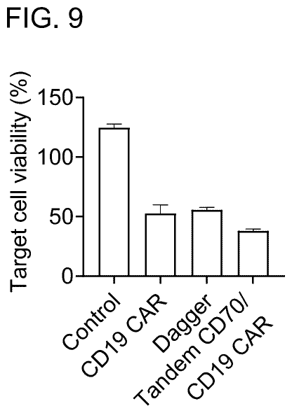

Whether the dagger would affect the cytotoxicity of CD19-CD70 tandem CAR T cells against CD19 expressing target cells was examined. The results in FIG. 9 show that the CD19-CD70 tandem CAR T cells exhibited robust cytotoxicity against the CD19 target cell line Raji, as determined by measuring residual luciferase activity 48 hours following co-culture of effector cells and luciferase-labeled target cells. In this experiment, the dagger cells alone, without the CD19 binding domain, also demonstrated cytotoxic activity due to the presence of CD70 on the Raji cells. The results show that the presence of the CD70 binding domain in the tandem CAR did not negatively impact the cytotoxic activity of the CD19 CAR. Similar results were observed when the CD70 dagger was expressed on separate T cells, and the presence of the separate CD70 dagger cells did not negatively affect the cytotoxicity of CAR T cells specific for another non-CD70 target in a long-term killing assay (data not shown). The data in this experiment show that the CD70 dagger, whether expressed on separate dagger cells or on the same CAR T cells (e.g., as part of a tandem CAR), did not negatively impact the cytotoxic activity of the CAR to its specific tumor antigen.

1-2. (canceled)

3. A method of lymphodepletion in a patient in need thereof, comprising the step of administering engineered immune cells to the patient, wherein the engineered immune cells comprise or functionally express a CD70 binding protein that comprises a CD70 binding domain and a transmembrane domain, and wherein the engineered immune cells inhibit proliferation and/or activity of CD70 positive cells in the patient.

4. The method of claim 3, wherein the CD70 binding domain comprises an anti-CD70 antibody, or a CD70 binding fragment thereof, optionally the anti-CD70 antibody comprises a scFv.

5. (canceled)

6. The method of claim 4, wherein the CD70 binding protein further comprises a hinge domain, optionally the hinge domain comprises a CD8 hinge.

7. The method of claim 4, wherein the CD70 binding protein further comprises an intracellular signaling domain selected from the group consisting of a CD3ζ signaling domain, a CD3δ signaling domain, a CD3γ signaling domain, a CD3ε signaling domain, and a variant thereof.

8. The method of claim 4, wherein the CD70 binding protein comprises a CD3ζ or a CD3γ signaling domain and does not comprise a costimulatory domain.

9-10. (canceled)

11. The method of claim 7, wherein the one or more intracellular domain comprises the amino acid sequence of one or more of SEQ ID NOs: 272, 275, 281, 282, 289-292, 312-314, 585-591, and 617.

12. (canceled)

13. The method of claim 4, wherein the CD70 positive cells are normal or non-cancerous lymphocytes in the patient selected from the group consisting of T cells, B cells, NK cells and activated T cells.

14-16. (canceled)

17. The method of claim 3, wherein the engineered immune cells are autologous or allogeneic to the patient.

18. The method of claim 3, wherein the engineered immune cells further comprise a genomic modification of one or more of an endogenous TCRa gene, and an endogenous CD52 gene, but not an endogenous CD70 gene, wherein the engineered immune cells exhibit reduced level of TCRa and/or, CD52 expression and/or activity as compared to a control immune cell without the genomic modification.

19. The method of claim 3, wherein the patient has or is expected to have a host v. graft rejection.

20. The method of claim 3, wherein the patient is in need for a bone marrow transplant, stem cell transplant, or tissue transplant, wherein the transplant exhibits longer persistence in the patient as compared to a patient not administered the engineered immune cells.

21. (canceled)

22. The method of claim 3, wherein the patient is receiving adoptive cell therapy, optionally wherein the adoptive cell therapy is chimeric antigen receptor (CAR) T cell therapy.

23-24. (canceled)

25. The method of claim 22, wherein the patient is further administered a chemotherapy agent before, simultaneously, or following receiving adoptive cell therapy, and optionally the chemotherapy agent is cyclophosphamide.

26. The method of claim 22, wherein the patient is not administered fludarabine before, simultaneously, or following receiving adoptive cell therapy.

27. (canceled)

28. The method of claim 3, wherein the engineered immune cells further comprise an additional antigen binding protein other than the CD70 binding protein.

29. The method of claim 28, wherein one protein comprises the additional antigen binding protein and the CD70 binding protein, and wherein the additional antigen binding domain comprises an antibody that binds to the target of interest, and optionally the additional antigen binding domain comprises a scFv.

30. The method of claim 29, wherein the one protein is a bispecific CAR.

31. The method of claim 28, wherein the additional antigen binding protein is a CAR.

32. (canceled)

33. The method of claim 31, wherein the CAR comprises an intracellular signaling domain selected from the group consisting of a CD3ζ signaling domain, a CD28 signaling domain and a 4-1BB signaling domain.

34-36. (canceled)

37. The method of claim 2, wherein the additional protein is selected from the group consisting of BCMA, EGFRvIII, Flt-3, WT-1, CD20, CD22, CD23, CD30, CD38, CD33, CD133, WT1, TSPAN10, MHC-PRAME, HER2, MSLN, PSMA, PSCA, GPC3, Liv1, ADAM10, CHRNA2, LeY, NKG2D, CS1, CD44v6, ROR1, CD19, Claudin-18.2, DLL3, Muc17, Muc3, Muc16, FAP alpha, Ly6G6D, or RNF43.

38-99. (canceled)

100. The method of claim 3, wherein the method comprises administering about 10³ or 10⁴ to about 10⁹ engineered immune cells per kg body weight of the patient, or about 10⁵ to about 10⁶ engineered immune cells per kg body weight of the patient, or between 0.1×10⁶ and 5×10⁶ engineered immune cells per kg body weight of the patient.

101-109. (canceled)


SEQUENCE LISTING (617 sequences)

SEQ ID NO: 1 - protein, 112 aa, synthetic construct
  REGION 1..112: note = Description of sequence: Light chain-31H1
DIVMTQNPLSSPVTLGQPASISCRSSQSLVHSDGNTYLSWLQQRPGQSPRLLIYKISNRFSGVPDRFSGSGAGTDFTLKISRVEAEDVGVYYCMQATQFPLTIGGGSKVEIK

SEQ ID NO: 2 - protein, 118 aa, synthetic construct
  REGION 1..118: note = Description of sequence: Heavy chain-31H1
QVQLVQSGAEVKKPGSSVKVSCKASGGTFSSYGFSWVRQAPGQGLEWMGGIIPIFGSANYAQKFQGRVTITADKSTSTVYMELISLRSEDTAVYYCARGGSSSPFAYWGQGTLVTVSS

SEQ ID NO: 3 - protein, 112 aa, synthetic construct
  REGION 1..112: note = Description of sequence: Light chain-63B2
DIVMTQTPLSSPVTLGQPASISCRSSQSLVHSDGNTYLSWLQQRPGQSPRLLIYKISNRFSGVPDRFSGSGAGTDFTLKISRVEAEDVGVYYCMQATQFPLTIGGGSKVEIK

SEQ ID NO: 4 - protein, 118 aa, synthetic construct
  REGION 1..118: note = Description of sequence: Heavy chain-63B2
QVQLVQSGAEVKKPGSSVKVSCKASGGTFSSYGFSWVRQAPGQGLEWMGGIIPIFGTANYAQKFQGRVTITADKSTSTVFMELISLRSEYTAVYYCARGGSSSPFAYWGQGTLVTVSS

SEQ ID NO: 5 - protein, 107 aa, synthetic construct
  REGION 1..107: note = Description of sequence: Light chain-40E3
DIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 6 - protein, 112 aa, synthetic construct
  REGION 1..112: note = Description of sequence: Heavy chain-40E3
QVQLQESGPGLVKPSETLSLTCTVSGGSISSYYWNWIRQPPGKGLEWIGYIYYSGSTNYNPSLKSRVTISVDTSKNQFSLKLRSVTAADTAVYYCARDIRTWGQGTLVTVSS

SEQ ID NO: 7 - protein, 112 aa, synthetic construct
  REGION 1..112: note = Description of sequence: Light chain-42C3
DVVMTQSPLSLPVTLGQPASISCRSSQSLVYSDENTYLNWFQQRPGQSLRRLIYQVSNRDSGVPDRFSGSGSGTDFTLKISRVEAEDVGVYFCMQGTYWPPTFGGGTKVEIK

SEQ ID NO: 8 - protein, 117 aa, synthetic construct
  REGION 1..117: note = Description of sequence: Heavy chain-42C3
EVQLVESGGGLVQPGGSLRLSCAASGFTFRNSWMSWVRQAPGKGLEWVANIKRDGSEKYYVDSVKGRFTISRDNAKNSLYLQMNSLRAEDTAVYYCARDQTGSFDYWGQGTLVTVSS

SEQ ID NO: 9 - protein, 106 aa, synthetic construct
  REGION 1..106: note = Description of sequence: Light chain-45F11
EIVMTQSPATLSMSLGERATLSCRASQSVSSSLAWYQQKPGQAPRLLIYGASTRATGIPARFGGSGSGTEFTLTISSLQSEDFAVYYCQQYINWPHFGGGTKVEIK

SEQ ID NO: 10 - protein, 115 aa, synthetic construct
  REGION 1..115: note = Description of sequence: Heavy chain-45F11
QVQLRGSGPGLVKPSETLSLTCTVSDDSISVYYWSWIRQPAGKGLEWIGRVYSSGNINYNPSLESRVTMSVDTSKSRFSLNLSSVTAADTAVYYCARGLDAFDIWGQGTMVTVSS

SEQ ID NO: 11 - protein, 107 aa, synthetic construct
  REGION 1..107: note = Description of sequence: Light chain-64F9
DIQMTQSPSSLSASVGDRVTITCQASQDISNYLNWYQQKPGKAPKILIYGASNLETGVPSRFSGSGSGTDFTFAISSLQPEDVATYYCQQYDNFPITFGQGTRLEIK

SEQ ID NO: 12 - protein, 115 aa, synthetic construct
  REGION 1..115: note = Description of sequence: Heavy chain-64F9
EVQLLESGGGLVQPGESLRLSCEVSGFTFTSYAMSWVRQVPGKGLEWVSIISGVAFTTYYADSVKGRFTISRDHSKNTLYLQMNGLRAEDTAVYYCVKVDGEVYWGQGTLVTVSS

SEQ ID NO: 13 - protein, 108 aa, synthetic construct
  REGION 1..108: note = Description of sequence: Light chain-72C2
EIVMTQSPDTLSVSPGERAILSCRASQSVSSNLAWYQQKPGQAPRLLIYSASTRASGIPARFSGSGSGTEFTLSISSLQSEDFAVYYCQQYDNWPPLTFGGGTKVEIK

SEQ ID NO: 14 - protein, 117 aa, synthetic construct
  REGION 1..117: note = Description of sequence: Heavy chain-72C2
QVQLVQSGAEVKKPGSSVKVSCEASGGTFITYAISWVRQAPGQGLEWMGGIIPFFGTANYAQKFQGRVTITADKSTSTASMELRSLRSEDTAMYYCAQWELFFFDFWGQGTPVTVSS

SEQ ID NO: 15 - protein, 108 aa, synthetic construct
  REGION 1..108: note = Description of sequence: Light chain-2F10
EIVLTQSPGTLSLSPGERATLSCRASQSVSSSYLAWYQQQPGQAPRLLIYGASSRATGIPDRFSGSGSGTDFTLTISRLEPEDFAIYYCQQYGSSPLTFGGGTKVEIK

SEQ ID NO: 16 - protein, 120 aa, synthetic construct
  REGION 1..120: note = Description of sequence: Heavy chain-2F10
AVQLVESGGGLVQPGGSLRLSCAASGFTFTYYSMNWVRQAPGKGLEWVSHISIRSSTIYFADSAKGRFTISRDNAKNSLYLQMNSLRDEDTAVYYCARGSGWYGDYFDYWGQGTLVTVSS

SEQ ID NO: 17 - protein, 107 aa, synthetic construct
  REGION 1..107: note = Description of sequence: Light chain-4F11
DIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 18 - protein, 123 aa, synthetic construct
  REGION 1..123: note = Description of sequence: Heavy chain-4F11
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSS

SEQ ID NO: 19 - protein, 107 aa, synthetic construct
  REGION 1..107: note = Description of sequence: Light chain-10H10
DIQMTQSPSSVSASVGDRVTITCRASQGISSWLAWYQQKPGKAPKVLIYAASSLQSGVPSRFSGSGSGTDFTLTISSLQPEDFATYYCQQAFSFPFTFGPGTKVDIK

SEQ ID NO: 20 - protein, 119 aa, synthetic construct
  REGION 1..119: note = Description of sequence: Heavy chain-10H10
EVQLVESGGGLVQPGGSLRLSCAVSGFTFSNHNIHWVRQAPGKGLEWISYISRSSSTIYYADSVKGRFTISRDNAKNSLYLQMNSLRDEDTAVYYCARDHAQWYGMDVWGQGTTVTVSS

SEQ ID NO: 21 - protein, 112 aa, synthetic construct
  REGION 1..112: note = Description of sequence: Light chain-17G6
DIVMTQSPDSLAVSLGERATINCKSSQSVLYSYNNKNYVAWYQQKPGQPPNLLIFWASTRESGVPDRFSGSGSGTDFTLTISSLQAEDVAVYYCQQYYSTLTFGGGTKVEIK

SEQ ID NO: 22 - protein, 124 aa, synthetic construct
  REGION 1..124: note = Description of sequence: Heavy chain-17G6
EVQLVESGGGLVQPGGSLRLSCVASGFTFSSYWMSWVRQAPGKGLEWVASIKQDGSEKYYVDSVKGRFTISRDNAKNSVYLQMNSLRAEDTGVYYCAREGVNWGWRLYWHFDLWGRGTLVTVSS

SEQ ID NO: 23 - protein, 108 aa, synthetic construct
  REGION 1..108: note = Description of sequence: Light chain-65E11
EIVLTQSPGTLSLSPGERVTLSCRASQSVSSSYLAWYQQKPGQAPRLLIYDASSRATGIPDRFSGSGSGTDFTLTISRLEPEDFAVYYCQQYGSSPLTFGGGTKVEIK

SEQ ID NO: 24 - protein, 120 aa, synthetic construct
  REGION 1..120: note = Description of sequence: Heavy chain-65E11
EVQVVESGGGLVQPGGSLRLSCAASGFTFSSYSMNWVRQAPGKGLEWVSHSSISRGNIYFADSVKGRFTISRDNAKNSLYLQMNSLRDEDTAVYYCARGSGWYGDYFDYWGQGTLVTVSS

SEQ ID NO: 25 - protein, 112 aa, synthetic construct
  REGION 1..112: note = Description of sequence: Light chain-P02B10
ELQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGVVFGGGTKLTVL

SEQ ID NO: 26 - protein, 120 aa, synthetic construct
  REGION 1..120: note = Description of sequence: Heavy chain-P02B10
EVQLLESGGGLVQPGGSLRLSCAASGFAFSNYAMSWVRQAPGKGLEWVSAIRGGGGSTYYADSVKGRFTISRDNSKNTLYLQMNSLRAEDTAVYYCARDFISGTWYPDYWGQGTLVTVSS

SEQ ID NO: 27 - protein, 112 aa, synthetic construct
  REGION 1..112: note = Description of sequence: Light chain-P07D03
ELQSVLTQPPSASGTPGQRVTISCSGSRSNIGSNYVYWYQQLPGTAPKLLIYRNNQRPSGVPDRFSGSKSGTSASLAISGLRSEDEADYYCASWDGSLSAVVFGTGTKLTVL

SEQ ID NO: 28 - protein, 122 aa, synthetic construct
  REGION 1..122: note = Description of sequence: Heavy chain-P07D03
EVQLVQSGAEVKKPGESLKISCKGSGYRFTSYWIGWVRQMPGKGLEWMGSIYPDDSDTRYSPSFQGQVTISADKSISTAYLQWSSLKASDTAMYYCASSTVDYPGYSYFDYWGQGTLVTVSS

SEQ ID NO: 29 - protein, 112 aa, synthetic construct
  REGION 1..112: note = Description of sequence: Light chain-P08A02
ELQSVLTQPPSASGTPGQRVTISCSGSSSNIGSNYVYWYQQLPGTAPKLLIYRNNQRPSGVPDRFSGSKSGTSASLAISGLRSEDEADYYCATWDDSLGSPVFGTGTKLTVL

SEQ ID NO: 30 - protein, 123 aa, synthetic construct
  REGION 1..123: note = Description of sequence: Heavy chain-P08A02
EVQLVQSGAEVKKPGESLKISCKGSGYTFTNYWIAWVRQMPGKGLEWMGIIYPDGSDTRYSPSFQGQVTISADKSISTAYLQWSSLKASDTAMYYCARDITSWYYGEPAFDIWGQGTLVTVSS

SEQ ID NO: 31 - protein, 110 aa, synthetic construct
  REGION 1..110: note = Description of sequence: Light chain-P08E02
ELDIQMTQSPSSLSASVGDRVTITCRASQSISRYLNWYQQKPGKAPKLLIYAASILQTGVPSRFSGSGSGTDFTLTISSLQPEDFATYYCQQSYSTTMWTFGQGTKVEIK

SEQ ID NO: 32 - protein, 122 aa, synthetic construct
  REGION 1..122: note = Description of sequence: Heavy chain-P08E02
EVQLVQSGAEVKKPGESLKISCKGSGYSFTSSWIGWVRQMPGKGLEWMGIIYPGDSDTRYSPSFQGQVTISADKSISTAYLQWSSLKASDTAMYYCAKGLSQAMTGFGFDYWGQGTLVTVSS

SEQ ID NO: 33 - protein, 113 aa, synthetic construct
  REGION 1..113: note = Description of sequence: Light chain-P08F08
ELQSVLTQPPSASGTPGQRVTISCSGSSSNIGSNYVNWYQQLPGTAPKLLIYGDYQRPSGVPDRFSGSKSGTSASLAISGLRSEDEADYYCATRDDSLSGSVVFGTGTKLTVL

SEQ ID NO: 34 - protein, 122 aa, synthetic construct
  REGION 1..122: note = Description of sequence: Heavy chain-P08F08
EVQLVQSGAEVKKPGESLKISCKGSGYGFTSYWIGWVRQMPGKGLEWMGIIHPDDSDTKYSPSFQGQVTISADKSISTAYLQWSSLKASDTAMYYCASSYLRGLWGGYFDYWGQGTLVTVSS

SEQ ID NO: 35 - protein, 110 aa, synthetic construct
  REGION 1..110: note = Description of sequence: Light chain-P08G02
ELDIQMTQSPSSLSASVGDRVTITCRASQSIYDYLHWYQQKPGKAPKLLIYDASNLQSGVPSRFSGSGSGTDFTLTISSLQPEDFATYYCQQSYTTPLFTFGQGTKVEIK

SEQ ID NO: 36 - protein, 126 aa, synthetic construct
  REGION 1..126: note = Description of sequence: Heavy chain-P08G02
EVQLVQSGAEVKKPGESLKISCKGSGYTFPSSWIGWVRQMPGKGLEWMGIIYPDTSHTRYSPSFQGQVTISADKSISTAYLQWSSLKASDTAMYYCARASYFDRGTGYSSWWMDVWGQGTLVTVSS

SEQ ID NO: 37 - protein, 110 aa, synthetic construct
  REGION 1..110: note = Description of sequence: Light chain-P12B09
ELDIQMTQSPSSLSASVGDRVTITCRASQYIGRYLNWYQQKRGKAPKLLIHGATSLASGVPSRFSGSGSGTDFTLTISSLQPEDFATYYCQQSYSTTSPTFGQGTKVEIK

SEQ ID NO: 38 - protein, 123 aa, synthetic construct
  REGION 1..123: note = Description of sequence: Heavy chain-P12B09
EVQLLESGGGLVQPGGSLRLSCAASGFTFSQYSMSWVRQAPGKGLEWVSAISGGGVSTYYADSVKGRFTISRDNSKNTLYLQMNSLRAEDTAVYYCASDISDSGGSHWYFDYWGQGTLVTVSS

SEQ ID NO: 39 - protein, 112 aa, synthetic construct
  REGION 1..112: note = Description of sequence: Light chain-P12F02
ELQSVLTQPPSASGTPGQRVTISCSGSTSNIGRNYVYWYQQLPGTAPKLLIYRTNQRPSGVPDRFSGSKSGTSASLAISGLRSEDEADYYCAAWDDSLSGRVFGTGTKLTVL

SEQ ID NO: 40 - protein, 121 aa, synthetic construct
  REGION 1..121: note = Description of sequence: Heavy chain-P12F02
EVQLLESGGGLVQPGGSLRLSCAASGFTFSSYAMSWVRQAPGKGLEWVSTISGTGGTTYYADSVKGRFTISRDNSKNTLYLQMNSLRAEDTAVYYCAKVRAGIDPTASDVWGQGTLVTVSS

SEQ ID NO: 41 - protein, 112 aa, synthetic construct
  REGION 1..112: note = Description of sequence: Light chain-P12G07
ELQSVLTQPPSASGTPGQRVTISCSGSSSNIGSNYVYWYQQLPGTAPKPLIYMNNQRPSGVPDRFSGSKSGTSASLAISGLRSEDEADYYCAAWDDSLSAVVFGTGTKLTVL

SEQ ID NO: 42 - protein, 124 aa, synthetic construct
  REGION 1..124: note = Description of sequence: Heavy chain-P12G07
EVQLLESGGGLVQPGGSLRLSCAASGFTFNNFAMSWVRQAPGKGLEWVSGISGSGDNTYYADSVKGRFTISRDNSKNTLYLQMNSLRAEDTAVYYCAKDRDIGLGWYSYYLDVWGQGTLVTVSS

SEQ ID NO: 43 - protein, 113 aa, synthetic construct
  REGION 1..113: note = Description of sequence: Light chain-P13F04
ELQSVLTQPPSASGTPGQRVTISCSGSNSNIGTNYVSWYQQLPGTAPKLLIYRSSRRPSGVPDRFSGSKSGTSASLAISGLRSEDEADYYCAAWDGSLSGHWVFGTGTKLTVL

SEQ ID NO: 44 - protein, 119 aa, synthetic construct
  REGION 1..119: note = Description of sequence: Heavy chain-P13F04
QVQLVQSGAEVKKPGSSVKVSCKASGGTFSSYAISWVRQAPGQGLEWMGEIIPIFGTASYAQKFQGRVTITADESTSTAYMELSSLRSEDTAVYYCARAGWDDSWFDYWGQGTLVTVSS

SEQ ID NO: 45 - protein, 110 aa, synthetic construct
  REGION 1..110: note = Description of sequence: Light chain-P15D02
ELDIQMTQSPSSLSASVGDRVTITCRASQSIDTYLNWYQQKPGKAPKLLIYSASSLHSGVPSRFSGSGSGTDFTLTISSLQPEDFATYYCQQSYSTTAWTFGQGTKVEIK

SEQ ID NO: 46 - protein, 122 aa, synthetic construct
  REGION 1..122: note = Description of sequence: Heavy chain-P15D02
EVQLVQSGAEVKKPGESLKISCKGSGYSFASYWIGWVRQMPGKGLEWMGVIYPGTSETRYSPSFQGQVTISADKSISTAYLQWSSLKASDTAMYYCAKGLSASASGYSFQYWGQGTLVTVSS

SEQ ID NO: 47 - protein, 109 aa, synthetic construct
  REGION 1..109: note = Description of sequence: Light chain-P16C05
ELDIQMTQSPSSLSASVGDRVTITCRASQSIGQSLNWYQQKPGKAPKLLIYGASSLQSGVPSRFSGSGSGTDFTLTISSLQPEDFATYYCQQSYSTPITFGQGTKVEIK

SEQ ID NO: 48 - protein, 124 aa, synthetic construct
  REGION 1..124: note = Description of sequence: Heavy chain-P16C05
EVQLVQSGAEVKKPGESLKISCKGSGYSFTDYWIGWVRQMPGKGLEWMGMISPGGSTTIYRPSFQGQVTISADKSISTAYLQWSSLKASDTAMYYCAREMYTGGYGGSWYFDYWGQGTLVTVSS

SEQ ID NO: 49 - protein, 5 aa, synthetic construct
  REGION 1..5: note = Description of sequence: Heavy chain-CDRH1:31H1 Kabat
SYGFS

SEQ ID NO: 50 - protein, 7 aa, synthetic construct
  REGION 1..7: note = Description of sequence: Heavy chain-CDRH1:31H1 Chothia
GGTFSSY

SEQ ID NO: 51 - protein, 10 aa, synthetic construct
  REGION 1..10: note = Description of sequence: Heavy chain-CDRH1:31H1 Extended
GGTFSSYGFS

SEQ ID NO: 52 - protein, 17 aa, synthetic construct
  REGION 1..17: note = Description of sequence: Heavy chain-CDRH2:31H1 Kabat
GIIPIFGSANYAQKFQG

SEQ ID NO: 53 - protein, 6 aa, synthetic construct
  REGION 1..6: note = Description of sequence: Heavy chain-CDRH2:31H1 Chothia
IPIFGS

SEQ ID NO: 54 - protein, 9 aa, synthetic construct
  REGION 1..9: note = Description of sequence: Heavy chain-CDRH3:31H1
GGSSSPFAY

SEQ ID NO: 55 - protein, 5 aa, synthetic construct
  REGION 1..5: note = Description of sequence: Heavy chain-CDRH1:63B2 Kabat
SYGFS

SEQ ID NO: 56 - protein, 7 aa, synthetic construct
  REGION 1..7: note = Description of sequence: Heavy chain-CDRH1:63B2 Chothia
GGTFSSY

SEQ ID NO: 57 - protein, 10 aa, synthetic construct
  REGION 1..10: note = Description of sequence: Heavy chain-CDRH1:63B2 Extended
GGTFSSYGFS

SEQ ID NO: 58 - protein, 17 aa, synthetic construct
  REGION 1..17: note = Description of sequence: Heavy chain-CDRH2:63B2 Kabat
GIIPIFGTANYAQKFQG

SEQ ID NO: 59 - protein, 6 aa, synthetic construct
  REGION 1..6: note = Description of sequence: Heavy chain-CDRH2:63B2 Chothia
IPIFGT

SEQ ID NO: 60 - protein, 9 aa, synthetic construct
  REGION 1..9: note = Description of sequence: Heavy chain-CDRH3:63B2
GGSSSPFAY

SEQ ID NO: 61 - protein, 5 aa, synthetic construct
  REGION 1..5: note = Description of sequence: Heavy chain-CDRH1:40000 kabat
SYYWN

SEQ ID NO: 62 - protein, 7 aa, synthetic construct
  REGION 1..7: note = Description of sequence: Heavy chain-CDRH1:40000 Chothia
GGSISSY

SEQ ID NO: 63 - protein, 10 aa, synthetic construct
  REGION 1..10: note = Description of sequence: Heavy chain-CDRH1:40000 Extended
GGSISSYYWN

SEQ ID NO: 64 - protein, 16 aa, synthetic construct
  REGION 1..16: note = Description of sequence: Heavy chain-CDRH2:40E3 Kabat
YIYYSGSTNYNPSLKS

SEQ ID NO: 65 - protein, 5 aa, synthetic construct
  REGION 1..5: note = Description of sequence: Heavy chain-CDRH2:40E3 Chothia
YYSGS

SEQ ID NO: 66 - protein, 5 aa, synthetic construct
  REGION 1..5: note = Description of sequence: Heavy chain-CDRH3:40E3
DIRTW

SEQ ID NO: 67 - protein, 5 aa, synthetic construct
  REGION 1..5: note = Description of sequence: Heavy chain-CDRH1:42C3 Kabat
NSWMS

SEQ ID NO: 68 - protein, 7 aa, synthetic construct
  REGION 1..7: note = Description of sequence: Heavy chain-CDRH1:42C3 Chothia
GFTFRNS

SEQ ID NO: 69 - protein, 10 aa, synthetic construct
  REGION 1..10: note = Description of sequence: Heavy chain-CDRH1:42C3 Extended
GFTFRNSWMS

SEQ ID NO: 70 - protein, 17 aa, synthetic construct
  REGION 1..17: note = Description of sequence: Heavy chain-CDRH2:42C3 Kabat
NIKRDGSEKYYVDSVKG

SEQ ID NO: 71 - protein, 6 aa, synthetic construct
  REGION 1..6: note = Description of sequence: Heavy chain-CDRH2:42C3 Chothia
KRDGSE

SEQ ID NO: 72 - protein, 8 aa, synthetic construct
  REGION 1..8: note = Description of sequence: Heavy chain-CDRH3:42C3
DQTGSFDY

SEQ ID NO: 73 - protein, 5 aa, synthetic construct
  REGION 1..5: note = Description of sequence: Heavy chain-CDRH1:45F11 Kabat
VYYWS

SEQ ID NO: 74 - protein, 7 aa, synthetic construct
  REGION 1..7: note = Description of sequence: Heavy chain-CDRH1:45F11 Chothia
DDSISVY

SEQ ID NO: 75 - protein, 10 aa, synthetic construct
  REGION 1..10: note = Description of sequence: Heavy chain-CDRH1:45F11 Extended
DDSISVYYWS

SEQ ID NO: 76 - protein, 15 aa, synthetic construct
  REGION 1..15: note = Description of sequence: Heavy chain-CDRH2:45F11 Kabat
VYSSGNINYNPSLES

SEQ ID NO: 77 - protein, 5 aa, synthetic construct
  REGION 1..5: note = Description of sequence: Heavy chain-CDRH2:45F11 Chothia
YSSGN

SEQ ID NO: 78 - protein, 7 aa, synthetic construct
  REGION 1..7: note = Description of sequence: Heavy chain-CDRH3:45F11
GLDAFDI

SEQ ID NO: 79 - protein, 5 aa, synthetic construct
  REGION 1..5: note = Description of sequence: Heavy chain-CDRH1:64F9 Kabat
SYAMS

SEQ ID NO: 80 - protein, 7 aa, synthetic construct
  REGION 1..7: note = Description of sequence: Heavy chain-CDRH1:64F9 Chothia
GFTFTSY

SEQ ID NO: 81 - protein, 10 aa, synthetic construct
  REGION 1..10: note = Description of sequence: Heavy chain-CDRH1:64F9 Extended
GFTFTSYAMS

SEQ ID NO: 82 - protein, 16 aa, synthetic construct
  REGION 1..16: note = Description of sequence: Heavy chain-CDRH2:64F9 Kabat
RVYSSGNINYNPSLES

SEQ ID NO: 83 - protein, 5 aa, synthetic construct
  REGION 1..5: note = Description of sequence: Heavy chain-CDRH2:64F9 Chothia
YSSGN

SEQ ID NO: 84 - protein, 7 aa, synthetic construct
  REGION 1..7: note = Description of sequence: Heavy chain-CDRH3:64F9
GLDAFDI

SEQ ID NO: 85 - protein, 5 aa, synthetic construct
  REGION 1..5: note = Description of sequence: Heavy chain-CDRH1:72C2 Kabat
TYAIS

SEQ ID NO: 86 - protein, 7 aa, synthetic construct
  REGION 1..7: note = Description of sequence: Heavy chain-CDRH1:72C2 Chothia
GGTFITY

SEQ ID NO: 87 - protein, 10 aa, synthetic construct
  REGION 1..10: note = Description of sequence: Heavy chain-CDRH1:72C2 Extended
GGTFITYAIS

SEQ ID NO: 88 - protein, 17 aa, synthetic construct
  REGION 1..17: note = Description of sequence: Heavy chain-CDRH2:72C2 Kabat
GIIPFFGTANYAQKFQG

SEQ ID NO: 89 - protein, 6 aa, synthetic construct
  REGION 1..6: note = Description of sequence: Heavy chain-CDRH2:72C2 Chothia
IPFFGT

SEQ ID NO: 90 - protein, 8 aa, synthetic construct
  REGION 1..8: note = Description of sequence: Heavy chain-CDRH3:72C2
WELFFFDF

SEQ ID NO: 91 - protein, 5 aa, synthetic construct
  REGION 1..5: note = Description of sequence: Heavy chain-CDRH1:2F10 Kabat
YYSMN

SEQ ID NO: 92 - protein, 7 aa, synthetic construct
  REGION 1..7: note = Description of sequence: Heavy chain-CDRH1:2F10 Chothia
GFTFTYY

SEQ ID NO: 93 - protein, 10 aa, synthetic construct
  REGION 1..10: note = Description of sequence: Heavy chain-CDRH1:2F10 Extended
GFTFTYYSMN

SEQ ID NO: 94 - protein, 17 aa, synthetic construct
  REGION 1..17: note = Description of sequence: Heavy chain-CDRH2:2F10 Kabat
HISIRSSTIYFADSAKG

SEQ ID NO: 95 - protein, 6 aa, synthetic construct
  REGION 1..6: note = Description of sequence: Heavy chain-CDRH2:2F10 Chothia
SIRSST

SEQ ID NO: 96 - protein, 11 aa, synthetic construct
  REGION 1..11: note = Description of sequence: Heavy chain-CDRH3:2F10
GSGWYGDYFDY

SEQ ID NO: 97 - protein, 7 aa, synthetic construct
  REGION 1..7: note = Description of sequence: Heavy chain-CDRH1:4F11 Kabat
NARMGVT

SEQ ID NO: 98 - protein, 9 aa, synthetic construct
  REGION 1..9: note = Description of sequence: Heavy chain-CDRH1:4F11 Chothia
GFSLSNARM

SEQ ID NO: 99 - protein, 12 aa, synthetic construct
  REGION 1..12: note = Description of sequence: Heavy chain-CDRH1:4F11 Extended
GFSLSNARMGVT

SEQ ID NO: 100 - protein, 16 aa, synthetic construct
  REGION 1..16: note = Description of sequence: Heavy chain-CDRH2:4F11 Kabat
HIFSNDEKSYSTSLKS

SEQ ID NO: 101 - protein, 5 aa, synthetic construct
  REGION 1..5: note = Description of sequence: Heavy chain-CDRH2:4F11 Chothia
FSNDE

SEQ ID NO: 102 - protein, 13 aa, synthetic construct
  REGION 1..13: note = Description of sequence: Heavy chain-CDRH3:4F11
IRDYYDISSYYDY

SEQ ID NO: 103 - protein, 5 aa, synthetic construct
  REGION 1..5: note = Description of sequence: Heavy chain-CDRH1:10H10 Kabat
NHNIH

SEQ ID NO: 104 - protein, 7 aa, synthetic construct
  REGION 1..7: note = Description of sequence: Heavy chain-CDRH1:10H10 Chothia
GFTFSNH

SEQ ID NO: 105 - protein, 10 aa, synthetic construct
  REGION 1..10: note = Description of sequence: Heavy chain-CDRH1:10H10 Extended
GFTFSNHNIH

SEQ ID NO: 106 - protein, 17 aa, synthetic construct
  REGION 1..17: note = Description of sequence: Heavy chain-CDRH2:10H10 Kabat
YISRSSSTIYYADSVKG

SEQ ID NO: 107 - protein, 6 aa, synthetic construct
  REGION 1..6: note = Description of sequence: Heavy chain-CDRH2:10H10 Chothia
SRSSST

SEQ ID NO: 108 - protein, 10 aa, synthetic construct
  REGION 1..10: note = Description of sequence: Heavy chain-CDRH3:10H10
DHAQWYGMDV

SEQ ID NO: 109 - protein, 5 aa, synthetic construct
  REGION 1..5: note = Description of sequence: Heavy chain-CDRH1:17G6 Kabat
SYWMS

SEQ ID NO: 110 - protein, 7 aa, synthetic construct
  REGION 1..7: note = Description of sequence: Heavy chain-CDRH1:17G6 Chothia
GFTFSSY

SEQ ID NO: 111 - protein, 10 aa, synthetic construct
  REGION 1..10: note = Description of sequence: Heavy chain-CDRH1:17G6 Extended
GFTFSSYWMS

SEQ ID NO: 112 - protein, 17 aa, synthetic construct
  REGION 1..17: note = Description of sequence: Heavy chain-CDRH2:17G6 Kabat
SIKQDGSEKYYVDSVKG

SEQ ID NO: 113 - protein, 6 aa, synthetic construct
  REGION 1..6: note = Description of sequence: Heavy chain-CDRH2:17G6 Chothia
KQDGSE

SEQ ID NO: 114 - protein, 15 aa, synthetic construct
  REGION 1..15: note = Description of sequence: Heavy chain-CDRH3:17G6
EGVNWGWRLYWHFDL

SEQ ID NO: 115 - protein, 5 aa, synthetic construct
  REGION 1..5: note = Description of sequence: Heavy chain-CDRH1:6.5E kabat
SYSMN

SEQ ID NO: 116 - protein, 7 aa, synthetic construct
  REGION 1..7: note = Description of sequence: Heavy chain-CDRH1:6.5E Chothia
GFTFSSY

SEQ ID NO: 117 - protein, 10 aa, synthetic construct
  REGION 1..10: note = Description of sequence: Heavy chain-CDRH1:6.5E Extended
GFTFSSYSMN

SEQ ID NO: 118 - protein, 17 aa, synthetic construct
  REGION 1..17: note = Description of sequence: Heavy chain-CDRH2:65E11 Kabat
HSSISRGNIYFADSVKG

SEQ ID NO: 119 - protein, 6 aa, synthetic construct
  REGION 1..6: note = Description of sequence: Heavy chain-CDRH2:65E11 Chothia
SISRGN

SEQ ID NO: 120 - protein, 11 aa, synthetic construct
  REGION 1..11: note = Description of sequence: Heavy chain-CDRH3:65E11
GSGWYGDYFDY

SEQ ID NO: 121 - protein, 5 aa, synthetic construct
  REGION 1..5: note = Description of sequence: Heavy chain-CDRH1:P02B10 Kabat
NYAMS

SEQ ID NO: 122 - protein, 7 aa, synthetic construct
  REGION 1..7: note = Description of sequence: Heavy chain-CDRH1:P02B10 Chothia
GFAFSNY

SEQ ID NO: 123 - protein, 10 aa, synthetic construct
  REGION 1..10: note = Description of sequence: Heavy chain-CDRH1:P02B10 Extended
GFAFSNYAMS

SEQ ID NO: 124 - protein, 17 aa, synthetic construct
  REGION 1..17: note = Description of sequence: Heavy chain-CDRH2:P02B10 Kabat
AIRGGGGSTYYADSVKG

SEQ ID NO: 125 - protein, 6 aa, synthetic construct
  REGION 1..6: note = Description of sequence: Heavy chain-CDRH2:P02B10 Chothia
RGGGGS

SEQ ID NO: 126 - protein, 11 aa, synthetic construct
  REGION 1..11: note = Description of sequence: Heavy chain-CDRH3:P02B10
DFISGTWYPDY

SEQ ID NO: 127 - protein, 5 aa, synthetic construct
  REGION 1..5: note = Description of sequence: Heavy chain-CDRH1:P07D03 Kabat
SYWIG

SEQ ID NO: 128 - protein, 7 aa, synthetic construct
  REGION 1..7: note = Description of sequence: Heavy chain-CDRH1:P07D03 Chothia
GYRFTSY

SEQ ID NO: 129 - protein, 10 aa, synthetic construct
  REGION 1..10: note = Description of sequence: Heavy chain-CDRH1:P07D03 Extended
GYRFTSYWIG

SEQ ID NO: 130 - protein, 17 aa, synthetic construct
  REGION 1..17: note = Description of sequence: Heavy chain-CDRH2:P07D03 Kabat
SIYPDDSDTRYSPSFQG

SEQ ID NO: 131 - protein, 6 aa, synthetic construct
  REGION 1..6: note = Description of sequence: Heavy chain-CDRH2:P07D03 Chothia
YPDDSD

SEQ ID NO: 132 - protein, 13 aa, synthetic construct
  REGION 1..13: note = Description of sequence: Heavy chain-CDRH3:P07D03
STVDYPGYSYFDY

SEQ ID NO: 133 - protein, 5 aa, synthetic construct
  REGION 1..5: note = Description of sequence: Heavy chain-CDRH1:P08A02 Kabat
NYWIA

SEQ ID NO: 134 - protein, 7 aa, synthetic construct
  REGION 1..7: note = Description of sequence: Heavy chain-CDRH1:P08A02 Chothia
GYTFTNY

SEQ ID NO: 135 - protein, 10 aa, synthetic construct
  REGION 1..10: note = Description of sequence: Heavy chain-CDRH1:P08A02 Extended
GYTFTNYWIA

SEQ ID NO: 136 - protein, 17 aa, synthetic construct
  REGION 1..17: note = Description of sequence: Heavy chain-CDRH2:P08A02 Kabat
IIYPDGSDTRYSPSFQG

SEQ ID NO: 137 - protein, 6 aa, synthetic construct
  REGION 1..6: note = Description of sequence: Heavy chain-CDRH2:P08A02 Chothia
YPDGSD

SEQ ID NO: 138 - protein, 14 aa, synthetic construct
  REGION 1..14: note = Description of sequence: Heavy chain-CDRH3:P08A02
DITSWYYGEPAFDI

SEQ ID NO: 139 - protein, 5 aa, synthetic construct
  REGION 1..5: note = Description of sequence: Heavy chain-CDRH1:P08E02 Kabat
SSWIG

SEQ ID NO: 140 - protein, 7 aa, synthetic construct
  REGION 1..7: note = Description of sequence: Heavy chain-CDRH1:P08E02 Chothia
GYSFTSS

SEQ ID NO: 141 - protein, 10 aa, synthetic construct
  REGION 1..10: note = Description of sequence: Heavy chain-CDRH1:P08E02 Extended
GYSFTSSWIG

SEQ ID NO: 142 - protein, 17 aa, synthetic construct
  REGION 1..17: note = Description of sequence: Heavy chain-CDRH2:P08E02 Kabat
IIYPGDSDTRYSPSFQG

SEQ ID NO: 143 - protein, 6 aa, synthetic construct
  REGION 1..6: note = Description of sequence: Heavy chain-CDRH2:P08E02 Chothia
YPGDSD

SEQ ID NO: 144 - protein, 13 aa, synthetic construct
  REGION 1..13: note = Description of sequence: Heavy chain-CDRH3:P08E02
GLSQAMTGFGFDY

SEQ ID NO: 145 - protein, 5 aa, synthetic construct
  REGION 1..5: note = Description of sequence: Heavy chain-CDRH1:P08F08 Kabat
SYWIG

SEQ ID NO: 146 - protein, 7 aa, synthetic construct
  REGION 1..7: note = Description of sequence: Heavy chain-CDRH1:P08F08 Chothia
GYGFTSY

SEQ ID NO: 147 - protein, 10 aa, synthetic construct
  REGION 1..10: note = Description of sequence: Heavy chain-CDRH1:P08F08 Extended
GYGFTSYWIG

SEQ ID NO: 148 - protein, 17 aa, synthetic construct
  REGION 1..17: note = Description of sequence: Heavy chain-CDRH2:P08F08 Kabat
IIHPDDSDTKYSPSFQG

SEQ ID NO: 149 - protein, 6 aa, synthetic construct
  REGION 1..6: note = Description of sequence: Heavy chain-CDRH2:P08F08 Chothia
HPDDSD

SEQ ID NO: 150 - protein, 13 aa, synthetic construct
  REGION 1..13: note = Description of sequence: Heavy chain-CDRH3:P08F08
SYLRGLWGGYFDY

SEQ ID NO: 151 - protein, 5 aa, synthetic construct
  REGION 1..5: note = Description of sequence: Heavy chain-CDRH1:P08G02 Kabat
SSWIG

SEQ ID NO: 152 - protein, 7 aa, synthetic construct
  REGION 1..7: note = Description of sequence: Heavy chain-CDRH1:P08G02 Chothia
GYTFPSS

SEQ ID NO: 153 - protein, 10 aa, synthetic construct
  REGION 1..10: note = Description of sequence: Heavy chain-CDRH1:P08G02 Extended
GYTFPSSWIG

SEQ ID NO: 154 - protein, 16 aa, synthetic construct
  REGION 1..16: note = Description of sequence: Heavy chain-CDRH2:P08G02 Kabat
IIYPDTSHTRYSPSFQ

SEQ ID NO: 155 - protein, 6 aa, synthetic construct
  REGION 1..6: note = Description of sequence: Heavy chain-CDRH2:P08G02 Chothia
YPDTSH

SEQ ID NO: 156 - protein, 17 aa, synthetic construct
  REGION 1..17: note = Description of sequence: Heavy chain-CDRH3:P08G02
ASYFDRGTGYSSWWMDV

SEQ ID NO: 157 - protein, 5 aa, synthetic construct
  REGION 1..5: note = Description of sequence: Heavy chain-CDRH1:P12B09 Kabat
QYSMS

SEQ ID NO: 158 - protein, 7 aa, synthetic construct
  REGION 1..7: note = Description of sequence: Heavy chain-CDRH1:P12B09 Chothia
GFTFSQY

SEQ ID NO: 159 - protein, 10 aa, synthetic construct
  REGION 1..10: note = Description of sequence: Heavy chain-CDRH1:P12B09 Extended
GFTFSQYSMS

SEQ ID NO: 160 - protein, 17 aa, synthetic construct
  REGION 1..17: note = Description of sequence: Heavy chain-CDRH2:P12B09 Kabat
AISGGGVSTYYADSVKG

SEQ ID NO: 161 - protein, 6 aa, synthetic construct
  REGION 1..6: note = Description of sequence: Heavy chain-CDRH2:P12B09 Chothia
SGGGVS

SEQ ID NO: 162 - protein, 14 aa, synthetic construct
  REGION 1..14: note = Description of sequence: Heavy chain-CDRH3:P12B09
DISDSGGSHWYFDY

SEQ ID NO: 163 - protein, 5 aa, synthetic construct
  REGION 1..5: note = Description of sequence: Heavy chain-CDRH1:P12F02 Kabat
SYAMS

SEQ ID NO: 164 - protein, 7 aa, synthetic construct
  REGION 1..7: note = Description of sequence: Heavy chain-CDRH1:P12F02 Chothia
GFTFSSY

SEQ ID NO: 165 - protein, 10 aa, synthetic construct
  REGION 1..10: note = Description of sequence: Heavy chain-CDRH1:P12F02 Extended
GFTFSSYAMS

SEQ ID NO: 166 - protein, 17 aa, synthetic construct
  REGION 1..17: note = Description of sequence: Heavy chain-CDRH2:P12F02 Kabat
TISGTGGTTYYADSVKG

SEQ ID NO: 167 - protein, 6 aa, synthetic construct
  REGION 1..6: note = Description of sequence: Heavy chain-CDRH2:P12F02 Chothia
SGTGGT

SEQ ID NO: 168 - protein, 12 aa, synthetic construct
  REGION 1..12: note = Description of sequence: Heavy chain-CDRH3:P12F02
VRAGIDPTASDV

SEQ ID NO: 169 - protein, 5 aa, synthetic construct
  REGION 1..5: note = Description of sequence: Heavy chain-CDRH1:P12G07 Kabat
NFAMS

SEQ ID NO: 170 - protein, 7 aa, synthetic construct
  REGION 1..7: note = Description of sequence: Heavy chain-CDRH1:P12G07 Chothia
GFTFNNF

SEQ ID NO: 171 - protein, 10 aa, synthetic construct
  REGION 1..10: note = Description of sequence: Heavy chain-CDRH1:P12G07 Extended
GFTFNNFAMS

SEQ ID NO: 172 - protein, 17 aa, synthetic construct
  REGION 1..17: note = Description of sequence: Heavy chain-CDRH2:P12G07 Kabat
GISGSGDNTYYADSVKG

SEQ ID NO: 173 - protein, 6 aa, synthetic construct
  REGION 1..6: note = Description of sequence: Heavy chain-CDRH2:P12G07 Chothia
SGSGDN

SEQ ID NO: 174 - protein, 15 aa, synthetic construct
  REGION 1..15: note = Description of sequence: Heavy chain-CDRH3:P12G07
DRDIGLGWYSYYLDV

SEQ ID NO: 175 - protein, 5 aa, synthetic construct
  REGION 1..5: note = Description of sequence: Heavy chain-CDRH1:P13F04 Kabat
SYAIS

SEQ ID NO: 176 - protein, 7 aa, synthetic construct
  REGION 1..7: note = Description of sequence: Heavy chain-CDRH1:P13F04 Chothia
GGTFSSY

SEQ ID NO: 177 - protein, 10 aa, synthetic construct
  REGION 1..10: note = Description of sequence: Heavy chain-CDRH1:P13F04 Extended
GGTFSSYAIS

SEQ ID NO: 178 - protein, 17 aa, synthetic construct
  REGION 1..17: note = Description of sequence: Heavy chain-CDRH2:P13F04 Kabat
EIIPIFGTASYAQKFQG

SEQ ID NO: 179 - protein, 6 aa, synthetic construct
  REGION 1..6: note = Description of sequence: Heavy chain-CDRH2:P13F04 Chothia
IPIFGT

SEQ ID NO: 180 - protein, 10 aa, synthetic construct
  REGION 1..10: note = Description of sequence: Heavy chain-CDRH3:P13F04
AGWDDSWFDY

SEQ ID NO: 181 - protein, 5 aa, synthetic construct
  REGION 1..5: note = Description of sequence: Heavy chain-CDRH1:P15D02 Kabat
SYWIG

SEQ ID NO: 182 - protein, 7 aa, synthetic construct
  REGION 1..7: note = Description of sequence: Heavy chain-CDRH1:P15D02 Chothia
GYSFASY

SEQ ID NO: 183 - protein, 10 aa, synthetic construct
  REGION 1..10: note = Description of sequence: Heavy chain-CDRH1:P15D02 Extended
GYSFASYWIG

SEQ ID NO: 184 - protein, 17 aa, synthetic construct
  REGION 1..17: note = Description of sequence: Heavy chain-CDRH2:P15D02 Kabat
VIYPGTSETRYSPSFQG

SEQ ID NO: 185 - protein, 6 aa, synthetic construct
  REGION 1..6: note = Description of sequence: Heavy chain-CDRH2:P15D02 Chothia
YPGTSE

SEQ ID NO: 186 - protein, 13 aa, synthetic construct
  REGION 1..13: note = Description of sequence: Heavy chain-CDRH3:P15D02
GLSASASGYSFQY

SEQ ID NO: 187 - protein, 5 aa, synthetic construct
  REGION 1..5: note = Description of sequence: Heavy chain-CDRH1:P16C05 Kabat
DYWIG

SEQ ID NO: 188 - protein, 7 aa, synthetic construct
  REGION 1..7: note = Description of sequence: Heavy chain-CDRH1:P16C05 Chothia
GYSFTDY

SEQ ID NO: 189 - protein, 10 aa, synthetic construct
  REGION 1..10: note = Description of sequence: Heavy chain-CDRH1:P16C05 Extended
GYSFTDYWIG

SEQ ID NO: 190 - protein, 17 aa, synthetic construct
  REGION 1..17: note = Description of sequence: Heavy chain-CDRH2:P16C05 Kabat
MISPGGSTTIYRPSFQG

SEQ ID NO: 191 - protein, 6 aa, synthetic construct
  REGION 1..6: note = Description of sequence: Heavy chain-CDRH2:P16C05 Chothia
SPGGST

SEQ ID NO: 192 - protein, 14 aa, synthetic construct
  REGION 1..14: note = Description of sequence: Heavy chain-CDRH3:P16C05
MYTGGYGGSWYFDY

SEQ ID NO: 193 - protein, 16 aa, synthetic construct
  REGION 1..16: note = Description of sequence: Light Chain:31H1 CDRL1
RSSQSLVHSDGNTYLS

SEQ ID NO: 194 - protein, 7 aa, synthetic construct
  REGION 1..7: note = Description of sequence: Light Chain:31H1 CDRL2
KISNRFS

SEQ ID NO: 195 - protein, 9 aa, synthetic construct
  REGION 1..9: note = Description of sequence: Light Chain:31H1 CDRL3
MQATQFPLT

SEQ ID NO: 196 - protein, 16 aa, synthetic construct
  REGION 1..16: note = Description of sequence: Light Chain:63B2 CDRL1
RSSQSLVHSDGNTYLS

SEQ ID NO: 197 - protein, 7 aa, synthetic construct
  REGION 1..7: note = Description of sequence: Light Chain:63B2 CDRL2
KISNRFS

SEQ ID NO: 198 - protein, 9 aa, synthetic construct
  REGION 1..9: note = Description of sequence: Light Chain:63B2 CDRL3
MQATQFPLT

SEQ ID NO: 199 - protein, 11 aa, synthetic construct
  REGION 1..11: note = Description of sequence: Light Chain:40E3 CDRL1
RASQGISNYLA

SEQ ID NO: 200 - protein, 7 aa, synthetic construct
  REGION 1..7: note = Description of sequence: Light Chain:40E3 CDRL2
AASSLQS

SEQ ID NO: 201 - protein, 9 aa, synthetic construct
  REGION 1..9: note = Description of sequence: Light Chain:40E3 CDRL3
QQYNSYPLT

SEQ ID NO: 202 - protein, 16 aa, synthetic construct
  REGION 1..16: note = Description of sequence: Light Chain:42C3 CDRL1
RSSQSLVYSDENTYLN

SEQ ID NO: 203 - protein, 7 aa, synthetic construct
  REGION 1..7: note = Description of sequence: Light Chain:42C3 CDRL2
QVSNRDS

SEQ ID NO: 204 - protein, 9 aa, synthetic construct
  REGION 1..9: note = Description of sequence: Light Chain:42C3 CDRL3
MQGTYWPPT

SEQ ID NO: 205 - protein, 11 aa, synthetic construct
  REGION 1..11: note = Description of sequence: Light Chain:45F11 CDRL1
RASQSVSSSLA

SEQ ID NO: 206 - protein, 7 aa, synthetic construct
  REGION 1..7: note = Description of sequence: Light Chain:45F11 CDRL2
GASTRAT

SEQ ID NO: 207 - protein, 8 aa, synthetic construct
  REGION 1..8: note = Description of sequence: Light Chain:45F11 CDRL3
QQYINWPH

SEQ ID NO: 208 - protein, 11 aa, synthetic construct
  REGION 1..11: note = Description of sequence: Light Chain:64F9 CDRL1
QASQDISNYLN

SEQ ID NO: 209 - protein, 7 aa, synthetic construct
  REGION 1..7: note = Description of sequence: Light Chain:64F9 CDRL2
GASNLET

SEQ ID NO: 210 - protein, 9 aa, synthetic construct
  REGION 1..9: note = Description of sequence: Light Chain:64F9 CDRL3
QQYDNFPIT

SEQ ID NO: 211 - protein, 11 aa, synthetic construct
  REGION 1..11: note = Description of sequence: Light Chain:72C2 CDRL1
RASQSVSSNLA

SEQ ID NO: 212 - protein, 7 aa, synthetic construct
  REGION 1..7: note = Description of sequence: Light Chain:72C2 CDRL2
SASTRAS

SEQ ID NO: 213 - protein, 10 aa, synthetic construct
  REGION 1..10: note = Description of sequence: Light Chain:72C2 CDRL3
QQYDNWPPLT

SEQ ID NO: 214 - protein, 12 aa, synthetic construct
  REGION 1..12: note = Description of sequence: Light Chain:2F10 CDRL1
RASQSVSSSYLA

SEQ ID NO: 215 - protein, 7 aa, synthetic construct
  REGION 1..7: note = Description of sequence: Light Chain:2F10 CDRL2
GASSRAT

SEQ ID NO: 216 - protein, 9 aa, synthetic construct
  REGION 1..9: note = Description of sequence: Light Chain:2F10 CDRL3
QQYGSSPLT

SEQ ID NO: 217 - protein, 11 aa, synthetic construct
  REGION 1..11: note = Description of sequence: Light Chain:4F11 CDRL1
RASQDISNYLA

SEQ ID NO: 218 - protein, 7 aa, synthetic construct
  REGION 1..7: note = Description of sequence: Light Chain:4F11 CDRL2
AASSLQS

SEQ ID NO: 219 - protein, 9 aa, synthetic construct
  REGION 1..9: note = Description of sequence: Light Chain:4F11 CDRL3
LQLNSFPFT

SEQ ID NO: 220 - protein, 11 aa, synthetic construct
  REGION 1..11: note = Description of sequence: Light Chain:10H10 CDRL1
RASQGISSWLA

SEQ ID NO: 221 - protein, 7 aa, synthetic construct
  REGION 1..7: note = Description of sequence: Light Chain:10H10 CDRL2
AASSLQS

SEQ ID NO: 222 - protein, 9 aa, synthetic construct
  REGION 1..9: note = Description of sequence: Light Chain:10H10 CDRL3
QQAFSFPFT

SEQ ID NO: 223 - protein, 17 aa, synthetic construct
  REGION 1..17: note = Description of sequence: Light Chain:17G6 CDRL1
KSSQSVLYSYNNKNYVA

SEQ ID NO: 224 - protein, 7 aa, synthetic construct
  REGION 1..7: note = Description of sequence: Light Chain:17G6 CDRL2
WASTRES

SEQ ID NO: 225 - protein, 8 aa, synthetic construct
  REGION 1..8: note = Description of sequence: Light Chain:17G6 CDRL3
QQYYSTLT

SEQ ID NO: 226 - protein, 12 aa, synthetic construct
  REGION 1..12: note = Description of sequence: Light Chain:65E11 CDRL1
RASQSVSSSYLA

SEQ ID NO: 227 - protein, 7 aa, synthetic construct
  REGION 1..7: note = Description of sequence: Light Chain:65E11 CDRL2
DASSRAT

SEQ ID NO: 228 - protein, 9 aa, synthetic construct
  REGION 1..9: note = Description of sequence: Light Chain:65E11 CDRL3
QQYGSSPLT

SEQ ID NO: 229 - protein, 13 aa, synthetic construct
  REGION 1..13: note = Description of sequence: Light Chain:P02B10 CDRL1
SGSSSNIGSNYVY

SEQ ID NO: 230 - protein, 7 aa, synthetic construct
  REGION 1..7: note = Description of sequence: Light Chain:P02B10 CDRL2
RNNQRPS

SEQ ID NO: 231 - protein, 11 aa, synthetic construct
  REGION 1..11: note = Description of sequence: Light Chain:P02B10 CDRL3
AAWDDSLSGVV

SEQ ID NO: 232 - protein, 13 aa, synthetic construct
  REGION 1..13: note = Description of sequence: Light Chain:P07D03 CDRL1
SGSRSNIGSNYVY

SEQ ID NO: 233 - protein, 7 aa, synthetic construct
  REGION 1..7: note = Description of sequence: Light Chain:P07D03 CDRL2
RNNQRPS

SEQ ID NO: 234 - protein, 11 aa, synthetic construct
  REGION 1..11: note = Description of sequence: Light Chain:P07D03 CDRL3
ASWDGSLSAVV

SEQ ID NO: 235 - protein, 13 aa, synthetic construct
  REGION 1..13: note = Description of sequence: Light Chain:P08A02 CDRL1
SGSSSNIGSNYVY

SEQ ID NO: 236 - protein, 7 aa, synthetic construct
  REGION 1..7: note = Description of sequence: Light Chain:P08A02 CDRL2
RNNQRPS

SEQ ID NO: 237 - protein, 11 aa, synthetic construct
  REGION 1..11: note = Description of sequence: Light Chain:P08A02 CDRL3
ATWDDSLGSPV

SEQ ID NO: 238 - protein, 11 aa, synthetic construct
  REGION 1..11: note = Description of sequence: Light Chain:P08E02 CDRL1
RASQSISRYLN

SEQ ID NO: 239 - protein, 7 aa, synthetic construct
  REGION 1..7: note = Description of sequence: Light Chain:P08E02 CDRL2
AASILQT

SEQ ID NO: 240 - protein, 10 aa, synthetic construct
  REGION 1..10: note = Description of sequence: Light Chain:P08E02 CDRL3
QQSYSTTMWT

SEQ ID NO: 241 - protein, 13 aa, synthetic construct
  REGION 1..13: note = Description of sequence: Light Chain:P08F08 CDRL1
SGSSSNIGSNYVN

SEQ ID NO: 242 - protein, 7 aa, synthetic construct
  REGION 1..7: note = Description of sequence: Light Chain:P08F08 CDRL2
GDYQRPS

SEQ ID NO: 243 - protein, 12 aa, synthetic construct
  REGION 1..12: note = Description of sequence: Light Chain:P08F08 CDRL3
ATRDDSLSGSVV

SEQ ID NO: 244 - protein, 11 aa, synthetic construct
  REGION 1..11: note = Description of sequence: Light Chain:P08G02 CDRL1
RASQSIYDYLH

SEQ ID NO: 245 - protein, 7 aa, synthetic construct
  REGION 1..7: note = Description of sequence: Light Chain:P08G02 CDRL2
DASNLQS

SEQ ID NO: 246 - protein, 10 aa, synthetic construct
  REGION 1..10: note = Description of sequence: Light Chain:P08G02 CDRL3
QQSYTTPLFT

SEQ ID NO: 247 - protein, 11 aa, synthetic construct
  REGION 1..11: note = Description of sequence: Light Chain:P12B09 CDRL1
RASQYIGRYLN

SEQ ID NO: 248 - protein, 7 aa, synthetic construct
  REGION 1..7: note = Description of sequence: Light Chain:P12B09 CDRL2
GATSLAS

SEQ ID NO: 249 - protein, 10 aa, synthetic construct
  REGION 1..10: note = Description of sequence: Light Chain:P12B09 CDRL3
QQSYSTTSPT

SEQ ID NO: 250 - protein, 13 aa, synthetic construct
  REGION 1..13: note = Description of sequence: Light Chain:P12F02 CDRL1
SGSTSNIGRNYVY

SEQ ID NO: 251 - protein, 7 aa, synthetic construct
  REGION 1..7: note = Description of sequence: Light Chain:P12F02 CDRL2
RTNQRPS

SEQ ID NO: 252 - protein, 11 aa, synthetic construct
  REGION 1..11: note = Description of sequence: Light Chain:P12F02 CDRL3
AAWDDSLSGRV

SEQ ID NO: 253 - protein, 13 aa, synthetic construct
  REGION 1..13: note = Description of sequence: Light Chain:P12G07 CDRL1
SGSSSNIGSNYVY

SEQ ID NO: 254 - protein, 7 aa, synthetic construct
  REGION 1..7: note = Description of sequence: Light Chain:P12G07 CDRL2
MNNQRPS

SEQ ID NO: 255 - protein, 11 aa, synthetic construct
  REGION 1..11: note = Description of sequence: Light Chain:P12G07 CDRL3
AAWDDSLSAVV

SEQ ID NO: 256 - protein, 13 aa, synthetic construct
  REGION 1..13: note = Description of sequence: Light Chain:P13F04 CDRL1
SGSNSNIGTNYVS

SEQ ID NO: 257 - protein, 7 aa, synthetic construct
  REGION 1..7: note = Description of sequence: Light Chain:P13F04 CDRL2
RSSRRPS

SEQ ID NO: 258 - protein, 12 aa, synthetic construct
  REGION 1..12: note = Description of sequence: Light Chain:P13F04 CDRL3
AAWDGSLSGHWV

SEQ ID NO: 259 - protein, 11 aa, synthetic construct
  REGION 1..11: note = Description of sequence: Light Chain:P15D02 CDRL1
RASQSIDTYLN

SEQ ID NO: 260 - protein, 7 aa, synthetic construct
  REGION 1..7: note = Description of sequence: Light Chain:P15D02 CDRL2
SASSLHS

SEQ ID NO: 261 - protein, 10 aa, synthetic construct
  REGION 1..10: note = Description of sequence: Light Chain:P15D02 CDRL3
QQSYSTTAWT

SEQ ID NO: 262 - protein, 11 aa, synthetic construct
  REGION 1..11: note = Description of sequence: Light Chain:P16C05 CDRL1
RASQSIGQSLN

SEQ ID NO: 263 - protein, 7 aa, synthetic construct
  REGION 1..7: note = Description of sequence: Light Chain:P16C05 CDRL2
GASSLQS

SEQ ID NO: 264 - protein, 9 aa, synthetic construct
  REGION 1..9: note = Description of sequence: Light Chain:P16C05 CDRL3
QQSYSTPIT

SEQ ID NO: 265 - protein, 225 aa, synthetic construct
  REGION 1..225: note = Description of sequence: Domain: CD3zeta zeta concatenated cytoplasmic domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 266 - protein, 21 aa, synthetic construct
  REGION 1..21: note = Description of sequence: Domain: CD8-alpha signal peptide
MALPVTALLLPLALLLHAARP

SEQ ID NO: 267 - protein, 16 aa, synthetic construct
  REGION 1..16: note = Description of sequence: Domain: Fc-Gamma-RIII-alpha hinge
GLAVSTISSFFPPGYQ

SEQ ID NO: 268 - protein, 45 aa, synthetic construct
  REGION 1..45: note = Description of sequence: Domain: CD8-alpha hinge
TTTPAPRPPTPAPTIASQPLSLRPEACRPAAGGAVHTRGLDFACD

SEQ ID NO: 269 - protein, 231 aa, synthetic construct
  REGION 1..231: note = Description of sequence: Domain: IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 24 aa, synthetic construct
  REGION 1..24: note = Description of sequence: Domain: CD8-alpha transmembrane (TM) domain
IYIWAPLAGTCGVLLLSLVITLYC

SEQ ID NO: 271 - protein, 42 aa, synthetic construct
  REGION 1..42: note = Description of sequence: Domain: 41BB intracellular signaling domain (ISD)
KRGRKKLLYIFKQPFMRPVQTTQEEDGCSCRFPEEEEGGCEL

SEQ ID NO: 272 - protein, 112 aa, synthetic construct
  REGION 1..112: note = Description of sequence: Domain: CD3-zeta intracellular signaling domain (ISD)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 273 - protein, 52 aa, synthetic construct
  REGION 1..52: note = Description of sequence: Domain: Fc-Epsilon-RI -alpha-TM-IC (Fc-Epsilon-RI -alpha chain transmembrane and intracellular domain)
FFIPLLVVILFAVDTGLFISTQQQVTFLLKIKRTRKGFRLLNPHPKPNPKNN

SEQ ID NO: 274 - protein, 215 aa, synthetic construct
  REGION 1..215: note = Description of sequence: Domain: Fc-Epsilon-RI-Beta-Delta-ITAM (Fc-Epsilon-RI -Beta chain without ITAM)
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPE

SEQ ID NO: 275 - protein, 45 aa, synthetic construct
  REGION 1..45: note = Description of sequence: Domain: CD3 delta cytoplasmic domain
GHETGRLSGAADTQALLRNDQVYQPLRDRDDAQYSHLGGNWARNK

SEQ ID NO: 276 - protein, 41 aa, synthetic construct
  REGION 1..41: note = Description of sequence: Domain: CD28-IC (CD28 co-stimulatory domain)
RSKRSRGGHSDYMNMTPRRPGPTRKHYQPYAPPRDFAAYRS

SEQ ID NO: 277 - protein, 18 aa, synthetic construct
  REGION 1..18: note = Description of sequence: Domain: Fc-Epsilon-RI-Gamma-SP (signal peptide)
MIPAVVLLLLLLVEQAAA

SEQ ID NO: 278 - protein, 43 aa, synthetic construct
  REGION 1..43: note = Description of sequence: Domain: Fc-Epsilon-RI -Gamma-Delta-ITAM (Fc-Epsilon-RI -Gamma chain without ITAM)
LGEPQLCYILDAILFLYGIVLTLLYCRLKIQVRKAAITSYEKS

SEQ ID NO: 279 - protein, 22 aa, synthetic construct
  REGION 1..22: note = Description of sequence: Domain: GSG-P2A (GSG-P2A ribosomal skip polypeptide)
GSGATNFSLLKQAGDVEENPGP

SEQ ID NO: 280 - protein, 21 aa, synthetic construct
  REGION 1..21: note = Description of sequence: Domain: GSG-T2A (GSG-T2A ribosomal skip polypeptide)
GSGEGRGSLLTCGDVEENPGP

SEQ ID NO: 281 - protein, 55 aa, synthetic construct
  REGION 1..55: note = Description of sequence: Domain: CD3 epsilon cytoplasmic domain
KNRKAKAKPVTRGAGAGGRQRGQNKERPPPVPNPDYEPIRKGQRDLYSGLNQRRI

SEQ ID NO: 282 - protein, 45 aa, synthetic construct
  REGION 1..45: note = Description of sequence: Domain: CD3 gamma cytoplasmic domain
GQDGVRQSRASDKQTLLPNDQLYQPLKDREDDQYSHLQGNQLRRN

SEQ ID NO: 283 - protein, 29 aa, synthetic construct
  REGION 1..29: note = Description of sequence: Domain: CD3 zeta ITAM zeta1
APAYQQGQNQLYNELNLGRREEYDVLDKR

SEQ ID NO: 284 - protein, 28 aa, synthetic construct
  REGION 1..28: note = Description of sequence: Domain: CD3 zeta ITAM zeta2
PRRKNPQEGLYNELQKDKMAEAYSEIGM

SEQ ID NO: 285 - protein, 29 aa, synthetic construct
  REGION 1..29: note = Description of sequence: Domain: CD3 zeta ITAM zeta3
ERRRGKGHDGLYQGLSTATKDTYDALHMQ

SEQ ID NO: 286 - protein, 29 aa, synthetic construct
  REGION 1..29: note = Description of sequence: Domain: CD3 delta ITAM delta
DTQALLRNDQVYQPLRDRDDAQYSHLGGN

SEQ ID NO: 287 - protein, 28 aa, synthetic construct
  REGION 1..28: note = Description of sequence: Domain: CD3 epsilon ITAM epsilon
ERPPPVPNPDYEPIRKGQRDLYSGLNQR

SEQ ID NO: 288 - protein, 29 aa, synthetic construct
  REGION 1..29: note = Description of sequence: Domain: CD3 gamma ITAM gamma
DKQTLLPNDQLYQPLKDREDDQYSHLQGN

SEQ ID NO: 289 - protein, 112 aa, synthetic construct
  REGION 1..112: note = Description of sequence: Domain: CD3 zeta variant (dzz)
RVKFSRSADDTQALLRNDQVYQPLRDRDDAQYSHLGGNRGRDPEMGGKPRRKNPQEGLYNELQKDKMAEAYSEIGMKGERRRGKGHDGLYQGLSTATKDTYDALHMQALPPR

SEQ ID NO: 290 - protein, 111 aa, synthetic construct
  REGION 1..111: note = Description of sequence: Domain: CD3 zeta variant (ezz)
RVKFSRSADERPPPVPNPDYEPIRKGQRDLYSGLNQRRGRDPEMGGKPRRKNPQEGLYNELQKDKMAEAYSEIGMKGERRRGKGHDGLYQGLSTATKDTYDALHMQALPPR

SEQ ID NO: 291 - protein, 112 aa, synthetic construct
  REGION 1..112: note = Description of sequence: Domain: CD3 zeta variant (gzz)
RVKFSRSADDKQTLLPNDQLYQPLKDREDDQYSHLQGNRGRDPEMGGKPRRKNPQEGLYNELQKDKMAEAYSEIGMKGERRRGKGHDGLYQGLSTATKDTYDALHMQALPPR

SEQ ID NO: 292 - protein, 112 aa, synthetic construct
  REGION 1..112: note = Description of sequence: Domain: CD3 zeta variant (deg)
RVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 293 - protein, 225 aa, synthetic construct
  REGION 1..225: note = Description of sequence: Domain: CD3 zeta zeta (zdzezg)
RVKFSRSADAPAYQQGQNQLYNELNLGRREEYDVLDKRRGRDPEMGGKDTQALLRNDQVYQPLRDRDDAQYSHLGGNKGPRRKNPQEGLYNELQKDKMAEAYSEIGMALPPRGRVKFSRSADERPPPVPNPDYEPIRKGQRDLYSGLNQRRGRDPEMGGKERRRGKGHDGLYQGLSTATKDTYDALHMQKGDKQTLLPNDQLYQPLKDREDDQYSHLQGNALPPR

SEQ ID NO: 294 - protein, 225 aa, synthetic construct
  REGION 1..225: note = Description of sequence: Domain: CD3 zeta zeta (dzezgz)
RVKFSRSADDTQALLRNDQVYQPLRDRDDAQYSHLGGNRGRDPEMGGKAPAYQQGQNQLYNELNLGRREEYDVLDKRKGERPPPVPNPDYEPIRKGQRDLYSGLNQRALPPRGRVKFSRSADPRRKNPQEGLYNELQKDKMAEAYSEIGMRGRDPEMGGKDKQTLLPNDQLYQPLKDREDDQYSHLQGNKGERRRGKGHDGLYQGLSTATKDTYDALHMQALPPR

SEQ ID NO: 295 - protein, 225 aa, synthetic construct
  REGION 1..225: note = Description of sequence: Domain: CD3 zeta zeta (zzzdeg)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGRVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPR

SEQ ID NO: 296 - protein, 15 aa, synthetic construct
  REGION 1..15: note = Description of sequence: Domain: (GGGGS)3
GGGGSGGGGSGGGGS

SEQ ID NO: 297 - protein, 369 aa, synthetic construct
  REGION 1..369: note = Description of sequence: 4F11 heavy chain variable region
CAGGTCACCTTGAAGGAGTCTGGTCCTGTGCTGGTGAAACCCACAGAGACCCTCACGCTGACCTGCACCGTCTCTGGGTTCTCACTCAGTAATGCTAGAATGGGTGTGACCTGGATCCGTCAGCCCCCAGGGAAGGCCCTGGAGTGGCTTGCACACATTTTTTCGAATGACGAAAAATCCTACAGTACATCTCTGAAGAGCAGGCTCACCATCTCCAAGGACACTTCCAAAACCCAGGTGGTCCTTACCATGACCAACATGGACCCTGTGGACACAGCCACATATTACTGTGCACGGATACGAGATTACTATGACATTAGTAGTTATTATGACTACTGGGGCCAGGGAACCCTGGTCAGCGTCTCCTCA

SEQ ID NO: 298 - protein, 321 aa, synthetic construct
  REGION 1..321: note = Description of sequence: 4F11 light chain variable region
GACATCCAGATGACCCAGTCTCCATCTGCCATGTCTGCATCTGTAGGAGACAGAGTCACCATCACTTGTCGGGCGAGTCAGGACATTAGCAATTATTTAGCCTGGTTTCAGCAGAAACCAGGGAAAGTCCCTAAGCGCCTGATCTATGCTGCATCCAGTTTGCAAAGTGGGGTCCCATCAAGGTTCAGCGGCAGTGGATCGGGGACAGAATTCACTCTCACAATCAGCAGCCTGCTGCCTGAAGATTTTGCAACTTATTACTGTCTACAGCTTAATAGTTTCCCGTTCACTTTTGGCGGAGGGACCAAGGTGGAGATCAAC

SEQ ID NO: 299 - protein, 372 aa, synthetic construct
  REGION 1..372: note = Description of sequence: 17G6 heavy chain variable region
GAGGTGCAGCTGGTGGAGTCTGGGGGAGGCTTGGTCCAGCCTGGGGGGTCCCTGAGACTCTCCTGTGTAGCCTCTGGATTCACCTTTAGTAGTTATTGGATGAGCTGGGTCCGCCAGGCTCCAGGGAAGGGGCTGGAGTGGGTGGCCAGCATAAAGCAAGATGGAAGTGAGAAATACTATGTGGACTCTGTGAAGGGCCGATTCACCATCTCCAGAGACAACGCCAAGAACTCAGTGTATCTGCAAATGAACAGCCTGAGAGCCGAGGACACGGGTGTGTATTACTGTGCGAGAGAAGGAGTCAACTGGGGATGGAGACTCTACTGGCACTTCGATCTCTGGGGCCGTGGAACCCTGGTCACTGTCTCCTCA

SEQ ID NO: 300 - protein, 336 aa, synthetic construct
  REGION 1..336: note = Description of sequence: 17G6 light chain variable region
GACATCGTGATGACCCAGTCTCCAGACTCCCTGGCTGTGTCTCTGGGCGAGAGGGCCACCATCAACTGCAAGTCCAGCCAGAGTGTTTTATACAGCTACAACAATAAGAACTACGTAGCTTGGTACCAGCAGAAACCAGGACAACCTCCTAACCTACTCATTTTCTGGGCATCTACCCGGGAATCCGGGGTCCCTGACCGATTCAGTGGCAGCGGGTCTGGGACAGATTTCACTCTCACCATCAGCAGCCTGCAGGCTGAAGATGTGGCAGTTTACTACTGTCAGCAATATTATAGTACGCTCACTTTCGGCGGAGGGACCAAGGTGGAGATCAAA

SEQ ID NO: 301 - protein, 357 aa, synthetic construct
  REGION 1..357: note = Description of sequence: 10H10 heavy chain variable region
GAGGTGCAGCTGGTGGAGTCTGGGGGAGGCTTGGTACAGCCTGGGGGGTCCCTGAGACTCTCCTGTGCAGTCTCTGGATTCACCTTCAGTAACCATAACATACACTGGGTCCGCCAGGCTCCAGGGAAGGGGCTGGAGTGGATTTCATACATTAGTCGAAGTAGTAGTACCATATATTACGCAGACTCTGTGAAGGGCCGATTCACAATCTCCAGAGACAATGCCAAGAACTCACTGTATCTGCAAATGAACAGCCTGAGAGACGAAGACACGGCTGTGTATTACTGTGCGAGAGATCACGCTCAGTGGTACGGTATGGACGTTTGGGGCCAAGGGACCACGGTCACCGTCTCCTCA

SEQ ID NO: 302 - protein, 321 aa, synthetic construct
  REGION 1..321: note = Description of sequence: 10H10 light chain variable region
GACATCCAGATGACCCAGTCTCCATCTTCCGTGTCTGCATCGGTAGGAGACAGAGTCACCATCACTTGTCGGGCGAGTCAGGGTATTAGCAGCTGGTTAGCCTGGTATCAGCAGAAACCAGGGAAAGCCCCTAAGGTCCTGATCTATGCTGCATCCAGTTTGCAAAGTGGGGTCCCATCAAGGTTCAGCGGCAGTGGATCTGGGACAGATTTCACTCTCACCATCAGCAGCCTGCAGCCTGAAGATTTTGCAACTTACTATTGTCAACAGGCTTTCAGTTTCCCATTCACTTTCGGCCCTGGGACCAAAGTGGATATCAAA

SEQ ID NO: 303 - protein, 366 aa, synthetic construct
  REGION 1..366: note = Description of sequence: P07D03 heavy chain variable region
GAAGTGCAGCTTGTCCAGAGCGGAGCCGAAGTGAAGAAGCCTGGCGAGAGCCTGAAGATCAGCTGCAAGGGCTCCGGATATCGCTTCACAAGTTACTGGATAGGGTGGGTGCGCCAGATGCCTGGTAAGGGACTGGAATGGATGGGCTCTATATATCCTGATGATTCCGACACACGTTATAGCCCAAGCTTTCAGGGCCAGGTCACAATCAGCGCTGACAAGAGCATCAGCACCGCCTACCTTCAGTGGTCGTCTCTGAAGGCCAGCGACACCGCAATGTACTACTGCGCCTCTAGCACAGTTGACTACCCGGGATACAGTTACTTCGACTACTGGGGCCAAGGTACACTGGTCACCGTCAGCAGC

SEQ ID NO: 304 - protein, 336 aa, synthetic construct
  REGION 1..336: note = Description of sequence: P07D03 light chain variable region
GAGCTCCAGAGCGTGCTGACCCAGCCTCCTAGCGCAAGCGGCACCCCTGGACAGCGTGTGACAATTAGCTGTAGCGGAAGTCGTAGCAATATCGGATCAAACTATGTGTATTGGTATCAGCAATTGCCCGGTACAGCACCCAAATTGCTCATATATAGAAATAATCAGAGACCTAGCGGAGTGCCTGATCGTTTTAGCGGTAGCAAAAGCGGCACCAGCGCATCACTGGCAATTTCAGGCCTGCGTAGCGAAGATGAGGCGGATTATTACTGTGCGAGTTGGGATGGTTCGCTGAGTGCTGTTGTGTTCGGCACCGGTACAAAACTGACCGTTCTG

SEQ ID NO: 305 - protein, 378 aa, synthetic construct
  REGION 1..378: note = Description of sequence: P08G02 heavy chain variable region
GAAGTGCAGCTTGTCCAGAGCGGAGCCGAAGTGAAGAAGCCTGGCGAGAGCCTGAAGATCAGCTGCAAGGGCTCCGGATACACCTTTCCTTCATCATGGATAGGTTGGGTGCGCCAGATGCCTGGTAAGGGACTGGAATGGATGGGCATCATATACCCTGATACTAGCCATACCCGTTACAGCCCAAGCTTTCAGGGCCAGGTCACAATCAGCGCTGACAAGAGCATCAGCACCGCCTACCTTCAGTGGTCGTCTCTGAAGGCCAGCGACACCGCAATGTACTACTGTGCCCGTGCGAGCTATTTCGATCGTGGAACAGGGTATAGTTCTTGGTGGATGGATGTGTGGGGCCAAGGTACACTGGTCACCGTCAGCAGC

SEQ ID NO: 306 - protein, 330 aa, synthetic construct
  REGION 1..330: note = Description of sequence: P08G02 light chain variable region
GAGCTCGATATTCAGATGACCCAGAGCCCTAGCAGCCTGAGCGCAAGCGTGGGCGATAGAGTGACCATTACCTGTAGGGCCTCACAATCCATATACGACTATTTGCACTGGTATCAGCAGAAACCCGGGAAAGCACCCAAACTGCTGATTTACGATGCTTCCAACCTACAGAGTGGCGTTCCTTCACGTTTTAGCGGTAGCGGTTCAGGCACCGATTTCACCCTGACCATTAGCAGCCTTCAGCCCGAAGATTTCGCTACGTATTATTGCCAGCAATCATACACCACGCCGTTGTTTACATTCGGCCAGGGTACCAAAGTGGAAATCAAA

SEQ ID NO: 307 - protein, 366 aa, synthetic construct
  REGION 1..366: note = Description of sequence: P08F08 heavy chain variable region
GAAGTGCAGCTTGTCCAGAGCGGAGCCGAAGTGAAGAAGCCTGGCGAGAGCCTGAAGATCAGCTGCAAGGGCTCCGGATACGGATTCACAAGTTATTGGATAGGTTGGGTGCGCCAGATGCCTGGTAAGGGACTGGAATGGATGGGTATCATTCATCCCGATGATAGCGACACCAAATACAGCCCAAGCTTTCAGGGCCAGGTCACAATCAGCGCTGACAAGAGCATCAGCACCGCCTACCTTCAGTGGTCGTCTCTGAAGGCCAGCGACACCGCAATGTACTACTGTGCCTCTAGCTATTTGCGTGGCTTGTGGGGAGGCTATTTTGACTATTGGGGCCAAGGTACACTGGTCACCGTCAGCAGC

SEQ ID NO: 308 - protein, 339 aa, synthetic construct
  REGION 1..339: note = Description of sequence: P08F08 light chain variable region
GAGCTCCAGAGCGTGCTGACCCAGCCTCCTAGCGCAAGCGGCACCCCTGGACAGCGTGTGACAATTAGCTGTAGCGGATCAAGCTCAAACATTGGCTCAAATTATGTGAATTGGTATCAGCAATTGCCCGGTACAGCACCCAAACTGCTCATTTATGGAGATTATCAACGACCTAGCGGAGTGCCTGATCGTTTTAGCGGTAGCAAAAGCGGCACCAGCGCATCACTGGCAATTTCAGGCCTGCGTAGCGAAGATGAGGCGGATTATTACTGTGCTACCCGCGACGATTCGTTATCTGGGTCTGTCGTTTTTGGCACCGGTACAAAACTGACCGTGCTG

SEQ ID NO: 309 - protein, 366 aa, synthetic construct
  REGION 1..366: note = Description of sequence: P15D02 heavy chain variable region
GAAGTGCAGCTTGTCCAGAGCGGAGCCGAAGTGAAGAAGCCTGGCGAGAGCCTGAAGATCAGCTGCAAGGGCTCCGGATACAGTTTTGCCTCATACTGGATCGGTTGGGTGCGCCAGATGCCTGGTAAGGGACTGGAATGGATGGGCGTAATTTACCCCGGAACTAGCGAGACACGTTACAGCCCAAGCTTTCAGGGCCAGGTCACAATCAGCGCTGACAAGAGCATCAGCACCGCCTACCTTCAGTGGTCGTCTCTGAAGGCCAGCGACACCGCAATGTACTACTGCGCTAAAGGGTTGAGTGCGAGTGCAAGTGGATATTCTTTCCAATATTGGGGCCAAGGTACACTGGTCACCGTCAGCAGC

SEQ ID NO: 310 - protein, 330 aa, synthetic construct
  REGION 1..330: note = Description of sequence: P15D032 light chain variable region
GAGCTCGATATTCAGATGACCCAGAGCCCTAGCAGCCTGAGCGCAAGCGTGGGCGATAGAGTGACCATTACCTGTAGGGCCTCACAAAGCATCGACACATATTTAAACTGGTATCAGCAGAAACCCGGGAAAGCACCCAAACTGCTGATTTATTCAGCTAGTAGCCTACACAGTGGCGTTCCTTCACGTTTTAGCGGTAGCGGTTCAGGCACCGATTTCACCCTGACCATTAGCAGCCTTCAGCCCGAAGATTTCGCTACGTATTATTGCCAACAATCATACAGCACAACTGCTTGGACATTCGGCCAGGGTACCAAAGTGGAAATCAAA

SEQ ID NO: 311 - protein, 225 aa, synthetic construct
  REGION 1..225: note = Description of sequence: Domain: CD3 zeta zeta (degzzz)
RVKFSRSADDTQALLRNDQVYQPLRDRDDAQYSHLGGNRGRDPEMGGKERPPPVPNPDYEPIRKGQRDLYSGLNQRKGDKQTLLPNDQLYQPLKDREDDQYSHLQGNALPPRGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 312 - protein, 112 aa, synthetic construct
  REGION 1..112: note = Description of sequence: Domain: CD3 zeta YA
RVKFSRSADAPAYQQGQNQLYAELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 313 - protein, 112 aa, synthetic construct
  REGION 1..112: note = Description of sequence: Domain: CD3 zeta YAYAYA
RVKFSRSADAPAYQQGQNQLYAELNLGRREEYDVLDKRRGRDPEMGGKPRRKNPQEGLYAELQKDKMAEAYSEIGMKGERRRGKGHDGLYAGLSTATKDTYDALHMQALPPR

SEQ ID NO: 314 - protein, 43 aa, synthetic construct
  REGION 1..43: note = Description of sequence: Domain: CD3 zeta YAtrunc
RVKFSRSADAPAYQQGQNQLYAELNLGRREEYDVLDKRALPPR

SEQ ID NO: 315 - protein, 225 aa, synthetic construct
  REGION 1..225: note = Description of sequence: Domain: CD3 zeta zeta (zdzezg-6xYA)
RVKFSRSADAPAYQQGQNQLYAELNLGRREEYDVLDKRRGRDPEMGGKDTQALLRNDQVYAPLRDRDDAQYSHLGGNKGPRRKNPQEGLYAELQKDKMAEAYSEIGMALPPRGRVKFSRSADERPPPVPNPDYAPIRKGQRDLYSGLNQRRGRDPEMGGKERRRGKGHDGLYAGLSTATKDTYDALHMQKGDKQTLLPNDQLYAPLKDREDDQYSHLQGNALPPR

SEQ ID NO: 316 - protein, 225 aa, synthetic construct
  REGION 1..225: note = Description of sequence: Domain: CD3 zeta zeta (dzezgz-6xYA)
RVKFSRSADDTQALLRNDQVYAPLRDRDDAQYSHLGGNRGRDPEMGGKAPAYQQGQNQLYAELNLGRREEYDVLDKRKGERPPPVPNPDYAPIRKGQRDLYSGLNQRALPPRGRVKFSRSADPRRKNPQEGLYAELQKDKMAEAYSEIGMRGRDPEMGGKDKQTLLPNDQLYAPLKDREDDQYSHLQGNKGERRRGKGHDGLYAGLSTATKDTYDALHMQALPPR

SEQ ID NO: 317 - protein, 29 aa, synthetic construct
  REGION 1..29: note = Description of sequence: Domain: CD3 zeta ITAM zeta1 YA
APAYQQGQNQLYAELNLGRREEYDVLDKR

SEQ ID NO: 318 - protein, 28 aa, synthetic construct
  REGION 1..28: note = Description of sequence: Domain: CD3 zeta ITAM zeta2 YA
PRRKNPQEGLYAELQKDKMAEAYSEIGM

SEQ ID NO: 319 - protein, 29 aa, synthetic construct
  REGION 1..29: note = Description of sequence: Domain: CD3 zeta ITAM zeta3 YA
ERRRGKGHDGLYAGLSTATKDTYDALHMQ

SEQ ID NO: 320 - protein, 29 aa, synthetic construct
  REGION 1..29: note = Description of sequence: Domain: CD3 delta ITAM delta YA
DTQALLRNDQVYAPLRDRDDAQYSHLGGN

SEQ ID NO: 321 - protein, 28 aa, synthetic construct
  REGION 1..28: note = Description of sequence: Domain: CD3 epsilon ITAM epsilon YA
ERPPPVPNPDYAPIRKGQRDLYSGLNQR

SEQ ID NO: 322 - protein, 29 aa, synthetic construct
  REGION 1..29: note = Description of sequence: Domain: CD3 gamma ITAM gamma YA
DKQTLLPNDQLYAPLKDREDDQYSHLQGN

SEQ ID NO: 323 - protein, 29 aa, synthetic construct
  REGION 1..29: note = Description of sequence: Domain: CD3 zeta ITAM zeta1 YA
APAYQQGQNQLYAELNLGRREEYDVLDKR

SEQ ID NO: 324 - protein, 28 aa, synthetic construct
  REGION 1..28: note = Description of sequence: Domain: CD3 zeta ITAM zeta2 YA
PRRKNPQEGLYAELQKDKMAEAYSEIGM

SEQ ID NO: 325 - protein, 29 aa, synthetic construct
  REGION 1..29: note = Description of sequence: Domain: CD3 zeta ITAM zeta3 YA
ERRRGKGHDGLYAGLSTATKDTYDALHMQ

SEQ ID NO: 326 - protein, 29 aa, synthetic construct
  REGION 1..29: note = Description of sequence: Domain: CD3 delta ITAM delta YA
DTQALLRNDQVYAPLRDRDDAQYSHLGGN

SEQ ID NO: 327 - protein, 28 aa, synthetic construct
  REGION 1..28: note = Description of sequence: Domain: CD3 epsilon ITAM epsilon YA
ERPPPVPNPDYAPIRKGQRDLYSGLNQR

SEQ ID NO: 328 - protein, 29 aa, synthetic construct
  REGION 1..29: note = Description of sequence: Domain: CD3 gamma ITAM gamma YA
DKQTLLPNDQLYAPLKDREDDQYSHLQGN

SEQ ID NO: 329 - protein, 29 aa, synthetic construct
  REGION 1..29: note = Description of sequence: Domain: CD3 zeta ITAM zeta1 YA
APAYQQGQNQLYAELNLGRREEYDVLDKR

SEQ ID NO: 330 - protein, 28 aa, synthetic construct
  REGION 1..28: note = Description of sequence: Domain: CD3 zeta ITAM zeta2 YA
PRRKNPQEGLYAELQKDKMAEAYSEIGM

SEQ ID NO: 331 - protein, 29 aa, synthetic construct
  REGION 1..29: note = Description of sequence: Domain: CD3 zeta ITAM zeta3 YA
ERRRGKGHDGLYAGLSTATKDTYDALHMQ

SEQ ID NO: 332 - protein, 29 aa, synthetic construct
  REGION 1..29: note = Description of sequence: Domain: CD3 delta ITAM delta YA
DTQALLRNDQVYAPLRDRDDAQYSHLGGN

SEQ ID NO: 333 - protein, 28 aa, synthetic construct
  REGION 1..28: note = Description of sequence: Domain: CD3 epsilon ITAM epsilon YA
ERPPPVPNPDYAPIRKGQRDLYSGLNQR

SEQ ID NO: 334 - protein, 29 aa, synthetic construct
  REGION 1..29: note = Description of sequence: Domain: CD3 gamma ITAM gamma YA
DKQTLLPNDQLYAPLKDREDDQYSHLQGN

SEQ ID NO: 335 - protein, 29 aa, synthetic construct
  REGION 1..29: note = Description of sequence: Domain: CD3 zeta ITAM zeta1 YA
APAYQQGQNQLYAELNLGRREEYDVLDKR

SEQ ID NO: 336 - protein, 41 aa, synthetic construct
  REGION 1..41: note = Description of sequence: Domain: CD28 intracellular
RSKRSRLLHSDYMNMTPRRPGPTRKHYQPYAPPRDFAAYRS

SEQ ID NO: 337 - protein, 41 aa, synthetic construct
  REGION 1..41: note = Description of sequence: Domain: CD28.YMFM intracellular
RSKRSRLLHSDYMFMTPRRPGPTRKHYQPYAPPRDFAAYRS

SEQ ID NO: 338 - protein, 107 aa, synthetic construct
  REGION 1..107: note = Description of sequence: Light chain-10A1
DIQMTQSPSTLSASVGDRVTITCRASQSISTWLAWYQQKPGKAPKVLIYKASSLESGVPSRFSGSGSGTEFILTINSLQPDDFASYYCQQYKSYSHTFGQGTKLEIK

SEQ ID NO: 339 - protein, 118 aa, synthetic construct
  REGION 1..118: note = Description of sequence: Heavy chain-10A1
QVQLQESGPGLVKPSETLSLTCTVSGGSISYYYWTWIRQPPGKGLEWIGHIYYSGSTNYNPSLKSRVTISIDTSKNLFSLKLSSVTAADTAVYYCARAEGSLDAFDFWGQGTMVTVSS

SEQ ID NO: 340 - protein, 107 aa, synthetic construct
  REGION 1..107: note = Description of sequence: Light chain-10E2
DIQMTQSPSTLSASVGDRVTITCRASQSISSWLAWYQQKPGKAPKVLIYKASSLESGVPSRFSGSGSGTEFTLTINSLQPDDFATYYCQQYKSFSLTFGQGTKLEIK

SEQ ID NO: 341 - protein, 113 aa, synthetic construct
  REGION 1..113: note = Description of sequence: Heavy chain-10E2
EVQLVESGGGLIQPGGSLRLSCAASGFTVSSNYMTWVRQAPGKGLEWVSVIYSGGSTYYADSVKGRFTISRDNSKNTLYLQMNSLRAEDTAVYYCARNWGDYWGQGTLVTVSS

SEQ ID NO: 342 - protein, 107 aa, synthetic construct
  REGION 1..107: note = Description of sequence: Light chain-11A1
DIQMTQSPSTLSASVGDRVTITCRASQSISSWLAWYQQKPGKAPKVLIYKASTLESGVPSRFSGSGSGTEFTLTISSLQPDDFATYYCQQYNSYSYTFGHGTKLEIK

SEQ ID NO: 343 - protein, 118 aa, synthetic construct
  REGION 1..118: note = Description of sequence: Heavy chain-11A1
QVQLQESGPGLVKPSGTLSLTCTVSGGSIDYYFWNWFRQSPVKGLEWIGHVYDIGNTKYNPSLKSRVTISIDTSENQFSLKLNSVTAADTAVYYCARGEGALDAFDIWGQGTMVTVSS

SEQ ID NO: 344 - protein, 107 aa, synthetic construct
  REGION 1..107: note = Description of sequence: Light chain-11C1
DIQMTQSPSILSASVGDRVTITCRASQSVSSWLAWYQQKPGKAPKVLIYKASSLESGVPSRFSGTGSGTEFTLTISSLQSDDFATYYCQQYNTYSHTFGQGTKLEIK

SEQ ID NO: 345 - protein, 118 aa, synthetic construct
  REGION 1..118: note = Description of sequence: Heavy chain-11C1
QVQLQESGPGLVKPSETLSLNCTVSGGSISYYYWTWIRQPPGKGLEWIGHVIYSGTTNYNPSLKSRVTISVDTSKNQFSLKLNSVTAADTAVYYCVRAEGSLDAFDLWGQGTMVTVSS

SEQ ID NO: 346 - protein, 107 aa, synthetic construct
  REGION 1..107: note = Description of sequence: Light chain-11D1
AIQMTQSPSSLSASVGDRVTITCRASQGIRNDLGWYQQKPGKAPKLLIYAASSLQSGVPSRFSGSGSGTDFTLTISSLQPEDFATYYCLQDYNYPFTFGPGTKVDIK

SEQ ID NO: 347 - protein, 120 aa, synthetic construct
  REGION 1..120: note = Description of sequence: Heavy chain-11D1
QVQLVESGGGVVQPGRSLRLSCVASGFTFSDYGIHWVRQAPGMGQEWVAVIWYDGSIKKYSDSVKGRFIISRDNSENTVYLQMNSLRGEDTAIYYCARDEVGTFGAFDFWGQGTKVTVSS

SEQ ID NO: 348 - protein, 107 aa, synthetic construct
  REGION 1..107: note = Description of sequence: Light chain-11E1
DIQMTQSPSSLSASVGDSITITCRASQDIDNYLAWYQQKTGKVPKVLIYAASALQSGVPSRFSGSGSGTDFTLTISSLQPEDVATYYCQNYNSGPRTFGQGTKVEIK

SEQ ID NO: 349 - protein, 118 aa, synthetic construct
  REGION 1..118: note = Description of sequence: Heavy chain-11E1
QVQLQESGPGLVKPLQTLSLTCTVSGGSISSDGYYWSWIRQNPGKGLEWIGYMYYSGSTYYNPSLKSRVTISVDTSKNQFSLKLRSVTAADTAVYYCTRDFGWYFDLWGRGTLVTVSS

SEQ ID NO: 350 - protein, 107 aa, synthetic construct
  REGION 1..107: note = Description of sequence: Light chain-12A2
DIQMTQSPSSLSASVGDRVTITCRASQDISNYLTWYQQKPGRVPEVLIYAASALQSGVPSRFSGSGSGTDFTLTISSLQPEDVATYYCQNYNSAPRTFGQGTKVEIK

SEQ ID NO: 351 - protein, 118 aa, synthetic construct
  REGION 1..118: note = Description of sequence: Heavy chain-12A2
QVQLQESGPGLVKPSQSLSLTCSVSGGSVSSDGYYWSWIRQHPGKGLEWIGYIYYRRITDYNPSLKSRVNISLDTSKNQFSLKLSSVTAADTAVYYCARDFGWYFDLWGRGTLVAVSS

SEQ ID NO: 352 - protein, 112 aa, synthetic construct
  REGION 1..112: note = Description of sequence: Light chain-12C4
DIVMTQSPLSLPVTPGEPASISCRSSQSLLHSNGYNYLDWYLQKPGQSPQVLILLGSNRASGVPDRVSASGSGTDFTLKISRMQAEDVGIYYCMQTLQTPFTFGQGTKLEIK

SEQ ID NO: 353 - protein, 122 aa, synthetic construct
  REGION 1..122: note = Description of sequence: Heavy chain-12C4
QVQLVQSGAEVKKPGASVKVSCKASGYTFTGYYLHWVRQAPGQGLEWMGWINPNSGGTNYAQKFQGRVTMTRDTSITTAYMELSRLRIDDTAVYYCARDRGVTMIVDGMDDWGQGTTVTVSS

SEQ ID NO: 354 - protein, 107 aa, synthetic construct
  REGION 1..107: note = Description of sequence: Light chain-12C5
DIQLTQSPSFLSASVGDRVIITCRASQGINSHLAWYQQKPGKAPKLLIYYASTLPSGVPSRFSGSGSGTEFTLTVTSLQPEDFATYYCQQLNHYPITFGQGTRLDIN

SEQ ID NO: 355 - protein, 120 aa, synthetic construct
  REGION 1..120: note = Description of sequence: Heavy chain-12C5
EVELVESGGGMVQPGRSLRLSCAASGFTFSDYGMHWVRQAPGMGLEWVTVIWYDGSNKYYADSVKGRFTISRDNSKNTVFLQMNSLRAEDTAVYYCARDEVGFVGAFDIWGQGTMVTVSS

SEQ ID NO: 356 - protein, 107 aa, synthetic construct
  REGION 1..107: note = Description of sequence: Light chain-12D3
DIQMTQSPSSLSASVGDRVTITCRASQGISNYLAWYQQKPGKVPKLLIYAASTLHSGVPSRFSGSGSGTDFTLTISSLQPEDVATYYCQKYNSAPRTFGQGTKVEIK

SEQ ID NO: 357 - protein, 118 aa, synthetic construct
  REGION 1..118: note = Description of sequence: Heavy chain-12D3
QVQLQESGPGLVKPSQTLSLTCTVSGGSISSDGYYWSWIRQHPGKGLEWIGYMYYSGITYHNPSLKSRVTISVDTSKNQFSLRLSSVTAADTAVYYCARDFGWYFDLWGRGTLVTVSS

SEQ ID NO: 358 - protein, 107 aa, synthetic construct
  REGION 1..107: note = Description of sequence: Light chain-12D6
DIQMTQSPSSLSASVGDRVTITCRASQDISNYLAWYQQKPGKVPKLLIYAASTLHSGVPSRFSGSGSGTDFTLTISSLQPDDFAAYYCQKYNSAPRTFGQGTKVEIK

SEQ ID NO: 359 - protein, 118 aa, synthetic construct
  REGION 1..118: note = Description of sequence: Heavy chain-12D6
QVQLQESGPGLVKPSQTLSLTCTVSGGSISSDAYYWSWIRQHPGKGLEWIGYMYYSGITYYNPSLKSRVTISVDTSKNQFSLKLSSVTAADTAVYYCARDFGWYFDLWGRGTLVTVSS

SEQ ID NO: 360 - protein, 107 aa, synthetic construct
  REGION 1..107: note = Description of sequence: Light chain-12D7
DIQLTQSPSFLSASVGDRVSITCRASQDISSFLAWYQQKPGKAPVLLIYVASTLQSGVPSRFSGSGSGTEFTLTVSSLQPEDFATYYCQQLHVYPITFGQGTRLEIR

SEQ ID NO: 361 - protein, 120 aa, synthetic construct; identical to SEQ ID NO: 347
  REGION 1..120: note = Description of sequence: Heavy chain-12D7

SEQ ID NO: 362 - protein, 113 aa, synthetic construct
  REGION 1..113: note = Description of sequence: Light chain-12F5
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPFTFGPGTKVDIK

SEQ ID NO: 363 - protein, 122 aa, synthetic construct
  REGION 1..122: note = Description of sequence: Heavy chain-12F5
EVQLVESGGGLVKPGGSLRLSCAASGFTFSNAWMSWVRQAPGKGLEWVGRIKSKTGGGTTDYAAPVKGRFTISRDDSKNTLYLQMNSLKTEDTAVYYCTSLIVGAISLFDYWGQGTLVTVSS

SEQ ID NO: 364 - protein, 107 aa, synthetic construct
  REGION 1..107: note = Description of sequence: Light chain-12H4
DIQMTQSPSALSASVGDRVAITCRASQTISTWLAWYQQKPGKAPKVLIYKASNLESGVPSRFSGSGSGTEFTLTINSLQPDDFATYYCQQYQTFSHTFGQGTKLEIK

SEQ ID NO: 365 - protein, 118 aa, synthetic construct
  REGION 1..118: note = Description of sequence: Heavy chain-12H4
QVQLRESGPGLVKPSETLSLTCTISGGSISYYFWTWIRQPPGRGLEWIGQIYYSGNTNSNPSLKSRVTISIDTSKNQFSLKLTSVTVADTAVYYCVRAEGSLDAFDIWGQGTMVAVSS

SEQ ID NO: 366 - protein, 107 aa, synthetic construct
  REGION 1..107: note = Description of sequence: Light chain-8C8
DMQMTQSPSSLSASVGDRVTLTCRASQGISNYLAWFQLKPGKVPKLLIYAASTLQSGVPSRFSGSGSGTDFALTISSLQPEDVATYYCQKYNSAPLTFGGGTKVEIK

SEQ ID NO: 367 - protein, 122 aa, synthetic construct
  REGION 1..122: note = Description of sequence: Heavy chain-8C8
EVQLVESGGGLVKPGGSLRLSCVASGFTFSSYSMNWVRQFPGKGLEWVSSISTSSNYIHYADSLQGRFTISRDNAKNSLYLQMSSLRVEDTAVYYCVRDKGTTLTNWYFDLWGRGTLVTVSS

SEQ ID NO: 368 - protein, 112 aa, synthetic construct
  REGION 1..112: note = Description of sequence: Light chain-8F7
DIVMTQSPLSLPVTPGEPASISCRSSQTLVHSNGYNYLNWYLQKPGQSPQLLIYLGSNRASGVPDRFSGSGSGSDFTLKISRMEAEDVGVYYCMQAIQTPYTFGQGTNVEIK

SEQ ID NO: 369 - protein, 121 aa, synthetic construct
  REGION 1..121: note = Description of sequence: Heavy chain-8F7
QVQLVESGGGVVQPGRSLRLSCGASGFTFSSYGMHWVRQAPGKGLEWVAVIWYDGSNKYYADSLKGRFTISRDNSKNTLYLQMNSLRAEDTAVYYCARDGYSGSSDAFDIWGQGTMVTVSS

SEQ ID NO: 370 - protein, 107 aa, synthetic construct
  REGION 1..107: note = Description of sequence: Light chain-8F8
DIQMTQSPSTLSASVGDRVTITCRASQSISSWLAWYQQKPGKAPKVLIYKASNLESGVPSRFSGSGSGTEFTLTISSLQPDDFATYYCQQYNSYSCTFGQGTKLEIK

SEQ ID NO: 371 - protein, 118 aa, synthetic construct
  REGION 1..118: note = Description of sequence: Heavy chain-8F8
QVQLQESGPGLVQPSETLSLTCTVSGGSISYYYWSWIRQPPGKGLEWIGNINYMGNTIYNPSLKSRVTISVDTSKDQFSLKLTSVSAADTAVYYCVRAEGSLDAFDFWGQGTLVAVSL

SEQ ID NO: 372 - protein, 107 aa, synthetic construct
  REGION 1..107: note = Description of sequence: Light chain-9D8
DIQMTQSPSSLSASVGDRIIFTCQASQDINNYLHWYQQKPGKAPKLLIYDASDWETGVPSRFSGSGSGTDFTFTISSLQPEDIATYYCQQYDHLPITFGQGTRVEIK

SEQ ID NO: 373 - protein, 123 aa, synthetic construct
  REGION 1..123: note = Description of sequence: Heavy chain-9D8
QVQLVQSGAEVTKPGASVKVSCKASGYIFTGYYIYWVRQAPGQGLEWMGWINPSSGGTNYAQKFQGRVTMARDTSISTAYMELSSLRSDDTAVYYCARDRKREYYYNFGMDVWGQGTTVTVST

SEQ ID NO: 374 - protein, 107 aa, synthetic construct
  REGION 1..107: note = Description of sequence: Light chain-9E10
DIQMTQSPSSLSASVGDRVILTCQASQDISNYLHWYQQKPGKAPKLLIYDASDLETGVPSRFSGSGSGADFTFTISNLQPEDFATYYCQQYDHLPITFGQGTRLEIK

SEQ ID NO: 375 - protein, 123 aa, synthetic construct
  REGION 1..123: note = Description of sequence: Heavy chain-9E10
QVQLVQSGAEVTKPGASVKVSCKASGYTFTSHYIYWVRQAPGQGLEWMGWINPNSGGTNYAQKFQDRVTMARDTSISTAYMELSRLRSDDTAVYYCAKDRKREYYYNFGMDVWGQGTTVTVSA

SEQ ID NO: 376 - protein, 107 aa, synthetic construct; identical to SEQ ID NO: 374
  REGION 1..107: note = Description of sequence: Light chain-9E5

SEQ ID NO: 377 - protein, 123 aa, synthetic construct
  REGION 1..123: note = Description of sequence: Heavy chain-9E5
QVQLVQFGVEVRKPGASVKVSCKVSGFTFTSHYIYWVRQAPGQGLEWMGWINPNSGGTKYAQKFQDRVTMARDTSISTAYMELSRLRSDDTSVYYCVKDRKREYYYNFGMDVWGQGTTVTVSS

SEQ ID NO: 378 - protein, 107 aa, synthetic construct
  REGION 1..107: note = Description of sequence: Light chain-9F4
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 379 - protein, 119 aa, synthetic construct
  REGION 1..119: note = Description of sequence: Heavy chain-9F4
EVQMLESGGGLIQPGGSLRLSCKTSGFTLSIYAIHWVRQAPGRGLEWVSSFGGRGSSTYFADSVKGRFTISRDASENSLYLHMNSLRAEDTAVYYCAKEKDWGRGFDYWGQGTLVTVSS

SEQ ID NO: 380 - protein, 112 aa, synthetic construct
  REGION 1..112: note = Description of sequence: Light chain-9F8
DIVMTQSPLSLPVTPGEPASISCRSSQSLLYSNGYNYLDWYLQKPGQSPQLLIFLNSNRASGVPDRFSGSGSGTDFTLKISRVEAEDVGVYFCMQALQTPLTFGGGTKVEIK

SEQ ID NO: 381 - protein, 122 aa, synthetic construct
  REGION 1..122: note = Description of sequence: Heavy chain-9F8
EVQLVESGGGLVKPGGSLRLSCAASGFTFSNYSMNWVRQAPGKGLEWVSSISSSTIYIYYADSVKGRFTISRDNAKKSLYLQMNSLRAEDTAVYYCARDIGWEVFTLGFDYWGQGTQVTVSS

SEQ ID NO: 382 - protein, 5 aa, synthetic construct
  REGION 1..5: note = Description of sequence: Heavy chain-CDRH1:10A1 Kabat
YYYWT

SEQ ID NO: 383 - protein, 7 aa, synthetic construct
  REGION 1..7: note = Description of sequence: Heavy chain-CDRH1:10A1 Chothia
GGSISYY

SEQ ID NO: 384 - protein, 10 aa, synthetic construct
  REGION 1..10: note = Description of sequence: Heavy chain-CDRH1:10A1 Extended
GGSISYYYWT

SEQ ID NO: 385 - protein, 16 aa, synthetic construct
  REGION 1..16: note = Description of sequence: Heavy chain-CDRH2:10A1 Kabat
HIYYSGSTNYNPSLKS

SEQ ID NO: 386 - protein, 5 aa, synthetic construct
  REGION 1..5: note = Description of sequence: Heavy chain-CDRH2:10A1 Chothia
YYSGS

SEQ ID NO: 387 - protein, 10 aa, synthetic construct
  REGION 1..10: note = Description of sequence: Heavy chain-CDRH3:10A1
AEGSLDAFDF

SEQ ID NO: 388 - protein, 5 aa, synthetic construct
  REGION 1..5: note = Description of sequence: Heavy chain-CDRH1:1000 kabat
SNYMT

SEQ ID NO: 389 - protein, 7 aa, synthetic construct
  REGION 1..7: note = Description of sequence: Heavy chain-CDRH1:1000 Chothia
GFTVSSN

SEQ ID NO: 390 - protein, 10 aa, synthetic construct
  REGION 1..10: note = Description of sequence: Heavy chain-CDRH1:1000 Extended
GFTVSSNYMT

SEQ ID NO: 391 - protein, 16 aa, synthetic construct
  REGION 1..16: note = Description of sequence: Heavy chain-CDRH2:10E2 Kabat
VIYSGGSTYYADSVKG

SEQ ID NO: 392 - protein, 5 aa, synthetic construct
  REGION 1..5: note = Description of sequence: Heavy chain-CDRH2:10E2 Chothia
YSGGS

SEQ ID NO: 393 - protein, 6 aa, synthetic construct
  REGION 1..6: note = Description of sequence: Heavy chain-CDRH3:10E2
NWGDYW

SEQ ID NO: 394 - protein, 5 aa, synthetic construct
  REGION 1..5: note = Description of sequence: Heavy chain-CDRH1:11A1 Kabat
YYFWN

SEQ ID NO: 395 - protein, 7 aa, synthetic construct
  REGION 1..7: note = Description of sequence: Heavy chain-CDRH1:11A1 Chothia
GGSIDYY

SEQ ID NO: 396 - protein, 10 aa, synthetic construct
  REGION 1..10: note = Description of sequence: Heavy chain-CDRH1:11A1 Extended
GGSIDYYFWN

SEQ ID NO: 397 - protein, 16 aa, synthetic construct
  REGION 1..16: note = Description of sequence: Heavy chain-CDRH2:11A1 Kabat
HVYDIGNTKYNPSLKS

SEQ ID NO: 398 - protein, 5 aa, synthetic construct
  REGION 1..5: note = Description of sequence: Heavy chain-CDRH2:11A1 Chothia
YDIGN

SEQ ID NO: 399 - protein, 10 aa, synthetic construct
  REGION 1..10: note = Description of sequence: Heavy chain-CDRH3:11A1
GEGALDAFDI

SEQ ID NO: 400 - protein, 5 aa, synthetic construct
  REGION 1..5: note = Description of sequence: Heavy chain-CDRH1:11C1 Kabat
YYYWT

SEQ ID NO: 401 - protein, 7 aa, synthetic construct
  REGION 1..7: note = Description of sequence: Heavy chain-CDRH1:11C1 Chothia
GGSISYY

SEQ ID NO: 402 - protein, 10 aa, synthetic construct
  REGION 1..10: note = Description of sequence: Heavy chain-CDRH1:11C1 Extended
GGSISYYYWT

SEQ ID NO: 403 - protein, 16 aa, synthetic construct
  REGION 1..16: note = Description of sequence: Heavy chain-CDRH2:11C1 Kabat
HVIYSGTTNYNPSLKS

SEQ ID NO: 404 - protein, 5 aa, synthetic construct
  REGION 1..5: note = Description of sequence: Heavy chain-CDRH2:11C1 Chothia
IYSGT

SEQ ID NO: 405 - protein, 10 aa, synthetic construct
  REGION 1..10: note = Description of sequence: Heavy chain-CDRH3:11C1
AEGSLDAFDL

SEQ ID NO: 406 - protein, 5 aa, synthetic construct
  REGION 1..5: note = Description of sequence: Heavy chain-CDRH1:11D1 Kabat
DYGIH

SEQ ID NO: 407 - protein, 7 aa, synthetic construct
  REGION 1..7: note = Description of sequence: Heavy chain-CDRH1:11D1 Chothia
GFTFSDY

SEQ ID NO: 408 - protein, 10 aa, synthetic construct
  REGION 1..10: note = Description of sequence: Heavy chain-CDRH1:11D1 Extended
GFTFSDYGIH

SEQ ID NO: 409 - protein, 17 aa, synthetic construct
  REGION 1..17: note = Description of sequence: Heavy chain-CDRH2:11D1 Kabat
VIWYDGSIKKYSDSVKG

SEQ ID NO: 410 - protein, 6 aa, synthetic construct
  REGION 1..6: note = Description of sequence: Heavy chain-CDRH2:11D1 Chothia
WYDGSI

SEQ ID NO: 411 - protein, 11 aa, synthetic construct
  REGION 1..11: note = Description of sequence: Heavy chain-CDRH3:11D1
DEVGTFGAFDF

SEQ ID NO: 412 - protein, 7 aa, synthetic construct
  REGION 1..7: note = Description of sequence: Heavy chain-CDRH1:110 kabat
SDGYYWS

SEQ ID NO: 413 - protein, 9 aa, synthetic construct
  REGION 1..9: note = Description of sequence: Heavy chain-CDRH1:110 Chothia
GGSISSDGY

SEQ ID NO: 414 - protein, 12 aa, synthetic construct
  REGION 1..12: note = Description of sequence: Heavy chain-CDRH1:110 Extended
GGSISSDGYYWS

SEQ ID NO: 415 - protein, 16 aa, synthetic construct
  REGION 1..16: note = Description of sequence: Heavy chain-CDRH2:11E1 Kabat
YMYYSGSTYYNPSLKS

SEQ ID NO: 416 - protein, 5 aa, synthetic construct
  REGION 1..5: note = Description of sequence: Heavy chain-CDRH2:11E1 Chothia
YYSGS

SEQ ID NO: 417 - protein, 8 aa, synthetic construct
  REGION 1..8: note = Description of sequence: Heavy chain-CDRH3:11E1
DFGWYFDL

SEQ ID NO: 418 - protein, 7 aa, synthetic construct
  REGION 1..7: note = Description of sequence: Heavy chain-CDRH1:12A2 Kabat
SDGYYWS

SEQ ID NO: 419 - protein, 9 aa, synthetic construct
  REGION 1..9: note = Description of sequence: Heavy chain-CDRH1:12A2 Chothia
GGSVSSDGY

SEQ ID NO: 420 - protein, 12 aa, synthetic construct
  REGION 1..12: note = Description of sequence: Heavy chain-CDRH1:12A2 Extended
GGSVSSDGYYWS

SEQ ID NO: 421 - protein, 16 aa, synthetic construct
  REGION 1..16: note = Description of sequence: Heavy chain-CDRH2:12A2 Kabat
YIYYRRITDYNPSLKS

SEQ ID NO: 422 - protein, 5 aa, synthetic construct
  REGION 1..5: note = Description of sequence: Heavy chain-CDRH2:12A2 Chothia
YYRRI

SEQ ID NO: 423 - protein, 8 aa, synthetic construct
  REGION 1..8: note = Description of sequence: Heavy chain-CDRH3:12A2
DFGWYFDL

SEQ ID NO: 424 - protein, 5 aa, synthetic construct
  REGION 1..5: note = Description of sequence: Heavy chain-CDRH1:12C4 Kabat
GYYLH

SEQ ID NO: 425 - protein, 7 aa, synthetic construct
  REGION 1..7: note = Description of sequence: Heavy chain-CDRH1:12C4 Chothia
GYTFTGY

SEQ ID NO: 426 - protein, 10 aa, synthetic construct
  REGION 1..10: note = Description of sequence: Heavy chain-CDRH1:12C4 Extended
GYTFTGYYLH

SEQ ID NO: 427 - protein, 17 aa, synthetic construct
  REGION 1..17: note = Description of sequence: Heavy chain-CDRH2:12C4 Kabat
WINPNSGGTNYAQKFQG

SEQ ID NO: 428 - protein, 6 aa, synthetic construct
  REGION 1..6: note = Description of sequence: Heavy chain-CDRH2:12C4 Chothia
NPNSGG

SEQ ID NO: 429 - protein, 13 aa, synthetic construct
  REGION 1..13: note = Description of sequence: Heavy chain-CDRH3:12C4
DRGVTMIVDGMDD

SEQ ID NO: 430 - protein, 5 aa, synthetic construct
  REGION 1..5: note = Description of sequence: Heavy chain-CDRH1:12C5 Kabat
DYGMH

SEQ ID NO: 431 - protein, 7 aa, synthetic construct
  REGION 1..7: note = Description of sequence: Heavy chain-CDRH1:12C5 Chothia
GFTFSDY

SEQ ID NO: 432 - protein, 10 aa, synthetic construct
  REGION 1..10: note = Description of sequence: Heavy chain-CDRH1:12C5 Extended
GFTFSDYGMH

SEQ ID NO: 433 - protein, 17 aa, synthetic construct
  REGION 1..17: note = Description of sequence: Heavy chain-CDRH2:12C5 Kabat
VIWYDGSNKYYADSVKG

SEQ ID NO: 434 - protein, 6 aa, synthetic construct
  REGION 1..6: note = Description of sequence: Heavy chain-CDRH2:12C5 Chothia
WYDGSN

SEQ ID NO: 435 - protein, 11 aa, synthetic construct
  REGION 1..11: note = Description of sequence: Heavy chain-CDRH3:12C5
DEVGFVGAFDI

SEQ ID NO: 436 - protein, 7 aa, synthetic construct
  REGION 1..7: note = Description of sequence: Heavy chain-CDRH1:12D3 Kabat
SDGYYWS

SEQ ID NO: 437 - protein, 9 aa, synthetic construct
  REGION 1..9: note = Description of sequence: Heavy chain-CDRH1:12D3 Chothia
GGSISSDGY

SEQ ID NO: 438 - protein, 12 aa, synthetic construct
  REGION 1..12: note = Description of sequence: Heavy chain-CDRH1:12D3 Extended
GGSISSDGYYWS

SEQ ID NO: 439 - protein, 16 aa, synthetic construct
  REGION 1..16: note = Description of sequence: Heavy chain-CDRH2:12D3 Kabat
YMYYSGITYHNPSLKS

SEQ ID NO: 440 - protein, 5 aa, synthetic construct
  REGION 1..5: note = Description of sequence: Heavy chain-CDRH2:12D3 Chothia
YYSGI

SEQ ID NO: 441 - protein, 8 aa, synthetic construct
  REGION 1..8: note = Description of sequence: Heavy chain-CDRH3:12D3
DFGWYFDL

SEQ ID NO: 442 - protein, 7 aa, synthetic construct
  REGION 1..7: note = Description of sequence: Heavy chain-CDRH1:12D6 Kabat
SDAYYWS

SEQ ID NO: 443 - protein, 9 aa, synthetic construct
  REGION 1..9: note = Description of sequence: Heavy chain-CDRH1:12D6 Chothia
GGSISSDAY

SEQ ID NO: 444 - protein, 12 aa, synthetic construct
  REGION 1..12: note = Description of sequence: Heavy chain-CDRH1:12D6 Extended
GGSISSDAYYWS

SEQ ID NO: 445 - protein, 16 aa, synthetic construct
  REGION 1..16: note = Description of sequence: Heavy chain-CDRH2:12D6 Kabat
YMYYSGITYYNPSLKS

SEQ ID NO: 446 - protein, 5 aa, synthetic construct
  REGION 1..5: note = Description of sequence: Heavy chain-CDRH2:12D6 Chothia
YYSGI

SEQ ID NO: 447 - protein, 8 aa, synthetic construct
  REGION 1..8: note = Description of sequence: Heavy chain-CDRH3:12D6
DFGWYFDL

SEQ ID NO: 448 - protein, 5 aa, synthetic construct
  REGION 1..5: note = Description of sequence: Heavy chain-CDRH1:12D7 Kabat
DYGIH

SEQ ID NO: 449 - protein, 7 aa, synthetic construct
  REGION 1..7: note = Description of sequence: Heavy chain-CDRH1:12D7 Chothia
GFTFSDY

SEQ ID NO: 450 - protein, 10 aa, synthetic construct
  REGION 1..10: note = Description of sequence: Heavy chain-CDRH1:12D7 Extended
GFTFSDYGIH

SEQ ID NO: 451 - protein, 17 aa, synthetic construct
  REGION 1..17: note = Description of sequence: Heavy chain-CDRH2:12D7 Kabat
VIWYDGSIKKYSDSVKG

SEQ ID NO: 452 - protein, 6 aa, synthetic construct
  REGION 1..6: note = Description of sequence: Heavy chain-CDRH2:12D7 Chothia
WYDGSI

SEQ ID NO: 453 - protein, 11 aa, synthetic construct
  REGION 1..11: note = Description of sequence: Heavy chain-CDRH3:12D7
DEVGTFGAFDF

SEQ ID NO: 454 - protein, 5 aa, synthetic construct
  REGION 1..5: note = Description of sequence: Heavy chain-CDRH1:12F5 Kabat
NAWMS

SEQ ID NO: 455 - protein, 7 aa, synthetic construct
  REGION 1..7: note = Description of sequence: Heavy chain-CDRH1:12F5 Chothia
GFTFSNA

SEQ ID NO: 456 - protein, 10 aa, synthetic construct
  REGION 1..10: note = Description of sequence: Heavy chain-CDRH1:12F5 Extended
GFTFSNAWMS

SEQ ID NO: 457 - protein, 19 aa, synthetic construct
  REGION 1..19: note = Description of sequence: Heavy chain-CDRH2:12F5 Kabat
RIKSKTGGGTTDYAAPVKG

SEQ ID NO: 458 - protein, 8 aa, synthetic construct
  REGION 1..8: note = Description of sequence: Heavy chain-CDRH2:12F5 Chothia
KSKTGGGT

SEQ ID NO: 459 - protein, 11 aa, synthetic construct
  REGION 1..11: note = Description of sequence: Heavy chain-CDRH3:12F5
LIVGAISLFDY

SEQ ID NO: 460 - protein, 5 aa, synthetic construct
  REGION 1..5: note = Description of sequence: Heavy chain-CDRH1:12H4 Kabat
YYFWT

SEQ ID NO: 461 - protein, 7 aa, synthetic construct
  REGION 1..7: note = Description of sequence: Heavy chain-CDRH1:12H4 Chothia
GGSISYY

SEQ ID NO: 462 - protein, 10 aa, synthetic construct
  REGION 1..10: note = Description of sequence: Heavy chain-CDRH1:12H4 Extended
GGSISYYFWT

SEQ ID NO: 463 - protein, 16 aa, synthetic construct
  REGION 1..16: note = Description of sequence: Heavy chain-CDRH2:12H4 Kabat
QIYYSGNTNSNPSLKS

SEQ ID NO: 464 - protein, 5 aa, synthetic construct
  REGION 1..5: note = Description of sequence: Heavy chain-CDRH2:12H4 Chothia
YYSGN

SEQ ID NO: 465 - protein, 10 aa, synthetic construct
  REGION 1..10: note = Description of sequence: Heavy chain-CDRH3:12H4
AEGSLDAFDI

SEQ ID NO: 466 - protein, 5 aa, synthetic construct
  REGION 1..5: note = Description of sequence: Heavy chain-CDRH1:8C8 Kabat
SYSMN

SEQ ID NO: 467 - protein, 7 aa, synthetic construct
  REGION 1..7: note = Description of sequence: Heavy chain-CDRH1:8C8 Chothia
GFTFSSY

SEQ ID NO: 468 - protein, 10 aa, synthetic construct
  REGION 1..10: note = Description of sequence: Heavy chain-CDRH1:8C8 Extended
GFTFSSYSMN

SEQ ID NO: 469 - protein, 17 aa, synthetic construct
  REGION 1..17: note = Description of sequence: Heavy chain-CDRH2:8C8 Kabat
SISTSSNYIHYADSLQG

SEQ ID NO: 470 - protein, 6 aa, synthetic construct
  REGION 1..6: note = Description of sequence: Heavy chain-CDRH2:8C8 Chothia
STSSNY

SEQ ID NO: 471 - protein, 13 aa, synthetic construct
  REGION 1..13: note = Description of sequence: Heavy chain-CDRH3:8C8
DKGTTLTNWYFDL

SEQ ID NO: 472 - protein, 5 aa, synthetic construct
  REGION 1..5: note = Description of sequence: Heavy chain-CDRH1:8F7 Kabat
SYGMH

SEQ ID NO: 473 - protein, 7 aa, synthetic construct
  REGION 1..7: note = Description of sequence: Heavy chain-CDRH1:8F7 Chothia
GFTFSSY

SEQ ID NO: 474 - protein, 10 aa, synthetic construct
  REGION 1..10: note = Description of sequence: Heavy chain-CDRH1:8F7 Extended
GFTFSSYGMH

SEQ ID NO: 475 - protein, 17 aa, synthetic construct
  REGION 1..17: note = Description of sequence: Heavy chain-CDRH2:8F7 Kabat
VIWYDGSNKYYADSLKG

SEQ ID NO: 476 - protein, 6 aa, synthetic construct
  REGION 1..6: note = Description of sequence: Heavy chain-CDRH2:8F7 Chothia
WYDGSN

SEQ ID NO: 477 - protein, 12 aa, synthetic construct
  REGION 1..12: note = Description of sequence: Heavy chain-CDRH3:8F7
DGYSGSSDAFDI

SEQ ID NO: 478 - protein, 5 aa, synthetic construct
  REGION 1..5: note = Description of sequence: Heavy chain-CDRH1:8F8 Kabat
YYYWS

SEQ ID NO: 479 - protein, 7 aa, synthetic construct
  REGION 1..7: note = Description of sequence: Heavy chain-CDRH1:8F8 Chothia
GGSISYY

SEQ ID NO: 480 - protein, 10 aa, synthetic construct
  REGION 1..10: note = Description of sequence: Heavy chain-CDRH1:8F8 Extended
GGSISYYYWS

SEQ ID NO: 481 - protein, 16 aa, synthetic construct
  REGION 1..16: note = Description of sequence: Heavy chain-CDRH2:8F8 Kabat
NINYMGNTIYNPSLKS

SEQ ID NO: 482 - protein, 5 aa, synthetic construct
  REGION 1..5: note = Description of sequence: Heavy chain-CDRH2:8F8 Chothia
NYMGN

SEQ ID NO: 483 - protein, 10 aa, synthetic construct
  REGION 1..10: note = Description of sequence: Heavy chain-CDRH3:8F8
AEGSLDAFDF

SEQ ID NO: 484 - protein, 5 aa, synthetic construct
  REGION 1..5: note = Description of sequence: Heavy chain-CDRH1:9D8 Kabat
GYYIY

SEQ ID NO: 485 - protein, 7 aa, synthetic construct
  REGION 1..7: note = Description of sequence: Heavy chain-CDRH1:9D8 Chothia
GYIFTGY

SEQ ID NO: 486 - protein, 10 aa, synthetic construct
  REGION 1..10: note = Description of sequence: Heavy chain-CDRH1:9D8 Extended
GYIFTGYYIY

SEQ ID NO: 487 - protein, 17 aa, synthetic construct
  REGION 1..17: note = Description of sequence: Heavy chain-CDRH2:9D8 Kabat
WINPSSGGTNYAQKFQG

SEQ ID NO: 488 - protein, 6 aa, synthetic construct
  REGION 1..6: note = Description of sequence: Heavy chain-CDRH2:9D8 Chothia
NPSSGG

SEQ ID NO: 489 - protein, 14 aa, synthetic construct
  REGION 1..14: note = Description of sequence: Heavy chain-CDRH3:9D8
DRKREYYYNFGMDV

SEQ ID NO: 490 - protein, 5 aa, synthetic construct
  REGION 1..5: note = Description of sequence: Heavy chain-CDRH1:90000000000 kabat
SHYIY

SEQ ID NO: 491 - protein, 7 aa, synthetic construct
  REGION 1..7: note = Description of sequence: Heavy chain-CDRH1:90000000000 Chothia
GYTFTSH

SEQ ID NO: 492 - protein, 10 aa, synthetic construct
  REGION 1..10: note = Description of sequence: Heavy chain-CDRH1:90000000000 Extended
GYTFTSHYIY

SEQ ID NO: 493 - protein, 17 aa, synthetic construct
  REGION 1..17: note = Description of sequence: Heavy chain-CDRH2:9E10 Kabat
WINPNSGGTNYAQKFQD

SEQ ID NO: 494 - protein, 6 aa, synthetic construct
  REGION 1..6: note = Description of sequence: Heavy chain-CDRH2:9E10 Chothia
NPNSGG

SEQ ID NO: 495 - protein, 14 aa, synthetic construct
  REGION 1..14: note = Description of sequence: Heavy chain-CDRH3:9E10
DRKREYYYNFGMDV

SEQ ID NO: 496 - protein, 5 aa, synthetic construct
  REGION 1..5: note = Description of sequence: Heavy chain-CDRH1:900000 kabat
SHYIY

SEQ ID NO: 497 - protein, 7 aa, synthetic construct
  REGION 1..7: note = Description of sequence: Heavy chain-CDRH1:900000 Chothia
GFTFTSH

SEQ ID NO: 498 - protein, 10 aa, synthetic construct
  REGION 1..10: note = Description of sequence: Heavy chain-CDRH1:900000 Extended
GFTFTSHYIY

SEQ ID NO: 499 - protein, 17 aa, synthetic construct
  REGION 1..17: note = Description of sequence: Heavy chain-CDRH2:9E5 Kabat
WINPNSGGTKYAQKFQD

SEQ ID NO: 500 - protein, 6 aa, synthetic construct
  REGION 1..6: note = Description of sequence: Heavy chain-CDRH2:9E5 Chothia
NPNSGG

SEQ ID NO: 501 - protein, 14 aa, synthetic construct
  REGION 1..14: note = Description of sequence: Heavy chain-CDRH3:9E5
DRKREYYYNFGMDV

SEQ ID NO: 502 - protein, 5 aa, synthetic construct
  REGION 1..5: note = Description of sequence: Heavy chain-CDRH1:9F4 Kabat
IYAIH

SEQ ID NO: 503 - protein, 7 aa, synthetic construct
  REGION 1..7: note = Description of sequence: Heavy chain-CDRH1:9F4 Chothia
GFTLSIY

SEQ ID NO: 504 - protein, 10 aa, synthetic construct
  REGION 1..10: note = Description of sequence: Heavy chain-CDRH1:9F4 Extended
GFTLSIYAIH

SEQ ID NO: 505 - protein, 17 aa, synthetic construct
  REGION 1..17: note = Description of sequence: Heavy chain-CDRH2:9F4 Kabat
SFGGRGSSTYFADSVKG

SEQ ID NO: 506 - protein, 6 aa, synthetic construct
  REGION 1..6: note = Description of sequence: Heavy chain-CDRH2:9F4 Chothia
GGRGSS

SEQ ID NO: 507 - protein, 10 aa, synthetic construct
  REGION 1..10: note = Description of sequence: Heavy chain-CDRH3:9F4
EKDWGRGFDY

SEQ ID NO: 508 - protein, 5 aa, synthetic construct
  REGION 1..5: note = Description of sequence: Heavy chain-CDRH1:9F8 Kabat
NYSMN

SEQ ID NO: 509 - protein, 7 aa, synthetic construct
  REGION 1..7: note = Description of sequence: Heavy chain-CDRH1:9F8 Chothia
GFTFSNY

SEQ ID NO: 510 - protein, 10 aa, synthetic construct
  REGION 1..10: note = Description of sequence: Heavy chain-CDRH1:9F8 Extended
GFTFSNYSMN

SEQ ID NO: 511 - protein, 17 aa, synthetic construct
  REGION 1..17: note = Description of sequence: Heavy chain-CDRH2:9F8 Kabat
SISSSTIYIYYADSVKG

SEQ ID NO: 512 - protein, 6 aa, synthetic construct
  REGION 1..6: note = Description of sequence: Heavy chain-CDRH2:9F8 Chothia
SSSTIY

SEQ ID NO: 513 - protein, 13 aa, synthetic construct
  REGION 1..13: note = Description of sequence: Heavy chain-CDRH3:9F8
DIGWEVFTLGFDY

SEQ ID NO: 514 - protein, 11 aa, synthetic construct
  REGION 1..11: note = Description of sequence: Light Chain:10A1 CDRL1
RASQSISTWLA

SEQ ID NO: 515 - protein, 7 aa, synthetic construct
  REGION 1..7: note = Description of sequence: Light Chain:10A1 CDRL2
KASSLES

SEQ ID NO: 516 - protein, 9 aa, synthetic construct
  REGION 1..9: note = Description of sequence: Light Chain:10A1 CDRL3
QQYKSYSHT

SEQ ID NO: 517 - protein, 11 aa, synthetic construct
  REGION 1..11: note = Description of sequence: Light Chain:10E2 CDRL1
RASQSISSWLA

SEQ ID NO: 518 - protein, 7 aa, synthetic construct
  REGION 1..7: note = Description of sequence: Light Chain:10E2 CDRL2
KASSLES

SEQ ID NO: 519 - protein, 9 aa, synthetic construct
  REGION 1..9: note = Description of sequence: Light Chain:10E2 CDRL3
QQYKSFSLT

SEQ ID NO: 520 - protein, 11 aa, synthetic construct
  REGION 1..11: note = Description of sequence: Light Chain:11A1 CDRL1
RASQSISSWLA

SEQ ID NO: 521 - protein, 7 aa, synthetic construct
  REGION 1..7: note = Description of sequence: Light Chain:11A1 CDRL2
KASTLES

SEQ ID NO: 522 - protein, 9 aa, synthetic construct
  REGION 1..9: note = Description of sequence: Light Chain:11A1 CDRL3
QQYNSYSYT

SEQ ID NO: 523 - protein, 11 aa, synthetic construct
  REGION 1..11: note = Description of sequence: Light Chain:11C1 CDRL1
RASQSVSSWLA

SEQ ID NO: 524 - protein, 7 aa, synthetic construct
  REGION 1..7: note = Description of sequence: Light Chain:11C1 CDRL2
KASSLES

SEQ ID NO: 525 - protein, 9 aa, synthetic construct
  REGION 1..9: note = Description of sequence: Light Chain:11C1 CDRL3
QQYNTYSHT

SEQ ID NO: 526 - protein, 11 aa, synthetic construct
  REGION 1..11: note = Description of sequence: Light Chain:11D1 CDRL1
RASQGIRNDLG

SEQ ID NO: 527 - protein, 7 aa, synthetic construct
  REGION 1..7: note = Description of sequence: Light Chain:11D1 CDRL2
AASSLQS

SEQ ID NO: 528 - protein, 9 aa, synthetic construct
  REGION 1..9: note = Description of sequence: Light Chain:11D1 CDRL3
LQDYNYPFT

SEQ ID NO: 529 - protein, 11 aa, synthetic construct
  REGION 1..11: note = Description of sequence: Light Chain:11E1 CDRL1
RASQDIDNYLA

SEQ ID NO: 530 - protein, 7 aa, synthetic construct
  REGION 1..7: note = Description of sequence: Light Chain:11E1 CDRL2
AASALQS

SEQ ID NO: 531 - protein, 9 aa, synthetic construct
  REGION 1..9: note = Description of sequence: Light Chain:11E1 CDRL3
QNYNSGPRT

SEQ ID NO: 532 - protein, 11 aa, synthetic construct
  REGION 1..11: note = Description of sequence: Light Chain:12A2 CDRL1
RASQDISNYLT

SEQ ID NO: 533 - protein, 7 aa, synthetic construct
  REGION 1..7: note = Description of sequence: Light Chain:12A2 CDRL2
AASALQS

SEQ ID NO: 534 - protein, 9 aa, synthetic construct
  REGION 1..9: note = Description of sequence: Light Chain:12A2 CDRL3
QNYNSAPRT

SEQ ID NO: 535 - protein, 16 aa, synthetic construct
  REGION 1..16: note = Description of sequence: Light Chain:12C4 CDRL1
RSSQSLLHSNGYNYLD

SEQ ID NO: 536 - protein, 7 aa, synthetic construct
  REGION 1..7: note = Description of sequence: Light Chain:12C4 CDRL2
LGSNRAS

SEQ ID NO: 537 - protein, 9 aa, synthetic construct
  REGION 1..9: note = Description of sequence: Light Chain:12C4 CDRL3
MQTLQTPFT

SEQ ID NO: 538 - protein, 11 aa, synthetic construct
  REGION 1..11: note = Description of sequence: Light Chain:12C5 CDRL1
RASQGINSHLA

SEQ ID NO: 539 - protein, 7 aa, synthetic construct
  REGION 1..7: note = Description of sequence: Light Chain:12C5 CDRL2
YASTLPS

SEQ ID NO: 540 - protein, 9 aa, synthetic construct
  REGION 1..9: note = Description of sequence: Light Chain:12C5 CDRL3
QQLNHYPIT

SEQ ID NO: 541 - protein, 11 aa, synthetic construct
  REGION 1..11: note = Description of sequence: Light Chain:12D3 CDRL1
RASQGISNYLA

SEQ ID NO: 542 - protein, 7 aa, synthetic construct
  REGION 1..7: note = Description of sequence: Light Chain:12D3 CDRL2
AASTLHS

SEQ ID NO: 543 - protein, 9 aa, synthetic construct
  REGION 1..9: note = Description of sequence: Light Chain:12D3 CDRL3
QKYNSAPRT

SEQ ID NO: 544 - protein, 11 aa, synthetic construct
  REGION 1..11: note = Description of sequence: Light Chain:12D6 CDRL1
RASQDISNYLA

SEQ ID NO: 545 - protein, 7 aa, synthetic construct
  REGION 1..7: note = Description of sequence: Light Chain:12D6 CDRL2
AASTLHS

SEQ ID NO: 546 - protein, 9 aa, synthetic construct
  REGION 1..9: note = Description of sequence: Light Chain:12D6 CDRL3
QKYNSAPRT

SEQ ID NO: 547 - protein, 11 aa, synthetic construct
  REGION 1..11: note = Description of sequence: Light Chain:12D7 CDRL1
RASQDISSFLA

SEQ ID NO: 548 - protein, 7 aa, synthetic construct
  REGION 1..7: note = Description of sequence: Light Chain:12D7 CDRL2
VASTLQS

SEQ ID NO: 549 - protein, 9 aa, synthetic construct
  REGION 1..9: note = Description of sequence: Light Chain:12D7 CDRL3
QQLHVYPIT

SEQ ID NO: 550 - protein, 17 aa, synthetic construct
  REGION 1..17: note = Description of sequence: Light Chain:12F5 CDRL1
RSSQSLLDSDDGNTYLD

SEQ ID NO: 551 - protein, 7 aa, synthetic construct
  REGION 1..7: note = Description of sequence: Light Chain:12F5 CDRL2
TLSYRAS

SEQ ID NO: 552 - protein, 9 aa, synthetic construct
  REGION 1..9: note = Description of sequence: Light Chain:12F5 CDRL3
MQRIEFPFT

SEQ ID NO: 553 - protein, 11 aa, synthetic construct
  REGION 1..11: note = Description of sequence: Light Chain:12H4 CDRL1
RASQTISTWLA

SEQ ID NO: 554 - protein, 7 aa, synthetic construct
  REGION 1..7: note = Description of sequence: Light Chain:12H4 CDRL2
KASNLES

SEQ ID NO: 555 - protein, 9 aa, synthetic construct
  REGION 1..9: note = Description of sequence: Light Chain:12H4 CDRL3
QQYQTFSHT

SEQ ID NO: 556 - protein, 11 aa, synthetic construct
  REGION 1..11: note = Description of sequence: Light Chain:8C8 CDRL1
RASQGISNYLA

SEQ ID NO: 557 - protein, 7 aa, synthetic construct
  REGION 1..7: note = Description of sequence: Light Chain:8C8 CDRL2
AASTLQS

SEQ ID NO: 558 - protein, 9 aa, synthetic construct
  REGION 1..9: note = Description of sequence: Light Chain:8C8 CDRL3
QKYNSAPLT

SEQ ID NO: 559 - protein, 16 aa, synthetic construct
  REGION 1..16: note = Description of sequence: Light Chain:8F7 CDRL1
RSSQTLVHSNGYNYLN

SEQ ID NO: 560 - protein, 7 aa, synthetic construct
  REGION 1..7: note = Description of sequence: Light Chain:8F7 CDRL2
LGSNRAS

SEQ ID NO: 561 - protein, 9 aa, synthetic construct
  REGION 1..9: note = Description of sequence: Light Chain:8F7 CDRL3
MQAIQTPYT

SEQ ID NO: 562 - protein, 11 aa, synthetic construct
  REGION 1..11: note = Description of sequence: Light Chain:8F8 CDRL1
RASQSISSWLA

SEQ ID NO: 563 - protein, 7 aa, synthetic construct
  REGION 1..7: note = Description of sequence: Light Chain:8F8 CDRL2
KASNLES

SEQ ID NO: 564 - protein, 9 aa, synthetic construct
  REGION 1..9: note = Description of sequence: Light Chain:8F8 CDRL3
QQYNSYSCT

SEQ ID NO: 565 - protein, 11 aa, synthetic construct
  REGION 1..11: note = Description of sequence: Light Chain:9D8 CDRL1
QASQDINNYLH

SEQ ID NO: 566 - protein, 7 aa, synthetic construct
  REGION 1..7: note = Description of sequence: Light Chain:9D8 CDRL2
DASDWET

SEQ ID NO: 567 - protein, 9 aa, synthetic construct
  REGION 1..9: note = Description of sequence: Light Chain:9D8 CDRL3
QQYDHLPIT

SEQ ID NO: 568 - protein, 11 aa, synthetic construct
  REGION 1..11: note = Description of sequence: Light Chain:9E10 CDRL1
QASQDISNYLH

SEQ ID NO: 569 - protein, 7 aa, synthetic construct
  REGION 1..7: note = Description of sequence: Light Chain:9E10 CDRL2
DASDLET

SEQ ID NO: 570 - protein, 9 aa, synthetic construct
  REGION 1..9: note = Description of sequence: Light Chain:9E10 CDRL3
QQYDHLPIT

SEQ ID NO: 571 - protein, 11 aa, synthetic construct
  REGION 1..11: note = Description of sequence: Light Chain:9E5 CDRL1
QASQDISNYLH

SEQ ID NO: 572 - protein, 7 aa, synthetic construct
  REGION 1..7: note = Description of sequence: Light Chain:9E5 CDRL2
DASDLET

SEQ ID NO: 573 - protein, 9 aa, synthetic construct
  REGION 1..9: note = Description of sequence: Light Chain:9E5 CDRL3
QQYDHLPIT

SEQ ID NO: 574 - protein, 11 aa, synthetic construct
  REGION 1..11: note = Description of sequence: Light Chain:9F4 CDRL1
QASQDISNYLN

SEQ ID NO: 575 - protein, 7 aa, synthetic construct
  REGION 1..7: note = Description of sequence: Light Chain:9F4 CDRL2
DASNLET

SEQ ID NO: 576 - protein, 9 aa, synthetic construct
  REGION 1..9: note = Description of sequence: Light Chain:9F4 CDRL3
QQYDNLPYT

SEQ ID NO: 577 - protein, 16 aa, synthetic construct
  REGION 1..16: note = Description of sequence: Light Chain:9F8 CDRL1
RSSQSLLYSNGYNYLD

SEQ ID NO: 578 - protein, 7 aa, synthetic construct
  REGION 1..7: note = Description of sequence: Light Chain:9F8 CDRL2
LNSNRAS

SEQ ID NO: 579 - protein, 9 aa, synthetic construct
  REGION 1..9: note = Description of sequence: Light Chain:9F8 CDRL3
MQALQTPLT

SEQ ID NO: 580 - protein, 41 aa, synthetic construct
  REGION 1..41: note = Description of sequence: Domain: CD28.AYAA intracellular
RSKRSRLLHSDYMNMTPRRPGPTRKHYQAYAAPRDFAAYRS

SEQ ID NO: 581 - protein, 41 aa, synthetic construct
  REGION 1..41: note = Description of sequence: Domain: CD28 variant
RSKRSRLLHSDFMNMTARRAGPTRKHYQPYAPPRDFAAYRS

SEQ ID NO: 582 - protein, 116 aa, synthetic construct
  REGION 1..116: note = Description of sequence: Domain: CD2 intracellular (full);
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 583 - protein, 85 aa, synthetic construct
  REGION 1..85: note = Description of sequence: Domain: CD2 intracellular (truncated)
KRKKQTPQNPATSQHPPPPPGHRSQAPSHRPPPPGHRVQHQPQKRPPAPSGTQVHQQKGPPLPRPRVQPKPPHGAAENSLSPSSN

SEQ ID NO: 584 - protein, 42 aa, synthetic construct
  REGION 1..42: note = Description of sequence: Domain: OX40 intracellular
ALYLLRRDQRLPPDAHKPPGGGSFRTPIQEEQADAHSTLAKI

SEQ ID NO: 585 - protein, 112 aa, synthetic construct
  REGION 1..112: note = Description of sequence: Domain: CD3 zeta variant (1XX)
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 586 - protein, 112 aa, synthetic construct
  REGION 1..112: note = Description of sequence: Domain: CD3 zeta variant (XX3)
RVKFSRSADAPAYQQGQNQLFNELNLGRREEFDVLDKRRGRDPEMGGKPRRKNPQEGLFNELQKDKMAEAFSEIGMKGERRRGKGHDGLYQGLSTATKDTYDALHMQALPPR

SEQ ID NO: 587 - protein, 48 aa, synthetic construct
  REGION 1..48: note = Description of sequence: Domain: CD27 intracellular
QRRKYRSNKGESPVEPAEPCHYSCPREEEGSTIPIQEDYRKPEPACSP

SEQ ID NO: 588 - protein, 112 aa, synthetic construct
  REGION 1..112: note = Description of sequence: Domain: CD3 zeta variant (X2X)
RVKFSRSADAPAYQQGQNQLFNELNLGRREEFDVLDKRRGRDPEMGGKPRRKNPQEGLYNELQKDKMAEAYSEIGMKGERRRGKGHDGLFQGLSTATKDTFDALHMQALPPR

SEQ ID NO: 589 - protein, 48 aa, synthetic construct
  REGION 1..48: note = Description of sequence: Domain: CD3 zeta variant (delta12)
RVKFSRSADERRRGKGHDGLYQGLSTATKDTYDALHMQRGRDPEMGGK

SEQ ID NO: 590 - protein, 48 aa, synthetic construct
  REGION 1..48: note = Description of sequence: Domain: CD3 zeta variant (delta23)
RVKFSRSADAPAYQQGQNQLYNELNLGRREEYDVLDKRRGRDPEMGGK

SEQ ID NO: 591 - protein, 37 aa, synthetic construct
  REGION 1..37: note = Description of sequence: Domain: CD3 zeta variant (delta13)
RVKFSRSADGVYNALQKDKMAEAYSEIRGRDPEMGGK

SEQ ID NO: 592 - protein, 9 aa, synthetic construct
  REGION 1..9: note = Description of sequence: Domain: R- rituximab mimotope
CPYSNPSLC

SEQ ID NO: 593 - protein, 16 aa, synthetic construct
  REGION 1..16: note = Description of sequence: Domain: Q- QBEND-10 epitope
ELPTQGTFSNVSTNVS

SEQ ID NO: 594 - protein, 24 aa, synthetic construct
  REGION 1..24: note = Description of sequence: Domain: Q- QBEND-10 epitope
ELPTQGTFSNVSTNVSPAKPTTTA

SEQ ID NO: 595 - protein, 55 aa, synthetic construct
  REGION 1..55: note = Description of sequence: Domain: QR3
GSGGGGSCPYSNPSLCSGGGGSELPTQGTFSNVSTNVSPAKPTTTACPYSNPSLC

SEQ ID NO: 596 - protein, 62 aa, synthetic construct
  REGION 1..62: note = Description of sequence: Domain: QQ
GSGGGGSELPTQGTFSNVSTNVSPAKPTTTAGSGGGGSELPTQGTFSNVSTNVSPAKPTTTA

SEQ ID NO: 597 - protein, 39 aa, synthetic construct
  REGION 1..39: note = Description of sequence: Domain: CD28 hinge
IEVMYPPPYLDNEKSNGTIIHVKGKHLCPSPLFPGPSKP

SEQ ID NO: 598 - protein, 27 aa, synthetic construct
  REGION 1..27: note = Description of sequence: Domain: CD28 TM
FWVLVVVGGVLACYSLLVTVAFIIFWV

SEQ ID NO: 599 - protein, 250 aa, synthetic construct
  REGION 1..250: note = Description of sequence: Domain: 4F11 scFv
QVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEIN

SEQ ID NO: 600 - protein, 245 aa, synthetic construct
  REGION 1..245: note = Description of sequence: Domain: 8F8 scFv
QVQLQESGPGLVQPSETLSLTCTVSGGSISYYYWSWIRQPPGKGLEWIGNINYMGNTIYNPSLKSRVTISVDTSKDQFSLKLTSVSAADTAVYYCVRAEGSLDAFDFWGQGTLVAVSLGGGGSGGGGSGGGGSGGGGSDIQMTQSPSTLSASVGDRVTITCRASQSISSWLAWYQQKPGKAPKVLIYKASNLESGVPSRFSGSGSGTEFTLTISSLQPDDFATYYCQQYNSYSCTFGQGTKLEIK

SEQ ID NO: 601 - protein, 193 aa, synthetic construct
  REGION 1..193: note = Description of sequence: Domain: CD70
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 602 - protein, 20 aa, synthetic construct
  REGION 1..20: note = Description of sequence: Domain: (GGGGS)4
GGGGSGGGGSGGGGSGGGGS

SEQ ID NO: 603 - protein, 18 aa, synthetic construct
  REGION 1..18: note = Description of sequence: Domain: Witlow linker
GSTSGSGKPGSGEGSTKG

SEQ ID NO: 604 - protein, 5 aa, synthetic construct
  REGION 1..5: note = Description of sequence: Domain: GGGGS linker
GGGGS

SEQ ID NO: 605 - protein, 107 aa, synthetic construct
  REGION 1..107: note = Description of sequence: Light chain-12C6
DIQLTQSPSFLSASVGDRVIITCRASQGINSHLAWYQQKPGKAPKLLIYYASTLPSGVPSRFSGSGSGTEFTLTVTSLQPEDFATYYCQQLNHYPITFGQGTRLEIK

SEQ ID NO: 606 - protein, 120 aa, synthetic construct; identical to SEQ ID NO: 355
  REGION 1..120: note = Description of sequence: Heavy chain-12C6

SEQ ID NO: 607 - protein, 5 aa, synthetic construct
  REGION 1..5: note = Description of sequence: Heavy chain-CDRH1:12C6 Kabat
DYGMH

SEQ ID NO: 608 - protein, 7 aa, synthetic construct
  REGION 1..7: note = Description of sequence: Heavy chain-CDRH1:12C6 Chothia
GFTFSDY

SEQ ID NO: 609 - protein, 10 aa, synthetic construct
  REGION 1..10: note = Description of sequence: Heavy chain-CDRH1:12C6 Extended
GFTFSDYGMH

SEQ ID NO: 610 - protein, 17 aa, synthetic construct
  REGION 1..17: note = Description of sequence: Heavy chain-CDRH2:12C6 Kabat
VIWYDGSNKYYADSVKG

SEQ ID NO: 611 - protein, 6 aa, synthetic construct
  REGION 1..6: note = Description of sequence: Heavy chain-CDRH2:12C6 Chothia
WYDGSN

SEQ ID NO: 612 - protein, 11 aa, synthetic construct
  REGION 1..11: note = Description of sequence: Heavy chain-CDRH3:12C6
DEVGFVGAFDI

SEQ ID NO: 613 - protein, 11 aa, synthetic construct
  REGION 1..11: note = Description of sequence: Light Chain:12C6 CDRL1
RASQGINSHLA

SEQ ID NO: 614 - protein, 7 aa, synthetic construct
  REGION 1..7: note = Description of sequence: Light Chain:12C6 CDRL2
YASTLPS

SEQ ID NO: 615 - protein, 9 aa, synthetic construct
  REGION 1..9: note = Description of sequence: Light Chain:12C6 CDRL3
QQLNHYPIT

SEQ ID NO: 616 - protein, 40 aa, synthetic construct
  REGION 1..40: note = Description of sequence: Domain: 41BB intracellular signaling domain (ISD)
GRKKLLYIFKQPFMRPVQTTQEEDGCSCRFPEEEEGGCEL

SEQ ID NO: 617 - protein, 112 aa, synthetic construct
  REGION 1..112: note = Description of sequence: Domain: CD3-zeta intracellular signaling domain (ISD)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR